SEQUENCE LISTING (125 sequences)

SEQ ID NO: 1 - protein, 762 aa, synthetic construct
  source 1..762: note = Cas3 protein amino acid sequence of I-A-1
LEEQFQLVTGHSPAEHQRECGEALATGKSVILRAPTGSGKSEAVWIPFLRCRGKRLPMRMIHALPMRGLANQLEERMKDYAGPGLRVSAMHGQRPESVLFYADAIFATIDQVVASYACAPLSLSVRHGNIPAGAVASSFLVFDEVHTFEPRLGLQSILVLAERAHQMGMPFVIMSATLPKNFIRSLAERLGAAPIEGGRLKSKEGEPRHVTLRVLPEKLSARTILDYAPKVNRTVVVVNTVQRALGLYEQVRDEFRCPVILAHSRFYDEDRRTKEQQIEALFGKKAAQGRCLLIATQVVEVGLDISCDLLITELAPVDALVQRAGRCARWGGKGDVIVLTELDTKRPYDETLVAVTERALQEHNVDGQELTWEVETALVDTVLDPHFKEWAKPDAAGKVLASLAEAAFTGNSTKAEQAVRETLTVEVALHDTPQALGPAILRLPRCRLHPGVFQQFVRKQRPNVWQVVVDRDPDDDYRTRIEFLSVNGKSRLIPGGHYIVDPQFGCYDAERGLRLGVPGQSAEPFAPGQSRDRLKGELQIELWQDHIREVVKAFERYVLPKERMAFEALSRWLGKTQDELLSVARAVLVLHDLGKLARQWQGKIQAGLEGKLSQGSFLAHRGGSVSGLPPHATVSAWVATPCLRRLAGTDWEQTLAVPALAAIAHHHSVRADITPEFEMTDGCFEVVADCARGVAGLEVKRDDFNTKPPQGSGSCGVGLNFLLPEGYTSYVLLSRWLRLADRIATGGGEETIFQYEKWMGDS

SEQ ID NO: 2 - protein, 231 aa, synthetic construct
  source 1..231: note = Cas5a protein amino acid sequence of I-A-1
AEWLQAEVEFASFYSYRVPDLSPSFALCSPVPSPAAIRLAVVDATIRHTGDVNEGHAVFELMKRARLELQPPSRVAVMKFFIKRLKPEKPTKGKRASVIESTGIREYCLPWGPMVFWIESDQPERIAQSLQWLRRLGTTDSLASCTVGAGTPNFASCIRPANGLTLQTTNFAQRPVFTLHELKPETQFNQVNPFADERPGKPFEKRLYVLPLVREKVGENWVIYHHEPFAA

SEQ ID NO: 3 - protein, 352 aa, synthetic construct
  source 1..352: mol_type = protein note
EYRLIKSGLEMFDTARAYGLAQLLQVLAGGRAAPRILSQGGVFTLTISTKPNPATLKSSDLWRGAFGESNWQKVFLTYKRAWSSQRDKVKRSLESHSADIFGKAETDGLAVVFGGNFALPGPLDPVGFKGLKGLTAGSYSEGQTTVDEFNWALGCLGAAAAQRYKIQKAVGNKWEYYVTLPVPEEVQFGDFHAVRQLVYDKGLSYNGVRNAAAHFSLLLASAIREKAQGNPHFPVRFSNVLYFSLFQSGQQFKPAIGGAVNVGRLIEIALARPEVALEMFKTWDYLFRRGSAQGNEDLAQAITELVMAPSLDTYYRHARIFNRYVVDSTKRVRPEYLYDETALKEVLNYAEQ

SEQ ID NO: 4 - protein, 320 aa, synthetic construct
  source 1..320: note = Cas7 protein amino acid sequence of I-A-1
ADSPVFEVAILGRVVWNLHSLNNEGTVGNVSEPRTVVLADGSKSDGISGEMLKHIHAQNVWLVAEDKSQLCEPCRTLNPQKADKNPAVLGVKTAKAKVAAESMSVAISSCALCDLHGFLVQRPTIARASTVEFGWAVALRDGYHRDIHLHARHAVEGRAETTEGQQEGPAEVSGQMIYHRPTRSGTYAFVSVFQPWRIGLNEVNYEYVEGVDREARNKLAIEAYKATFARTGGAMTSTRLPHVEALEGVVLVSSRNFPVPVTSPLQDDYREKTEKVGQAVEGLEVQRFGALPELYVILNALAKRRLFALQMGGTSKKGKQ

SEQ ID NO: 5 - protein, 342 aa, synthetic construct
  source 1..342: note = Cas6 protein amino acid sequence of I-A-1
GDVLGLHSLRVGLFRFRLVPEQPLEVPALNKGNMLRGGFGHGFRKLCCIPECRDARLCPLAAICPYKAVFEPSPPPGSERLAKNQDIPRPFVFRAPHTNQTRFQKGEAFEFGLVLIGRAVDYLPYFVLSFRELANEGLGLNRAKCALERVEQRRTSANGLGRATGEGRLVYSKDSGVFHSTENEGVDSYVNSRLRELSSPNGDQSRQNVTIRFLTPTFLKANGEVIRRPEFHHLFKRLRDRINALCTFFGDGALDLDFRGVGTRAEKVQSVSARTEWVERCRTSSKTGQRHELSGFMGEATYEGNVEEFLPLLALGELVHVGKHTAWGNGRIELQSGTGVKC

SEQ ID NO: 6 - protein, 144 aa, synthetic construct
  source 1..144: note = Csa5 (Cas11) protein amino acid sequence of I-A-1
LSSDSKLSEVFAEESVKSFGKCLRYALWRDEDYASLIEFENAETPTQFADAVRKFLRRYRSGGFMDQTQRSRASEMRKQNRWDGLKKLLRQYEVGPRPSEGQLERLMQLANDTNGVRLVQSAIISYGLTKREPYKEVEELEKEN

SEQ ID NO: 7 - protein, 762 aa, synthetic construct
  source 1..762: note = Cas3 protein amino acid sequence of I-A-2
VACDTFFAPMTDFNLARHQSECAGALASGKSVILRAPTGSGKSEAVWLPFLSLRGKTLPCRLIHTLPMRALVNQLESRMRTYANGRMRVAAMHGQRPESVLFYADAIFATLDQVVTSYACAPLSLSVRQGNIPAGAVAGSFLVFDEVHTFEPHLGLQSLLVLAERAHQMGIPFVIMSATLPTNFIRRLSERFGATIVEGTRLEGKNRRQRRVVLRVSSEKLSIETILELTRNVERTLVVVNTVQRAQNLYEQLLGKIGCPVILAHSRFYDDDRRTKEKQIEAQFGKTAEGQCLLIATQVVEVGLDISCDLLVTELAPIDAIVQRAGRCARWGGQGEVVVFTGLETTRPYDRTLVEATEKALREKNLNGQELTWEIERALVDTVLEPQFSKWAEPEAAGKVLASLAEAAFTGDSAKAERAVREGLTVEVALHGSPDTLGVGALRLPRCRIHPGGFQQFVHKQQPEAWRVVVDRTAADDYRTRVEFLHVDSNSKAAPYGYYIIHPQYGSYDVERGLRLGIRGSPAQSRDELIQRKSRLEGELQIEKWQDHIEKVVKAFAEHVLPKERIAFEALSRRLGKTHEDLLSLTHLVLIFHDLGKLAQQWQRKIQAGLESVLPPGTFLAHRGGSLRDLPPHATVSASLATPCLCRVAGPDWQQTLAIPALAAIAHHHSVRADMTPQFDMSEGWFDVVADCARRLAGVDVTVNDFSRWRGGGSCGVALNFLLPDGYTSYILLSRWLRLADRIATGGGENAILNYEDWMSSS

SEQ ID NO: 8 - protein, 231 aa, synthetic construct
  source 1..231: note = Cas5a protein amino acid sequence of I-A-2
AEWIQAEIEFASFYSYRVPDLSPSYALSSLVPSPAAIRLAVVDAVIRHTGVVDEGESIFELVKRAKLEVQPPARIAVMKFFVKRLKPENPEKGKRASVIESTGIREYCLPSGPLVLWLETEEPERIGQALQWLRRLGTSDSLATCKIGHGAPDTALCIKPANGLAIQAKNFAQRAVFTLHELKPDANFSEVNPFADGRRGDPFEKRLYVLPCVREQAGENWVLYRREPFAN

SEQ ID NO: 9 - protein, 346 aa, unidentified
  source 1..346: note = Cas8a protein amino acid sequence of I-A-2
EYLVVKSGLPTLDAARAYGLAQLLQVLANGKASPYITDQGGVFAVSLNAELTHDALTRSDMWRAAFADSNWQRVFLTYKKAWSAQRDRVKRTLEEQVAAVVTRAGDGLCVDFAGKFALPGPLDPVGFKGLKGLTAGNYSEGQTYLDEQNGALACLGATIAQRYKFGKREYFVTLPIPQMVQFNDFHQIRHLVYDKGLAYLGVRTAAAHFALIFADAIRERAAGNPYFPLSFSNVLYFSLFQSGQQFKPSVGGSINLARLLDIALSRPQAAAEMFKTWDYLFRRGSVKGNEALAEAITDLLMAPSGESYYRHARIFNRYIVDSSKRVNSEFLYDEAALMEVMAYVEQ

SEQ ID NO: 10 - protein, 316 aa, synthetic construct
  source 1..316: note = Cas7 protein amino acid sequence of I-A-2
AGNSVFEISILGRSVWNLHSLNNEGTVGNVSEPRTVILADGSKSDGISGEMLKHIHAQNVWLVATDRSVFCEPCQTLQPQKADKNPDVTGVKAARAKLASEGMNVAIAACALCDLHGFLVQKPTIARASTVEFGWAVAVRNGFHRDIHLHARHAVEGRTEGQQEAGEVAAQMIYHRPTRSGTYALASVFQPWRIGLNEVNYEYVAGVDREARYRLAIEAYKATFARTDGAMTSTRLPHPEAFEGVVLVSSRNFPVPVTSPLQDDYREKLQQLSRATEGLEPQPFNSLTELYGILNELAKRPLFNLQLARSSKREKK

SEQ ID NO: 11 - protein, 343 aa, synthetic construct
  source 1..343: note = Cas6 protein amino acid sequence of I-A-2
SQAHCECSLRVRRFRFVIAPREPLLVPAINKGNMLRGGFGHAFRCLCCIPQCRDARTCPVGMSCPYKAIFEPSPPPEAEALSKNQDIPRPFVFRAPKTQQTRFETGQPFEFELVLIGRALDFLPYFVLSFRELAAEGLGLNRAKCSLERVEQVDLTSEAADASNYEAMVIYTAEDQVFRNAATSETGEWIGRRIRNRSTSRDNDSVQQVSIRFSTPTFLKADGEIIRQPEFHHVFKRLRDRINALSTFFGEGPIEADFRGLGERAEKIRTVSARTDWVERFRTSSKTKQRHELSGFVGEVTYEGNLNEFLPWLTLGELVHVGKHTAWGNGWMELEHEVSRGCV

SEQ ID NO: 12 - protein, 144 aa, synthetic construct
  source 1..144: note = Csa5 (Cas11) protein amino acid sequence of I-A-2
SNSEISLASVFAEESIKSFGKCLRYALWRDDDYASLIEFENAETPLQFAEAVRKFLRRYRSGGFMDEALRTQASEMRKHNRWDELRRTLRQNEIGPRPTEGNLERLTQLANNAQGVRLVRAAIISYGLTKRDPHKELEEVERGS

SEQ ID NO: 13 - protein, 757 aa, synthetic construct
  source 1..757: note = Cas3 protein amino acid sequence of I-A-3
NKLFKKLIGAKPYDYQKIAMENLLDGKSIIMRAPTGSGKTEIALIPFLYGFNDLLPSQLIYSLPTRTLVESIGERAVKYASFRKLRVAIHHGKNATSSLFEEDVVVTTIDQAVGAYLSTPLSMSKRSGNIFVGSVGSALTVFDEVHTLDPEKGLQTSLAISMQSAKLGLPTLIMSATLPDIFIETAKDRISKKGGDIEFIDVKDEFEIKSRKNRFVELINRLEEELNAEKVLEEVEHGKRIIIVINTVNRAQELYLELRNKTELPILLLHSRFLEKDRQEKELLLEETFGKNGNGKCIFIATQIVEVGMDISSPKVLSEIAPIDALIQRAGRCARWSGKGEFHVFGYNTNSKSPHAPYNKDIVEATKSEINNKGKSFTLDWNTEVELVNKILTKHFSEFMNSMIFYQRLGELARAVYEGSRAKVEQNVREVFSCDVTLHENPKSMNSVEILHLPRLRLDARTLMGKVEKIAEMGIDTYRLEENTIIFDDDEDEYVPVLVNNREEIIPFELYVLCGASYSSDTGLVFDDFPNALKSFDPEEKEILSSKQFDNRLKVETWVEHAKNTLKVLDNYMIPRYRYSIENFAENYGYNYGEFLDIIRCTVSLHDIGKLNKKWQKRIKWNDETPLAHSNDNTIKRLPAHATVSAKALQPYLEDLFDDEDIFKAFYLAIAHHHQPWSKSYNEYELVPKYDESLKEIWIIPKNFIQEQNPAGRLDFSYLDIIDENEAYRLYGFLSKLMRISDRLATGGNTYESLFSG

SEQ ID NO: 14 - protein, 223 aa, synthetic construct
  source 1..223: note = Cas5a protein amino acid sequence of I-A-3
QWLKFTLHFPSFFSYRIPDYSSQYALGIPLPSPSTLKLGVISSAIKSTGKVSEGEKVFNVVKDAEVCVAPPEKIAINSFLIKRLKKRKEDLKLIPTFGIRDYVFFPDDIDIFVGSENIDSVAEYFSKMNYIGSSDSMVYVKSIEPKTPSENVIKAVDIDEFSDAAEKESYLVYPVKDINKNATFDQINSYSSKSSRKILDQKYYLINAKVSKGKNWKILDTRN

SEQ ID NO: 15 - protein, 354 aa, synthetic construct
  source 1..354: mol_type = protein note
NHYFLAKSGWEFFDVSKAYGLGLVIQTLTGNASITDRGGFYLIESKNETKFDKIEEISKYFDDSELKTTLITIQRSTKSEMKPPVKKVKGKCLETLTDKESMITVIKNYENLNSPSIIGTDKQTLYQTMDLAATKGIRNEILLKKNYSDGTNIKISDKDFALSLLGHINFTIKKFSDFGLILVAPTPLKTELKNVRQIYANLKGNVKVAHKAGWFPTITQIAINLVSEEIMVKDGGKFAPKFGSLIYSIMRKTGNQWKPSTGGIFPLDFLHQIADSDNAINILNKWKKIFGWTSRKNGHEDLPTSLAEFIANPNLFNYQRYVNFHLRNEIDKDNIKFGDYKKEDFLEVMKNVGI

SEQ ID NO: 16 - protein, 328 aa, synthetic construct
  source 1..328: note = Cas7 protein amino acid sequence of I-A-3
MVNETEIYEIAILGRATWQLHSLNNEGTVGNVTEPRSVTIIDPNTKNPITTDGISGEMLKHIHTGLMWTLTDKNNLCDACKVLNPEKFNVTSGRGSTVEEVLENALNKCDICDLHGFLITRPTVSRKSTIEFGWALGIPEIYRDIHTHARHALGGKTTENEESKGVNTPNSSEDKEEAVGTSTQMVYHRPTRSGVYAVISMFQPWRIGLNETRQDQYTYDTGNNEKRIERYKNALKAYQILFTRPKGAMSTTRLPHVEDFEGVIVFSTDQIPLPLISPLKQDYVKEITDISKKIDNSINVEEFKTLSEFVDKIGDLIDKKPYKLKLGE

SEQ ID NO: 17 - protein, 239 aa, synthetic construct
  source 1..239: note = Cas6 protein amino acid sequence of I-A-3
RLKISLTSNNGNYLIPYNYNHILSAIIYRKIADLDLAAKLHFSKDFKFFTFSQIYFSDWKRTKNGIISKDGKLSFYISSPNEQLIKSLVEGHLENTEVDFKGKKLLVEQIELLKSPSFKENIKLKTMSPVAASIKREVDGKLKIWDLGPGDERFYESVQKNLVNKYTSFYGDYDGDKWVRIKPDMKTAKRRRIEIKGDFHRGYMMEFEMEADPRLVEFAYDCGLGEKNSMGFGMVNIYE

SEQ ID NO: 18 - protein, 113 aa, synthetic construct
  source 1..113: note = Csa5 (Cas11) protein amino acid sequence of I-A-3
SEFRLKDVFEHESIKSFGKTLRKMIRPPKEGNKEKWASDYASIVELGYVETKDQFAEVIKKLLRRYDVIAKKHQLKRPTEKNLEELMELIDKYGVKPVRAALISYALVKKDEE

SEQ ID NO: 19 - protein, 686 aa, synthetic construct
  source 1..686: note = Cas3 protein amino acid sequence of I-A-4
KYKEIFEKLKLNNLTEVQQKISELEGSKNILVVSSCGSGKTEASYFKMLEYNRKTIIIEPMKTLTNSIHGRVDIYNKKLGLEKVSIQHSSSQEDRFLQNKYTVTTIDQVLVGYLAMGKQAYIKGKNIVMSNLIFDEVQLFDTDTMLLTTINMLDEIYKLGNKFIIMTATMPQFLIEFLGERYDMEIVITEKIREDRNVKLFYEEELDYNKVRNYKDKQIIICNSIKQLKEIHKKLPNSRVITLHSTFLGSNRLKLEKQVERYFGKHSEQNDKILLTTQIVEVGMDISCDRLYTTACKIDNLVQRDGRCCRWGGDGQVIVFKNDDNIYEKELVEETIKYIKNNQGIAFNWTIQKQWINEILNEYYKNKINEYNLRKNKFNFNGCNRSRLIRDIQNINVIVVNKEEFTKQDFNRESVSLHINKLKELSQANEIYILNKNKIEKVKYNKVEIGDTVIIRGKNCRYDDLGFRYEEDSAKNMPKCRDFPMTNKSNNNQFRDYIEETWIHHAETVRDLMSYRLNQEQFNDYIIINGKKIAFYGGLHDLGKLDLEWSRKYKSAIPLAHFPFVKGSMGEKRTHELISGEILKEIIDDDIIYNMMIQHHKRLYDDIDIDYKGIEWELHKDTYKILTTYGFKDDIQLQSDAKTLKRNNIMSPCDNEWTTLLYLVGTFMECEIQAINEYIDNYKQAI

SEQ ID NO: 20 - protein, 252 aa, synthetic construct
  source 1..252: note = Cas5a protein amino acid sequence of I-A-4
KKVTYKLSNIFSLKKYNDNNLNCQSYEYPTIYGIRCAILGAIIQVDGIDKVQELFNKIKNSNIYIQYPKEFKVNGIKQKRYANSYYNSCYTEEEYNKLSPSTQSKTYCVLDRDKLVGSNWKTTMGFRQYVKMDNIVFYIDNLIPEIDMYLKNIDWLGTAKSMVYLSDVEEVNKLDNVLTRWNKESYVDTFEQHDWNSKTTFDTIYMYSKKYKHFHDTFMCGIGDIILPSWLWYTRYTFILYFKLWLVNLYEN

SEQ ID NO: 21 - protein, 419 aa, synthetic construct
  source 1..419: note = Cas8a protein amino acid sequence of I-A-4
NEYEFKVIKTANDIEDICISYGICKILSDNRIKFKLKDNKSMYSIYTKEFDIQNDIFYNDFNIENVWNLNSGLNQKETVRALDDMNKFLSENIHDILEHLLNGKVLNYKKESAKGIGNCFYSLGVRASTFGKTLEISPIKKYLSFLGWIYGCSYCYKEKSFEITAILKPYNTDEIAKPFNFSYVDKETGDKKILTKIKKASEINMMSILYIETLKKYKMLSDEYSNVIFMQNIIAGQKPLYDKTTNIKIYKLSQKYLDDLLKKLTWSNVSEDVKDITARYVLNIDKYKEFSKLIKIYSKDGNSKINNDFKGEILSMYNEMIKKIYNDETINKIGKGFNRLLRDNKGFEIQTKLYNVANEKHLVKVLKMIIDLYSRNYKSAILNNDELNKLINTIEDKEYAKICSDAILSIGKVFIIIKK

SEQ ID NO: 22 - protein, 293 aa, synthetic construct
  source 1..293: note = Cas7 protein amino acid sequence of I-A-4
NKIAMMMRLKLTGEALNNEGTIGNVIQPRQIEFPNGEVRQAISGEMLKHYHSRNLRLLADENELCDTCKIFSPMKNGKVKESDSKLSPSGNKVKECIVDDVEGFMNAGKGANEKRTSCVKFSYAIATEENEYQIMLHTRVDVTQDNNKKKQEKETTEGEGNTNKDQNTQMLFHRPLRNNEYAITVQVDLDRIGFDDEKLIYALDEDTIKSRQEKCIKALLNMFVDMEGAMCSTRLPHIEGIEGIIVKKTDKNQVLSKYSALKDDYKEVNEKISDDSIIFNNIIEFSEVMKGLI

SEQ ID NO: 23 - protein, 217 aa, synthetic construct
  source 1..217: note = Cas6 protein amino acid sequence of I-A-4
RINLQGTIIEGQSSIKTNYNHEMYSMILTNISTERANYIHEKKRFKRLFTFSNLYISDNKVHFYVSGQDELIKDFINCIMFNQMVRVGDRVISITNIEPMKNSLETKKEYIFKSNFIVNQKENDRVCLSKDMGYVMKRISDIVKDKYKEIYKEEINENLNVEILNSKQKYTKYKDHHLNSYQATLKVRGNKKLIDLLYNVGIGENTASGHGFVWEVS

SEQ ID NO: 24 - protein, 191 aa, synthetic construct
  source 1..191: note = Csa5 (Cas11) protein amino acid sequence of I-A-4
NNEIKIVKCIDSLYPTVKLTIGKLYKVKESENDKFYRVIADDNNEEQLCYKYRFELVDINEIKELTLQDIFNEEEGIKYNRINGGSGIYTIQNETLIIGEHIKPVLNKRIMDSKFVKVKVERLVSFSDVINSDYKCKVKHYRVEGLIQEESSYTWLEEYQDLKDIMLALSEEFNTIALKEIINKGQWYLEN

SEQ ID NO: 25 - DNA, 2286 bp, synthetic construct
  source 1..2286: mol_type = other DNA; note = Nucleotide sequence encoding I-A-1 Cas3
ctggaggaacagtttcagctggtgaccgggcactcaccggcagaacaccagagggagtgcggagaggcgctggccacgggaaagagtgtgatcctgagggctccgaccggctccggcaaatccgaagccgtgtggattccgttccttcgctgcagaggcaaaaggcttccgatgaggatgatccacgccctgccaatgagagggctcgccaaccagctcgaagagaggatgaaggactacgccgggccgggcctgcgcgtttctgctatgcacggccaaagaccggagtccgtgctgttctacgcggacgcaatcttcgcgaccattgaccaagtggtggccagctacgcctgcgccccgctttccctgtccgtgaggcacggcaacatcccggccggcgctgtggcttcttctttcttggtgtttgatgaggtgcacaccttcgagccgaggctggggttgcagtccatcctggtccttgctgaacgcgcgcaccaaatggggatgcccttcgtgattatgtccgctacgcttccgaagaactttatccgctccctggccgagcgcctgggcgctgctccaatcgagggcggtcgcctgaagtccaaggagggcgagccgaggcacgtcaccctgagggtgcttccggagaagctgagcgccaggaccatccttgactacgcccccaaagtgaatcgcaccgtggtggtggtgaacaccgtccagagagctttgggcttgtacgagcaagtgcgggatgaatttaggtgccccgtgattctggcgcactccagattctacgatgaggacaggagaaccaaggagcagcagatcgaggccctcttcgggaagaaggccgcgcaaggcaggtgcctgctgattgcaacgcaagtggtggaagtgggcctggacatctcctgcgacctgctgataaccgagctggccccggtggacgccctcgttcagagagctgggaggtgcgcgcggtggggtggtaagggagacgtgattgtgctgaccgagcttgacacgaagaggccgtacgacgagacgttggtggccgtcaccgagagggccctgcaggagcataatgtggacgggcaagagctgacgtgggaggtggagacggcgctggtggacaccgtgctggacccgcacttcaaggagtgggcgaagccggacgcggcgggaaaggtgctggcctctctcgcggaggcggcctttaccggcaacagcactaaggcagagcaagccgtgagggagaccctgactgttgaggtggcgctgcacgacaccccacaagccctgggcccggctatcctgaggctgccaagatgtaggctgcacccgggggtgttccagcaattcgtgaggaaacaaagacccaacgtgtggcaggtggttgtcgatagagacccggacgacgattacaggaccaggatcgagttcctgagcgtgaacggcaagagtaggctgatcccgggcgggcactacatcgtggacccgcagttcgggtgttacgatgccgagaggggcctgaggcttggcgtgccaggccagagcgcggaaccatttgcaccgggccagagcagggacagattgaagggcgagctgcagatagagctctggcaggatcacattagagaggtggtgaaagcgtttgagaggtacgtgctgccgaaggagaggatggccttcgaggcgttgtcgcggtggttggggaagacgcaggacgagttgttgagcgtggcgagggcggtgttggtgttgcatgatttggggaagttggcgaggcagtggcaggggaagattcaggcggggctggaggggaagttgagccagggctcctttttggcgcacaggggggggtcggtgtctgggttgccgccacacgccaccgtgagcgcttgggtggcgaccccatgcttgaggaggttggccgggacggactgggagcagacgcttgctgtgccggccttggcggcgatcgcgcatcatcactccgttagggccgacattaccccggagtttgagatgaccgacggctgcttcgaggtggtggccgactgcgccaggggggttgctggtcttgaggtgaagagggacgattttaacaccaagccgccgcagggctcgggctcttgtggcgttggcttgaattttctgcttccagaggggtatacgagctatgtgttgttgtctaggtggttgaggttggcggataggatcgcgacggggggcggcgaagagaccattttccagtatgagaagtggatgggggactct

SEQ ID NO: 26 - DNA, 693 bp, synthetic construct
  source 1..693: mol_type = other DNA; note = Nucleotide sequence encoding I-A-1 Cas5a
gccgagtggctgcaggctgaggtggagttcgccagcttctattcctacagagtgccggatctgtccccgtcctttgcgctgtgctccccggtgccgagcccagctgctattaggttggccgtggtggatgccaccattaggcacaccggggacgttaacgagggccacgccgtctttgagctcatgaagagggccaggctggagctccagccaccgtccagggttgccgtcatgaaattcttcatcaagaggctgaagccagaaaagccgacgaaaggaaagagggccagtgtgattgaatccacggggataagggagtattgtctcccatggggcccgatggtgttctggatcgagtccgaccagccggagaggatcgcgcagtccctgcagtggctgaggaggttgggcaccacggactccttggcctcctgcaccgtgggcgccggtaccccaaacttcgcctcctgcattaggccggccaacgggctgaccctgcagaccaccaacttcgcccaaaggccggtcttcaccctgcacgagctgaagcccgaaacccagttcaaccaagtcaacccgttcgccgacgagagaccaggcaaaccgttcgaaaaaaggctgtacgtgctgccactcgtgcgcgagaaggtgggggaaaactgggtgatatatcaccacgagccgttcgccgcg

SEQ ID NO: 27 - DNA, 1056 bp, synthetic construct
  source 1..1056: mol_type = other DNA; note = Nucleotide sequence encoding I-A-1 Cas8a
gagtacaggctgattaagagcgggctggagatgtttgatacggccagggcctacggcctggcgcagcttctgcaggtgctggccgggggaagggcggctccaagaattctgagccaggggggggtgtttaccctgacaatcagcacgaagccgaacccagcaaccctgaagtcctccgatctctggcgcggcgcgttcggggagagtaactggcaaaaagtgttccttacgtacaagagggcctggtccagccaaagggacaaagtgaaacgcagcctggagtcccacagcgccgacatcttcggcaaggccgagacggatggcctggccgtcgtgtttggcggcaacttcgcgttgccggggccgttggacccggtgggttttaaggggttgaaggggctgacagccggcagttactctgagggccagacgacagtggatgagttcaattgggcgttggggtgtcttggggccgcggctgctcagaggtacaagattcagaaggcggtgggcaacaagtgggagtattacgtgactcttccggtgccggaggaggtgcagttcggcgactttcacgccgtgaggcagctggtgtacgataaaggcctgtcctacaacggggtgaggaatgcagccgcgcacttctccctgctgcttgcctccgccattcgcgagaaagcccaggggaacccgcacttcccggtcaggttctccaacgtgctgtatttctccctgtttcagtccggccagcagttcaagcccgccatcggcggcgccgtgaacgtgggtaggctcattgagatcgccctggcccgcccagaggtcgctcttgagatgtttaagacctgggactacctctttcggcgcggctccgcccaggggaatgaggatcttgcgcaggcgatcaccgaactggtgatggccccctccctggacacctactacaggcacgcgcggatcttcaacaggtacgtggtcgactctaccaaaagggtgaggccggagtacctctacgacgagacggccctgaaagaggtgcttaactacgccgagcag

SEQ ID NO: 28 - DNA, 960 bp, synthetic construct
  source 1..960: mol_type = other DNA; note = Nucleotide sequence encoding I-A-1 Cas7
gcggacagcccggttttcgaggtggccatcctggggcgcgtggtgtggaacctgcacagcctgaacaacgagggcaccgtgggcaacgtgagcgagccgaggaccgtggtgctggcggatgggagcaaatccgacgggatatcaggcgagatgctgaagcatatccacgcccaaaacgtctggctggtggcggaagacaagagccagttgtgcgaaccgtgcaggaccctgaacccgcagaaagccgacaagaacccggccgtgctgggggtgaaaacagcgaaggccaaggtggcggcggagagcatgagcgtggcgatctcctcctgcgccctgtgcgacctgcacggcttcctcgtgcaaagaccgaccatcgccagggccagtaccgtcgaattcgggtgggccgttgcccttagggacggctaccatagggacatccacctgcacgctaggcacgccgtcgagggccgtgctgagaccaccgagggccaacaggagggcccggctgaggtttccgggcaaatgatttaccacaggccgacccgctccggcacctacgctttcgtttccgtcttccagccatggaggatcgggctgaacgaggtgaactacgagtatgtcgaaggcgtggacagggaagcgaggaacaaactggccatcgaggcctacaaggccaccttcgcgaggaccggcggcgctatgacgtccaccaggctgccgcatgtggaggcgttggagggggtggtgttggtgagcagtaggaacttccccgttccggtgacctcccccttgcaggacgattacagggagaagaccgagaaggtgggccaagccgttgaggggctggaggtgcaaaggttcggggccctgccggagctgtacgtgatcctgaatgcgctggccaaaaggaggctgttcgccttgcaaatgggcggcacgtctaaaaaggggaagcag

SEQ ID NO: 29 - DNA, 1023 bp, synthetic construct
  source 1..1023: mol_type = other DNA; note = Nucleotide sequence encoding I-A-1 Cas6
ggcgacgtgttggggttgcactccttgagggtgggcttgttccggttccgcttggtgccggagcagccgttggaggtgccggctttgaacaagggcaacatgttgaggggcgggttcgggcatgggtttaggaagttgtgttgtattccggagtgtcgggatgccaggctttgcccacttgcggctatttgtccgtataaggccgtgttcgagccgagcccgccgccaggttccgagagattggcgaagaaccaggacattccgaggccgttcgtgtttagagcgccccacaccaaccaaaccaggttccagaagggcgaggccttcgagttcggcctcgtcctgatcggcagggcagtggattacctcccatacttcgtcttgtccttcagggagctggccaatgaaggactgggcctcaacagggcgaagtgcgcgctggagcgggttgagcagaggaggaccagcgcgaacgggcttggcagggccaccggtgaggggagactggtgtatagtaaggacagcggggtgtttcacagcacggagaacgagggggtggatagctatgtgaacagcaggctgagggagctgagtagcccgaacggggaccagagcaggcagaacgtgacgatcaggtttttgacgccgaccttcctgaaggcgaacggggaggtgattaggaggccggagttccaccacctgtttaaaagactgagggacaggattaacgcattgtgcacctttttcggcgacggcgccctggacctggactttaggggggtggggaccagggcggagaaggtgcagagcgtctccgcgaggaccgagtgggtggagaggtgcaggaccagcagcaagaccggccaaagacatgaactctctggctttatgggcgaggcgacgtacgaggggaacgtggaggagttcctgccgctgctggcgctgggcgagctggttcacgtcgggaagcacacggcctggggcaacggccgtattgagttgcagtccgggacgggggtgaag

SEQ ID NO: 30 - DNA, 432 bp, synthetic construct
  source 1..432: mol_type = other DNA; note = Nucleotide sequence encoding I-A-1 Csa5 (Cas11)
ctgagcagcgacagcaagctgagtgaggtgttcgcggaggagagcgtgaagagcttcgggaagtgcctgaggtacgccctgtggagggacgaggactacgcgagtctgatagagttcgagaacgcggagacgccgacccagtttgcggatgccgtgaggaagttcctgaggaggtataggtccggcgggtttatggaccagacgcagaggagcagggcctcagagatgaggaagcagaacagatgggacggcctgaaaaagctgctgagacagtacgaggtggggccgaggccaagcgaggggcaactggagaggctgatgcagctggcgaacgacaccaacggggtgaggctcgtgcagagtgccatcattagctacgggcttacaaaaagggagccgtataaggaggtggaggagctggagaaggagaac

SEQ ID NO: 31 - DNA, 2289 bp, synthetic construct
  source 1..2289: mol_type = other DNA; note = Nucleotide sequence encoding I-A-2 Cas3
gtggcctgcgataccttcttcgcccccatgaccgacttcaacctggcgagacaccagagcgagtgcgcgggggcattggcgagtgggaagagtgtgattcttagggccccgaccggctctggcaagtccgaagccgtttggctcccgttcctgtccctgagggggaaaacactgccgtgccgtttgattcacacccttccgatgcgcgccctggtgaaccagctggagtcccggatgagaacctacgcaaatgggcggatgagggttgccgccatgcacgggcaacgccccgagtctgtcctgttctacgccgacgccatcttcgccaccctcgaccaggtcgttacctcttacgcctgcgccccgctgtccctctccgtgagacagggtaacatcccggccggagccgttgccgggtccttccttgtcttcgacgaggttcacaccttcgagccacatctgggcctccagtcccttctcgtcctcgccgagcgtgcccaccaaatgggcatcccgtttgtgatcatgtctgcgaccctccccaccaacttcattcgtaggctgtccgagcgcttcggcgcgaccatcgtcgagggcaccaggttggagggtaagaaccggaggcagaggagagttgtgctgagggtttcttccgagaagctgtctatcgagactatcctggagctgaccaggaacgtcgaaaggaccttggttgtggtgaacacggtgcagagggcccagaacttgtacgagcagctgttggggaagattgggtgcccggtgatcctggcccactctaggttctacgacgacgataggaggacaaaggagaagcagatagaggcgcaattcggtaaaaccgccgaggggcaatgcctgctgatcgccacgcaggtcgttgaggtggggcttgacatcagctgcgacctgctggtgacggagctggccccgattgacgcgatagtccagagggccgggaggtgcgcgaggtggggtggtcaaggggaggtggtggtgtttacggggcttgagacgacgaggccgtatgacaggacgttggtggaggcgacggagaaggccctgagggagaagaacttgaacgggcaggagctgacgtgggagattgagagggccttggtggataccgtgctggagccgcagttttccaaatgggcggagccggaggcggcgggaaaggttctggcgtccctggccgaggccgcgtttaccggagacagcgcgaaggcggagagggccgtgagagagggcctgacagtcgaggtggccttgcacgggtcaccggataccctgggggtgggcgctcttaggttgccgaggtgtaggattcatccgggggggttccaacagtttgtgcataagcagcagccggaagcctggagggtggttgtggataggacggcggcggatgattatagaacgagggtggagttcttgcacgtcgactccaatagcaaggccgccccgtacgggtactacattattcacccgcaatacgggtcttatgatgtggagagggggctgaggctggggatcagggggagcccagcgcagagcagggacgagctgatccagaggaagagcaggctggagggcgagctgcagattgagaagtggcaggaccacattgagaaggtggtgaaggccttcgcggagcacgtcctgccgaaggagaggatagcgttcgaggcattgagcagaaggttggggaagacccacgaagacttgttgagcctcacgcatttggtgctcattttccacgacctggggaagctcgcccaacaatggcagaggaagatccaggccggtttggagagcgtgcttccgccgggcaccttccttgcgcaccgcggtggttccctgagggacctgcctccgcacgccacggtgtctgcctccctcgctaccccgtgcctctgcagggttgctggcccggattggcagcagacgctggccatccctgcccttgctgctatcgcccaccaccactccgttagggccgacatgaccccgcagttcgacatgtccgaggggtggttcgacgtggtggccgactgcgccaggaggctcgctggtgtggacgtgaccgtgaacgacttctcccgctggaggggcggcggctcttgcggtgttgccttgaactttttgcttccggacgggtatacctcttacatcttgttgtccaggtggcttcggctggcggataggatcgcgacgggcggtggtgagaatgctattctgaattatgaggattggatgtcctcttcttct

SEQ ID NO: 32 - DNA, 693 bp, synthetic construct
  source 1..693: mol_type = other DNA; note = Nucleotide sequence encoding I-A-2 Cas5a
gcggagtggattcaagcggagattgagttcgccagcttctacagctacagggtgccggacctgagcccgtcctatgcgctgtcctccctggtgccgagcccggctgctatcaggctggccgtggtggacgccgttattcgccacactggggtggtcgacgagggggagagtattttcgaattggtgaagagggccaagttggaggtgcagccaccggcccggattgccgtgatgaaattttttgtgaagaggctgaagccggaaaacccggagaaggggaagagggccagcgtgattgagagtaccgggatcagggagtactgtctcccgtccggtcctctggtgctgtggctggagacggaggagccggaaaggataggccaggccctgcagtggctgaggaggctcgggacgagcgactccttggccacctgtaagattggccacggcgcgccggacacggccttgtgcattaaaccggcgaacgggctggcgattcaagccaaaaacttcgcccagagggccgtgttcaccctgcacgagctcaaaccggacgccaacttctccgaggttaacccgtttgcggacggcaggaggggagacccgtttgagaagaggctgtacgtcctgccgtgcgtgagagagcaggccggcgaaaactgggtgctgtatagacgggagccgttcgccaac

SEQ ID NO: 33 - DNA, 1038 bp, synthetic construct
  source 1..1038: mol_type = other DNA; note = Nucleotide sequence encoding I-A-2 Cas8a
gagtatttggtggtgaagagtgggctgccgacgctggacgccgccagagcttacgggttggctcagttgttgcaggtgttggccaatggcaaggcaagcccgtatattaccgaccaggggggcgtgtttgcggtgagccttaacgccgagttgacccacgacgcgctgaccaggtctgatatgtggagggccgcgtttgccgacagtaactggcagagagtgttcctgacctacaagaaagcgtggagtgcgcagagggacagagtgaagcgcaccctggaagagcaggtggccgccgtggtgacaagagccggggatgggctgtgcgtggacttcgccggcaagttcgcgctgccggggccattggaccctgtgggtttcaaggggctcaaaggcctgacggccgggaattattccgagggccaaacgtacttggatgagcagaatggggcccttgcgtgcctgggtgctactatcgcccagaggtacaagttcgggaagagggagtacttcgtgacccttccgatcccgcagatggtgcagttcaacgactttcaccagatcaggcatttggtgtacgacaaggggctggcctatctcggggtgcgcactgccgcggcacacttcgctctgatcttcgccgacgccataagggagagggccgccggtaacccctacttcccgctctctttctccaacgtgctgtatttttccctgttccagtccggccagcagtttaagccctccgtgggcggttccattaacctcgctcgcttgctggacatcgccctctcccgcccccaggctgctgctgaaatgttcaagacctgggactacctctttcgccgcggctccgtgaaaggcaatgaggctctcgccgaagccattaccgacctcctgatggccccgtccggcgaatcttactataggcacgcgaggatattcaaccggtacatcgtcgattccagcaagagggtgaactccgaatttctctacgacgaggccgccctgatggaagtgatggcctacgtggagcag

SEQ ID NO: 34 - DNA, 948 bp, synthetic construct
  source 1..948: mol_type = other DNA; note = Nucleotide sequence encoding I-A-2 Cas7
gcggggaacagcgtgttcgagatcagcatcctggggaggtcagtctggaacctgcacagcctgaacaacgaggggacggtgggcaacgtgagcgagccgaggaccgttatattggccgatgggagcaagtccgacgggatcagcggcgaaatgttgaagcatattcacgcccagaacgtttggctggtggcgaccgataggagcgtgttctgcgaaccgtgccagaccctgcagccgcagaaggccgataaaaacccggacgtgaccggcgtgaaggccgccagagccaagctggcgtcggagggaatgaacgtggcgatcgccgcctgtgccctgtgtgacctgcacggcttcctcgtgcaaaaaccaaccatcgccagggcctccaccgtggaattcggctgggccgtggccgtgaggaacgggttccatagggacatccatctgcacgccaggcacgccgtcgagggcagaaccgagggccaacaagaggccggggaagtggccgcccagatgatctaccataggccgaccaggagcgggacctacgccttggcgtccgtgttccaaccgtggagaattgggctgaacgaagtgaattacgagtacgtggcgggcgttgatagagaagccaggtacaggttggccatcgaggcatataaagcgaccttcgcccgcaccgacggcgccatgacctccactaggctgccgcatccggaggcattcgagggagtggtgctggtgagctccaggaacttcccggtgccggtgacctccccacttcaagacgactacagggaaaagctgcagcagctctccagggccaccgagggtctggagccgcagccatttaactccctgaccgagctgtacggcatcctgaacgaactggccaagaggccactgttcaacctgcaactggccaggtcctccaagagggagaagaag

SEQ ID NO: 35 - DNA, 1029 bp, synthetic construct
  source 1..1029: mol_type = other DNA; note = Nucleotide sequence encoding I-A-2 Cas6
tcccaggcccactgcgagtgctccctgagggtgaggaggttcaggttcgtgatcgcgccgagggagccgttgttggtgccggctatcaacaaggggaacatgctgagaggggggtttggccacgccttcaggtgtctgtgctgcatcccgcagtgcagggacgccaggacctgcccagtggggatgagctgcccgtacaaagccatcttcgagccgtccccgccgccggaagccgaagctctttccaagaaccaagacatcccgagaccattcgtgttcagggcaccaaaaacccagcagacccgcttcgagaccggacagcccttcgagttcgagctggtcctgataggaagggccctcgacttcctgccgtacttcgttctctccttcagggagctggccgccgagggcttgggtctgaatagggccaagtgctccctcgagagggtggagcaagtggacctgacgtccgaagccgccgacgcctccaactacgaggccatggttatctataccgccgaagaccaggtgttccgcaacgccgccacctccgagaccggcgaatggattggcaggagaatccgcaacaggtccaccagcagggacaacgactccgtgcagcaggtgtccatcagattttccacaccgaccttcctgaaagccgacggcgaaatcattaggcagccggagttccaccacgtcttcaagaggctgagggacagaatcaacgccctttccaccttctttggcgagggcccgatcgaagccgacttcaggggtttgggggagagggcggagaagatcaggacggtctccgccaggaccgactgggtggagaggttcaggacctcctctaagaccaagcaaagacatgaactgagcgggttcgtgggcgaggtgacgtatgagggcaacctgaacgagttcctgccgtggctgacgctgggggagttggtgcacgtcggcaagcacaccgcctggggcaacggctggatggagctggagcatgaggtgtctaggggctgtgtg

SEQ ID NO: 36 - DNA, 432 bp, synthetic construct
  source 1..432: mol_type = other DNA; note = Nucleotide sequence encoding I-A-2 Csa5 (Cas11)
agcaacagcgagataagcctggcgagcgtgttcgcggaggagagcataaagagcttcgggaagtgcctgaggtacgcgctgtggagggacgacgactacgcgagtttgatcgagttcgagaacgcggagaccccgcttcaattcgccgaggccgtgaggaagttcctgaggcggtataggtccggcgggttcatggatgaggccctgaggacgcaggccagcgagatgaggaagcacaacaggtgggacgaactgaggcgcaccttgagacagaacgagatcggcccgaggccgacggaggggaatctggagaggttgacgcaattggcaaacaacgcgcagggggtgaggctggtgagggcggctataataagctatggcctgacgaagagggacccgcataaggagctggaggaggtggagagggggagc

SEQ ID NO: 37 - DNA, 2271 bp, synthetic construct
  source 1..2271: mol_type = other DNA; note = Nucleotide sequence encoding I-A-3 Cas3
aacaaactgttcaagaagctcattggcgcgaagccgtacgactatcagaagatcgccatggaaaacttgctggacggcaaaagcatcatcatgagggcgccaactgggtctggcaagacggaaattgccctgatccccttcctttacgggttcaacgacttgctcccgtcccagctgatctactcccttccaacccgcaccctggtggagagcatcggcgagcgtgctgtgaagtacgcctcctttcgcaagctgcgcgtggccatccatcacggcaagaacgccacttcctccctcttcgaggaggacgtggtggtgactaccattgaccaggcggtgggcgcgtacctgtccacgccactgtccatgtccaaaaggtccggcaacatcttcgtggggtccgtgggctccgcgctgaccgttttcgatgaggtgcacaccctggacccagaaaaggggcttcagacctccctcgccatctccatgcagtccgcgaagctgggcctgccgaccttgatcatgtctgccactcttccagatatcttcatcgaaaccgccaaagaccgcatctccaagaaggggggcgacatcgagttcatcgacgtcaaggatgagttcgagatcaaatccaggaagaaccgctttgtggagctcattaacaggctggaggaggagctgaacgccgagaaggtgctggaagaggtggagcacgggaaaaggattataattgtgatcaacacggtgaacagggcccaggagctgtatctggaactgaggaacaaaacggagctgccgatcctgctgctgcatagcaggtttttggagaaggacaggcaagagaaggagctgctgctcgaagagaccttcggcaaaaacggcaacggcaagtgcatattcatcgccacgcaaattgtggaggtgggcatggacatctccagcccgaaggtgctgagcgagatcgcgccgatcgacgccttgatccagagggcaggcaggtgcgctaggtggtccggcaaaggggagttccacgttttcggttataacaccaacagcaagtccccgcatgctccgtacaacaaagacatcgtggaggccaccaagtccgagattaacaacaaaggcaagtccttcacgctggactggaacaccgaggtggagctggtgaacaagatcctcactaaacacttctccgagttcatgaacagcatgatattctaccaaaggctgggcgaactcgcgcgcgccgtctacgaagggagcagggcaaaagtggaacaaaacgtgagggaggtgttcagctgcgacgtcacgctccacgagaacccgaagtccatgaactccgtggagatactccacctgccgaggctgaggctggacgcgaggacgctgatgggcaaggtggagaagattgccgaaatgggcattgacacctacagactggaggagaacaccatcatctttgacgacgacgaggacgagtacgtgccggtgctggtgaacaacagggaggagatcattccgttcgagctgtacgtgctttgcggcgcctcctactccagcgacaccggcttggtgtttgacgactttccgaatgcgctgaagtccttcgatccggaggagaaggagatactgtcctccaagcagtttgacaatcgccttaaggtggagacctgggtggaacacgcgaagaataccctgaaagtgctggacaattacatgattccgaggtacaggtacagcatagagaacttcgccgaaaactacgggtacaactacggggaatttctggacattataagatgtaccgtgtccctgcacgatatcgggaagctcaacaaaaaatggcagaagaggatcaagtggaacgacgagaccccgctggcgcactccaacgacaacaccatcaagcgcctgccggcccacgccaccgtgtcagctaaggcgctccagccatacctggaggacctgttcgacgacgaggatatcttcaaagccttctacctcgccatcgcccaccaccaccaaccatggtccaaatcctataacgagtacgagctggtgccgaaatacgacgaatccctgaaggagatatggatcattcccaagaactttatccaggaacagaacccggccggccgcctggacttttcctacctggacatcatcgacgaaaacgaggcctaccgcctgtacggcttcctgagcaagctgatgcgcatctccgacaggctggccaccggaggcaacacctacgagagcctgttttccggc

SEQ ID NO: 38 - DNA, 669 bp, synthetic construct
  source 1..669: mol_type = other DNA; note = Nucleotide sequence encoding I-A-3 Cas5a
cagtggctgaaattcaccctgcacttcccgtccttcttcagctacagaataccagactactcctctcagtacgcgctgggcatccccctgccgtccccatctaccctgaagctcggcgtcatatccagcgccatcaagtccaccggcaaggtgtccgaaggcgaaaaggtgtttaacgtggtgaaggacgccgaagtctgcgtcgccccccccgaaaagatcgccatcaactccttcctcatcaagcggctcaagaagaggaaagaagacctgaaactgataccgaccttcggcattagggactatgttttctttccggatgacatcgacatcttcgtggggtccgaaaacatcgactccgtggctgaatacttctcaaagatgaactacatcggctcctccgactccatggtgtacgtgaagtccatcgagcccaagaccccgtccgagaacgttattaaggccgtggacattgacgagttcagcgacgccgccgagaaagagtcctacctcgtctacccggtgaaggatatcaataagaacgccaccttcgaccagatcaacagctactcatccaaatcatccaggaagattctggaccaaaaatactaccttatcaacgccaaggtgtccaagggcaaaaactggaagatcctggacacccgcaac

SEQ ID NO: 39 - DNA, 1062 bp, synthetic construct
  source 1..1062: mol_type = other DNA; note = Nucleotide sequence encoding I-A-3 Cas8a
aaccactacttcctggcgaagagcgggtgggagttcttcgacgtgtccaaagcctacgggctcggcctggtgatccaaaccctcacaggcaacgccagcatcaccgacagggggggcttctacttgatcgaatccaaaaacgagaccaagttcgacaagattgaggagatctccaagtacttcgacgacagcgaactcaagaccaccctcatcaccatccaaagaagcacgaagtccgagatgaaaccgccggtgaaaaaagtgaagggcaagtgcctggagacgctgaccgacaaggagagcatgatcaccgtgatcaaaaactacgagaacctgaacagcccgagcatcatcggcaccgacaagcaaaccctgtaccagaccatggacctcgctgcgaccaaaggcataaggaacgaaatcctcctcaagaaaaactactccgacggcaccaacatcaaaatctccgacaaagacttcgccctctccctcctcggacacatcaacttcaccatcaaaaaattctccgacttcggcctgatcctcgtggccccgaccccactgaagaccgaactcaagaacgtgaggcaaatttacgccaacctgaaaggcaacgtcaaggtggcccacaaggcaggctggttcccgaccattacccagatcgccatcaacctcgtttccgaagagataatggtgaaagatgggggcaagttcgccccaaaattcggctccctgatctattcaataatgaggaaaacgggcaaccagtggaaaccaagcaccggcggcatcttcccgctggatttcctgcaccaaattgcggattccgacaacgccatcaacatcctcaacaagtggaagaaaatattcggatggacgtcccgcaagaacggccacgaggacctgccaacctccctcgccgagttcatcgccaacccgaacctcttcaactaccaaaggtacgtcaacttccacctgcgcaacgaaatcgacaaggacaacatcaaattcggcgactacaaaaaagaagacttcctggaggtcatgaagaacgtgggcatc

SEQ ID NO: 40 - DNA, 984 bp, synthetic construct
  source 1..984: mol_type = other DNA; note = Nucleotide sequence encoding I-A-3 Cas7
atggtgaacgagacggagatctacgagattgcgatcttggggagggcgacgtggcagctgcacagcctgaacaacgaggggacggtggggaacgtgacggagccgaggagcgtgacgatcattgacccgaacaccaagaacccgatcaccaccgacgggataagtggcgagatgctcaaacacatccacaccgggctgatgtggacgctgacggacaagaacaacctgtgtgacgcctgcaaggtgctgaacccggaaaagttcaacgtgaccagtggcaggggcagcaccgttgaagaggtgctcgaaaatgccctgaataagtgcgacatatgcgacctgcatggctttctgatcacgaggcctaccgtctccaggaagtccaccatcgagttcgggtgggccctcgggattcctgagatctaccgggatattcacacccacgccaggcacgccctgggcggtaagaccaccgagaacgaagagagtaagggcgtgaacacaccgaacagcagcgaggataaagaggaggccgtgggcaccagcacccagatggtgtaccacaggccaacgcggagcggggtgtatgcagtgatcagcatgttccagccgtggaggatcggcctcaacgagacgaggcaggaccagtacacgtatgacacgggcaacaacgagaagaggatcgagaggtacaaaaatgcgctgaaagcctaccagatcctgttcaccaggccgaagggggccatgagcacaacgaggctgccgcacgtcgaggacttcgagggcgtgatagtcttctccaccgaccagatcccgcttcccctgatctccccactgaagcaggactacgtcaaagaaatcaccgacatatccaagaagattgacaacagcataaacgtggaggagttcaagaccctgtccgagttcgtggacaaaatcggcgacctgatcgacaaaaaaccgtacaagctgaagctgggcgag

SEQ ID NO: 41 - DNA, 717 bp, synthetic construct
  source 1..717: mol_type = other DNA; note = Nucleotide sequence encoding I-A-3 Cas6
aggctgaagatctccctgacctccaacaacggcaactacctgatcccgtacaactacaaccacatcctgtccgccatcatctacaggaagatcgccgacctggacctggccgctaagctgcatttctccaaggacttcaagttcttcaccttctcccagatctacttctccgactggaagcgcaccaagaatggcatcatcagcaaggacggcaagctgagcttctacatctcctcccccaatgagcagctgatcaagtctctggtcgaggggcacctggagaacacggaggtggacttcaagggcaagaagctgctggtggagcagatcgagctgctgaagagcccgagcttcaaggagaacatcaagctgaagacgatgagcccggtggcagccagcatcaaaagggaggtggacgggaagctgaagatctgggatttgggcccgggggatgaaaggttctacgagtccgtgcagaagaacctggtgaacaagtacacgtccttctacggggactacgacggggacaagtgggtgaggatcaagccggacatgaagacggcgaagaggaggaggatcgagattaagggggacttccacagggggtacatgatggagttcgagatggaggccgacccgaggctggtggagtttgcgtacgactgcgggctgggcgaaaagaacagcatggggttcgggatggtgaacatttacgag

SEQ ID NO: 42 - DNA, 339 bp, synthetic construct
  source 1..339: mol_type = other DNA; note = Nucleotide sequence encoding I-A-3 Csa5 (Cas11)
tctgagttcaggctgaaggacgtgtttgagcacgagagcatcaagtccttcggcaagacgctgaggaaaatgatcagaccgccgaaggaggggaacaaggagaagtgggcgagcgactacgccagcatcgtcgaactggggtacgtggagacgaaggaccagttcgcggaggtgatcaaaaagctgctgaggaggtacgacgtgatagcgaagaagcaccagctgaagagaccgactgagaaaaacctggaggagttgatggagctgatcgacaagtatggcgtgaagccggtgagggcggccttgataagctacgccctggtgaaaaaagacgaagag

SEQ ID NO: 43 - DNA, 2058 bp, synthetic construct
  source 1..2058: mol_type = other DNA; note = Nucleotide sequence encoding I-A-4 Cas3
aagtacaaggagattttcgagaagctgaagctgaacaacctgaccgaggtgcagcagaagatctccgagctggagggcagcaagaacatcctcgtggtgagctcctgcggctctggcaagactgaggcttcctacttcaagatgctggagtacaacaggaagacgatcatcatcgagccgatgaagaccctgacgaacagcatccacggcagggtggacatctacaacaagaagctgggcctggagaaggtgtccatccagcacagcagcagccaggaggacaggttcctgcagaacaagtacaccgtgaccaccatcgaccaggtgctggttggctacctggcgatgggcaagcaggcttacatcaagggcaagaacatcgtgatgtccaacctgattttcgacgaggtgcagctgttcgacaccgacaccatgctgctcacgaccattaatatgctcgatgagatctacaagctgggcaacaagttcatcatcatgaccgccaccatgccccagttcctgattgagttcctgggcgagaggtacgacatggagattgtgatcaccgagaagatcagggaggacaggaacgtcaagctgttctacgaggaggagctggattacaacaaggttaggaattacaaggataagcagatcatcatctgcaactccatcaagcagctgaaggagatccacaagaagctgccaaactccagggtcatcacgctgcactccaccttcctcggttccaacaggctgaagctggagaagcaggtggagaggtacttcggcaagcactccgagcagaacgacaagatcctgctgacaacccagatcgtggaggtgggcatggacatcagctgcgacaggctgtacacaaccgcgtgcaagatcgacaacctggtgcagagggacggcaggtgctgcaggtggggcggcgatggccaggttattgtgttcaagaacgacgacaacatctacgagaaggagctggtcgaggagacgatcaagtacatcaagaataaccagggcatcgcgttcaactggacgatccagaagcagtggattaacgagatcctgaacgagtactacaagaacaagatcaacgagtacaacctgaggaagaacaagttcaacttcaacggctgcaacaggtcccgcctgatcagggatatccagaacatcaacgtgatcgtggtgaacaaggaggagttcacgaagcaggacttcaacagggagtccgtcagcctgcacatcaacaagctcaaggagctctcccaggccaacgagatctacatcctcaacaagaacaagattgagaaggtgaagtacaacaaggtcgagatcggcgacacggtgattatccgcggcaagaactgcaggtacgacgaccttggcttcaggtacgaggaggacagcgccaagaacatgccgaagtgcagggacttcccaatgaccaacaagagcaacaacaaccagttcagggattacatcgaggagacctggattcatcacgccgagaccgtgagggacctgatgtcatacaggctgaaccaggagcagttcaacgactacatcatcatcaacggcaagaagatcgcgttctacggcggcctgcacgaccttggcaagctggatctggagtggtcccgcaagtacaagagcgccatcccactcgctcacttcccgttcgtgaagggctccatgggcgagaagaggacccacgagctgatctccggcgagattctgaaggagattattgacgacgacatcatctacaacatgatgatccagcaccacaagcgcctgtacgacgatattgacatcgactacaagggcatcgagtgggagctgcataaggacacctacaagatcctgaccacctacggcttcaaggacgacatccagctgcagagcgacgccaagactctgaagcgcaacaacattatgagcccgtgcgacaacgagtggaccacccttctctacctggtgggcaccttcatggagtgcgagattcaggccatcaacgagtacatcgataactacaagcaggccatc

SEQ ID NO: 44 - DNA, 756 bp, synthetic construct
  source 1..756: mol_type = other DNA; note = Nucleotide sequence encoding I-A-4 Cas5a
aagaaggtgacctacaagctgagcaatatcttctcccttaagaagtacaacgacaacaacctgaactgccagagctacgagtaccccaccatctacggcatcaggtgcgccatcctgggcgctattatccaggtggacggcatcgacaaggttcaggagctgttcaacaagatcaagaacagcaacatctacatccagtaccccaaggagttcaaggtgaacggcatcaagcagaagaggtacgccaactcctactacaactcctgctacaccgaggaggagtacaacaagctgtccccgtccacgcagtccaagacctactgcgtgctggacagggacaagctggtgggctcaaactggaagaccaccatgggcttccgccagtacgtgaagatggacaacatcgtgttctacatcgacaacctcatcccggagatcgacatgtacctgaagaacatcgactggctgggcaccgcgaaatctatggtttacctttctgacgtggaggaggtgaacaagctggacaacgtgctgaccaggtggaacaaggagagctacgtggacaccttcgagcagcacgactggaattccaagaccaccttcgatacgatctacatgtactccaagaagtacaagcacttccacgataccttcatgtgcggcatcggcgacatcatcctgccctcttggctctggtacacgaggtacaccttcatcctttacttcaagctgtggctggtgaatctgtacgagaac

SEQ ID NO: 45 - DNA, 1257 bp, synthetic construct
  source 1..1257: mol_type = other DNA; note = Nucleotide sequence encoding I-A-4 Cas8a
aatgagtacgagttcaaggtgattaagacggccaacgacatcgaggacatctgcatctcctacggcatttgcaagatcctgagcgacaacaggatcaagttcaagctgaaggataacaagtccatgtactctatctacaccaaggagttcgacatccagaacgacatcttctacaatgacttcaacatcgagaacgtctggaacctcaactccggcctcaaccagaaggagaccgtgagggctctggacgacatgaacaagttcctctccgagaacatccacgacatcctggagcacctgctgaacggcaaggtgcttaactacaagaaggagagcgccaagggcattggcaattgcttctactccctgggcgtgagggcctctaccttcggcaagacactggagatctcccccatcaagaagtacctgtccttcctcggctggatctacggctgcagctactgctacaaggagaagtccttcgagattaccgccatcctgaagccctacaacaccgacgagatcgccaagcccttcaacttctcctacgtcgacaaggagaccggcgacaagaagatcctgaccaagatcaagaaggccagcgagatcaacatgatgtccatcctctacatcgagaccctcaagaagtacaagatgctgtccgacgagtacagcaacgtgatcttcatgcagaacatcatcgccggccagaagccactgtacgacaagaccaccaacatcaagatctacaagctgtcccagaagtacctggacgacctgctgaagaagctgacctggtccaacgtgtccgaggacgtgaaggacatcaccgccaggtacgtgctgaacatcgacaagtacaaggagttctccaagctgatcaagatctactccaaggacggcaactccaagatcaacaacgacttcaagggcgagatcctgtccatgtacaacgagatgatcaagaagatctacaacgacgagaccatcaacaagatcggcaagggcttcaacaggctgctgagggacaacaagggcttcgagatccagacgaagctgtacaacgtcgccaacgagaagcacctggtgaaggtgctgaagatgatcatcgacctgtactccaggaactacaagtctgccatcctgaacaacgacgagctgaacaagctgatcaacaccatcgaggataaggagtacgccaagatctgctccgacgccatcctgtccatcggcaaggtgttcatcatcatcaagaag

SEQ ID NO: 46 - DNA, 879 bp, synthetic construct
  source 1..879: mol_type = other DNA; note = Nucleotide sequence encoding I-A-4 Cas7
aacaagatcgcgatgatgatgaggctgaagctgacgggcgaggcccttaacaacgagggcactatcggcaacgtgatccagccgaggcagatcgagttcccgaatggcgaggtgaggcaggctatttctggcgagatgctgaagcactaccacagcaggaacctgaggctgctggcggatgagaacgagctgtgcgacacctgcaagatcttcagcccgatgaagaacggcaaggtcaaggagagcgatagcaagctgagcccgtccggcaacaaggtgaaggagtgcatcgtggacgacgtggagggcttcatgaacgccggcaagggcgctaacgagaagaggactagctgcgtgaagttctcctacgccatcgccaccgaggagaacgagtaccagatcatgctgcataccagggtggacgtgacgcaggacaacaacaagaagaagcaggagaaggagacgaccgagggcgagggcaataccaacaaggaccagaacacccagatgctgttccaccggccgctgaggaacaatgagtacgccatcacggtgcaggtggacctggataggatcggcttcgacgacgagaagctgatctacgccctggacgaggacacaatcaagtcccgccaggagaagtgcatcaaggccctgctgaacatgttcgtggacatggagggcgccatgtgctccactaggctgccacatatcgagggcatcgagggcattatcgtgaagaagaccgacaagaaccaggtgctctccaagtactccgccctgaaggatgactacaaggaggtgaacgagaagatctcagacgactccatcatcttcaacaacattatcgagttctccgaggtgatgaagggcctcatt

SEQ ID NO: 47 - DNA, 648 bp, synthetic construct
  source 1..648: mol_type = other DNA; note = Nucleotide sequence encoding I-A-4 Cas6
aggatcaacctgcagggcaccatcatcgagggccagtcttccatcaagacaaactacaaccacgagatgtacagcatgatcctgaccaacatctccaccgagcgcgccaactacatccacgagaagaagaggttcaagaggctgttcaccttctccaacctgtacatctccgacaacaaggtgcacttctacgtctccggccaggacgagctcatcaaggacttcatcaactgcatcatgttcaaccagatggtgagggtgggcgaccgcgttatctccatcaccaacattgagccgatgaagaactccctcgagacgaagaaggagtacatcttcaagtccaacttcatcgtgaaccagaaggagaacgatagggtgtgcctgagcaaagatatgggttacgtgatgaagcgcatctccgacatcgtgaaggacaagtacaaggagatctacaaggaggagattaacgagaacctgaacgtggagatcctgaatagcaagcagaagtacacgaagtacaaggaccaccacctgaacagctaccaggcgacgctgaaggtgaggggcaacaagaagctgatcgacctgctgtacaacgtgggcatcggcgagaacaccgcctcaggccatggcttcgtgtgggaggtt

SEQ ID NO: 48 - DNA, 573 bp, synthetic construct
  source 1..573: mol_type = other DNA note
aacaacgagatcaagatcgtgaagtgcatcgacagcctgtacccgaccgtgaagctgacgattggcaagctgtacaaggtgaaggagagcgagaacgacaagttctacagggtgatcgccgacgacaacaacgaggagcagctgtgctacaagtacaggttcgagctggtggatatcaacgagatcaaggagctgacgctgcaggacatcttcaacgaggaggagggcattaagtacaacaggatcaacggcggctcaggcatttacacgatccagaacgagaccctgattatcggcgagcacatcaagccggtgcttaacaagaggatcatggactccaagttcgtcaaggtcaaggtggagaggctggtgtccttcagcgacgtgatcaactcagactacaagtgcaaggtgaagcactacagggtggagggcctcatccaggaggagtctagctacacctggctggaggagtaccaggaccttaaggacatcatgctggccctgtcagaggagttcaacaccatcgccctgaaggagatcattaacaagggccagtggtacctggagaac

SEQ ID NO: 49 - RNA, 37 nt, synthetic construct
  source 1..37: mol_type = other RNA; note = Prototype direct repeat sequence of I-A-1
gttccagagccttccccgatgaagaggggactgaaag

SEQ ID NO: 50 - DNA, 37 bp, synthetic construct
  source 1..37: mol_type = other DNA; note = Nucleotide sequence encoding prototype direct repeat sequence of I-A-1
gttccagagccttccccgatgaagaggggactgaaag

SEQ ID NO: 51 - RNA, 29 nt, synthetic construct
  source 1..29: mol_type = other RNA; note = I-A-1 mature direct repeat sequence - first region containing stem-loop structure
gttccagagccttccccgatgaagagggg

SEQ ID NO: 52 - skipped

SEQ ID NO: 53 - RNA, 37 nt, synthetic construct
  source 1..37: mol_type = other RNA note
gttgaagagccttccccgatgaagaggggactgaaag

SEQ ID NO: 54 - DNA, 37 bp, synthetic construct
  source 1..37: mol_type = other DNA; note = Nucleotide sequence encoding prototype direct repeat sequence of I-A-2
gttgaagagccttccccgatgaagaggggactgaaag

SEQ ID NO: 55 - RNA, 29 nt, synthetic construct
  source 1..29: mol_type = other RNA; note = I-A-2 mature direct repeat sequence - first region containing stem-loop structure
gttgaagagccttccccgatgaagagggg

SEQ ID NO: 56 - skipped

SEQ ID NO: 57 - RNA, 30 nt, synthetic construct
  source 1..30: mol_type = other RNA note
gctcaaatcagactattttaggattgaaat

SEQ ID NO: 58 - DNA, 30 bp, synthetic construct
  source 1..30: mol_type = other DNA; note = Nucleotide sequence encoding prototype direct repeat sequence of I-A-3
gctcaaatcagactattttaggattgaaat

SEQ ID NO: 59 - RNA, 22 nt, synthetic construct
  source 1..22: mol_type = other RNA; note = I-A-3 mature direct repeat sequence - first region containing stem-loop structure
gctcaaatcagactattttagg

SEQ ID NO: 60 - skipped

SEQ ID NO: 61 - RNA, 29 nt, synthetic construct
  source 1..29: mol_type = other RNA; note = Prototype direct repeat sequence of I-A-4
attaagatttaacaatcatgttatttaaa

SEQ ID NO: 62 - DNA, 29 bp, synthetic construct
  source 1..29: mol_type = other DNA; note = Nucleotide sequence encoding prototype direct repeat sequence of I-A-4
attaagatttaacaatcatgttatttaaa

SEQ ID NO: 63 - RNA, 21 nt, synthetic construct
  source 1..21: mol_type = other RNA; note = I-A-4 mature direct repeat sequence - first region containing stem-loop structure
attaagatttaacaatcatgt

SEQ ID NO: 64 - skipped

SEQ ID NO: 65 - protein, 8 aa, synthetic construct
  source 1..8: note = NLS amino acid sequence
MPKKKRKV

SEQ ID NO: 66 - protein, 10 aa, synthetic construct
  source 1..10: note = linker-1 amino acid sequence
GIHGVPAAGS

SEQ ID NO: 67 - skipped

SEQ ID NO: 68 - protein, 780 aa, synthetic construct
  source 1..780: note = Amino acid sequence of NLS-(linker 1)-(I-A-1 Cas3) fusion protein
MPKKKRKVGIHGVPAAGSLEEQFQLVTGHSPAEHQRECGEALATGKSVILRAPTGSGKSEAVWIPFLRCRGKRLPMRMIHALPMRGLANQLEERMKDYAGPGLRVSAMHGQRPESVLFYADAIFATIDQVVASYACAPLSLSVRHGNIPAGAVASSFLVFDEVHTFEPRLGLQSILVLAERAHQMGMPFVIMSATLPKNFIRSLAERLGAAPIEGGRLKSKEGEPRHVTLRVLPEKLSARTILDYAPKVNRTVVVVNTVQRALGLYEQVRDEFRCPVILAHSRFYDEDRRTKEQQIEALFGKKAAQGRCLLIATQVVEVGLDISCDLLITELAPVDALVQRAGRCARWGGKGDVIVLTELDTKRPYDETLVAVTERALQEHNVDGQELTWEVETALVDTVLDPHFKEWAKPDAAGKVLASLAEAAFTGNSTKAEQAVRETLTVEVALHDTPQALGPAILRLPRCRLHPGVFQQFVRKQRPNVWQVVVDRDPDDDYRTRIEFLSVNGKSRLIPGGHYIVDPQFGCYDAERGLRLGVPGQSAEPFAPGQSRDRLKGELQIELWQDHIREVVKAFERYVLPKERMAFEALSRWLGKTQDELLSVARAVLVLHDLGKLARQWQGKIQAGLEGKLSQGSFLAHRGGSVSGLPPHATVSAWVATPCLRRLAGTDWEQTLAVPALAAIAHHHSVRADITPEFEMTDGCFEVVADCARGVAGLEVKRDDFNTKPPQGSGSCGVGLNFLLPEGYTSYVLLSRWLRLADRIATGGGEETIFQYEKWMGDS

SEQ ID NO: 69 - protein, 242 aa, synthetic construct
  source 1..242: note = Amino acid sequence of NLS-(linker 2)-(I-A-1 Cas5a) fusion protein
MPKKKRKVGSGAEWLQAEVEFASFYSYRVPDLSPSFALCSPVPSPAAIRLAVVDATIRHTGDVNEGHAVFELMKRARLELQPPSRVAVMKFFIKRLKPEKPTKGKRASVIESTGIREYCLPWGPMVFWIESDQPERIAQSLQWLRRLGTTDSLASCTVGAGTPNFASCIRPANGLTLQTTNFAQRPVFTLHELKPETQFNQVNPFADERPGKPFEKRLYVLPLVREKVGENWVIYHHEPFAA

SEQ ID NO: 70 - protein, 363 aa, synthetic construct
  source 1..363: note = Amino acid sequence of NLS-(linker 2)-(I-A-1 Cas8a) fusion protein
MPKKKRKVGSGEYRLIKSGLEMFDTARAYGLAQLLQVLAGGRAAPRILSQGGVFTLTISTKPNPATLKSSDLWRGAFGESNWQKVFLTYKRAWSSQRDKVKRSLESHSADIFGKAETDGLAVVFGGNFALPGPLDPVGFKGLKGLTAGSYSEGQTTVDEFNWALGCLGAAAAQRYKIQKAVGNKWEYYVTLPVPEEVQFGDFHAVRQLVYDKGLSYNGVRNAAAHFSLLLASAIREKAQGNPHFPVRFSNVLYFSLFQSGQQFKPAIGGAVNVGRLIEIALARPEVALEMFKTWDYLFRRGSAQGNEDLAQAITELVMAPSLDTYYRHARIFNRYVVDSTKRVRPEYLYDETALKEVLNYAEQ

SEQ ID NO: 71 - protein, 331 aa, synthetic construct
  source 1..331: note = Amino acid sequence of NLS-(linker 2)-(I-A-1 Cas7) fusion protein
MPKKKRKVGSGADSPVFEVAILGRVVWNLHSLNNEGTVGNVSEPRTVVLADGSKSDGISGEMLKHIHAQNVWLVAEDKSQLCEPCRTLNPQKADKNPAVLGVKTAKAKVAAESMSVAISSCALCDLHGFLVQRPTIARASTVEFGWAVALRDGYHRDIHLHARHAVEGRAETTEGQQEGPAEVSGQMIYHRPTRSGTYAFVSVFQPWRIGLNEVNYEYVEGVDREARNKLAIEAYKATFARTGGAMTSTRLPHVEALEGVVLVSSRNFPVPVTSPLQDDYREKTEKVGQAVEGLEVQRFGALPELYVILNALAKRRLFALQMGGTSKKGKQ

SEQ ID NO: 72 - protein, 353 aa, synthetic construct
  source 1..353: note = Amino acid sequence of NLS-(linker 2)-(I-A-1 Cas6) fusion protein
MPKKKRKVGSGGDVLGLHSLRVGLFRFRLVPEQPLEVPALNKGNMLRGGFGHGFRKLCCIPECRDARLCPLAAICPYKAVFEPSPPPGSERLAKNQDIPRPFVFRAPHTNQTRFQKGEAFEFGLVLIGRAVDYLPYFVLSFRELANEGLGLNRAKCALERVEQRRTSANGLGRATGEGRLVYSKDSGVFHSTENEGVDSYVNSRLRELSSPNGDQSRQNVTIRFLTPTFLKANGEVIRRPEFHHLFKRLRDRINALCTFFGDGALDLDFRGVGTRAEKVQSVSARTEWVERCRTSSKTGQRHELSGFMGEATYEGNVEEFLPLLALGELVHVGKHTAWGNGRIELQSGTGVKC

SEQ ID NO: 73 - protein, 155 aa, synthetic construct
  source 1..155: note = Amino acid sequence of NLS-(linker 2)-(I-A-1 Csa5(Cas11)) fusion protein
MPKKKRKVGSGLSSDSKLSEVFAEESVKSFGKCLRYALWRDEDYASLIEFENAETPTQFADAVRKFLRRYRSGGFMDQTQRSRASEMRKQNRWDGLKKLLRQYEVGPRPSEGQLERLMQLANDTNGVRLVQSAIISYGLTKREPYKEVEELEKEN

SEQ ID NO: 74 - protein, 780 aa, synthetic construct
  source 1..780: note = Amino acid sequence of NLS-(linker 1)-(I-A-2 Cas3) fusion protein
MPKKKRKVGIHGVPAAGSVACDTFFAPMTDFNLARHQSECAGALASGKSVILRAPTGSGKSEAVWLPFLSLRGKTLPCRLIHTLPMRALVNQLESRMRTYANGRMRVAAMHGQRPESVLFYADAIFATLDQVVTSYACAPLSLSVRQGNIPAGAVAGSFLVFDEVHTFEPHLGLQSLLVLAERAHQMGIPFVIMSATLPTNFIRRLSERFGATIVEGTRLEGKNRRQRRVVLRVSSEKLSIETILELTRNVERTLVVVNTVQRAQNLYEQLLGKIGCPVILAHSRFYDDDRRTKEKQIEAQFGKTAEGQCLLIATQVVEVGLDISCDLLVTELAPIDAIVQRAGRCARWGGQGEVVVFTGLETTRPYDRTLVEATEKALREKNLNGQELTWEIERALVDTVLEPQFSKWAEPEAAGKVLASLAEAAFTGDSAKAERAVREGLTVEVALHGSPDTLGVGALRLPRCRIHPGGFQQFVHKQQPEAWRVVVDRTAADDYRTRVEFLHVDSNSKAAPYGYYIIHPQYGSYDVERGLRLGIRGSPAQSRDELIQRKSRLEGELQIEKWQDHIEKVVKAFAEHVLPKERIAFEALSRRLGKTHEDLLSLTHLVLIFHDLGKLAQQWQRKIQAGLESVLPPGTFLAHRGGSLRDLPPHATVSASLATPCLCRVAGPDWQQTLAIPALAAIAHHHSVRADMTPQFDMSEGWFDVVADCARRLAGVDVTVNDFSRWRGGGSCGVALNFLLPDGYTSYILLSRWLRLADRIATGGGENAILNYEDWMSSS

SEQ ID NO: 75 - protein, 242 aa, synthetic construct
  source 1..242: note = Amino acid sequence of NLS-(linker 2)-(I-A-2 Cas5a) fusion protein
MPKKKRKVGSGAEWIQAEIEFASFYSYRVPDLSPSYALSSLVPSPAAIRLAVVDAVIRHTGVVDEGESIFELVKRAKLEVQPPARIAVMKFFVKRLKPENPEKGKRASVIESTGIREYCLPSGPLVLWLETEEPERIGQALQWLRRLGTSDSLATCKIGHGAPDTALCIKPANGLAIQAKNFAQRAVFTLHELKPDANFSEVNPFADGRRGDPFEKRLYVLPCVREQAGENWVLYRREPFAN

SEQ ID NO: 76 - protein, 357 aa, synthetic construct
  source 1..357: note = Amino acid sequence of NLS-(linker 2)-(I-A-2 Cas8a) fusion protein
MPKKKRKVGSGEYLVVKSGLPTLDAARAYGLAQLLQVLANGKASPYITDQGGVFAVSLNAELTHDALTRSDMWRAAFADSNWQRVFLTYKKAWSAQRDRVKRTLEEQVAAVVTRAGDGLCVDFAGKFALPGPLDPVGFKGLKGLTAGNYSEGQTYLDEQNGALACLGATIAQRYKFGKREYFVTLPIPQMVQFNDFHQIRHLVYDKGLAYLGVRTAAAHFALIFADAIRERAAGNPYFPLSFSNVLYFSLFQSGQQFKPSVGGSINLARLLDIALSRPQAAAEMFKTWDYLFRRGSVKGNEALAEAITDLLMAPSGESYYRHARIFNRYIVDSSKRVNSEFLYDEAALMEVMAYVEQ

SEQ ID NO: 77 - protein, 327 aa, synthetic construct
  source 1..327: note = Amino acid sequence of NLS-(linker 2)-(I-A-2 Cas7) fusion protein
MPKKKRKVGSGAGNSVFEISILGRSVWNLHSLNNEGTVGNVSEPRTVILADGSKSDGISGEMLKHIHAQNVWLVATDRSVFCEPCQTLQPQKADKNPDVTGVKAARAKLASEGMNVAIAACALCDLHGFLVQKPTIARASTVEFGWAVAVRNGFHRDIHLHARHAVEGRTEGQQEAGEVAAQMIYHRPTRSGTYALASVFQPWRIGLNEVNYEYVAGVDREARYRLAIEAYKATFARTDGAMTSTRLPHPEAFEGVVLVSSRNFPVPVTSPLQDDYREKLQQLSRATEGLEPQPFNSLTELYGILNELAKRPLFNLQLARSSKREKK

SEQ ID NO: 78 - protein, 354 aa, synthetic construct
  source 1..354: note = Amino acid sequence of NLS-(linker 2)-(I-A-2 Cas6) fusion protein
MPKKKRKVGSGSQAHCECSLRVRRFRFVIAPREPLLVPAINKGNMLRGGFGHAFRCLCCIPQCRDARTCPVGMSCPYKAIFEPSPPPEAEALSKNQDIPRPFVFRAPKTQQTRFETGQPFEFELVLIGRALDFLPYFVLSFRELAAEGLGLNRAKCSLERVEQVDLTSEAADASNYEAMVIYTAEDQVFRNAATSETGEWIGRRIRNRSTSRDNDSVQQVSIRFSTPTFLKADGEIIRQPEFHHVFKRLRDRINALSTFFGEGPIEADFRGLGERAEKIRTVSARTDWVERFRTSSKTKQRHELSGFVGEVTYEGNLNEFLPWLTLGELVHVGKHTAWGNGWMELEHEVSRGCV

SEQ ID NO: 79 - protein, 155 aa, synthetic construct
  source 1..155: note = Amino acid sequence of NLS-(linker 2)-(I-A-2 Csa5(Cas11)) fusion protein
MPKKKRKVGSGSNSEISLASVFAEESIKSFGKCLRYALWRDDDYASLIEFENAETPLQFAEAVRKFLRRYRSGGFMDEALRTQASEMRKHNRWDELRRTLRQNEIGPRPTEGNLERLTQLANNAQGVRLVRAAIISYGLTKRDPHKELEEVERGS

SEQ ID NO: 80 - protein, 775 aa, synthetic construct
  source 1..775: note = Amino acid sequence of NLS-(linker 1)-(I-A-3 Cas3) fusion protein
MPKKKRKVGIHGVPAAGSNKLFKKLIGAKPYDYQKIAMENLLDGKSIIMRAPTGSGKTEIALIPFLYGFNDLLPSQLIYSLPTRTLVESIGERAVKYASFRKLRVAIHHGKNATSSLFEEDVVVTTIDQAVGAYLSTPLSMSKRSGNIFVGSVGSALTVFDEVHTLDPEKGLQTSLAISMQSAKLGLPTLIMSATLPDIFIETAKDRISKKGGDIEFIDVKDEFEIKSRKNRFVELINRLEEELNAEKVLEEVEHGKRIIIVINTVNRAQELYLELRNKTELPILLLHSRFLEKDRQEKELLLEETFGKNGNGKCIFIATQIVEVGMDISSPKVLSEIAPIDALIQRAGRCARWSGKGEFHVFGYNTNSKSPHAPYNKDIVEATKSEINNKGKSFTLDWNTEVELVNKILTKHFSEFMNSMIFYQRLGELARAVYEGSRAKVEQNVREVFSCDVTLHENPKSMNSVEILHLPRLRLDARTLMGKVEKIAEMGIDTYRLEENTIIFDDDEDEYVPVLVNNREEIIPFELYVLCGASYSSDTGLVFDDFPNALKSFDPEEKEILSSKQFDNRLKVETWVEHAKNTLKVLDNYMIPRYRYSIENFAENYGYNYGEFLDIIRCTVSLHDIGKLNKKWQKRIKWNDETPLAHSNDNTIKRLPAHATVSAKALQPYLEDLFDDEDIFKAFYLAIAHHHQPWSKSYNEYELVPKYDESLKEIWIIPKNFIQEQNPAGRLDFSYLDIIDENEAYRLYGFLSKLMRISDRLATGGNTYESLFSG

SEQ ID NO: 81 - protein, 234 aa, synthetic construct
  source 1..234: note = Amino acid sequence of NLS-(linker 2)-(I-A-3 Cas5a) fusion protein
MPKKKRKVGSGQWLKFTLHFPSFFSYRIPDYSSQYALGIPLPSPSTLKLGVISSAIKSTGKVSEGEKVFNVVKDAEVCVAPPEKIAINSFLIKRLKKRKEDLKLIPTFGIRDYVFFPDDIDIFVGSENIDSVAEYFSKMNYIGSSDSMVYVKSIEPKTPSENVIKAVDIDEFSDAAEKESYLVYPVKDINKNATFDQINSYSSKSSRKILDQKYYLINAKVSKGKNWKILDTRN

SEQ ID NO: 82 - protein, 365 aa, synthetic construct
  source 1..365: note = Amino acid sequence of NLS-(linker 2)-(I-A-3 Cas8a) fusion protein
MPKKKRKVGSGNHYFLAKSGWEFFDVSKAYGLGLVIQTLTGNASITDRGGFYLIESKNETKFDKIEEISKYFDDSELKTTLITIQRSTKSEMKPPVKKVKGKCLETLTDKESMITVIKNYENLNSPSIIGTDKQTLYQTMDLAATKGIRNEILLKKNYSDGTNIKISDKDFALSLLGHINFTIKKFSDFGLILVAPTPLKTELKNVRQIYANLKGNVKVAHKAGWFPTITQIAINLVSEEIMVKDGGKFAPKFGSLIYSIMRKTGNQWKPSTGGIFPLDFLHQIADSDNAINILNKWKKIFGWTSRKNGHEDLPTSLAEFIANPNLFNYQRYVNFHLRNEIDKDNIKFGDYKKEDFLEVMKNVGI

SEQ ID NO: 83 - protein, 339 aa, synthetic construct
  source 1..339: note = Amino acid sequence of NLS-(linker 2)-(I-A-3 Cas7) fusion protein
MPKKKRKVGSGMVNETEIYEIAILGRATWQLHSLNNEGTVGNVTEPRSVTIIDPNTKNPITTDGISGEMLKHIHTGLMWTLTDKNNLCDACKVLNPEKFNVTSGRGSTVEEVLENALNKCDICDLHGFLITRPTVSRKSTIEFGWALGIPEIYRDIHTHARHALGGKTTENEESKGVNTPNSSEDKEEAVGTSTQMVYHRPTRSGVYAVISMFQPWRIGLNETRQDQYTYDTGNNEKRIERYKNALKAYQILFTRPKGAMSTTRLPHVEDFEGVIVFSTDQIPLPLISPLKQDYVKEITDISKKIDNSINVEEFKTLSEFVDKIGDLIDKKPYKLKLGE

SEQ ID NO: 84 - protein, 250 aa, synthetic construct
  source 1..250: note = Amino acid sequence of NLS-(linker 2)-(I-A-3 Cas6) fusion protein
MPKKKRKVGSGRLKISLTSNNGNYLIPYNYNHILSAIIYRKIADLDLAAKLHFSKDFKFFTFSQIYFSDWKRTKNGIISKDGKLSFYISSPNEQLIKSLVEGHLENTEVDFKGKKLLVEQIELLKSPSFKENIKLKTMSPVAASIKREVDGKLKIWDLGPGDERFYESVQKNLVNKYTSFYGDYDGDKWVRIKPDMKTAKRRRIEIKGDFHRGYMMEFEMEADPRLVEFAYDCGLGEKNSMGFGMVNIYE

SEQ ID NO: 85 - protein, 234 aa, synthetic construct; identical to SEQ ID NO: 81
  source 1..234: note = Amino acid sequence of NLS-(linker 2)-(I-A-3 Csa5(Cas11)) fusion protein

SEQ ID NO: 86 - protein, 704 aa, synthetic construct
  source 1..704: note = Amino acid sequence of NLS-(linker 1)-(I-A-4 Cas3) fusion protein
MPKKKRKVGIHGVPAAGSKYKEIFEKLKLNNLTEVQQKISELEGSKNILVVSSCGSGKTEASYFKMLEYNRKTIIIEPMKTLTNSIHGRVDIYNKKLGLEKVSIQHSSSQEDRFLQNKYTVTTIDQVLVGYLAMGKQAYIKGKNIVMSNLIFDEVQLFDTDTMLLTTINMLDEIYKLGNKFIIMTATMPQFLIEFLGERYDMEIVITEKIREDRNVKLFYEEELDYNKVRNYKDKQIIICNSIKQLKEIHKKLPNSRVITLHSTFLGSNRLKLEKQVERYFGKHSEQNDKILLTTQIVEVGMDISCDRLYTTACKIDNLVQRDGRCCRWGGDGQVIVFKNDDNIYEKELVEETIKYIKNNQGIAFNWTIQKQWINEILNEYYKNKINEYNLRKNKFNFNGCNRSRLIRDIQNINVIVVNKEEFTKQDFNRESVSLHINKLKELSQANEIYILNKNKIEKVKYNKVEIGDTVIIRGKNCRYDDLGFRYEEDSAKNMPKCRDFPMTNKSNNNQFRDYIEETWIHHAETVRDLMSYRLNQEQFNDYIIINGKKIAFYGGLHDLGKLDLEWSRKYKSAIPLAHFPFVKGSMGEKRTHELISGEILKEIIDDDIIYNMMIQHHKRLYDDIDIDYKGIEWELHKDTYKILTTYGFKDDIQLQSDAKTLKRNNIMSPCDNEWTTLLYLVGTFMECEIQAINEYIDNYKQAI

SEQ ID NO: 87 - protein, 263 aa, synthetic construct
  source 1..263: note = Amino acid sequence of NLS-(linker 2)-(I-A-4 Cas5a) fusion protein
MPKKKRKVGSGKKVTYKLSNIFSLKKYNDNNLNCQSYEYPTIYGIRCAILGAIIQVDGIDKVQELFNKIKNSNIYIQYPKEFKVNGIKQKRYANSYYNSCYTEEEYNKLSPSTQSKTYCVLDRDKLVGSNWKTTMGFRQYVKMDNIVFYIDNLIPEIDMYLKNIDWLGTAKSMVYLSDVEEVNKLDNVLTRWNKESYVDTFEQHDWNSKTTFDTIYMYSKKYKHFHDTFMCGIGDIILPSWLWYTRYTFILYFKLWLVNLYEN

SEQ ID NO: 88 - protein, 430 aa, synthetic construct
  source 1..430: note = Amino acid sequence of NLS-(linker 2)-(I-A-4 Cas8a) fusion protein
MPKKKRKVGSGNEYEFKVIKTANDIEDICISYGICKILSDNRIKFKLKDNKSMYSIYTKEFDIQNDIFYNDFNIENVWNLNSGLNQKETVRALDDMNKFLSENIHDILEHLLNGKVLNYKKESAKGIGNCFYSLGVRASTFGKTLEISPIKKYLSFLGWIYGCSYCYKEKSFEITAILKPYNTDEIAKPFNFSYVDKETGDKKILTKIKKASEINMMSILYIETLKKYKMLSDEYSNVIFMQNIIAGQKPLYDKTTNIKIYKLSQKYLDDLLKKLTWSNVSEDVKDITARYVLNIDKYKEFSKLIKIYSKDGNSKINNDFKGEILSMYNEMIKKIYNDETINKIGKGFNRLLRDNKGFEIQTKLYNVANEKHLVKVLKMIIDLYSRNYKSAILNNDELNKLINTIEDKEYAKICSDAILSIGKVFIIIKK

SEQ ID NO: 89 - protein, 304 aa, synthetic construct
  source 1..304: note = Amino acid sequence of NLS-(linker 2)-(I-A-4 Cas7) fusion protein
MPKKKRKVGSGNKIAMMMRLKLTGEALNNEGTIGNVIQPRQIEFPNGEVRQAISGEMLKHYHSRNLRLLADENELCDTCKIFSPMKNGKVKESDSKLSPSGNKVKECIVDDVEGFMNAGKGANEKRTSCVKFSYAIATEENEYQIMLHTRVDVTQDNNKKKQEKETTEGEGNTNKDQNTQMLFHRPLRNNEYAITVQVDLDRIGFDDEKLIYALDEDTIKSRQEKCIKALLNMFVDMEGAMCSTRLPHIEGIEGIIVKKTDKNQVLSKYSALKDDYKEVNEKISDDSIIFNNIIEFSEVMKGLI

SEQ ID NO: 90 - protein, 228 aa, synthetic construct
  source 1..228: note = Amino acid sequence of NLS-(linker 2)-(I-A-4 Cas6) fusion protein
MPKKKRKVGSGRINLQGTIIEGQSSIKTNYNHEMYSMILTNISTERANYIHEKKRFKRLFTFSNLYISDNKVHFYVSGQDELIKDFINCIMFNQMVRVGDRVISITNIEPMKNSLETKKEYIFKSNFIVNQKENDRVCLSKDMGYVMKRISDIVKDKYKEIYKEEINENLNVEILNSKQKYTKYKDHHLNSYQATLKVRGNKKLIDLLYNVGIGENTASGHGFVWEVS

SEQ ID NO: 91 - protein, 202 aa, synthetic construct
  source 1..202: note = Amino acid sequence of NLS-(linker 2)-(I-A-4 Csa5(Cas11)) fusion protein
MPKKKRKVGSGNNEIKIVKCIDSLYPTVKLTIGKLYKVKESENDKFYRVIADDNNEEQLCYKYRFELVDINEIKELTLQDIFNEEEGIKYNRINGGSGIYTIQNETLIIGEHIKPVLNKRIMDSKFVKVKVERLVSFSDVINSDYKCKVKHYRVEGLIQEESSYTWLEEYQDLKDIMLALSEEFNTIALKEIINKGQWYLEN

SEQ ID NO: 92 - protein, 18 aa, synthetic construct
  source 1..18: note = Amino acid sequence of T2A cleavage peptide
EGRGSLLTCGDVEENPGP

SEQ ID NO: 93 - protein, 166 aa, synthetic construct
  source 1..166: note = Amino acid sequence of TadA8e protein
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSIN

SEQ ID NO: 94 - DNA, 498 bp, synthetic construct
  source 1..498: mol_type = other DNA; note = Nucleotide sequence encoding TadA8e protein
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggatggagaaattctaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaac

SEQ ID NO: 95 - protein, 32 aa, synthetic construct
  source 1..32: note = Amino acid sequence of linker-3 between TadA8e and I-A Cas8a fusion protein
SGGSSGGSSGSETPGTSESATPESSGGSSGGS

SEQ ID NO: 96 - protein, 561 aa, synthetic construct
  source 1..561: note = Amino acid sequence of NLS-(linker 2)-TadA8e-(linker3)-(I-A-1 Cas8a) fusion protein
MPKKKRKVGSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSEYRLIKSGLEMFDTARAYGLAQLLQVLAGGRAAPRILSQGGVFTLTISTKPNPATLKSSDLWRGAFGESNWQKVFLTYKRAWSSQRDKVKRSLESHSADIFGKAETDGLAVVFGGNFALPGPLDPVGFKGLKGLTAGSYSEGQTTVDEFNWALGCLGAAAAQRYKIQKAVGNKWEYYVTLPVPEEVQFGDFHAVRQLVYDKGLSYNGVRNAAAHFSLLLASAIREKAQGNPHFPVRFSNVLYFSLFQSGQQFKPAIGGAVNVGRLIEIALARPEVALEMFKTWDYLFRRGSAQGNEDLAQAITELVMAPSLDTYYRHARIFNRYVVDSTKRVRPEYLYDETALKEVLNYAEQ

SEQ ID NO: 97 - protein, 555 aa, synthetic construct
  source 1..555: note = Amino acid sequence of NLS-(linker 2)-TadA8e-(linker3)-(I-A-2 Cas8a) fusion protein
MPKKKRKVGSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSEYLVVKSGLPTLDAARAYGLAQLLQVLANGKASPYITDQGGVFAVSLNAELTHDALTRSDMWRAAFADSNWQRVFLTYKKAWSAQRDRVKRTLEEQVAAVVTRAGDGLCVDFAGKFALPGPLDPVGFKGLKGLTAGNYSEGQTYLDEQNGALACLGATIAQRYKFGKREYFVTLPIPQMVQFNDFHQIRHLVYDKGLAYLGVRTAAAHFALIFADAIRERAAGNPYFPLSFSNVLYFSLFQSGQQFKPSVGGSINLARLLDIALSRPQAAAEMFKTWDYLFRRGSVKGNEALAEAITDLLMAPSGESYYRHARIFNRYIVDSSKRVNSEFLYDEAALMEVMAYVEQ

SEQ ID NO: 98 - protein, 563 aa, synthetic construct
  source 1..563: note = Amino acid sequence of NLS-(linker 2)-TadA8e-(linker3)-(I-A-3 Cas8a) fusion protein
MPKKKRKVGSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSNHYFLAKSGWEFFDVSKAYGLGLVIQTLTGNASITDRGGFYLIESKNETKFDKIEEISKYFDDSELKTTLITIQRSTKSEMKPPVKKVKGKCLETLTDKESMITVIKNYENLNSPSIIGTDKQTLYQTMDLAATKGIRNEILLKKNYSDGTNIKISDKDFALSLLGHINFTIKKFSDFGLILVAPTPLKTELKNVRQIYANLKGNVKVAHKAGWFPTITQIAINLVSEEIMVKDGGKFAPKFGSLIYSIMRKTGNQWKPSTGGIFPLDFLHQIADSDNAINILNKWKKIFGWTSRKNGHEDLPTSLAEFIANPNLFNYQRYVNFHLRNEIDKDNIKFGDYKKEDFLEVMKNVGI

SEQ ID NO: 99 - protein, 628 aa, synthetic construct
  source 1..628: note = Amino acid sequence of NLS-(linker 2)-TadA8e-(linker3)-(I-A-4 Cas8a) fusion protein
MPKKKRKVGSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSNEYEFKVIKTANDIEDICISYGICKILSDNRIKFKLKDNKSMYSIYTKEFDIQNDIFYNDFNIENVWNLNSGLNQKETVRALDDMNKFLSENIHDILEHLLNGKVLNYKKESAKGIGNCFYSLGVRASTFGKTLEISPIKKYLSFLGWIYGCSYCYKEKSFEITAILKPYNTDEIAKPFNFSYVDKETGDKKILTKIKKASEINMMSILYIETLKKYKMLSDEYSNVIFMQNIIAGQKPLYDKTTNIKIYKLSQKYLDDLLKKLTWSNVSEDVKDITARYVLNIDKYKEFSKLIKIYSKDGNSKINNDFKGEILSMYNEMIKKIYNDETINKIGKGFNRLLRDNKGFEIQTKLYNVANEKHLVKVLKMIIDLYSRNYKSAILNNDELNKLINTIEDKEYAKICSDAILSIGKVFIIIKK

SEQ ID NO: 100 - DNA, 7355 bp, synthetic construct
  source 1..7355: mol_type = other DNA; note = Nucleotide sequence for PAM consumption experiment for Type I-A-1
tttacactttatgcttccggctcgtatgttaggaggtctttatcatgggtgatgtcttaggactgcattccctgcgcgtcggtctgttccgattcaggctcgtgcccgagcaaccgctagaagtgccggctctgaataagggcaacatgcttcgcggtggttttggccacggttttcggaagctgtgctgtattcccgagtgcagagacgcgaggctttgcccacttgcagctatttgcccctacaaggccgtgttcgagccttctccgccgccgggatcggagcgcttggcgaagaatcaagacatcccgcggccctttgttttccgcgctcctcacacgaaccaaacccgttttcaaaagggcgaagcgtttgagttcgggcttgttctaatcggacgggctgttgattacttgccatacttcgtgctgtcgttcagagaactcgccaatgaggggttaggcctgaatcgggcgaagtgcgctttggaacgtgtcgagcaaaggcgaacctccgccaatggtctcgggcgtgccactggtgaggggaggctggtctatagcaaggattctggagttttccactcgactgaaaacgagggcgtcgacagctacgtgaactcacggctgcgagaattgagttctccgaatggcgaccaatctcgacagaacgtgaccatccggttcctaacaccgacatttctgaaagccaacggggaagtgatccggcgaccggagtttcatcatctttttaagcgactccgcgaccggatcaatgcactctgcacgttttttggggacggcgcgcttgatttggattttcgtggtgtcggcacgcgggccgaaaaagttcagagcgtttccgcccgaacggagtgggtggaacgctgccgcacatcctcgaaaacggggcagcgtcatgagctttcgggcttcatgggcgaagccacttacgagggcaacgtggaggagttcttgccgttgctggcgcttggcgaactggttcacgtcggaaaacacacagcctggggcaacggccggatcgaactgcagtcaggtacgggagtcaagtgctagaggagcagttccaattggtcactgggcatagccccgctgagcatcagcgcgagtgtggggaggcactagccacagggaaatccgtcattcttcgagcccctactggctcaggaaagtccgaggcagtgtggataccgttccttcgttgtcggggtaagagactccccatgcgcatgatccatgcgttgccgatgcgtgggttggcgaatcaactggaagaacgaatgaaggactatgccggtcccggcctgcgcgtatcggccatgcacggccagcgtccggagagtgtcttgttttacgctgacgccatctttgccaccatagatcaggtggtcgcctcttatgcatgtgctcccctcagcttaagtgtacgccatggcaacatcccggctggcgctgtcgctagcagttttttggtttttgacgaagtacacacatttgagcctcgactcggcctgcaatcgattctggtgctggctgagcgagcccaccagatgggcatgccttttgtaatcatgtcggcaactctaccgaagaatttcattcgaagtttggccgaacgattgggtgccgcgccaattgagggcggtcggttgaaaagtaaggaaggagaacctcgccacgtgaccctgcgagtgttgccagaaaaactgagtgctcgaacgattttggactacgccccaaaggtcaatcggacggtcgttgtcgtgaacactgtgcagcgtgcactcggtctgtatgagcaggtgcgagatgaattccggtgtccggtgattctagcgcactcgcgcttctacgacgaagaccggcggaccaaagagcaacagatcgaagcactgtttgggaagaaagcagcgcaaggtcggtgtctgctgatcgccactcaggtcgtggaggttggactggacatttcctgcgaccttctgattacagaactagctcccgtagacgccctggtgcagcgtgctgggcgctgcgcccgctggggagggaagggtgacgtcattgttcttacggagctcgacacgaagagaccctacgacgagaccctagtggccgtgacggagcgagccctccaagagcacaacgtggacggccaggaactgacatgggaagttgaaacagcccttgtcgacacagtgcttgatccccatttcaaggaatgggcaaagccggatgcggctggcaaggttttagcatcgttggctgaggctgccttcacaggaaactcaacgaaggcagaacaagcggtgcgcgaaaccctgaccgtcgaggtcgctctacacgatactccccaggccttggggccagctatcctcagacttccccgatgccgcttgcatccaggggttttccaacagttcgtccgcaagcagaggcctaatgtttggcaagtggtcgtagatcgagaccctgacgatgactatcgaaccagaatcgagtttctatctgtgaacgggaaatcgagactgatacctggcggccactatatcgttgacccgcagtttggttgttacgatgccgagcgcggtttgcgtctcggggtccccggccaatcagcagagccgtttgccccgggacaatcgagagaccgattgaaaggtgagctgcaaatcgaactgtggcaggaccacataagagaagtcgtcaaggccttcgaaaggtatgtgcttcccaaggagcgaatggcttttgaagccttgagtcgatggttggggaagactcaagacgaactgttaagcgtcgctagggctgttctcgttttacacgaccttggcaagctcgcccggcaatggcaaggaaagattcaggcgggactggaaggcaaactttctcaggggtcttttctggcacaccgcgggggttccgtcagcggactcccaccccatgcgactgtctccgcttgggttgcgaccccctgcctccgccgtctggctggtactgactgggagcagacgttggcggtgccggccttagcggcgattgcccaccaccacagcgtgcgagccgatattactcctgaatttgagatgaccgatggatgcttcgaagtagtcgcagactgcgcgcgaggcgtcgctggccttgaggttaaacgcgacgatttcaatacgaaaccaccgcaaggcagcggctcctgtggggttggtttgaactttctgttgcctgagggctacacgtcctacgtgttgctttcccgatggctccgcttggcggaccgaatcgccacgggaggtggggaagaaacgatctttcaatatgagaaatggatgggcgattcctaagggacaggttaattattgcgagccttgccgctaggatgagagcatgaccggctgccaagtgcgtatgcttctagatctcgcttgggatgtcagcgcacgtgtcgataatgagcgtgttgttcagtggacgcagctccagggattgtcgcctctagatgttggcttccgagtgagaattgctgagaagttcatctgcggccgacttctgatagttcccaagaccctgcctttgtacgcaaagaatttcttgcggctatgcgaaggcgaaaagagacgttttgaccagttgggggagccactccccagcctgaatgtcgttgaccagcgccggtacattgaaaaactagtttggctcggtggtttggactcaaggggaaaacgctatgtggatcaactgtatggtttcagtccattcaccgcgctgaagcaagtcgaattggctgagcaagagcatctaagccctcccgcgatcgccaagagagtgagtgcaatcaacaggagattggtcgctgctgctcacctaagccagcttcagcctatcgaacgcctgtatgccttcttgcgttaggagtcgaagctttggagtacagacttataaaaagcggattggaaatgttcgatacggctcgagcttacgggttggctcaacttctgcaagtgctggccggaggaagggccgcgccgagaatcctgagtcaaggaggtgtttttacgctgaccatttcgacaaagccaaatcccgccactttgaagagctcagacctttggcgcggcgcatttggcgagagcaactggcaaaaggtcttcctaacctacaagagggcttggtcaagtcagcgcgacaaggtgaagaggagcttggaaagccactcggcagatatttttggtaaggccgagacagacggactcgcagtggtcttcggtggcaatttcgcattgccggggccgttggacccagtcggattcaaaggactgaaaggcctgacggcaggtagctattcggaaggtcaaaccaccgtagatgaattcaattgggctttgggttgcttgggtgctgccgcggcccagcggtacaagatccaaaaggctgtaggcaataagtgggaatactacgtgaccctgcctgttccggaggaagtccaatttggtgacttccatgcagttcggcagctagtctatgacaagggactcagctacaacggcgtacgcaacgccgccgctcacttttctctcctcttggcgagcgccatccgtgaaaaagcgcaaggcaacccgcactttccagtgcggttttcaaacgtattgtacttctctctttttcaatccggccagcaattcaagcccgctattggaggcgctgtgaatgtagggagacttatcgagatcgctctggctcggccagaggtggcgttggagatgttcaagacttgggattacttgtttcgccggggcagtgctcaagggaacgaagacttagctcaggccattacggaactggtgatggcaccttcgctcgatacttattaccgccacgcacgaatctttaatcgctatgtagtggattcgacgaagcgcgtaagacccgagtatttgtacgacgaaaccgcactgaaggaggtgctcaactatgctgagcagtgactcgaaattgtcagaggtgttcgcagaagagagcgtcaagtcctttggaaaatgcttgcgatacgcactctggcgtgacgaagactacgcttctctcatagagttcgagaatgcagaaactccaactcaatttgccgatgcggtgaggaagtttcttcgaagatatcggagtggcggcttcatggatcaaactcagcgaagccgggcttccgagatgcgcaagcaaaaccgctgggatggacttaaaaagcttcttcggcagtacgaagtgggacctcgacccagtgaggggcagcttgagaggttgatgcaactggcgaatgacactaacggagtgcgattagttcagtcggccatcatttcctatggactcaccaagagggagccatataaggaagttgaggaactggagaaggagaactgaaatggcagacagcccagtttttgaggtcgcgattcttggacgtgtagtttggaacctacattcactgaataacgaaggcactgtgggcaatgtcagtgagccgcgcacagttgttttggcggacgggtcgaagtccgatggcatctcgggggagatgctcaagcatatccatgcgcagaacgtctggctggtcgcggaagacaagagccaactctgcgaaccgtgcagaacactgaaccctcagaaagctgacaagaatccggctgtactaggagttaagactgcgaaagcgaaagtagcagcagaaagcatgagcgtcgcaatctccagttgtgcgctgtgcgatttacacggcttcttggttcaaaggcccacgatagcgcgggcctctacagtggaatttgggtgggctgtagctctgcgtgacgggtaccatcgggatatccatcttcatgcgcgtcacgctgttgaggggcgcgccgaaacgaccgaaggtcagcaagagggaccagccgaagtatccggccagatgatttatcaccggcccacgcgttcgggtacctatgccttcgtcagtgtcttccaaccctggcgaatcggcttgaacgaggtgaactacgaatacgttgagggtgtcgatcgggaggcgcgcaataagctggcaatcgaagcttataaggcaacttttgcgcgcactggtggcgcaatgacttccactcggctgccgcacgtggaagctcttgagggtgttgtgttggtttccagtcgcaacttcccggtccctgtgacgagccctcttcaggatgactatcgagagaaaactgaaaaagtcgggcaagcggtagaggggttagaggttcagcgtttcggtgctctgccggaactttacgtcatcttaaatgcgcttgcaaaacgacgtttgttcgcgttgcaaatgggaggaacgtcgaagaaaggaaaacagtaaaccatggccgagtggctccaagctgaagttgagttcgccagtttctacagctaccgagtgcctgacctttctccgagtttcgcgctatgctccccggtgccgagcccggcagctatccggctggcagtcgtagacgctactattcggcacactggagatgttaacgaaggccatgcagtttttgagttaatgaagcgagccagattggaacttcaaccaccaagccgtgtcgctgtgatgaagttttttataaagcgactgaagccagagaagccgaccaagggaaaacgcgcgagtgtgatcgaatcgaccggtattcgagaatattgcttgccatggggcccgatggtcttttggattgagagcgatcagccagaacgcatcgcccaatccctacagtggctgcgccgcctcggaacgaccgactcacttgcgagttgcaccgtgggagcaggcactccaaattttgcgtcctgcattagaccggcgaatggcttgacacttcagaccactaatttcgcgcagcgcccggtctttaccctccacgagctcaaaccagagacgcaattcaatcaggtgaatccgttcgcggacgaaagaccaggtaaaccttttgagaagcggctttatgttcttcccttggttcgagaaaaggttggcgaaaactgggtgatctatcatcacgagccgtttgcggcttgacaaataaaacgaaaggctcagtcgaaagactgggcctttcgttttatctgttgtttgtcggtgaacgctctcctgagtaggacaaatttgacagctagctcagtcctaggtataatgctagcgttccagagccttccccgatgaagaggggactgaaagggtataacaacttcgacgagctctacaaagcttggcgttccagagccttccccgatgaagaggggactgaaagagaaggccatcctgacggatggcctttg

SEQ ID NO: 101 - DNA, 6646 bp, synthetic construct
  source 1..6646: mol_type = other DNA; note = Nucleotide sequence for PAM consumption experiment for Type I-A-2
tttacactttatgcttccggctcgtatgttaggaggtctttatcatgctttccggtgcctttgttgcatcccgcaatgcagagatgctcggacctgcccagtgggaatgtcgtgcccgtacaaggcaatcttcgagccttcccctccgccggaagcagaggctctttccaagaatcaggacatcccgcggccgttcgtgttccgagccccaaagacgcagcagacgcgatttgaaacaggtcagccgttcgaattcgagctggtgcttatcggtcgcgcgctcgatttcctgccatacttcgtgctgtcgtttcgagagctagcggctgaggggttggggctcaatcgcgccaagtgcagtcttgaaagggtcgagcaggtggacctgaccagcgaagcagcagacgcttcgaactacgaggctatggttatctatacggccgaggaccaggttttccgaaatgcggcgacttccgagacgggcgaatggatagggaggcggatacggaaccgctcgacatcccgagataacgattcagtgcagcaggtcagcatccgattttcgacgccaacgttcctgaaggccgatggcgaaatcatccgacagccggagttccaccacgttttcaagcgcctgcgggacaggatcaatgccttgagcacgttttttggtgaggggccaatcgaagcggatttccggggccttggcgagcgcgcggaaaagattcgaacggtttcggcccgcaccgattgggttgagcgtttccgcacgtcgtcaaaaacgaaacaacgccacgaattgtcgggctttgtcggtgaagttacttacgaaggtaacttgaatgagtttttgccctggctcacgctcggtgagctggtgcatgtcggcaagcacacggcatggggaaacggctggatggagctagaacacgaggtgtctcgtggttgcgtgtgacaccttcttcgcgccgatgaccgacttcaatctggctcggcatcagtctgaatgcgctggggcgctcgcgagtggcaaatcggtcatcttgcgggcgccgacaggctcaggaaagtcggaggcggtatggctaccgtttctctctctgcgggggaagacgcttccttgtcggctcattcatacgcttcccatgcgtgctcttgtaaaccaacttgagagccggatgcgaacttatgcgaatggcaggatgagggtggcagccatgcacggccagcggccggagagcgtcttgttttacgcggacgccatattcgcgacgcttgaccaagtggtcacttcttatgcgtgcgctcctctaagtctgagtgtgcgacaaggcaatattccggcaggggccgtcgccggcagctttctggtatttgatgaagtccacactttcgagcctcaccttggcctgcaatcgttgctcgtccttgctgaacgagcccaccagatgggcatcccatttgtgatcatgtcggctactctgccgacgaattttatccgtcgtttgtcggaaagattcggcgcaaccatcgtcgagggcacacgactggaaggcaagaaccgacgacaacgccgcgtcgtactcagggtctcgtcagagaagctgagcatcgagacgattctggagctgacacgaaacgtggagcggacgcttgttgtcgtcaacaccgtgcaacgtgcgcagaacctatatgagcaactcctgggcaaaatcggatgtccagtgattttggcgcactcgcgcttctacgatgacgaccgaaggaccaaggaaaagcagatcgaagcccaatttggcaagacggcggaaggccagtgtctactgatcgccacgcaggttgtggaggtggggttggacatttcctgcgacctcctggtcacggaattagccccaatcgacgccatagtgcagcgagccggccgatgcgcccgctggggtggacagggcgaggttgttgtctttacaggcttggaaacaacgcgaccgtatgatcggaccctcgtggaagcaactgagaaggcgctccgagagaagaacttaaacgggcaggaattgacatgggaaattgagagagctcttgtcgatacggtccttgagccacagttcagcaaatgggccgagccggaggccgctggcaaggtcttggcctcattggctgaggcggccttcactggcgactcagctaaggcagaacgagcggtgcgcgaaggtctgaccgttgaggtcgctctgcacggttcgcctgacaccctcggagttggcgctctgaggctgccgcgttgtcgtatccatccagggggcttccagcagtttgtgcacaaacagcagcccgaagcctggcgagtggttgttgatcgaacggctgcagatgattaccgaactcgggtcgaattcctgcatgtggactcgaactccaaggccgcgccttatggctactacataattcacccgcaatatggctcctatgatgtggagcgtggcttgcgactagggatccgaggttcccctgcccaatctcgcgacgagctgatacagcggaagagccgactggagggtgaactgcaaattgaaaagtggcaggaccacattgaaaaggtggtgaaagcattcgccgaacacgtcctgccaaaggagagaatcgctttcgaggccctgtcccggcgacttgggaaaactcatgaagacttgctgtccctgactcaccttgtcttgatcttccacgaccttggcaaactggcacagcagtggcaacgaaagatccaggcaggattggaaagcgtccttcctccggggaccttcttagctcatcggggaggctcgctcagggacctgccacctcacgcaactgtttccgcttcgctggcgacgccctgtctatgccgtgtggctggacctgactggcagcagaccttggcgatacccgccttggccgcgatcgcgcaccaccatagcgtgcgcgcagacatgactccgcaattcgatatgagtgaagggtggttcgacgttgtcgccgactgtgcgcggcggttggctggggttgacgtcaccgttaacgatttcagtagatggcgaggagggggcagttgcggagttgctctcaacttcctgctgcctgatgggtacacatcttacattttgctgtcccggtggcttcgcctggctgatcggatcgcgactggcggtggcgaaaatgccattctgaattatgaagactggatgtcatccagttaagtgcgtcgtggaggagggtctttttttgtggagtacctagtcgtaaaaagcggtctgcccacccttgatgcggccagagcgtatggactagctcaactcctccaagtcctcgccaacggcaaagcctcaccctatattaccgaccagggtggtgtctttgccgtgagcctgaacgcagagctcactcatgatgcgctcacacgttcggatatgtggcgcgcggcgtttgctgatagcaactggcaaagagtgttcttgacgtacaagaaggcttggtccgcgcaacgggaccgggtcaagcgcacattggaggagcaggttgccgctgttgtcacccgcgcaggggacgggctttgcgtcgattttgctggaaagttcgcgctgcccggtcctctcgatcctgtgggattcaagggtctcaagggtctgacggccggaaattactcggaaggtcagacatatctcgacgagcagaatggagccctggcctgtttaggtgcgaccatcgcccagcgctacaaattcggcaaacgagagtacttcgtaacgctacccattccgcaaatggtacagttcaatgacttccatcagattcggcatttggtctacgacaaggggctggcttatctgggagtccgtactgccgcagcacactttgcgcttattttcgctgatgccattcgggaacgggccgcaggaaacccctattttccgctcagcttttcgaatgtgctttatttttcgttattccagtcaggtcaacagtttaagccttcggtgggcggcagtatcaatttggcgcgactcctcgatattgccctgtctcgaccacaagcagcggcggaaatgtttaagacctgggactatttgttccggcgaggcagcgtaaaaggcaatgaggccttggccgaggcaattaccgatttactgatggctccttcgggtgagagctattaccgtcacgcccgcattttcaaccgttacatagttgactcaagcaagcgcgtcaactccgagttcttgtacgacgaagcagcattgatggaggtgatggcttatgtcgaacagtgagatctccctggcttcggtttttgccgaggagagcattaagtcattcggcaagtgcctgcgatacgcgctctggcgagacgacgactacgcctcgctcatcgaattcgagaacgccgaaactccgcttcaatttgcggaagccgtgagaaagtttctccgacgatatcgcagtggcggttttatggatgaggctttgcgcacccaagcgtcagagatgcgtaagcacaaccgctgggacgagctcagaaggacgcttcggcagaacgaaattggcccgagacctacagagggaaatctggagcgcttgacgcaattggcaaacaacgcccagggcgtccggctcgtcagagcggcgattatttcttacggattgaccaaacgcgatccgcacaaagaacttgaggaagtggagagggggagctgatatggcagggaactcagtttttgagatttcgattttggggcgcagcgtctggaacctgcattcattgaacaatgaaggcaccgttgggaatgtcagcgaaccacgtaccgtaatcttagcagacggttcaaaatccgacggcatttcaggcgagatgctcaagcacatacacgcccagaatgtgtggttggtggccactgacaggtctgtcttttgtgagccctgccagacgctccagcctcagaaggctgacaagaacccggacgtcacaggtgttaaggcggcaagagccaaactggcctcagaaggtatgaatgtggccatcgctgcctgtgcgctatgcgatttgcacggattcttggtccaaaagcccacgatagctcgtgcctcaacggttgagtttggatgggctgtagcagtgcgtaacgggtttcaccgcgacatacatcttcacgcgcgccatgctgttgaggggcgcacagaaggtcaacaagaggcgggagaagtggccgcacagatgatttatcaccgcccgacgcgttctggcacctacgcgttggccagtgtttttcagccctggcgaatcggattgaatgaagtcaattacgaatacgtcgcaggagttgatcgggaggcccgctacagactcgctatcgaagcctataaagctacttttgcgcgcacggatggcgctatgacctctacccgtctcccccaccccgaggcttttgagggggtggtgcttgtttcgagtcgcaactttcctgttccggtaacgagccccctccaggatgactatcgtgagaagttacagcagttgtccagagccaccgaaggactggagccgcagccattcaattcgctgacggaactgtatgggatattgaacgaactcgccaaaaggccgctgttcaatttgcaacttgcccgctcctcgaagcgagagaagaagtgaaaaatggctgagtggatccaggccgaaatcgagttcgccagcttttacagctaccgggttccggacctgtctcccagctatgccctctcctcgctagtgccaagcccagcagcgattcggcttgccgtcgtggacgcggtgatccggcacaccggtgttgtggacgaaggcgaatcgatctttgagctggtaaagcgggccaaattggaagttcagccgccggctcgtattgccgtaatgaaattcttcgtcaaacgactgaagccggagaaccccgaaaagggcaagcgcgctagcgtgatcgagtccaccggcatacgagagtattgtttgccctccgggcccctcgtgctatggctagagacggaggaacccgagcgcattggtcaggcccttcagtggttgcgccgcctcggtaccagcgactcacttgcaacttgcaagattggccatggggccccggacaccgcgctgtgcattaagcctgcaaatgggctggctattcaggcgaagaattttgcgcagcgggcagtgttcacgctccacgaattgaaacccgacgcgaatttttccgaagtaaatccgtttgccgatggcaggcgcggcgatccttttgagaagcgtctgtatgtattgccatgtgttcgcgagcaagctggagagaattgggttctttatcggcgtgagccttttgctaactgacaaataaaacgaaaggctcagtcgaaagactgggcctttcgttttatctgttgtttgtcggtgaacgctctcctgagtaggacaaatttgacagctagctcagtcctaggtataatgctagcgttgaagagccttccccgatgaagaggggactgaaagggtataacaacttcgacgagctctacaaagcttggcggttgaagagccttccccgatgaagaggggactgaaagagaaggccatcctgacggatggcctttg

SEQ ID NO: 102 - DNA, 6339 bp, synthetic construct
  source 1..6339: mol_type = other DNA; note = Nucleotide sequence for PAM consumption experiment for Type I-A-3
tttacactttatgcttccggctcgtatgttaggaggtctttatcatgaggctgaagatctccctgacctccaacaacggcaactacctgatcccgtacaactacaaccacatcctgtccgccatcatctacaggaagatcgccgacctggacctggccgctaagctgcatttctccaaggacttcaagttcttcaccttctcccagatctacttctccgactggaagcgcaccaagaatggcatcatcagcaaggacggcaagctgagcttctacatctcctcccccaatgagcagctgatcaagtctctggtcgagggccatctggagaatacagaagtggattttaaagggaaaaaattgctggtggaacagattgagcttctaaaaagtccctcgtttaaggaaaacataaagctgaaaactatgtctccagtagcagccagcataaaaagagaagttgatggaaaacttaagatatgggatttaggacctggagacgaacgattctacgagagcgttcagaaaaatttggtgaataagtacacttccttctatggagattatgacggtgacaaatgggtaaggataaaacccgatatgaaaacagctaaaaggcgtagaattgagataaaaggagactttcaccgcggatatatgatggaatttgagatggaagctgatcctcgccttgtggaatttgcttatgactgtggactgggtgaaaagaatagtatggggtttgggatggtaaatatttatgaataaactgtttaaaaaattaataggagctaaaccatacgattatcagaaaatagctatggagaatttgcttgatggtaaatcaataataatgagagccccaacaggttcaggaaaaactgaaatagctttgattccttttctttatggattcaatgatttattaccttctcaattgatttattctctcccaacaagaactttggttgagagtattggtgaaagggctgttaaatatgcttcatttaggaaactaagagtggctattcaccatggaaaaaatgcaactagtagtctttttgaagaggacgtagtagtaactacaattgatcaagcggtaggcgcttatttgagcacgccactcagcatgtctaaaaggtctggtaacatattcgttggcagtgtaggttctgctttaacagtatttgatgaagttcacacacttgatccggaaaaaggacttcaaactagtttggctattagtatgcaatcggctaaacttggtttacctacactaataatgtccgctacattaccagatatttttatagaaactgctaaagataggatttcaaaaaaaggaggagatattgagtttatagatgtaaaagatgaattcgaaatcaaatcaagaaaaaatagatttgttgaattaataaatagacttgaagaagaattaaatgcagaaaaagtattagaagaagttgagcacggaaaaagaattataattgttattaacaccgtcaatagggctcaagaattgtatttagagttgagaaataaaacagaattacctatacttcttttacattcccgatttcttgagaaggatcgacaagagaaagaattactacttgaagaaacgtttggaaaaaatggcaatggcaaatgtattttcatcgcaactcaaattgttgaggtaggaatggatatttcatcacctaaagttttatcagagatagctcctatagatgctttgatacaaagagcaggaagatgtgccaggtggagcgggaaaggggaatttcatgtttttgggtacaatacaaactcaaaaagtccacacgcaccttataacaaagacattgtagaggctacaaaatcagaaatcaacaacaaagggaaaagtttcactctcgactggaatactgaggttgaactagtaaataaaattttaactaaacatttttcagaatttatgaattcaatgatattttatcaaaggttaggtgaacttgcaagagcggtttatgaaggtagtagggcaaaagtggaacaaaacgttagagaagttttctcctgcgatgttacactgcatgagaatcctaaatccatgaattctgttgaaatcctacatttaccccgactaagacttgacgctagaactttaatgggaaaggtagaaaaaattgctgaaatgggaattgacacatacagattagaggaaaatacaataatttttgatgatgatgaagatgaatacgtacctgttctggttaataatcgtgaagaaataattccgtttgagttatacgtattatgtggtgctagttattcatcagatacaggtttagtttttgatgatttcccaaatgcattaaaatcatttgatcctgaagaaaaagaaattttatccagtaaacagtttgataataggcttaaagttgaaacttgggttgaacatgcaaaaaatacgttaaaagttcttgataattatatgattcctagatatcgttattctatagagaattttgctgaaaattatggctataactatggtgagtttttggatattattaggtgtacggtgtcattgcacgatattggaaaattgaacaaaaaatggcaaaaaagaataaaatggaatgatgaaactcctttagctcattctaacgacaatacaattaaaaggctaccagcgcatgctactgtttccgccaaagcattacaaccatatttagaagatctatttgatgatgaagatatattcaaagccttttatctagctatcgctcatcaccatcaaccttggtcaaaatcatataatgaatatgaactagttccaaaatatgatgaatccctaaaggagatttggattattcctaaaaattttatacaagaacaaaatccagccggtaggcttgatttttcatatttagatattatcgatgaaaatgaagcttatagactatatggttttctttctaagttaatgagaatttcagatagacttgcaacgggaggtaatacttatgaatcattattttctggctaagagcggttgggaattttttgatgtttcaaaagcctatggactgggattagttatacaaacattaactggcaatgcttctataactgatcgagggggattttatttgattgaatcaaaaaatgaaactaagtttgataaaattgaagaaatatccaaatatttcgatgattcagaacttaaaactacattaataactattcaacgttctacaaaatcagaaatgaaacctccagttaaaaaagttaagggaaaatgcttggaaactttgactgataaagaaagcatgattacggtgattaagaactatgaaaatttgaactcaccttcgattataggcacagataaacagacattataccaaactatggatttagctgctacaaagggcattagaaatgaaattctgttaaagaagaattattcagacggaacaaacattaaaatttcagataaagattttgctttgtctcttttaggtcatattaattttactataaaaaaattctccgattttggattgattttagttgcacctacgccacttaaaacagaattaaagaatgtaaggcaaatttatgcaaatttaaaaggtaatgtaaaagtagcgcataaggcaggatggttccctactatcactcaaatagcaataaatttagtttcagaagaaatcatggttaaggatggtggaaagttcgccccaaaatttggttcattaatatatagcattatgaggaaaacagggaatcaatggaagccatctactgggggtattttccctctcgactttttacatcagatagcagattcagataatgcaataaacattttgaataaatggaagaagatatttggatggacatcacggaaaaatggccatgaggatttaccgacaagtctagcagagttcattgccaatccaaatttatttaattatcaaagatatgttaattttcacctcagaaatgaaattgataaagataatatcaaatttggtgattataaaaaagaagattttctggaagtgatgaaaaatgtcggaatttagattgaaagatgtatttgaacacgaatctataaagagtttcggaaagactctaagaaaaatgattaggcctccaaaagaaggaaataaggaaaaatgggcttcagactatgcttccatagtggaattggggtatgtggaaacaaaagaccagtttgcagaagtgattaagaaattattaagaagatatgatgtgatagcaaaaaaacatcaacttaaacgtcccacagaaaaaaatttagaagaattgatggaattaattgataaatacggtgtaaaacctgttagagctgcccttatcagttatgctcttgttaaaaaagatgaagaataaattaggagatgatatgatggtgaatgaaacagaaatttatgaaattgctattttgggaagagcaacatggcaattacacagcctaaataatgagggaactgttggaaatgttacggaacctcgaagtgttacaatcattgacccaaataccaagaatccaataacaaccgacggaatttctggagaaatgctaaaacatatccatacggggctgatgtggactttaacagataaaaataatctctgtgacgcatgtaaggtgttaaaccctgagaaatttaatgtaacatctggaaggggcagtactgttgaagaggttttagaaaacgctttaaataaatgcgatatctgcgatttacatggatttcttattacaaggccaactgtatccagaaaatcaaccatagaatttggttgggccttaggaatacctgaaatttatagagatattcatacacatgcaagacacgcgcttggtggaaaaacgactgaaaatgaagaatctaaaggtgtaaacaccccaaattcttctgaagataaagaagaagctgtcggcacttcaactcaaatggtttatcatcgtcctacacgctctggtgtttatgcagttatttcaatgtttcaaccttggagaataggattaaatgaaacaagacaagatcaatacacttacgatacgggaaataatgaaaagcgaattgaaagatataaaaatgcattgaaagcatatcaaattcttttcaccagacctaaaggtgcaatgagtactactaggttacctcatgtcgaagattttgaaggcgtaatcgttttctcgacggatcaaattcctttacccttaatatcaccacttaaacaggattacgttaaagaaataacagatatttccaaaaaaattgacaattcaataaatgtcgaagaattcaaaactctttctgagtttgtagacaaaataggagatttaattgacaaaaaaccgtacaaattaaagttaggtgaataataatgcagtggttaaaatttactctgcattttccatcatttttctcttatagaatacctgactactcttcacaatatgctttagggattccattaccctcaccttcaaccttgaagttgggagtaatttcatcagctataaaatcaactgggaaagttagtgaaggtgaaaaagtatttaacgttgtgaaagacgcagaagtatgtgttgccccaccagaaaagattgcaattaattcatttttaataaaaagattaaaaaagagaaaagaagatttaaaactaatacccacatttggaattagagattacgttttcttccctgatgatattgatatatttgttggaagtgaaaatattgattctgtggccgaatatttcagcaaaatgaactatataggctctagtgattcaatggtttatgtgaaatccatcgaacctaaaaccccctctgaaaatgtgattaaagctgtggatattgatgaattttcggatgctgcagaaaaagagtcatatcttgtttatccagtaaaagacattaataaaaatgcaacttttgaccaaataaattcttattccagcaaatctagtcgtaaaattttagatcagaaatattatcttatcaatgcaaaagtgagtaaaggcaaaaactggaaaatacttgatacccgaaactaacaaataaaacgaaaggctcagtcgaaagactgggcctttcgttttatctgttgtttgtcggtgaacgctctcctgagtaggacaaatttgacagctagctcagtcctaggtataatgctagcgctcaaatcagactattttaggattgaaatggtataacaacttcgacgagctctacaaagcttggcggctcaaatcagactattttaggattgaaatagaaggccatcctgacggatggcctttg

SEQ ID NO: 103 - DNA, 10862 bp, synthetic construct
  source 1..10862: mol_type = other DNA; note = Nucleotide sequence for PAM consumption experiment for Type I-A-4
tttacactttatgcttccggctcgtatgttaggaggtctttatcatgagaataaaccttcaaggaacaataatagaaggtcaatcatcaataaagacaaattataaccatgaaatgtacagtatgatattaacaaatattagtacagaaagagcaaattatatacacgaaaagaaaagattcaaaagattatttacattttcaaatttatacataagtgataataaagttcatttttatgtatctgggcaagacgagttaattaaagattttataaattgtattatgtttaatcaaatggttagagtaggtgatagagttattagtatcacaaacatagaaccaatgaaaaatagcttagaaactaaaaaggaatatatttttaaaagtaatttcatagtaaatcaaaaagaaaacgatagagtatgtttatcaaaagatatgggatatgtcatgaagagaatttcagacattgtaaaagataaatataaagaaatttataaagaagaaataaatgagaatttaaatgttgaaatacttaatagtaaacaaaaatatactaaatataaagaccatcatttaaactcatatcaagcaacattaaaggtaaggggtaataaaaagctaatagatttattatataacgtaggaattggggagaatacagctagtggtcatggttttgtttgggaggtatcctaatgaatgaatatgaattcaaagtgattaaaactgctaatgatatagaagatatatgtattagttatgggatatgtaagatattatcggataatagaattaaatttaaactaaaagataataaaagtatgtatagtatttatacaaaagaatttgatatacaaaacgatattttttataatgatttcaatattgaaaatgtatggaatttaaatagtggattgaatcagaaagaaaccgtaagagcattagacgatatgaataagtttttgtctgagaatatacatgatatattagaacatttacttaatggcaaagttttaaattataaaaaagaaagtgcaaagggcataggaaattgtttttattcgctaggtgtgagagcttctacttttggtaaaacattagagataagtcctattaaaaaatatttatcctttttgggatggatatatggatgttcttattgttataaagaaaaaagttttgaaattactgcaatattaaaaccttataatactgatgaaatagcaaaaccttttaatttttcatatgtagataaagaaacaggagataagaaaatattaaccaaaataaaaaaagcgtctgaaataaatatgatgtcaatactttatattgaaactttaaagaaatataaaatgttatcagatgaatatagtaatgtaatattcatgcaaaatataatagctgggcaaaaaccattatatgataagacaacaaatattaaaatatataaattatctcaaaaatatctagatgatttattaaagaaattaacttggagcaatgtatcggaagatgtaaaagatattactgctagatatgttttaaatattgataaatataaagaattttcaaaactaataaaaatatatagtaaagatggcaattcaaaaattaataatgattttaaaggagagatattaagtatgtataatgaaatgattaagaaaatttataatgatgaaactattaataaaataggtaaaggattcaataggttattaagagataataaaggttttgaaatccaaacaaaactatataatgttgcaaatgaaaaacatttagtaaaggtacttaaaatgataattgacttgtattcaaggaattataaaagtgcaatattaaataatgacgaattaaataagttgataaatacaattgaagataaagagtatgcaaaaatatgttcagatgcaatattatcaataggaaaagtatttataattattaaaaaataaattgtataaaccatataataaattaaaataatgagtgaaagaggtaataaaaatgaataaaatagcaatgatgatgagattaaaattaactggagaagctttaaacaatgaaggaacaataggaaatgtaatacaacctagacaaatagaatttccaaatggagaagtaagacaagcaataagtggagaaatgttaaagcactatcatagtagaaatttaagactattagctgatgaaaatgaactatgtgatacttgtaaaatatttagtcctatgaaaaatggaaaggttaaagaatctgatagcaaattaagtcctagcggaaacaaggttaaagaatgtatagtagatgatgttgaagggtttatgaacgctggaaaaggtgcaaacgaaaaaagaacaagttgtgttaaattctcatatgcaattgcaactgaagaaaatgaatatcaaataatgttacatactagagtagatgtaacacaagataataataagaaaaaacaagaaaaagaaactacggagggcgaaggtaacaccaataaagaccaaaatactcaaatgttatttcatagacctttaagaaataatgagtatgctataactgtacaagttgatttagatagaattggatttgatgatgaaaaattaatatacgcactagatgaggatactattaaatcaagacaagaaaaatgtattaaagcattattaaatatgtttgttgatatggaaggtgctatgtgttcaactagattaccacatattgaaggaattgagggaataatagttaagaaaactgataagaatcaagtgttaagtaaatatagtgccttgaaagatgactacaaagaagtaaatgaaaagatttcagatgatagtattatttttaataacattatagagttttcagaagttatgaaaggattaatataatttaccaagtagatttaataaactcagtagaatagctattgttgaaaagttgtaactgttggtaaaatattataataatggcttaaatactagagttatgggaaatatataaataagataatagctattttacaatgattaagatttaacaatcatgttatttaaaccgtataaatgtataatacaatactatttacgaaaatattaagatttaacaatcatgttatttaaactgacagctttagtttattagacaccaaaactggaaaattaagatttaacaatcgtgttatttaaacttaaagaaatttggattagatatggataaattatattaagattaaacaataatgttatttaaactcaataatctgtctttcactcctgctttttagttttattaagattaaacattaatgttattaaaacggcttaaacgttatggtatgaatagctttattactattaatattaaacaatagtgttatttaaatatttaacaagagcaacaatcaatcatgtttatatgaacattaatattaaacaatcatgttattaaaaattaatttttcctattttatctaatgtcatatttaattaatattaaacaatatgttattcaaattatggaggcaattacataaaaaaaggtgaaatgggattaatatttaacaataatgttataaacaaaataaataaagtgaggtaaataaaagatgaaaaaagtaacatataaactaagtaatatattctcattaaaaaaatacaatgataataatttaaactgtcaatcttacgaatatccaactatatacggaattagatgtgcaatattaggtgcaataattcaagttgatggaattgataaagttcaagaattattcaacaaaattaaaaattcaaatatttatattcaatatcctaaagagtttaaagttaatgggataaaacaaaaaagatatgcaaattcatattataactcttgttatacagaggaggaatacaataaattatcaccaagtactcaaagtaaaacatattgtgtattagatagagataaattagtaggttcaaactggaaaacaactatgggatttagacaatatgtaaaaatggataatatagtattttacatagataatttaatccctgagattgatatgtatttaaagaatattgattggttaggaactgctaagagtatggtttatttaagtgatgtagaagaagttaataaattagataacgttttaactagatggaataaagaatcctatgtagacacttttgaacaacatgattggaatagtaaaactacctttgatacaatttatatgtattctaaaaaatataaacactttcatgatacttttatgtgtggcattggagatataattctaccaagctgattgtgatataccagatatacgttcattctttattttaagctttggttggtaaacttatatgagaattaggctactataagagttttaaagattgttataaaataagaatgggtaatttactaagattaaaattaaatatagtattatttaaacttctctccaagagcaaatatcgcattatcataagattaaaattcaatatagtattatttaaacctaaaagatttatattgtcttattaatgtattaattattgacattgaatatggtattatttaaatccctcaaaggattcgatttcttctttttctttttcattaaaattaaatatagtattatttaaatagaaagttgtaactaaaatcaactctatttctccatattaaaattcaatataatattatttaaacactccacgaatggttgtgatagatgattatacaaattaaaattaaatataatattatttaaattacgcatcttgtagcctatgcatttgattatatatagattaatattaaacaattatgttattaaaatgttatgtcacaacttaaaatttccatgaatatattaatattaaacattattgttatttaaataataagataagactaaagaagataagacttatataatattaaacttatatattactattatataataacatattaaagaaaggaataaaaataatgaaatataaagaaatatttgaaaaactaaaattaaataacttaacagaagtacaacaaaaaataagtgaattagaagggagtaagaatatattagtagtatcttcatgtggaagtggaaaaactgaggctagttattttaaaatgctagaatacaatagaaaaacaataatcatagagcctatgaaaactttaactaattcaatacatggaagagtagatatatataataaaaaattaggattagaaaaagtatcaatacaacatagttcgtcccaagaggatagatttctacagaataaatatacagttactacaatagaccaagttcttgtaggatatctagctatgggaaagcaagcatacataaaaggtaaaaacatagtaatgagtaatttaatatttgatgaagtgcaattatttgatacagatacaatgctattaactactataaatatgttagatgagatatataaattaggaaataaatttataataatgacagctactatgccacaatttttaattgagtttcttggagaaagatatgatatggaaattgtaattactgaaaaaattagagaagatagaaatgtaaaattattttatgaagaagaactagattataataaagtaaggaattataaagataaacaaattataatatgtaattcaataaagcaattaaaagagatacataaaaaacttcctaatagtagagttattacattacatagcacatttttaggtagtaacagattaaaattagaaaaacaagtggaaagatatttcggaaagcattcagaacagaatgataaaatattattaacaacacaaattgttgaagtaggaatggatattagttgtgacagattgtatactacggcatgtaaaattgataatcttgtacaaagagatggcagatgttgtagatggggaggagatggacaagttattgtatttaaaaatgacgataatatatatgaaaaggaattagttgaagaaactattaaatatattaaaaacaatcaaggtatagcttttaactggacaattcaaaaacaatggattaatgaaatattaaatgaatactataagaataaaataaatgaatataatttaagaaaaaataaatttaattttaatggttgtaatagaagtaggttaattagagatattcaaaatataaatgtgatagttgtaaacaaagaagagttcaccaaacaagattttaatagagaatcagtaagcttacatatcaacaaactaaaagaattgtctcaggcaaatgaaatatacatattgaataaaaataagatagaaaaagtaaaatataataaagttgaaataggagatactgtaattataagaggtaaaaattgtagatatgatgacttaggatttagatatgaagaagactcagctaaaaatatgccaaaatgtagagattttcctatgacaaataagtcaaataataatcaatttagagattacatagaagaaacttggatacaccatgcagaaacagtaagagatttaatgtcttatagattaaatcaagagcaatttaatgattatataattattaatggtaaaaagatagccttttatggtggcttacatgacttaggtaagctagatttagaatggtcaagaaaatataagtcggctattccattagctcatttcccttttgtgaaaggttctatgggagaaaaaagaacccatgaactaattagtggagaaatactaaaggagataatagatgatgacattatttataacatgatgattcaacatcataagcgattatacgatgatatagatatagattataaggggatagaatgggaattacataaagatacatataaaatacttactacatatggatttaaagatgatatacaattacaaagtgatgctaaaacattgaaaagaaataatatcatgtctccatgtgataatgaatggactacattattatatttggtaggtacttttatggaatgtgaaattcaagcaattaatgaatacatagataattataagcaagctatataatacataaacaaagcaaataatatattaaaataaaaaacaaggtatataatttcaatattatggtataatatatattaagaggtgaagtatatatgaaagtaaaagaattaatggatattataaatttaaatttaaaagattttaaaaatataaaatatgagtcttctagtgagtatagtggtgtgatacaaaataaatttgaatcaattataaaacaaacagaattaaaatcattattaatatatgaatataatgaaatcagattactaaaaggaggaggaattttatttcaagtcgatgtatgttataaagaagatgcaagaattaaatctccagttaaaagaaaaggcactataaaatcggtaaatatttcactcgatgaggaattagttgtaagtttattagaatttgaatcgtgtgatttaccattatattttcgtaagaaacggataatagataatatagatgactgtaattccgacattaaagacataaaaattgagttattaaaattagaaaacaaaaaattagaatttgaaaaacaattagttcaaatttgtaaaaataaagcaaattaattatcagaaggtagcgataataccttctgataatatacaaaacaataaaatcacattttagaatatacaaaagcaaataaaatctgtaatttattaaaacttaataatatattaaaataaatatttaataaaaacgggtatataatttcaaataaatggtataatagtaatatgaaattatttgaaagaggtgtaatatatgagaaaaattactaaaatagaaattgaaagtcttatgaaatgtttagattattcaaaagatgaatatataggttttactatagttgacaatgatataagattacttcattctaatacaagtaaaggaatttattatcgaatagacggattcattgaaagatggaaaaataataaagatactttaaatgagatgctaggaaaaatacaagactatataaatgatgttatagagctatataatgaagggagtgttaatcatgaatagttataaagaagtttatgaagaatggtttgatagtgaagataatgtgattgtagtatcagaagatgatacaagacaattagtaattggtataattgaaaacattgtttttataggaaaaattataaaatttaataaaacctatgatataaatattattaaacaatttgaactacaatgttcttatccctttaatcaagatagaaaatattttttatatccagtattacaaatgtataaaatattattttatttagtttcttatatgcaaagtaaaaatattatatgcgatataaataatgatggaatagatagttatattttaaaattatctcagcatcctatattgttacaagattttataaataattttagaatactaataaacgagaatattaaaatgaaagatgtagctattttgaaataacttagggcatattaattgcccttttcttatataaaatcataaaaatacaaattctacaactagcttatagccacattctaaaagttgaatattgtgataaaatgacacttttctttggagttaaattattccagttaaaaactacataataaattaaaataatacttgacttatatttgagcgaagtgtatagtattaaatataagggaggtaaagaaatgttaaacaagcctagagaagaatggtcaaaagaagaaatcaatatgtattgcagaaataaagcaattaataaagttaaaaacagattaaaacaacaattgagggaaagggtgatagtgagatgttaagaaaatggttattaaaaaataagctacataaaaatgatttagaatggattgaaattacagaatatgcacttttagattataggcatcaagtacggggaaatgaaagtgaaagtacttggttacttgaaagaaaattaaatcgaaattatcaatttggtataattttagatgaaacagacaaaacaatacataaaaaatatggaatgttaacaataatatatagtaaagaaaatggtaagattatagggataactaatcataggggcaaatatagcaattgtgaaattgataaggaacttaaaaataagttaaataaaatttatggaataagttaggaggaaaataaatggacaagttaaaaataatagttttggtggcaaaatcttcagcaggaaaagataatatattaaataaggtagttgaattaaatccaaaagtaaaaacaatagtatcttatacttcacgtcctattaggcagggagaaattgatggaattacatatcattatatatcaaatgagaaagtaaataatatgttagctaatcaagaatttatagaaaataagatatacaatacagtaaatggtaagtgggtgtatggagttggaaagtcaagctttgacctttattcaaaaaatacttatattgtaatattagatttacaaggactaaaacaattagaaaattatttaaatgaaaataataaattagattgtttaatttcaatatacataaaagcaagtggacaaactagattattaagaagtttgcaacgtgaaggacaattaagtgataatcaatgtaaagagatatgcagaagatttatttcagatgaagaagatatggagtatgccgaaggttattgtaatataactttggtaaatgaagttgaggatgatttaaataagtgcatagaatatgtttaccatttaacaattaattaatggagggataaagcaaatggaattttcaaaagacgaattaaaggagattgctctaagtttaaatttgatttctgctgaacgtaatgcttatttattagatgatagtataaataaatataaagaaaataataataaatacttggaaatggataagatattgcttaataaaattaaattagaaataaaaagattaaaagaaggagtagaagaataatgaataatgagattaaaatagtcaaatgtatagatagtttatatcctacagtaaaactaactattggaaaattatataaagttaaagaatctgagaacgataaattttatagagtaatagctgatgataataatgaagaacaactttgttataaatatagatttgaattagtagatattaatgaaataaaggaattaacattacaagatatttttaatgaagaagaaggtattaaatataatagaataaatggtggaagtggaatatatacaatacaaaacgaaacattaattattggcgagcatattaaaccagttctcaataaaagaataatggattctaaatttgtaaaagtaaaagtagaaagactggtgagtttctcagatgttattaattcagattataaatgtaaagtaaaacattatagagttgaaggattaattcaagaagaaagttcatacacatggcttgaagaatatcaagatttgaaagacataatgttagcattatcggaagaatttaatactattgcattaaaagagataattaataaaggtcagtggtatttagagaattaacaaataaaacgaaaggctcagtcgaaagactgggcctttcgttttatctgttgtttgtcggtgaacgctctcctgagtaggacaaatttgacagctagctcagtcctaggtataatgctagcattaagatttaacaatcatgttatttaaaggtataacaacttcgacgagctctacaaagcttggcattaagatttaacaatcatgttatttaaaagaaggccatcctgacggatggcctttg

SEQ ID NO: 104 - DNA, 45 bp, synthetic construct
  source 1..45: mol_type = other DNA; note = PAM library sequence
nnnnnnnnggtataacaacttcgacgagctctacaaagcttggcg

SEQ ID NO: 105 - RNA, 111 nt, synthetic construct
  source 1..111: mol_type = other RNA; note = Guide RNA sequence in I-A-2 system targeting ROS1 (GRMZM2G422464) gene in Example 4
gttgaagagccttccccgatgaagaggggactgaaaggtaatccgtgtataccagctattgggtcaactgaactgttgaagagccttccccgatgaagaggggactgaaag

SEQ ID NO: 106 - RNA, 96 nt, synthetic construct
  source 1..96: mol_type = other RNA; note = Guide RNA sequence in I-A-3 system targeting ROS1 (GRMZM2G422464) gene in Example 4
gctcaaatcagactattttaggattgaaatgtaatccgtgtataccagctattgggtcaactgaacgctcaaatcagactattttaggattgaaat

SEQ ID NO: 107 - RNA, 183 nt, synthetic construct
  source 1..183: mol_type = other RNA; note = Dual-target guide RNA sequence in I-A-1 system targeting ROS1 (GRMZM2G422464) gene in Example 5
gttccagagccttccccgatgaagaggggactgaaaggaaagggcatggaagaagttgcgatacacagattgagttccagagccttccccgatgaagaggggactgaaaggtgaagaatgattacttgaagttttctaccaatagtgttccagagccttccccgatgaagaggggactgaaag

SEQ ID NO: 108 - RNA, 185 nt, synthetic construct
  source 1..185: mol_type = other RNA; note = Dual-target guide RNA sequence in I-A-2 system targeting ROS1 (GRMZM2G422464) gene in Example 5
gttgaagagccttccccgatgaagaggggactgaaaggaaagggcatggaagaagttgcgatacacagattgatgttgaagagccttccccgatgaagaggggactgaaaggtgaagaatgattacttgaagttttctaccaatagtagttgaagagccttccccgatgaagaggggactgaaag

SEQ ID NO: 109 - RNA, 162 nt, synthetic construct
  source 1..162: mol_type = other RNA; note = Dual-target guide RNA sequence in I-A-3 system targeting ROS1 (GRMZM2G422464) gene in Example 5
gctcaaatcagactattttaggattgaaatgaaagggcatggaagaagttgcgatacacagattgagctcaaatcagactattttaggattgaaatgtgaagaatgattacttgaagttttctaccaatagtgctcaaatcagactattttaggattgaaat

SEQ ID NO: 110 - RNA, 159 nt, synthetic construct
  source 1..159: mol_type = other RNA; note = Dual-target guide RNA sequence in I-A-4 system targeting ROS1 (GRMZM2G422464) gene in Example 5
attaagatttaacaatcatgttatttaaagaaagggcatggaagaagttgcgatacacagattgaattaagatttaacaatcatgttatttaaagtgaagaatgattacttgaagttttctaccaatagtattaagatttaacaatcatgttatttaaa

SEQ ID NO: 111 - DNA, 37 bp, synthetic construct
  source 1..37: mol_type = other DNA; note = Target sequence g in Example 4
gtaatccgtgtataccagctattgggtcaactgaact

SEQ ID NO: 112 - DNA, 37 bp, synthetic construct
  source 1..37: mol_type = other DNA; note = Target sequence g1 in Example 5
gaaagggcatggaagaagttgcgatacacagattgat

SEQ ID NO: 113 - DNA, 37 bp, synthetic construct
  source 1..37: mol_type = other DNA; note = Target sequence g2 in Example 5
gtgaagaatgattacttgaagttttctaccaatagta

SEQ ID NO: 114 - DNA, 17 bp, synthetic construct
  source 1..17: mol_type = other DNA; note = Nucleotide sequence of M13F primer
gtaaaacgacggccagt

SEQ ID NO: 115 - DNA, 17 bp, synthetic construct
  source 1..17: mol_type = other DNA; note = Nucleotide sequence of M13R primer
caggaaacagctatgac

SEQ ID NO: 116 - DNA, 37 bp, synthetic construct
  source 1..37: mol_type = other DNA; note = Target sequence g1 in Example 7
tgccagcggggaggtcaatgctgggagttggggcgcg

SEQ ID NO: 117 - DNA, 37 bp, synthetic construct
  source 1..37: mol_type = other DNA; note = Target sequence g2 in Example 7
gggcgtggagcgcggctactaccgggagttcttcgag

SEQ ID NO: 118 - RNA, 185 nt, synthetic construct
  source 1..185: mol_type = other RNA; note = Dual-target guide RNA sequence in I-A-2 system targeting GA2 (GRMZM2G368411) gene in Example 7
gttgaagagccttccccgatgaagaggggactgaaagtgccagcggggaggtcaatgctgggagttggggcgcggttgaagagccttccccgatgaagaggggactgaaaggggcgtggagcgcggctactaccgggagttcttcgaggttgaagagccttccccgatgaagaggggactgaaag

SEQ ID NO: 119 - DNA, 37 bp, synthetic construct
  source 1..37: mol_type = other DNA; note = Target sequence G1 in Example 8
acgagggcacccagaccgccaagctgaaggtgaccaa

SEQ ID NO: 120 - DNA, 37 bp, synthetic construct
  source 1..37: mol_type = other DNA; note = Target sequence G2 in Example 8
cgccgacatccccgattacaagaagctgtccttcccc

SEQ ID NO: 121 - RNA, 97 nt, synthetic construct
  source 1..97: mol_type = other RNA; note = Guide RNA sequence in I-A-3 system targeting Tdtomato gene G1 target site in Example 8
gctcaaatcagactattttaggattgaaatacgagggcacccagaccgccaagctgaaggtgaccaagctcaaatcagactattttaggattgaaat

SEQ ID NO: 122 - RNA, 97 nt, synthetic construct
  source 1..97: mol_type = other RNA; note = Guide RNA sequence in I-A-3 system targeting Tdtomato gene G2 target site in Example 8
gctcaaatcagactattttaggattgaaatcgccgacatccccgattacaagaagctgtccttccccgctcaaatcagactattttaggattgaaat

SEQ ID NO: 123 - DNA, 37 bp, synthetic construct
  source 1..37: mol_type = other DNA; note = Target sequence g1 in Example 9
cagggcccggccgccacctgccgcgtgggcctgaacc

SEQ ID NO: 124 - DNA, 37 bp, synthetic construct
  source 1..37: mol_type = other DNA; note = Target sequence g2 in Example 9
aagaaagaagcgattctatttcatattaggcattgta

SEQ ID NO: 125 - RNA, 185 nt, synthetic construct
  source 1..185: mol_type = other RNA; note = Dual-target guide RNA sequence in I-A-2 system targeting HPRT1 gene in Example 9
gttgaagagccttccccgatgaagaggggactgaaagcagggcccggccgccacctgccgcgtgggcctgaaccgttgaagagccttccccgatgaagaggggactgaaagaagaaagaagcgattctatttcatattaggcattgtagttgaagagccttccccgatgaagaggggactgaaag

(19) United States
(12) Patent Application Publication
(10) Pub. No.: US 2026/0159821 A1
(43) Pub. Date: Jun. 11, 2026

(54) CRISPR/CAS EFFECTOR PROTEIN AND SYSTEM

(71) Applicant: CHINA ARGRICULTURAL UNIVERSITY, Beijing (CN)

(72) Inventors: Jinsheng Lai, Beijing (CN); Yingying Wang, Beijing (CN); Zhimeng Li, Beijing (CN); Zhijia Yang, Beijing (CN); Jian Chen, Beijing (CN); Beibei Xin, Beijing (CN); Yingnan Li, Beijing (CN); Weibin Song, Beijing (CN); Haiming Zhao, Beijing (CN)

(21) Appl. No.: 19/306,912

(22) Filed: Aug. 21, 2025

Related U.S. Application Data
(63) Continuation of application No. PCT/CN2024/077872, filed on Feb. 21, 2024.

(30) Foreign Application Priority Data
Feb. 21, 2023 (CN) 202310187307.6

(51) Int. Cl. C12N 9/22; C12N 9/78; C12N 15/113; C12N 15/63

(52) U.S. Cl. CPC C12N 9/224; C12N 9/78; C12N 15/113; C12N 15/63; C12Y 305/04001; C12Y 305/04004; C12N 2310/20; C12N 2310/531

(57) ABSTRACT

The present invention relates to the technical field of clustered regularly interspaced short palindromic repeats (CRISPR). In particular, the present invention relates to a Type I-A CRISPR-Cas effector protein and system, a fusion protein comprising the protein, and nucleic acid molecules encoding same. The present invention also relates to a complex and composition for nucleic acid editing (e.g., gene or genome editing, large fragment deletion, single base editing, genomic structural variation), which comprises the protein or fusion protein of the present invention, or nucleic acid molecules encoding same. The present invention further relates to a method for nucleic acid editing (e.g., gene or genome editing, large fragment deletion, single base editing, genomic structural variation), which uses the protein or fusion protein comprised in the present invention.

### CROSS-REFERENCE TO RELATED APPLICATIONS

This application is a continuation of International Application No. PCT/CN2024/077872, filed Feb. 21, 2024, and which claims the benefit of priority to, the Chinese patent application with application number 202310187307.6 (filed on Feb. 21, 2023). The content of the Chinese patent application is incorporated herein in its entirety.

### SEQUENCE LISTING

The instant application contains a Sequence Listing which has been submitted electronically in XML file format and is hereby incorporated by reference in its entirety. Said XML file, created on Aug. 21, 2025, is named IEC232066PUS-Seql.xml and is 209,911 bytes in size.

### TECHNICAL FIELD

The present disclosure relates to the technical field of clustered regularly interspaced short palindromic repeats (CRISPR). Specifically, the present disclosure relates to a Type I-A CRISPR-Cas effector protein and system, a fusion protein comprising the protein, and nucleic acid molecules encoding same. The present disclosure also relates to a complex and composition for nucleic acid editing (e.g., gene or genome editing, large-fragment deletion, single base editing, genomic structural variation), which comprises the protein or fusion protein of the present disclosure, or nucleic acid molecules encoding same. The present disclosure also relates to a method for nucleic acid editing (e.g., gene or genome editing, large-fragment deletion, single base editing, genomic structural variation), using the protein or fusion protein in the present disclosure.

### BACKGROUND ART

CRISPR/Cas is a widely used gene editing technology, in which the target sequence on the genome is specifically bound through the RNA guidance, and DNA is cleaved to produce double-strand breaks, and then site-specific editing of the genome is achieved by repairing the breaks via non-homologous end joining or homologous recombination pathways in an organism. At present, based on the classification of existing CRISPR systems, it can be divided into two categories: Class 1 and Class 2 (Liu and Doudna 2020). Among them, the Class 2 system is mainly composed of a single effector protein, and the widely used CRISPR/Cas9 system belongs to the type II family in the Class 2 system. Although the CRISPR/Cas9 system has became a mature technology in the field of gene editing, its application in large-fragment deletion of genome or chromosome elimination remains highly challenging, as CRISPR/Cas9 primarily generates small-fragment deletions after genome editing.

The Class 1 system is mainly composed of multiple effector proteins. It is currently divided into three families: type I, type II, and type III. The research is relatively mature mainly for the E-type system within the type I family. The Class 1 system is similar to the Class 2 system, where under the guidance of guide RNA, it recognizes the PAM motif and then engages with the target sequence to achieve binding and cleavage of the substrate DNA. The type I-E system is mainly composed of two parts, one is the Cas3 protein with nuclease activity and the Cas5, Cas6, Cas7, Cas8e, and Cas11 proteins that form the Cascade complex. The guide RNA recognizes the substrate DNA and binds to the Cascade complex, and then further recruits the Cas3 protein to cleave the substrate DNA. Currently reported studies utilizing the type I-E system for editing human 293T cells have found that the type I-E system primarily induces long-range, large-fragment deletions in the genome. However, the length of these deletion fragments are random, which imposes limitations on its production applications. At the same time, there are few reports on the technology of using other Class 1 families for eukaryotic genome editing.

Therefore, given the current limitations of the CRISPR/Cas system in generating deletions of specific lengths during genome editing and the random fragment deletions produced by the Type I system, developing a more robust CRISPR/Cas system capable of achieving precise large-fragment deletions of the genome is of significant importance.

### CONTENTS OF THE PRESENT INVENTION

After extensive experimentation and repeated exploration, the inventors of the present application have unexpectedly developed a novel Type I-A CRISPR-Cas system or vector system as well as a method for applying the system, which can be used to achieve precise large-fragment deletion and/or other target nucleic acid editing (e.g., modifying genes, knocking out genes, altering the expression of gene products, repairing mutations, inserting polynucleotides, and/or single-base mutations, etc.) of target genes or genomes.

### I. Type I-A System—Protein Part

In one aspect, the present application provides a Type I-A CRISPR-Cas system, which comprises:
(1) a Cas5a protein or a nucleotide sequence encoding Cas5a protein, wherein the Cas5a protein has an amino acid sequence as set forth in any one of SEQ ID NOs: 2, 8, 14, 20 or ortholog, homolog, variant or functional fragment thereof,
(2) a Cas8a protein or a nucleotide sequence encoding Cas8a protein, wherein the Cas8a protein has an amino acid sequence as set forth in any one of SEQ ID NOs: 3, 9, 15, 21 or ortholog, homolog, variant or functional fragment thereof,
(3) a Cas7 protein or a nucleotide sequence encoding Cas7 protein, wherein the Cas7 protein has an amino acid sequence as set forth in any one of SEQ ID NOs: 4, 10, 16, 22 or ortholog, homolog, variant or functional fragment thereof,
(4) a Cas6 protein or a nucleotide sequence encoding Cas6 protein, wherein the Cas6 protein has an amino acid sequence as set forth in any one of SEQ ID NOs: 5, 11, 17, 23 or ortholog, homolog, variant or functional fragment thereof, and,
(5) a Csa5 protein or a nucleotide sequence encoding Csa5 protein, wherein the Csa5 protein has an amino acid sequence as set forth in any one of SEQ ID NOs: 6, 12, 18, 24 or ortholog, homolog, variant or functional fragment thereof,
wherein, in any one of (1) to (5), the ortholog, homolog, variant or functional fragment substantially retains the biological function of the sequence from which it is derived.

In some embodiments, the ortholog, homolog, variant has a substitution, deletion or addition of one or more amino acids (e.g., a substitution, deletion or addition of 1, 2, 3, 4, 5, 6, 7, 8, 9 or 10 amino acids), or has a sequence identity of at least 80%, at least 85%, at least 90%, at least 91%, at least 92%, at least 93%, at least 94%, at least 95%, at least 96%, at least 97%, at least 98%, or at least 99%, as compared to the sequence from which it is derived, and substantially retains the biological function of the sequence from which it is derived.

In some embodiments, the system further comprises: (6) a Cas3 protein or a nucleotide sequence encoding Cas3 protein, wherein the Cas3 protein has an amino acid sequence as set forth in any one of SEQ ID NOs: 1, 7, 13, 19 or its ortholog, homolog, variant or functional fragment;
wherein the ortholog, homolog, variant or functional fragment substantially retains the biological function of the sequence from which it is derived.

In certain embodiments, the ortholog, homolog, variant has a substitution, deletion or addition of one or more amino acids (e.g., a substitution, deletion or addition of 1, 2, 3, 4, 5, 6, 7, 8, 9 or 10 amino acids), or has a sequence identity of at least 80%, at least 85%, at least 90%, at least 91%, at least 92%, at least 93%, at least 94%, at least 95%, at least 96%, at least 97%, at least 98%, or at least 99%, as compared to the sequence from which it is derived, and substantially retains the biological function of the sequence from which it is derived.

In the present disclosure, the biological function of the above sequence refers to an activity of Cas effector protein, including but not limited to the activity of binding to guide RNA, the endonuclease activity, the activity of site-specific binding and cutting the target sequence or complementary sequence thereof under the guidance of guide RNA.

The protein of the present disclosure can be derivatized, for example, linked to another molecule (e.g., another polypeptide or protein). Generally, the derivatization (e.g., labeling) of a protein does not adversely affect the desired activity of the protein (e.g., the activity of binding to guide RNA, endonuclease activity, activity of site-specific binding and cutting the target sequence or complementary sequence thereof under the guidance of guide RNA). Therefore, the protein of the present disclosure is also intended to include such derivatized forms. For example, the protein of the present disclosure can be functionally linked (by chemical coupling, gene fusion, non-covalent linkage or other means) to one or more other moieties, such as another protein or polypeptide, a detection agent, a pharmaceutical agent, etc.

In particular, the protein of the present disclosure can be linked to an additional functional unit. For example, it can be linked to a nuclear localization signal (NLS) sequence to increase the ability of the protein of the present disclosure to enter the cell nucleus. For example, it can be linked to a targeting moiety to endow the protein of the present disclosure with targeting ability. For example, it can be linked to a detectable label to facilitate detection of the protein of the present disclosure. For example, it can be linked to an epitope tag to facilitate expression, detection, tracing and/or purification of the protein of the present disclosure.

In certain embodiments, any Cas protein in the system optionally comprises an additional protein or polypeptide, wherein the additional protein or polypeptide is selected from the group consisting of epitope tag, reporter gene sequence, nuclear localization signal (NLS) sequence, targeting moiety, transcriptional activation domain (e.g., VP64), transcriptional repression domain (e.g., KRAB domain or SID domain), nuclease domain (e.g., Fok1), adenosine deaminase (e.g., TadA8e), cytosine deaminase (e.g., APOBEC3), a domain having an activity selected from the following: methylase activity, demethylase activity, transcriptional activation activity, transcriptional repression activity, transcript release factor activity, histone modification activity, nuclease activity (e.g., single-stranded RNA cleavage activity, double-stranded RNA cleavage activity, single-stranded DNA cleavage activity, double-stranded DNA cleavage activity) and nucleic acid binding activity; and any combination thereof.

In certain embodiments, at least one Cas protein in the system comprises the additional protein or polypeptide; for example, the protein described in each of (1) to (6) comprises the additional protein or polypeptide.

In some embodiments, the additional protein or polypeptide is an NLS sequence. In some embodiments, the protein described in each of (1) to (6) comprises an NLS sequence.

In some embodiments, the NLS sequence is set forth in SEQ ID NO: 65.

In some embodiments, the NLS sequence is located at or near the terminus (e.g., N-terminus or C-terminus) of the protein.

In some embodiments, the additional protein or polypeptide is an adenosine deaminase (e.g., TadA8e) or a cytosine deaminase (e.g., APOBEC3). In some embodiments, one of the proteins described in any of (1) to (5) comprises an adenosine deaminase (e.g., TadA8e) or a cytosine deaminase (e.g., APOBEC3).

In some embodiments, the amino acid sequence of the adenosine deaminase or cytosine deaminase is located at or near the terminus (e.g., N-terminus or C-terminus) of the protein (e.g., Cas8a protein).

In certain embodiments, the amino acid sequence of the adenosine deaminase or cytosine deaminase is located at or near the N-terminus of the Cas8a protein.

In certain embodiments, the additional protein or polypeptide is connected to the protein through a linker or not through a linker.

In certain embodiments, the linker is a peptide linker or a non-peptide linker.

In certain embodiments, the peptide linker has a sequence as set forth in SEQ ID NO: 66, 67 or 95.

In certain embodiments, the protein of the present disclosure comprises an epitope tag. Such epitope tags are well known to those skilled in the art, and examples thereof include, but are not limited to, His, V5, FLAG, HA, Myc, VSV-G, Trx, etc., and those skilled in the art know how to select a suitable epitope tag according to the desired purpose (e.g., purification, detection or tracing).

In certain embodiments, the protein of the present disclosure comprises a reporter gene sequence. Such reporter genes are well known to those skilled in the art, and examples thereof include, but are not limited to, GST, HRP, CAT, GFP, HcRed, DsRed, CFP, YFP, BFP, etc.

In certain embodiments, the system does not comprise a Cas3 protein or a nucleotide sequence encoding Cas3 protein.

In some embodiments, a Cas protein (e.g., Cas5a protein, Cas8a protein, Cas7 protein, Cas6 protein, or Csa5 protein) in the system comprises an adenosine deaminase (e.g., TadA8e) or a cytosine deaminase (e.g., APOBEC3); for example, the amino acid sequence of the adenosine deaminase or cytosine deaminase is located at or near the terminus (e.g., N-terminus or C-terminus) of the Cas protein.

In some embodiments, the Cas8a protein in the system comprises an adenosine deaminase (e.g., TadA8e) or a cytosine deaminase (e.g., APOBEC3).

In some embodiments, the amino acid sequence of the adenosine deaminase or cytosine deaminase is located at or near the N-terminus of the Cas8a protein.

In some embodiments, the adenosine deaminase or cytosine deaminase is connected to the protein through a linker or not through a linker.

In some embodiments, the linker is a peptide linker or a non-peptide linker; In some embodiments, the peptide linker has a sequence as set forth in SEQ ID NO: 66, 67 or 95; In some embodiments, the peptide linker has a sequence as set forth in SEQ ID NO: 95.

In some embodiments, the Cas8a protein in the system comprises a TadA8e, and comprises a sequence as set forth in any one of SEQ ID NOs: 96-99.

### I-I. Type I-A-1 System

In some embodiments, in the system, the Cas5a protein, Cas8a protein, Cas7 protein, Cas6 protein, and Csa5 protein respectively comprise the amino acid sequences as set forth in SEQ ID NOs: 2-6.

In some embodiments, one or more (e.g., all 5) of the Cas5a protein, Cas8a protein, Cas7 protein, Cas6 protein, and Csa5 protein are connected to an NLS sequence (e.g., a sequence as set forth in SEQ ID NO: 65) through a linker or not through a linker.

In some embodiments, the Cas5a protein, Cas8a protein, Cas7 protein, Cas6 protein, and Csa5 protein connected to the NLS sequence respectively comprise the amino acid sequences as set forth in SEQ ID NOs: 69-73.

In some embodiments, the system further comprises: (6) a Cas3 protein or a nucleotide sequence encoding Cas3 protein;
wherein the Cas3 protein comprises an amino acid sequence as set forth in SEQ ID NO: 1.

In some embodiments, the Cas3 protein is connected to an NLS sequence (e.g., a sequence as set forth in SEQ ID NO: 65) through a linker or not through a linker.

In some embodiments, the Cas3 protein connected to the NLS sequence comprises an amino acid sequence as set forth in SEQ ID NO: 68.

In some embodiments, the system does not comprise a Cas3 protein or a nucleotide sequence encoding Cas3 protein.

In some embodiments, a Cas protein (e.g., Cas5a protein, Cas8a protein, Cas7 protein, Cas6 protein, or Csa5 protein) in the system comprises an adenosine deaminase (e.g., TadA8e) or a cytosine deaminase (e.g., APOBEC3); for example, the amino acid sequence of the adenosine deaminase or cytosine deaminase is located at or near the terminus (e.g., N-terminus or C-terminus) of the Cas protein.

In some embodiments, the Cas8a protein in the system comprises an adenosine deaminase (e.g., TadA8e) or a cytosine deaminase (e.g., APOBEC3).

In some embodiments, the amino acid sequence of the adenosine deaminase or cytosine deaminase is located at or near the N-terminus of the Cas8a protein.

In some embodiments, the adenosine deaminase or cytosine deaminase is connected to the protein through a linker or not through a linker.

For example, the linker is a peptide linker or a non-peptide linker.

For example, the peptide linker has a sequence as set forth in SEQ ID NO: 66, 67 or 95. In some embodiments, the peptide linker has a sequence as set forth in SEQ ID NO: 95.

For example, the Cas8a protein in the system comprises a TadA8e, and comprises a sequence as set forth in SEQ ID NO: 96.

### I-II. Type I-A-2 System

In some embodiments, in the system, the Cas5a protein, Cas8a protein, Cas7 protein, Cas6 protein, and Csa5 protein respectively comprise the amino acid sequences as set forth in SEQ ID NOs: 8-12.

In some embodiments, one or more (e.g., all 5) of the Cas5a protein, Cas8a protein, Cas7 protein, Cas6 protein, and Csa5 protein are connected to an NLS sequence (e.g., a sequence as set forth in SEQ ID NO: 65) through a linker or not through a linker.

In some embodiments, the Cas5a protein, Cas8a protein, Cas7 protein, Cas6 protein, and Csa5 protein connected to the NLS sequence respectively comprises the amino acid sequences as set forth in SEQ ID NOs: 75-79.

In some embodiments, the system further comprises: (6) a Cas3 protein or a nucleotide sequence encoding Cas3 protein;
wherein the Cas3 protein comprises an amino acid sequence as set forth in SEQ ID NO: 7.

In some embodiments, the Cas3 protein is connected to an NLS sequence (e.g., a sequence as set forth in SEQ ID NO: 65) through a linker or not through a linker.

In some embodiments, the Cas3 protein connected to the NLS sequence comprises an amino acid sequence as set forth in SEQ ID NO: 74.

In some embodiments, the system does not comprise a Cas3 protein or a nucleotide sequence encoding Cas3 protein.

In some embodiments, a Cas protein (e.g., Cas5a protein, Cas8a protein, Cas7 protein, Cas6 protein, or Csa5 protein) in the system comprises an adenosine deaminase (e.g., TadA8e) or a cytosine deaminase (e.g., APOBEC3); for example, the amino acid sequence of the adenosine deaminase or cytosine deaminase is located at or near the terminus (e.g., N-terminus or C-terminus) of the Cas protein.

In some embodiments, the Cas8a protein in the system comprises an adenosine deaminase (e.g., TadA8e) or a cytosine deaminase (e.g., APOBEC3).

In some embodiments, the amino acid sequence of the adenosine deaminase or cytosine deaminase is located at or near the N-terminus of the Cas8a protein.

In some embodiments, the adenosine deaminase or cytosine deaminase is connected to the protein through a linker or not through a linker.

In some embodiments, the linker is a peptide linker or a non-peptide linker.

In some embodiments, the peptide linker has a sequence as set forth in SEQ ID NO: 66, 67 or 95; in some embodiments, the peptide linker has a sequence as set forth in SEQ ID NO: 95.

In some embodiments, the Cas8a protein in the system comprises a TadA8e, and comprises the sequence as set forth in SEQ ID NO: 97.

### I-III. Type I-A-3 System

In some embodiments, in the system, the Cas5a protein, Cas8a protein, Cas7 protein, Cas6 protein, and Csa5 protein respectively comprise the amino acid sequences as set forth in SEQ ID NOs: 14-18.

In some embodiments, one or more (e.g., all 5) of the Cas5a protein, Cas8a protein, Cas7 protein, Cas6 protein, and Csa5 protein are connected to an NLS sequence (e.g., the sequence as set forth in SEQ ID NO: 65) through a linker or not through a linker.

In some embodiments, the Cas5a protein, Cas8a protein, Cas7 protein, Cas6 protein, and Csa5 protein connected to the NLS sequence respectively comprise the amino acid sequences as set forth in SEQ ID NOs: 81-85.

In some embodiments, the system further comprises: (6) a Cas3 protein or a nucleotide sequence encoding Cas3 protein;
wherein the Cas3 protein comprises an amino acid sequence as set forth in SEQ ID NO: 13.

In some embodiments, the Cas3 protein is connected to an NLS sequence (e.g., a sequence as set forth in SEQ ID NO: 65) through a linker or not through a linker.

In some embodiments, the Cas3 protein connected to the NLS sequence comprises an amino acid sequence as set forth in SEQ ID NO: 80.

In some embodiments, the system does not comprise a Cas3 protein or a nucleotide sequence encoding Cas3 protein.

In some embodiments, a Cas protein (e.g., Cas5a protein, Cas8a protein, Cas7 protein, Cas6 protein, or Csa5 protein) in the system comprises an adenosine deaminase (e.g., TadA8e) or a cytosine deaminase (e.g., APOBEC3); for example, the amino acid sequence of the adenosine deaminase or cytosine deaminase is located at or near the terminus (e.g., N-terminus or C-terminus) of the Cas protein.

In some embodiments, the Cas8a protein in the system comprises an adenosine deaminase (e.g., TadA8e) or a cytosine deaminase (e.g., APOBEC3).

In some embodiments, the amino acid sequence of the adenosine deaminase or cytosine deaminase is located at or near the N-terminus of the Cas8a protein.

In some embodiments, the adenosine deaminase or cytosine deaminase is connected to the protein through a linker or not through a linker.

In some embodiments, the linker is a peptide linker or a non-peptide linker.

In some embodiments, the peptide linker has a sequence as set forth in SEQ ID NO: 66, 67 or 95. In some embodiments, the peptide linker has a sequence as set forth in SEQ ID NO: 95.

In some embodiments, the Cas8a protein in the system comprises a TadA8e, and comprises a sequence as set forth in SEQ ID NO: 98.

### I-IV. Type I-A-4 System

In some embodiments, in the system, the Cas5a protein, Cas8a protein, Cas7 protein, Cas6 protein, and Csa5 protein respectively comprise the amino acid sequences as set forth in SEQ ID NOs: 20-24.

In some embodiments, one or more (e.g., all 5) of the Cas5a protein, Cas8a protein, Cas7 protein, Cas6 protein, and Csa5 protein are connected to an NLS sequence (e.g., a sequence as set forth in SEQ ID NO: 65) through a linker or not through a linker.

In some embodiments, the Cas5a protein, Cas8a protein, Cas7 protein, Cas6 protein, and Csa5 protein connected to the NLS sequence respectively comprise the amino acid sequences as set forth in SEQ ID NOs: 87-91.

In some embodiments, the system further comprises: (6) a Cas3 protein or a nucleotide sequence encoding Cas3 protein;
wherein the Cas3 protein comprises an amino acid sequence as set forth in SEQ ID NO: 19.

In some embodiments, the Cas3 protein is connected to an NLS sequence (e.g., a sequence as set forth in SEQ ID NO: 65) through a linker or not through a linker.

In some embodiments, the Cas3 protein connected to the NLS sequence comprises an amino acid sequence as set forth in SEQ ID NO: 86.

In some embodiments, the system does not comprise a Cas3 protein or a nucleotide sequence encoding Cas3 protein.

In some embodiments, a Cas protein (e.g., Cas5a protein, Cas8a protein, Cas7 protein, Cas6 protein, or Csa5 protein) in the system comprises an adenosine deaminase (e.g., TadA8e) or a cytosine deaminase (e.g., APOBEC3); for example, the amino acid sequence of the adenosine deaminase or cytosine deaminase is located at or near the terminus (e.g., N-terminus or C-terminus) of the Cas protein.

In some embodiments, the Cas8a protein in the system comprises an adenosine deaminase (e.g., TadA8e) or a cytosine deaminase (e.g., APOBEC3).

In some embodiments, the amino acid sequence of the adenosine deaminase or cytosine deaminase is located at or near the N-terminus of the Cas8a protein.

In some embodiments, the adenosine deaminase or cytosine deaminase is connected to the protein through a linker or not through a linker.

In some embodiments, the linker is a peptide linker or a non-peptide linker.

In some embodiments, the peptide linker has a sequence as set forth in SEQ ID NO: 66, 67 or 95. In some embodiments, the peptide linker has a sequence as set forth in SEQ ID NO: 95.

In some embodiments, the Cas8a protein in the system comprises a TadA8e, and comprises a sequence as set forth in SEQ ID NO: 99.

### II. Type I-A System—Protein and Guide RNA

In some embodiments, the system further comprises a guide RNA of a Type I-A CRISPR-Cas system or a nucleotide sequence encoding the guide RNA; wherein the guide RNA comprises a direct repeat sequence and a guide sequence capable of hybridizing with a target sequence.

In some embodiments, the direct repeat sequence comprises a stem-loop structure.

In some embodiments, the direct repeat sequence is capable of binding to one or more Cas proteins in the system; for example, the direct repeat sequence is capable of binding to one or more proteins selected from Cas5a protein, Cas8a protein, Cas7 protein, Cas6 protein, Csa5 protein; for example, the guide RNA is capable of binding to a Cascade complex formed by Cas5a protein, Cas8a protein, Cas7 protein, Cas6 protein, and Csa5 protein.

In some embodiments, when the target sequence is DNA, the protospacer adjacent motif (PAM) recognized by the system has a sequence represented by 5′CCN-. In some embodiments, the PAM has a sequence represented by 5′CCT- or 5′CCC-.

In some embodiments, the direct repeat sequence comprises a first region and a second region, and the first region comprises a stem-loop structure.

In some embodiments, the first region is located 5′ to the second region.

In some embodiments, there is or is not an extra nucleotide between the first region and the second region.

In some embodiments, the guide RNA comprises two copies of a direct repeat sequence, i.e., a first copy of direct repeat sequence and a second copy of direct repeat sequence, and a guide sequence located between the first copy of direct repeat sequence and the second copy of direct repeat sequence.

In some embodiments, the guide RNA comprises a second region of the first copy of direct repeat sequence, a guide sequence, and a first region of the second copy of direct repeat sequence.

In some embodiments, the guide sequence is located between the second region of the first copy of direct repeat sequence and the first region of the second copy of direct repeat sequence.

In some embodiments, the second region of the first copy of direct repeat sequence is located 5′ to the guide sequence, and the first region of the second copy of direct repeat sequence is located 3′ to the guide sequence.

In some embodiments, there is or is not an extra nucleotide between the second region of the first copy of direct repeat sequence and the guide sequence.

In some embodiments, there is or is not an extra nucleotide between the guide sequence and the first region of the second copy of direct repeat sequence.

In certain embodiments, when the Cas5a protein, Cas8a protein, Cas7 protein, Cas6 protein and Csa5 protein are as defined in Section I-I above, the direct repeat sequence comprises a sequence as set forth in SEQ ID NO: 49 or consists of the sequence as set forth in SEQ ID NO: 49. In certain embodiments, the first region of direct repeat sequence comprises a sequence as set forth in SEQ ID NO: 51 or consists of the sequence as set forth in SEQ ID NO: 51, the second region of direct repeat sequence comprises a sequence as set forth in SEQ ID NO: 52 or consists of the sequence as set forth in SEQ ID NO: 52.

In certain embodiments, when the Cas5a protein, Cas8a protein, Cas7 protein, Cas6 protein and Csa5 protein are as defined in Section I-II above, the direct repeat sequence comprises a sequence as set forth in SEQ ID NO: 53 or consists of the sequence as set forth in SEQ ID NO: 53. In certain embodiments, the first region of direct repeat sequence comprises a sequence as set forth in SEQ ID NO: 55 or consists of the sequence as set forth in SEQ ID NO: 55, the second region of direct repeat sequence comprises a sequence as set forth in SEQ ID NO: 56 or consists of the sequence as set forth in SEQ ID NO: 56.

In certain embodiments, when the Cas5a protein, Cas8a protein, Cas7 protein, Cas6 protein and Csa5 protein are as defined Section I-III above, the direct repeat sequence comprises a sequence as set forth in SEQ ID NO: 57 or consists of the sequence as set forth in SEQ ID NO: 57. In some embodiments, the first region of direct repeat sequence comprises a sequence as set forth in SEQ ID NO: 59 or consists of the sequence as set forth in SEQ ID NO: 59, the second region of direct repeat sequence comprises a sequence as set forth in SEQ ID NO: 60 or consists of the sequence as set forth in SEQ ID NO: 60.

In some embodiments, when the Cas5a protein, Cas8a protein, Cas7 protein, Cas6 protein and Csa5 protein are as defined in the Section I-IV above, the direct repeat sequence comprises a sequence as set forth in SEQ ID NO: 61 or consists of the sequence as set forth in SEQ ID NO: 61. In some embodiments, the first region of direct repeat sequence comprises a sequence as set forth in SEQ ID NO: 63 or consists of the sequence as set forth in SEQ ID NO: 63, the second region of direct repeat sequence comprises a sequence as set forth in SEQ ID NO: 64 or consists of the sequence as set forth in SEQ ID NO: 64.

### III. Type I-A System—Protein and Dual-Target Guide RNA

In some embodiments, the system further comprises one or more guide RNAs of the Type I-A CRISPR-Cas system or a nucleotide sequence encoding the one or more guide RNAs; wherein, the one or more guide RNAs comprise a direct repeat sequence, a first guide sequence capable of hybridizing with a first target sequence, and a second guide sequence capable of hybridizing with a second target sequence;
wherein, the first target sequence and the second target sequence are respectively located on the flanks of the region to be modified (e.g., the region to be deleted) in a double-stranded target nucleic acid molecule.

In some embodiments, the first target sequence and the second target sequence are respectively located on two single strands of the region to be modified; for example, the first target sequence and the second target sequence are respectively located 5′ to the region to be modified in each single strand.

In some embodiments, the direct repeat sequence comprises a stem-loop structure.

In some embodiments, the direct repeat sequence is capable of binding to one or more Cas proteins in the system; for example, the direct repeat sequence is capable of binding to one or more proteins selected from the group consisting of Cas5a protein, Cas8a protein, Cas7 protein, Cas6 protein, and Csa5 protein. In some embodiments, the guide RNA is capable of binding to a Cascade complex formed by Cas5a protein, Cas8a protein, Cas7 protein, Cas6 protein, and Csa5 protein.

In some embodiments, when the target sequence is DNA, the protospacer adjacent motif (PAM) recognized by the system has a sequence represented by 5′CCN-. In some embodiments, the PAM has a sequence represented by 5′CCT- or 5′CCC-.

In some embodiments, the direct repeat sequence comprises a first region and a second region, the first region comprises a stem-loop structure.

In some embodiments, the first region is located 5′ to the second region.

In some embodiments, there is or is not an extra nucleotide between the first region and the second region.

In some embodiments, when the Cas5a protein, Cas8a protein, Cas7 protein, Cas6 protein, and Csa5 protein are as defined in Section I-I above, the direct repeat sequence is set forth in SEQ ID NO: 69. In certain embodiments, the first region of the direct repeat sequence comprises a sequence as set forth in SEQ ID NO: 51 or consists of the sequence as set forth in SEQ ID NO: 51, the second region of the direct repeat sequence comprises a sequence as set forth in SEQ ID NO: 52 or consists of the sequence as set forth in SEQ ID NO: 52.

In certain embodiments, when the Cas5a protein, Cas8a protein, Cas7 protein, Cas6 protein and Csa5 protein are as defined in Section I-II above, the direct repeat sequence is set forth in SEQ ID NO: 53. In certain embodiments, the first region of the direct repeat sequence comprises a sequence as set forth in SEQ ID NO: 55 or consists of the sequence as set forth in SEQ ID NO: 55, the second region of the direct repeat sequence comprises a sequence as set forth in SEQ ID NO: 56 or consists of the sequence as set forth in SEQ ID NO: 56.

In certain embodiments, when the Cas5a protein, Cas8a protein, Cas7 protein, Cas6 protein and Csa5 protein are as defined in Section I-III above, the direct repeat sequence is set forth in SEQ ID NO: 57. In certain embodiments, the first region of the direct repeat sequence comprises a sequence as set forth in SEQ ID NO: 59 or consists of the sequence as set forth in SEQ ID NO: 59, the second region of the direct repeat sequence comprises a sequence as set forth in SEQ ID NO: 60 or consists of the sequence as set forth in SEQ ID NO: 60.

In certain embodiments, when the Cas5a protein, Cas8a protein, Cas7 protein, Cas6 protein and Csa5 protein are as defined in Section I-IV above, the direct repeat sequence is set forth in SEQ ID NO: 61. In certain embodiments, the first region of the direct repeat sequence comprises a sequence as set forth in SEQ ID NO: 63 or consists of the sequence as set forth in SEQ ID NO: 63, the second region of the direct repeat sequence comprises a sequence as set forth in SEQ ID NO: 64 or consists of the sequence as set forth in SEQ ID NO: 64.

In certain embodiments, the one or more guide RNAs comprise a guide RNA which comprises:
(i) a first copy of direct repeat sequence, a first guide sequence capable of hybridizing with a first target sequence, a second copy of direct repeat sequence, a second guide sequence capable of hybridizing with a second target sequence, and a third copy of direct repeat sequence; or,
(ii) a second region of a first copy of direct repeat sequence, a first guide sequence capable of hybridizing with a first target sequence, a second copy of direct repeat sequence, a second guide sequence capable of hybridizing with a second target sequence, and a first region of a third copy of direct repeat sequence.

In certain embodiments, in (i), the guide RNA comprises from 5′ to 3′ direction: the first copy of direct repeat sequence, the first guide sequence, the second copy of direct repeat sequence, the second guide sequence, and the third copy of direct repeat sequence. In certain embodiments, in (ii), the guide RNA comprises from 5′ to 3′ direction: the second region of the first copy of direct repeat sequence, the first guide sequence, the second copy of direct repeat sequence, the second guide sequence, and the first region of the third copy of direct repeat sequence.

In certain embodiments, the one or more guide RNAs comprise:
a first guide RNA comprising a direct repeat sequence and a first guide sequence capable of hybridizing to a first target sequence; and
a second guide RNA comprising a direct repeat sequence and a second guide sequence capable of hybridizing to a second target sequence.

In certain embodiments, the first guide RNA comprises two copies of direct repeat sequence, i.e., a first copy of direct repeat sequence and a second copy of direct repeat sequence, and a first guide sequence located between the two copies of repeat sequence; or, the first guide RNA comprises, from 5′ to 3′ direction, a second region of the first copy of direct repeat sequence, a first guide sequence, and a first region of the second copy of direct repeat sequence.

In certain embodiments, the second guide RNA comprises two copies of direct repeat sequence, i.e., a first copy of direct repeat sequence and a second copy of direct repeat sequence, and a second guide sequence located between the two copies of repeat sequence; or, the second guide RNA comprises, from 5′ to 3′ direction, a second region of the first copy of direct repeat sequence, a second guide sequence, and a first region of the second copy of direct repeat sequence.

### IV. Effector Protein of Type I-A System

In another aspect, the present application provides a Cas protein of Type I-A CRISPR-Cas system, which is selected from the group consisting of:
(1) a Cas5a protein, in which the Cas5a protein has an amino acid sequence as set forth in any one of SEQ ID NOs: 2, 8, 14, 20 or ortholog, homolog, variant or functional fragment thereof,
(2) a Cas8a protein, in which the Cas8a protein has an amino acid sequence as set forth in any one of SEQ ID NOs: 3, 9, 15, 21 or ortholog, homolog, variant or functional fragment thereof,
(3) a Cas7 protein, in which the Cas7 protein has an amino acid sequence as set forth in any one of SEQ ID NOs: 4, 10, 16, 22 or ortholog, homolog, variant or functional fragment thereof,
(4) a Cas6 protein, in which the Cas6 protein has an amino acid sequence as set forth in any one of SEQ ID NOs: 5, 11, 17, 23 or ortholog, homolog, variant or functional fragment thereof,
(5) a Csa5 protein, in which the Csa5 protein has an amino acid sequence as set forth in any one of SEQ ID NOs: 6, 12, 18, 24 or ortholog, homolog, variant or functional fragment thereof,
(6) Cas3 protein, in which the Cas3 protein has an amino acid sequence as set forth in any one of SEQ ID NOs: 1, 7, 13, 19 or ortholog, homolog, variant or functional fragment thereof, wherein, in any one of (1) to (6), the ortholog, homolog, variant or functional fragment substantially retains the biological function of the sequence from which it is derived.

In certain embodiments, the ortholog, homolog, variant has a substitution, deletion or addition of one or more amino acids (e.g., a substitution, deletion or addition of 1, 2, 3, 4, 5, 6, 7, 8, 9 or 10 amino acids), or has a sequence identity of at least 80%, at least 85%, at least 90%, at least 91%, at least 92%, at least 93%, at least 94%, at least 95%, at least 96%, at least 97%, at least 98%, or at least 99%, as compared to the sequence from which it is derived, and substantially retains the biological function of the sequence from which it is derived.

In the present disclosure, the biological function of the above sequence refers to the activity of the Cas effector protein, including but not limited to, the activity of binding to the guide RNA, the endonuclease activity, and the activity of site-specific binding and cutting the target sequence or complementary sequence thereof under the guidance of the guide RNA.

The protein of the present disclosure can be derivatized, for example, linked to another molecule (e.g., another polypeptide or protein). Generally, the derivatization (e.g., labeling) of a protein does not adversely affect the desired activity of the protein (e.g., activity of binding to guide RNA, endonuclease activity, activity of site-specific binding and cutting the target sequence or complementary sequence thereof under the guidance of a guide RNA). Therefore, the protein of the present disclosure is also intended to include such derivatized forms. For example, the protein of the present disclosure can be functionally linked (by chemical coupling, gene fusion, non-covalent linkage or other means) to one or more other moieties, such as another protein or polypeptide, a detection agent, a pharmaceutical agent, etc.

In particular, the protein of the present disclosure can be linked to an additional functional unit. For example, it can be linked to a nuclear localization signal (NLS) sequence to increase the ability of the protein of the present disclosure to enter the cell nucleus. For example, it can be linked to a targeting moiety to endow the protein of the present disclosure with targeting ability. For example, it can be linked to a detectable label to facilitate detection of the protein of the present disclosure. For example, it can be linked to an epitope tag to facilitate expression, detection, tracing and/or purification of the protein of the present disclosure.

In certain embodiments, the protein described in any one of (1) to (6) optionally comprises an additional protein or polypeptide, wherein the additional protein or polypeptide is selected from the group consisting of epitope tag, reporter gene sequence, nuclear localization signal (NLS) sequence, targeting moiety, transcriptional activation domain (e.g., VP64), transcriptional repression domain (e.g., KRAB domain or SID domain), nuclease domain (e.g., Fok1), adenosine deaminase (e.g., TadA8e), cytosine deaminase (e.g., APOBEC3), a domain having an activity selected from the following: methylase activity, demethylase activity, transcriptional activation activity, transcriptional repression activity, transcript release factor activity, histone modification activity, nuclease activity (e.g., single-stranded RNA cleavage activity, double-stranded RNA cleavage activity, single-stranded DNA cleavage activity, double-stranded DNA cleavage activity) and nucleic acid binding activity; and any combination thereof.

In some embodiments, at least one (e.g., at least 2, at least 3, at least 4, or all 5) of the proteins described in any one of (1) to (6) comprises the additional protein or polypeptide; for example, the protein described in each of (1) to (6) comprises the additional protein or polypeptide.

In some embodiments, the additional protein or polypeptide is an NLS sequence; for example, the protein described in each of (1) to (6) comprises an NLS sequence.

In some embodiments, the NLS sequence is set forth in SEQ ID NO: 65.

In some embodiments, the additional protein or polypeptide is connected to the protein through a linker or not through a linker.

In some embodiments, the linker is a peptide linker or a non-peptide linker.

In some embodiments, the peptide linker has a sequence as set forth in SEQ ID NO: 66, 67, or 95.

In some embodiments, the NLS sequence is located at or near the terminus (e.g., N-terminus or C-terminus) of the protein.

In certain embodiments, the additional protein or polypeptide is an adenosine deaminase (e.g., TadA8e) or a cytosine deaminase (e.g., APOBEC3). In certain embodiments, one of the proteins described in any one of (1) to (5) comprises an adenosine deaminase (e.g., TadA8e) or a cytosine deaminase (e.g., APOBEC3).

In certain embodiments, the amino acid sequence of the adenosine deaminase or cytosine deaminase is located at or near the terminus (e.g., N-terminus or C-terminus) of the protein (e.g., Cas8a protein).

For example, the amino acid sequence of the adenosine deaminase or cytosine deaminase is located at or near the N-terminus of the Cas8a protein.

In certain embodiments, the Cas5a protein comprises an NLS sequence, and comprises a sequence selected from the following, or consists of a sequence selected from the following: (i) a sequence as set forth in any one of SEQ ID NOs: 69, 75, 81, 87; (ii) a sequence having a substitution, deletion or addition of one or more amino acids (e.g., a substitution, deletion or addition of 1, 2, 3, 4, 5, 6, 7, 8, 9 or 10 amino acids) as compared with the sequence as set forth in any one of SEQ ID NOs: 69, 75, 81, 87; or (iii) a sequence having a sequence identity of at least 80%, at least 85%, at least 90%, at least 91%, at least 92%, at least 93%, at least 94%, at least 95%, at least 96%, at least 97%, at least 98%, or at least 99% as compared with the sequence as set forth in any one of SEQ ID NOs: 69, 75, 81, 87.

In certain embodiments, the Cas8a protein comprises an NLS sequence, and comprises a sequence selected from the following, or consists of a sequence selected from the following: (i) a sequence as set forth in any one of SEQ ID NOs: 70, 76, 82, 88; (ii) a sequence having a substitution, deletion or addition of one or more amino acids (e.g., a substitution, deletion or addition of 1, 2, 3, 4, 5, 6, 7, 8, 9 or 10 amino acids) as compared with the sequence as set forth in any one of SEQ ID NOs: 70, 76, 82, 88; or (iii) a sequence having a sequence identity of at least 80%, at least 85%, at least 90%, at least 91%, at least 92%, at least 93%, at least 94%, at least 95%, at least 96%, at least 97%, at least 98%, or at least 99% as compared with the sequence as set forth in any one of SEQ ID NOs: 70, 76, 82, 88.

In certain embodiments, the Cas7 protein comprises an NLS sequence, and comprises a sequence selected from the following, or consists of a sequence selected from the following: (i) a sequence as set forth in any one of SEQ ID NOs: 71, 77, 83, 89; (ii) a sequence having a substitution, deletion or addition of one or more amino acids (e.g., a substitution, deletion or addition of 1, 2, 3, 4, 5, 6, 7, 8, 9 or 10 amino acids) as compared with the sequence as set forth in any one of SEQ ID NOs: 71, 77, 83, 89; or (iii) a sequence having a sequence identity of at least 80%, at least 85%, at least 90%, at least 91%, at least 92%, at least 93%, at least 94%, at least 95%, at least 96%, at least 97%, at least 98%, or at least 99% as compared with the sequence as set forth in any one of SEQ ID NOs: 71, 77, 83, 89.

In certain embodiments, the Cas6 protein comprises an NLS sequence, and comprises a sequence selected from the following, or consists of a sequence selected from the following: (i) a sequence as set forth in any one of SEQ ID NOs: 72, 78, 84, 90; (ii) a sequence having a substitution, deletion or addition of one or more amino acids (e.g., a substitution, deletion or addition of 1, 2, 3, 4, 5, 6, 7, 8, 9 or 10 amino acids) as compared with the sequence as set forth in any one of SEQ ID NOs: 72, 78, 84, 90; or (iii) a sequence having a sequence identity of at least 80%, at least 85%, at least 90%, at least 91%, at least 92%, at least 93%, at least 94%, at least 95%, at least 96%, at least 97%, at least 98%, or at least 99% as compared with the sequence as set forth in any one of SEQ ID NOs: 72, 78, 84, 90.

In certain embodiments, the Csa5 protein comprises an NLS sequence, and comprises a sequence selected from the following, or consists of a sequence selected from the following: (i) a sequence as set forth in any one of SEQ ID NOs: 73, 79, 85, 91; (ii) a sequence having a substitution, deletion or addition of one or more amino acids (e.g., a substitution, deletion or addition of 1, 2, 3, 4, 5, 6, 7, 8, 9 or 10 amino acids) as compared with the sequence as set forth in any one of SEQ ID NOs: 73, 79, 85, 91; or (iii) a sequence having a sequence identity of at least 80%, at least 85%, at least 90%, at least 91%, at least 92%, at least 93%, at least 94%, at least 95%, at least 96%, at least 97%, at least 98%, or at least 99% as compared with the sequence as set forth in any one of SEQ ID NOs: 73, 79, 85, 91.

In certain embodiments, the Cas3 protein comprises an NLS sequence, and comprises a sequence selected from the following, or consists of a sequence selected from the following: (i) a sequence as set forth in any one of SEQ ID NOs: 68, 74, 80, 86; (ii) a sequence having a substitution, deletion or addition of one or more amino acids (e.g., a substitution, deletion or addition of 1, 2, 3, 4, 5, 6, 7, 8, 9 or 10 amino acids) as compared with the sequence as set forth in any one of SEQ ID NOs: 68, 74, 80, 86; or (iii) a sequence having a sequence identity of at least 80%, at least 85%, at least 90%, at least 91%, at least 92%, at least 93%, at least 94%, at least 95%, at least 96%, at least 97%, at least 98%, or at least 99% as compared with the sequence as set forth in any one of SEQ ID NOs: 68, 74, 80, 86.

### V. Nucleic Acid Molecules Related to Direct Repeat Sequence

In another aspect, the present application provides an isolated nucleic acid molecule, which comprises a sequence selected from the following, or consists of a sequence selected from the following:
(i) a sequence as set forth in any one of SEQ ID NOs: 49, 53, 57, and 61;
(ii) a sequence comprising the sequences as set forth in SEQ ID NOs: 51 and 52, a sequence comprising the sequences as set forth in SEQ ID NOs: 55 and 56, a sequence comprising the sequences as set forth in SEQ ID NOs: 59 and 60, or a sequence comprising the sequences as set forth in SEQ ID NOs: 63 and 64;
(iii) a sequence having a substitution, deletion, or addition of one or more bases (e.g., a substitution, deletion, or addition of 1, 2, 3, 4, 5, 6, 7, 8, 9, or 10 bases) as compared with the sequence as set forth in (i) or (ii);
(iv) a sequence having a sequence identity of at least 20%, at least 30%, at least 40%, at least 50%, at least 60%, at least 70%, at least 80%, at least 90%, at least 95% as compared with the sequence as set forth in (i) or (ii);
(v) a sequence capable of hybridizing with the sequence as described in any one of (i) to (iv) under a stringent condition; or
(vi) a complementary sequence of the sequence as described in any one of (i) to (iv);
and, the sequence as described in any one of (iii) to (vi) substantially retains the biological function of the sequence from which it is derived.

In some embodiments, the nucleic acid molecule is capable of binding to one or more of the Cas proteins as described in Section IV above. In some embodiments, the nucleic acid molecule is capable of binding to one or more proteins selected from the group consisting of the Cas5a protein, Cas8a protein, Cas7 protein, Cas6 protein, Csa5 protein.

In certain embodiments, the nucleic acid molecule comprises a sequence selected from the following, or consists of a sequence selected from the following:
(a) a nucleotide sequence as set forth in any one of SEQ ID NOs: 49, 53, 57, and 61;
(b) a sequence comprising the sequences as set forth in SEQ ID NOs: 51 and 52, a sequence comprising the sequences as set forth in SEQ ID NOs: 55 and 56, a sequence comprising the sequences as set forth in SEQ ID NOs: 59 and 60, or a sequence comprising the sequences as set forth in SEQ ID NOs: 63 and 64;
(c) a sequence capable of hybridizing to the sequence as described in (a) or (b) under a stringent condition; or
(d) a complementary sequence of the sequence as described in (a) or (b).

In certain embodiments, the isolated nucleic acid molecule is RNA.

In certain embodiments, the isolated nucleic acid molecule is a direct repeat sequence or fragment thereof in the CRISPR/Cas system.

### VI. Protein Expression-Related Nucleic Acid Molecule/Vector/Host Cell

In another aspect, the present application provides an isolated nucleic acid molecule, which encodes the protein as described in Section IV above.

In another aspect, the present application provides a vector, which comprises the isolated nucleic acid molecule as described in Section VI.

In another aspect, the present application provides a host cell, which comprises the isolated nucleic acid molecule or the vector as described in Section VI.

Such host cells include, but are not limited to, prokaryotic cells such as bacterial cells (e.g., E. coli cells), as well as eukaryotic cells such as fungal cells (e.g., yeast cells), insect cells, plant cells and animal cells (e.g., mammalian cells, such as mouse cells, human cells, etc.).

In some embodiments, the cell or progeny thereof is not capable of developing into a complete animal or plant.

In some embodiments, the host cell is a microorganism.

### VII. Type I-A Vector System—Protein Part

In another aspect, the present application provides a Type I-A CRISPR-Cas vector system, which comprises one or more vectors, wherein the one or more vectors comprise: a nucleotide sequence encoding a Cas protein in the Type I-A CRISPR-Cas system, wherein the Cas protein comprises: Cas5a protein, Cas8a protein, Cas7 protein, Cas6 protein and Csa5 protein;
wherein, the Cas5a protein, Cas8a protein, Cas7 protein, Cas6 protein and Csa5 protein are as defined in any items of Section I-IV above.

In some embodiments, the nucleotide sequence encoding the Cas protein is located in one or more expression cassettes.

In some embodiments, the nucleotide sequences encoding the Cas proteins located in the same expression cassette are arranged in any order.

In some embodiments, the nucleotide sequences encoding the Cas proteins located in the same expression cassette are connected to each other by a nucleotide sequence encoding a self-cleaving peptide (e.g., T2A).

In some embodiments, the expression cassettes each independently comprise a promoter, such as an inducible promoter.

In certain embodiments, the one or more vectors further comprise a nucleotide sequence encoding a Cas3 protein;
wherein, the Cas3 protein is as defined in any one of items of Section I-IV above.

In certain embodiments, the nucleotide sequences encoding Cas5a protein, Cas8a protein, Cas7 protein, Cas6 protein, Csa5 protein, and Cas3 protein are located in the same expression cassette.

In certain embodiments, the one or more vectors comprise:
a first expression cassette, which comprises a nucleotide sequence encoding Cas3 protein and Csa5 protein; and,
a second expression cassette, which comprises a nucleotide sequence encoding Cas7 protein, Cas5a protein, Cas6 protein, and Cas8a protein.

In certain embodiments, the one or more vectors do not comprise a nucleotide sequence encoding Cas3 protein;
wherein, the Cas proteins in the system are as defined in any one of items of Section I-IV above.

In certain embodiments, the one or more vectors comprise:
a first expression cassette, which comprises a nucleotide sequence encoding Cas8a protein; and,
a second expression cassette, which comprises a nucleotide sequence encoding Cas7 protein, Cas5a protein, Cas6 protein, and Csa5 protein.

In certain embodiments, the Cas8a protein is as defined in any one of items of Section I-IV above.

### VIII. Type I-A Vector System—Protein and Guide RNA

In certain embodiments, the one or more vectors further comprise: a nucleotide sequence encoding a guide RNA in the Type I-A CRISPR-Cas system, the guide RNA being as defined in Section II above.

In certain embodiments, the nucleotide sequence encoding the guide RNA in the Type I-A CRISPR-Cas system is located in an additional expression cassette. In certain embodiments, the additional expression cassette comprises a promoter, such as an inducible promoter.

In certain embodiments, the nucleotide sequences encoding the Cas proteins are all located on the same vector.

In certain embodiments, the nucleotide sequences encoding the Cas proteins and the nucleotide sequence encoding the guide RNA are all located on the same vector.

### IX. Type I-A Vector System—Protein and Dual-Target Guide RNA

In certain embodiments, the one or more vectors further comprise: a nucleotide sequence encoding one or more guide RNAs in the Type I-A CRISPR-Cas system, the one or more guide RNAs being as defined in Section III above.

In some embodiments, the nucleotide sequence encoding one or more guide RNAs in the Type I-A CRISPR-Cas system is located in an additional expression cassette. In some embodiments, the additional expression cassette comprises a promoter, such as an inducible promoter.

In some embodiments, the nucleotide sequences encoding the Cas proteins are all located on the same vector.

In some embodiments, the nucleotide sequences encoding the Cas proteins and the nucleotide sequence encoding the guide RNA are all located on the same vector.

### X. Type I-A System—Dual-Target Guide RNA Part

In another aspect, the present application provides a Type I-A CRISPR-Cas system, which comprises: one or more guide RNAs or a nucleotide sequence encoding the one or more guide RNAs; wherein the one or more guide RNAs comprise a direct repeat sequence, a first guide sequence capable of hybridizing with a first target sequence, and a second guide sequence capable of hybridizing with a second target sequence;
wherein the first target sequence and the second target sequence are respectively located on the flanks of the region to be modified (e.g., the region to be deleted) in the double-stranded target nucleic acid molecule.

In some embodiments, the first target sequence and the second target sequence are respectively located on two single strands of the region to be modified. In some embodiments, the first target sequence and the second target sequence are respectively located 5′ to the region to be modified in each single strand.

In some embodiments, the direct repeat sequence comprises a stem-loop structure.

In some embodiments, the direct repeat sequence is capable of binding to one or more Cas proteins in the Type I-A CRISPR-Cas system.

In some embodiments, when the target sequence is DNA, the protospacer adjacent motif (PAM) recognized by the system has a sequence represented by 5′CCN-. In some embodiments, the PAM has a sequence represented by 5′CCT- or 5′CCC-.

In some embodiments, the direct repeat sequence comprises a first region and a second region, and the first region comprises a stem-loop structure.

In some embodiments, the first region is located 5′ to the second region.

In some embodiments, there is or is not an extra nucleotide between the first region and the second region.

In some embodiments, the one or more guide RNAs comprise a guide RNA which comprises:
(i) a first copy of direct repeat sequence, a first guide sequence capable of hybridizing with a first target sequence, a second copy of direct repeat sequence, a second guide sequence capable of hybridizing with a second target sequence, and a third copy of direct repeat sequence; or,
(ii) a second region of a first copy of direct repeat sequence, a first guide sequence capable of hybridizing with a first target sequence, a second copy of direct repeat sequence, a second guide sequence capable of hybridizing with a second target sequence, and a first region of a third copy of direct repeat sequence.

In certain embodiments, in (i), the guide RNA comprises from 5′ to 3′ direction: the first copy of direct repeat sequence, the first guide sequence, the second copy of direct repeat sequence, the second guide sequence, and the third copy of direct repeat sequence.

In certain embodiments, in (ii), the guide RNA comprises from 5′ to 3′ direction: the second region of the first copy of direct repeat sequence, the first guide sequence, the second copy of direct repeat sequence, the second guide sequence, and the first region of the third copy of direct repeat sequence.

In certain embodiments, the one or more guide RNAs comprise:
a first guide RNA comprising a direct repeat sequence and a first guide sequence capable of hybridizing with a first target sequence; and
a second guide RNA comprising a direct repeat sequence and a second guide sequence capable of hybridizing with a second target sequence.

In some embodiments, the first guide RNA comprises two copies of direct repeat sequence, i.e., a first copy of direct repeat sequence and a second copy of direct repeat sequence, and a first guide sequence located between the two copies of repeat sequence; or, the first guide RNA comprises from 5′ to 3′ direction: a second region of a first copy of direct repeat sequence, a first guide sequence, and a first region of a second copy of direct repeat sequence.

In some embodiments, the second guide RNA comprises two copies of direct repeat sequence, i.e., a first copy of direct repeat sequence and a second copy of direct repeat sequence, and a second guide sequence located between the two copies of direct repeat sequence; or, the second guide RNA comprises from 5′ to 3′ direction: a second region of a first copy of direct repeat sequence, a second guide sequence, and a first region of a second copy of direct repeat sequence.

### XI. Type I-A System—Dual-Target Guide RNA and Protein

In some embodiments, the system further comprises: a Cas protein in the Type I-A CRISPR-Cas system or a nucleotide sequence encoding the Cas protein.

For example, each of the Cas proteins further comprises an additional protein or polypeptide selected from the group consisting of: epitope tag, reporter gene sequence, nuclear localization signal (NLS) sequence, targeting moiety, transcriptional activation domain (e.g., VP64), transcriptional repression domain (e.g., KRAB domain or SID domain), nuclease domain (e.g., Fok1), adenosine deaminase (e.g., TadA8e), cytosine deaminase (e.g., APOBEC3), a domain having an activity selected from the following: methylase activity, demethylase activity, transcriptional activation activity, transcriptional repression activity, transcript release factor activity, histone modification activity, nuclease activity (e.g., single-stranded RNA cleavage activity, double-stranded RNA cleavage activity, single-stranded DNA cleavage activity, double-stranded DNA cleavage activity) and nucleic acid binding activity; and any combination thereof.

In certain embodiments, the additional protein or polypeptide is an NLS sequence.

In certain embodiments, the additional protein or polypeptide is an adenosine deaminase (e.g., TadA8e) or a cytosine deaminase (e.g., APOBEC3).

In certain embodiments, the Cas protein comprises a Cas3 protein, a Cas5a protein, a Cas8a protein, a Cas6 protein, a Csa5 protein, and a Cas7 protein.

In certain embodiments, the Cas3 protein, the Cas5a protein, the Cas8a protein, the Cas6 protein, the Csa5 protein, and the Cas7 protein are as defined in any one of items of Section I-IV above.

### XII. Type I-A Vector System—Dual-Target Guide RNA Part

In another aspect, the present application provides a Type I-A CRISPR-Cas vector system, which comprises one or more vectors, and the one or more vectors comprise: a nucleotide sequence encoding one or more guide RNAs in the Type I-A CRISPR-Cas system, the one or more guide RNAs being as defined in Section III above.

### XIII. Type I-A Vector System—Dual-Target Guide RNA and Protein

In certain embodiments, the one or more vectors further comprise: a nucleotide sequence encoding a Cas protein in the Type I-A CRISPR-Cas system.

In certain embodiments, each of the Cas proteins further comprises an additional protein or polypeptide selected from the group consisting of epitope tag, reporter gene sequence, nuclear localization signal (NLS) sequence, targeting moiety, transcriptional activation domain (e.g., VP64), transcriptional repression domain (e.g., KRAB domain or SID domain), nuclease domain (e.g., Fok1), adenosine deaminase (e.g., TadA8e), cytosine deaminase (e.g., APOBEC3), a domain having an activity selected from the following: methylase activity, demethylase activity, transcriptional activation activity, transcriptional repression activity, transcript release factor activity, histone modification activity, nuclease activity (e.g., single-stranded RNA cleavage activity, double-stranded RNA cleavage activity, single-stranded DNA cleavage activity, double-stranded DNA cleavage activity) and nucleic acid binding activity; and any combination thereof.

In certain embodiments, the additional protein or polypeptide is an NLS sequence.

In certain embodiments, the additional protein or polypeptide is an adenosine deaminase (e.g., TadA8e) or a cytosine deaminase (e.g., APOBEC3).

In certain embodiments, the Cas protein comprises a Cas3 protein, a Cas5a protein, a Cas8a protein, a Cas6 protein, a Csa5 protein, and a Cas7 protein.

In certain embodiments, the Cas3 protein, the Cas5a protein, the Cas8a protein, the Cas6 protein, the Csa5 protein, and the Cas7 protein are as defined in any of items of Section I-IV above.

In certain embodiments, the nucleotide sequence encoding one or more guide RNAs in the Type I-A CRISPR-Cas system and the nucleotide sequence encoding the Cas protein in the Type I-A CRISPR-Cas system are located in different expression cassettes.

In certain embodiments, the nucleotide sequences encoding each Cas protein are all located on the same vector.

In certain embodiments, the nucleotide sequences encoding the Cas proteins and the nucleotide sequence encoding the one or more guide RNAs are all located on the same vector.

### XIV. Host Cell

In another aspect, the present application provides a host cell, which comprises a vector system as described in any one of items of Sections VII-IX, XII-XIII above.

Such host cells include, but are not limited to, prokaryotic cells such as bacterial cells (e.g., E. coli cells), and eukaryotic cells such as fungal cells (e.g., yeast cells), insect cells, plant cells and animal cells (e.g., mammalian cells, such as mouse cells, human cells, etc.).

In certain embodiments, the cell or progeny thereof is not capable of developing into a complete animal or plant.

### XV. Use

In another aspect, the present application provides a kit, which comprises the system as described in any one of Sections I to III above, the protein as described in Section IV above, the isolated nucleic acid molecule as described in Section V or Section VI above, the vector as described in Section VI above, the host cell as described in Section VI above, the vector system as described in any one of Sections VII to IX above, the system as described in any one of Sections X to XI above, the vector system as described in any one of Sections XII to XIII above, or the host cell as described in Section XIV above; and a instruction for using the system for nucleic acid editing (e.g., gene or genome editing, gene or genome large-fragment deletion, gene or genome base modification, genome structural variation).

In certain embodiments, the kit comprises the system as described in any one of Sections II to III above.

In certain embodiments, the kit comprises the vector system as described in any one of Sections VIII to IX above.

In certain embodiments, the kit comprises the system as described in Section XI above.

In certain embodiments, the kit comprises the vector system as described in Section XIII above.

In another aspect, the present application also provides a delivery composition, which comprises the system as described in any one of Sections I to III above, the vector system as described in any one of Sections VII to IX above, the system as described in any one of Sections X to XI above, or the vector system as described in any one of Sections XII to XIII above, and a delivery system.

In certain embodiments, the delivery system is selected from the group consisting of particle, vesicle, or viral vector.

In certain embodiments, the particle comprises lipid, sugar, metal, or protein.

In certain embodiments, the vesicle comprises exosome or liposome.

In certain embodiments, the viral vector comprises adenovirus, lentivirus, or adeno-associated virus.

In certain embodiments, the delivery composition comprises a system as described in any one of Sections II-III above.

In certain embodiments, the delivery composition comprises the vector system as described in any one of Sections VIII to IX above.

In certain embodiments, the delivery composition comprises the system as described in Section XI above.

In certain embodiments, the delivery composition comprises the vector system as described in Section XIII above.

In another aspect, the present application provides a method for inducing a deletion in a target genome, wherein the target genome comprises a first nucleic acid chain and a second nucleic acid chain that are complementary, and the method comprises: contacting the system described in any one of Sections II to III above, or the vector system described in any one of Sections VIII to IX above, the system described in Section XI above, or the vector system described in Section XIII above with the target genome, or delivering it to a cell comprising the target genome.

In certain embodiments, the one or more Cas proteins contained in the system or vector system are capable of forming a complex with a guide RNA, and after the complex binds to a target sequence and/or complementary sequence thereof, it induces a deletion of a region comprising the target sequence and/or complementary sequence thereof.

In certain embodiments, the method comprises: contacting the system described in Section III above, or the vector system described in Section IX above, the system described in Section XI above, or the vector system described in Section XIII above with the target genome, or delivering it to a cell comprising the target genome.

In some embodiments, the deletion is a large-fragment deletion, such as a fragment deletion greater than 0.1 kb, greater than 0.2 kb, greater than 0.5 kb, greater than 1 kb, greater than 1.5 kb, greater than 2 kb, greater than 10 kb, greater than 50 kb, greater than 100 kb, such as less than 500 kb, less than 400 kb, less than 300 kb, less than 200 kb.

In some embodiments, the one or more guide RNAs contained in the system or vector system comprise a direct repeat sequence, a first guide sequence capable of hybridizing with a first target sequence, and a second guide sequence capable of hybridizing with a second target sequence; wherein the first target sequence and the second target sequence are respectively located on the flanks of the region to be deleted in the target genome.

In some embodiments, the first target sequence is located on the first nucleic acid chain of the target genome, and the second target sequence is located on the second nucleic acid chain of the target genome; for example, in the first nucleic acid chain, the first target sequence is located 5′ to the region to be deleted, and, in the second nucleic acid chain, the second target sequence is located 5′ to the region to be deleted.

In some embodiments, the length of the region to be deleted is greater than 0.1 kb, for example, greater than 0.2 kb, greater than 0.3 kb, greater than 0.4 kb, greater than 0.5 kb; for example, the length of the region to be deleted is less than 500 kb, for example, less than 400 kb, less than 300 kb, less than 200 kb; for example, the length of the region to be deleted is 0.2 kb to 200 kb (e.g., 0.2 kb to 2 kb, 0.2 kb to 5 kb, 0.2 kb to 10 kb, 0.2 kb to 100 kb, 0.2 kb to 200 kb; for example, 0.5 kb to 1.5 kb, 0.5 kb to 2 kb, 0.5 kb to 10 kb).

In some embodiments, the target genome is present in a cell, or the target genome is present in a nucleic acid molecule (e.g., a plasmid) in vitro.

In some embodiments, the cell is a prokaryotic cell.

In some embodiments, the cell is a eukaryotic cell.

In some embodiments, the cell is selected from the group consisting of animal cell (e.g., mammalian cell, such as human cell), plant cell (e.g., corn cell, corn protoplast, rice cell, Arabidopsis cell, Arabidopsis protoplast).

In some embodiments, the method is used for chromosome elimination.

In another aspect, the present application provides a method for inducing genomic structural variation, wherein the genome comprises a first nucleic acid chain and a second nucleic acid chain that are complementary, and the method comprises: contacting the system as described in any one of Sections II to III above or the vector system as described in any one of Sections VIII to IX above, the system as described in Section XI above, or the vector system as described in Section XIII above with a target genome, or delivering it to a cell comprising the target genome.

In some embodiments, the one or more Cas proteins contained in the system or vector system are capable of forming a complex with a guide RNA, and after the complex binds to a target sequence and/or complementary sequence thereof, it induces a deletion of a region comprising the target sequence and/or complementary sequence thereof, thereby inducing genomic structural variation.

In certain embodiments, the genome comprises a first nucleic acid chain and a second nucleic acid chain that are complementary, and the method comprises: contacting the system as described in Section III above, or the vector system as described in Section IX above, or the system as described in Section XI above, or the vector system as described in Section XIII above with the target genome, or delivering it to a cell comprising the target genome.

In certain embodiments, the deletion is a large-fragment deletion, such as a fragment deletion of greater than 0.1 kb, greater than 0.2 kb, greater than 0.5 kb, greater than 1 kb, greater than 1.5 kb, greater than 2 kb, greater than 10 kb, greater than 50 kb, greater than 100 kb, for example, less than 500 kb, less than 400 kb, less than 300 kb, less than 200 kb.

In certain embodiments, the one or more guide RNAs contained in the system or vector system comprise a direct repeat sequence, a first guide sequence capable of hybridizing with a first target sequence, and a second guide sequence capable of hybridizing with a second target sequence; wherein the first target sequence and the second target sequence are respectively located on the flanks of the region to be deleted in the target genome.

In certain embodiments, the first target sequence is located in the first nucleic acid chain of the target genome, and the second target sequence is located in the second nucleic acid chain of the target genome; for example, in the first nucleic acid chain, the first target sequence is located 5′ to the region to be deleted, and in the second nucleic acid chain, the second target sequence is located 5′ to the region to be deleted.

In some embodiments, the length of the region to be deleted is greater than 0.1 kb, such as greater than 0.2 kb, greater than 0.3 kb, greater than 0.4 kb, greater than 0.5 kb; for example, the length of the region to be deleted is less than 500 kb, such as less than 400 kb, less than 300 kb, less than 200 kb; for example, the length of the region to be deleted is 0.2 kb to 200 kb (e.g., 0.2 kb to 2 kb, 0.2 kb to 5 kb, 0.2 kb to 10 kb, 0.2 kb to 100 kb, 0.2 kb to 200 kb; for example, 0.5 kb to 1.5 kb, 0.5 kb to 2 kb, 0.5 kb to 10 kb).

In some embodiments, the target genome is present in a cell, or the target genome is present in a nucleic acid molecule (e.g., a plasmid) in vitro.

In some embodiments, the cell is a prokaryotic cell.

In some embodiments, the cell is a eukaryotic cell.

In some embodiments, the cell is selected from the group consisting of animal cell (e.g., mammalian cell, such as human cell), plant cell (e.g., corn cell, corn protoplast, rice cell, Arabidopsis cell, Arabidopsis protoplast).

In another aspect, the present application provides a method for modifying a target nucleic acid molecule, comprising: contacting the system described in any one of Sections II to III above, the vector system described in any one of Sections VIII to IX above, the system described in Section XI above, or the vector system described in Section XIII above with the target nucleic acid molecule, or delivering it to a cell containing the target nucleic acid molecule.

In some embodiments, the one or more Cas proteins contained in the system or vector system are capable of forming a complex with a guide RNA, and after the complex binds to a target sequence and/or complementary sequence thereof, it induces modification of a target nucleic acid molecule containing the target sequence and/or complementary sequence thereof.

In some embodiments, the target nucleic acid molecule is RNA or DNA.

In some embodiments, the target nucleic acid molecule is double-stranded DNA.

In some embodiments, the target nucleic acid molecule is a gene or a genome.

In some embodiments, the target nucleic acid molecule is present in a cell, or the target nucleic acid molecule is present in a nucleic acid molecule (e.g., a plasmid) in vitro.

In some embodiments, the cell is a prokaryotic cell.

In some embodiments, the cell is a eukaryotic cell.

In some embodiments, the cell is selected from the group consisting of animal cell (e.g., mammalian cell, such as human cell), plant cell (e.g., corn cell, corn protoplast, rice cell, Arabidopsis cell, Arabidopsis protoplast).

In some embodiments, the modification refers to a large-fragment deletion of the target nucleic acid molecule.

In some embodiments, the modification refers to a break in the target nucleic acid molecule, such as a double-strand break in DNA; for example, the modification further comprises an insertion of an exogenous nucleic acid into the break.

In some embodiments, the modification refers to a change in a base (e.g., cytosine, adenine) in the target nucleic acid molecule.

In another aspect, the present application provides a method for inducing base mutation in a target nucleic acid molecule, comprising: contacting the system described in any one of Sections II to III above, the vector system described in any one of Sections VIII to IX above, the system described in Section XI above, or the vector system described in Section XIII above with the target nucleic acid molecule, or delivering it to a cell containing the target nucleic acid molecule.

In certain embodiments, the system or vector system does not contain a Cas3 protein or a nucleotide sequence encoding Cas3 protein.

In certain embodiments, the one or more Cas proteins contained in the system or vector system can form a complex with a guide RNA, and after the complex binds to a target sequence and/or complementary sequence thereof, it induces modification of a base in a target nucleic acid molecule containing the target sequence and/or complementary sequence thereof, and generates a base mutation during nucleic acid repair or replication.

In certain embodiments, the modification of the base refers to a modification that can change the base complementary pairing mode of the base to be modified. In certain embodiments, before the modification, the base to be modified is complementary to a first base, and after the modification, the modified base is complementary to a second base.

In some embodiments, the one or more Cas proteins contained in the system or vector system further comprise an adenosine deaminase (e.g., TadA8e) or a cytosine deaminase (e.g., APOBEC3).

In some embodiments, the one or more Cas proteins (e.g., Cas8a protein) contained in the system or vector system further comprise an adenosine deaminase (e.g., TadA8e), the base to be modified is adenine, before the modification, adenine is complementary to thymine, after modification, adenine is modified to hypoxanthine, and hypoxanthine is complementary to cytosine.

In some embodiments, the one or more Cas proteins (e.g., Cas8a protein) contained in the system or vector system further comprises a cytosine deaminase (e.g., APOBEC3), the base to be modified is cytosine, before modification, cytosine is complementary to guanine, after modification, cytosine is modified to uracil, and uracil is complementary to thymine.

In some embodiments, the target nucleic acid molecule is RNA or DNA.

In some embodiments, the target nucleic acid molecule is double-stranded DNA.

In some embodiments, the target nucleic acid molecule is a gene or a genome.

In some embodiments, the target nucleic acid molecule is present in a cell, or, the target nucleic acid molecule is present in a nucleic acid molecule (e.g., a plasmid) in vitro.

In some embodiments, the cell is a prokaryotic cell.

In some embodiments, the cell is a eukaryotic cell.

In some embodiments, the cell is selected from the group consisting of animal cell (e.g., mammalian cell, such as human cell), plant cell (e.g., corn cell, corn protoplast, rice cell, Arabidopsis cell, Arabidopsis protoplast).

In another aspect, the present application provides a method for changing the expression of a gene product, comprising: contacting the system as described in any one of Sections II to III above, the vector system as described in any one of Sections VIII to IX above, the system as described in Section XI above, or the vector system as described in Section XIII above with a target nucleic acid molecule encoding the gene product, or delivering it to a cell containing the target nucleic acid molecule.

In some embodiments, the one or more Cas proteins contained in the system or vector system are capable of forming a complex with a guide RNA, and after the complex binds to a target sequence and/or complementary sequence thereof, it induces modification of a target nucleic acid molecule containing the target sequence and/or complementary sequence thereof, thereby changing the expression of the gene product.

In some embodiments, the target nucleic acid molecule is present in a cell, or the target nucleic acid molecule is present in a nucleic acid molecule (e.g., a plasmid) in vitro.

In some embodiments, the cell is a prokaryotic cell.

In some embodiments, the cell is a eukaryotic cell.

In some embodiments, the cell is selected from the group consisting of animal cell (e.g., mammalian cell, such as human cell), plant cell (e.g., corn cell, corn protoplast, rice cell, Arabidopsis cell, an Arabidopsis protoplast).

In some embodiments, the expression of the gene product is altered (e.g., enhanced or reduced).

In some embodiments, the gene product is a protein.

In another aspect, the present application provides a method for producing a plant with a modified trait, the method comprising contacting a plant cell with the system as described in any one of Sections II to III above, the vector system as described in any one of Sections VIII to IX above, the system as described in Section XI above, or the vector system as described in Section XIII above, or allowing a plant cell to undergo the method as described in any one of the above, thereby modifying or editing a target gene or target nucleic acid molecule in the genome of the plant cell, and regenerating a plant from the plant cell.

In certain embodiments, the method comprises contacting the plant cell with the system as described in Section III above, or the vector system as described in Section IX above, the system as described in Section XI above, or the vector system as described in Section XIII above.

In certain embodiments, the plant is an agricultural plant, such as corn, barley, cotton, rice, soybean, wheat, or rice.

In certain embodiments, in the method as described in any one of the items above, the Cas protein or the nucleotide sequence encoding the Cas protein, the guide RNA or the nucleotide sequence encoding the guide RNA contained in the system or the vector system is present in a delivery system.

In certain embodiments, the delivery system is selected from the group consisting of particle, vesicle, or viral vector.

In certain embodiments, the particle comprises a lipid, a sugar, a metal, or a protein.

In certain embodiments, the vesicle comprises an exosome or a liposome.

In certain embodiments, the viral vector comprises an adenovirus, a lentivirus, or an adeno-associated virus.

In another aspect, the present application provides a use of the system as described in any one of Sections I to III above, the protein as described in Section IV above, the isolated nucleic acid molecule as described in Section V or Section VI above, the vector as described in Section VI above, the host cell as described in Section VI above, the vector system as described in any one of Sections VII to IX above, the system as described in any of Section X to XI above, the vector system as described in any one of Sections XII to XIII above, the host cell as described in Section XIV above, the kit as described in Section XV above, or the delivery composition as described in Section XV above, in nucleic acid editing, or in the manufacture of a preparation for nucleic acid editing.

In certain embodiments, the nucleic acid editing comprises gene editing or genome editing.

In some embodiments, the gene editing or genome editing comprises deletion of large nucleic acid fragment, modification of gene, knockout of gene, alteration of expression of gene product, repair of mutation, and/or insertion of polynucleotide, base mutation.

In some embodiments, the nucleic acid editing comprises inducing genomic structural variation or chromosome elimination.

In another aspect, the present application provides a use of the system as described in any one of Sections I to III above, the protein as described in Section IV above, the isolated nucleic acid molecule as described in Section V or Section VI above, the vector as described in Section VI above, the host cell as described in Section VI above, the vector system as described in any one of Sections VII to IX above, the system as described in any one of Sections X to XI above, the vector system as described in any one of Sections XII-XIII above, the host cell as described in Section XIV above, the kit as described in Section XV above, or the delivery composition as described in Section XV above, in the manufacture of a preparation for editing a target nucleotide sequence in a target locus to modify an organism or a non-human organism (e.g., a plant).

In another aspect, the present application provides a cell or progeny thereof obtained by any one of the methods described above, wherein the cell comprises a modification that is not present in its wild type.

In certain embodiments, the cell or progeny thereof is not capable of developing into a complete animal or plant.

In another aspect, the present application also provides a cell product of the cell or progeny thereof as described above.

### Definition of Terms

In the present disclosure, unless otherwise specified, the scientific and technical terms used herein have the meanings commonly understood by those skilled in the art. In addition, the virology, biochemistry, and immunology laboratory operation steps used herein are routine steps widely used in the corresponding fields. At the same time, in order to better understand the present invention, the definitions and explanations of relevant terms are provided below.

When the terms “for example”, “e.g.”, “such as”, “comprise”, “include” or variants thereof are used herein, these terms will not be considered as restrictive terms, but will be interpreted as meaning “but not limited to” or “not limited to”.

Unless otherwise specified herein or clearly contradicted by the context, the terms “a”, “an”, “the” and similar referents should be interpreted as covering the singular and plural in the context of describing the present invention (especially in the context of the following claims).

As used herein, the term “Type I-A CRISPR-CAS system” refers to a Class 1 CRISPR-CAS system comprising a multi-subunit crRNA-effector complex, more specifically to a Type I system, and even more specifically to a subtype I-A system. Subtype I-A systems may include multiple different CAS components, such as Cas3, Cas5 (e.g., Cas5a), Cas6, Csa5, Cas7, and Cas8 (e.g., Cas8a), and optionally other CAS components (see, for example, Makarova et al. 2020. Nature Reviews Microbiology 18 (2): 67-83. https://doi.org/10.1038/s41579-019-0299-x., Koonin, Makarova, and Zhang 2017. Current Opinion in Microbiology 37: 67-78. https://doi.org/10.1016/j.mib.2017.05.008., Koonin and Makarova 2019. Russian Veterinary Journal 2019 (2): 29-36. http://dx.doi.org/10.1098/rstb.2018.0087, and the contents of the aforementioned documents are incorporated herein by reference in their entirety). In certain embodiments, the CAS protein used in the present application is originated from or derived from a prokaryotic organism having a natural I-A system. However, it should be understood that CAS proteins (e.g., Cas3, Cas5 (e.g., Cas5a), Cas7, Cas6, Cas8 (e.g., Cas8a), Csa5) or derivatives thereof from any source may be used. In certain embodiments, the different CAS components used in the present application may be originated from or derived from the same organism or different organisms.

In certain embodiments, the amino acid sequence of the Cas3 protein may refer to SEQ ID NO: 1, 7, 13, 19. However, those skilled in the art would understand that mutations or variations (including but not limited to, substitutions, deletions and/or additions, such as Cas3 proteins in I-A CRISPR-CAS systems from different sources) may be naturally generated or artificially introduced into the amino acid sequence of the Cas3 protein without affecting its biological function. Therefore, in the present disclosure, the term “Cas3 protein” shall include all such sequences, including, for example, the sequences as set forth in SEQ ID NO: 1, 7, 13, 19 and natural or artificial variants thereof.

In certain embodiments, the amino acid sequence of the Cas5a protein may refer to SEQ ID NO: 2, 8, 14, 20. However, those skilled in the art would understand that mutations or variations (including but not limited to, substitutions, deletions and/or additions, such as Cas5a proteins in I-A CRISPR-CAS systems from different sources) may be naturally generated or artificially introduced into the amino acid sequence of the Cas5a protein, without affecting its biological function. Therefore, in the present disclosure, the term “Cas5a protein” shall include all such sequences, including, for example, the sequences as set forth in SEQ ID NO: 2, 8, 14, 20 and natural or artificial variants thereof.

In certain embodiments, the amino acid sequence of the Cas8a protein may refer to SEQ ID NO: 3, 9, 15, 21. However, those skilled in the art would understand that mutations or variations (including but not limited to, substitutions, deletions and/or additions, such as Cas8a proteins in I-A CRISPR-CAS systems from different sources) can be naturally generated or artificially introduced into the amino acid sequence of the Cas8a protein without affecting its biological function. Therefore, in the present disclosure, the term “Cas8a protein” shall include all such sequences, including, for example, the sequences as set forth in SEQ ID NOs: 3, 9, 15, 21 and natural or artificial variants thereof.

In certain embodiments, the amino acid sequence of the Cas7 protein may refer to SEQ ID NOs: 4, 10, 16, 22. However, those skilled in the art would understand that mutations or variations (including, but not limited to, substitutions, deletions and/or additions, such as Cas7 proteins in I-A CRISPR-CAS systems from different sources) may be naturally generated or artificially introduced into the amino acid sequence of the Cas7 protein without affecting its biological function. Therefore, in the present disclosure, the term “Cas7 protein” shall include all such sequences, including, for example, the sequences as set forth in SEQ ID NOs: 4, 10, 16, 22, and natural or artificial variants thereof.

In certain embodiments, the amino acid sequence of the Cas6 protein may refer to SEQ ID NOs: 5, 11, 17, 23. However, those skilled in the art would understand that mutations or variations (including but not limited to substitutions, deletions and/or additions, such as the Cas6 protein in the I-A CRISPR-CAS systems from different sources) can be naturally generated or artificially introduced into the amino acid sequence of the Cas6 protein without affecting its biological function. Therefore, in the present disclosure, the term “Cas6 protein” shall include all such sequences, including, for example, the sequences as set forth in SEQ ID NOs: 5, 11, 17, 23, and natural or artificial variants thereof.

In certain embodiments, the amino acid sequence of the Csa5 (Cas11) protein may be found in SEQ ID NOs: 6, 12, 18, 24. However, those skilled in the art would understand that mutations or variations (including but not limited to substitutions, deletions and/or additions, such as the Csa5 protein in the I-A CRISPR-CAS systems from different sources) can be naturally generated or artificially introduced into the amino acid sequence of the Csa5 protein without affecting its biological function. Therefore, in the present disclosure, the term “Csa5 protein” shall include all such sequences, including, for example, the sequences as set forth in SEQ ID NOs: 6, 12, 18, 24, and natural or artificial variants thereof.

In addition, the sequences as set forth in SEQ ID NOs: 1-24 of the present application do not contain amino acids (e.g., methionine (Met)) encoded by a start codon (e.g., ATG) at their N-terminus. It would be understood by those skilled in the art that in the process of preparing proteins by genetic engineering, due to the effect of start codon, the first position of the produced polypeptide chain is often the amino acid (e.g., Met) encoded by the start codon. The Cas protein of the present disclosure not only encompasses an amino acid sequence that does not contain an amino acid (e.g., Met) encoded by the start codon at its N-terminus, but also encompasses an amino acid sequence that contains an amino acid (e.g., Met) encoded by the start codon at its N-terminus. Therefore, sequences that further contain an amino acid (e.g., Met) encoded by the start codon at the N-terminus of the above amino acid sequence also fall within the scope of protection of the present disclosure.

As used herein, the terms “guide RNA” and “mature crRNA” are used interchangeably and have the meanings commonly understood by those skilled in the art. In general, a guide RNA may contain a direct repeat sequence and a guide sequence, or may consist essentially of or consist of a direct repeat sequence and a guide sequence (also referred to as a spacer in the context of an endogenous CRISPR system). In some cases, a guide sequence is any polynucleotide sequence that has sufficient complementarity to a target sequence to hybridize with the target sequence and guide the specific binding of the CRISPR/Cas complex to the target sequence or complementary sequence thereof. In certain embodiments, when optimally aligned, the degree of complementarity between the guide sequence and its corresponding target sequence is at least 50%, at least 60%, at least 70%, at least 80%, at least 90%, at least 95%, or at least 99%. Determining optimal alignment is within the capabilities of a person of ordinary skill in the art. For example, there are publicly available and commercially available alignment algorithms and programs, such as, but not limited to, ClustalW, Smith-Waterman in matlab, Bowtie, Geneious, Biopython, and SeqMan.

In some cases, the guide sequence has a length of at least 5, at least 10, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, or at least 50 nucleotides. In some cases, the guide sequence has a length of no more than 50, 45, 40, 35, 30, 25, 24, 23, 22, 21, 20, 15, 10 or less nucleotides. In certain embodiments, the guide sequence has a length of 10-50, or 15-40, or 20-40 nucleotides.

In some cases, the direct repeat sequence has a length of at least 10, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 56, at least 57, at least 58, at least 59, at least 60, at least 61, at least 62, at least 63, at least 64, at least 65, or at least 70 nucleotides. In some cases, the direct repeat sequence has a length of no more than 70, 65, 64, 63, 62, 61, 60, 59, 58, 57, 56, 55, 50, 45, 40, 35, 30, 29, 28, 27, 26, 25, 24, 23, 22, 21, 20, 15, 10 or less nucleotides. In certain embodiments, the direct repeat sequence has a length of 55-70 nucleotides, such as 55-65 nucleotides, such as 60-65 nucleotides, such as 62-65 nucleotides, such as 63-64 nucleotides. In certain embodiments, the direct repeat sequence has a length of 15-40 nucleotides, such as 15-38 nucleotides, such as 20-40 nucleotides, such as 22-38 nucleotides, such as 32 nucleotides. In certain embodiments, the direct repeat sequence has a length of not less than 30 nt, such as 30 nt-40 nt, such as 37 nt.

As used herein, the term “CRISPR/Cas complex” refers to a ribonucleoprotein complex formed by binding a guide RNA or a mature crRNA, which comprises a guide sequence capable of hybridizing to a target sequence, to a Cas protein. The ribonucleoprotein complex is capable of recognizing and/or cleaving a polynucleotide and/or complementary strand thereof that can hybridize with the guide RNA or the mature crRNA.

Therefore, in the case of forming a CRISPR/Cas complex, a “target sequence” refers to a polynucleotide targeted by a guide sequence designed to having targeting ability, such as a sequence having a complementarity to the guide sequence, wherein the hybridization between the target sequence and the guide sequence will promote the formation of a CRISPR/Cas complex. Complete complementarity is not required, as long as there is sufficient complementarity to cause hybridization and promote the formation of a CRISPR/Cas complex. The target sequence may comprise any polynucleotide, such as DNA or RNA. In some cases, the target sequence is located in the nucleus or cytoplasm of a cell. In some cases, the target sequence may be located in an organelle such as mitochondria or chloroplast of a eukaryotic cell.

In the present disclosure, the expression “target sequence” or “target polynucleotide” may be any polynucleotide endogenous or exogenous to a cell (e.g., a eukaryotic cell). For example, the target polynucleotide may be a polynucleotide present in the nucleus of a eukaryotic cell. The target polynucleotide may be a sequence encoding a gene product (e.g., a protein) or a non-coding sequence (e.g., a regulatory polynucleotide or a junk DNA). In some cases, it is believed that the target sequence should be associated with a protospacer adjacent motif (PAM). The exact sequence and length requirements for the PAM vary depending on the Cas effector enzyme used, but the PAM is typically a 2-5 base pair sequence adjacent to the protospacer sequence (i.e., the target sequence). Those skilled in the art are able to identify the PAM sequence associated with a given Cas effector protein.

As used herein, the term “adenosine deaminase” refers to a protein that catalyzes the hydrolytic deamination of adenine or adenosine. In some embodiments, the adenosine deaminase catalyzes the hydrolytic deamination of adenine or adenosine to inosine in deoxyribonucleic acid (DNA). In some embodiments, the adenosine deaminase is TadA8e. In certain embodiments, the amino acid sequence of the adenosine deaminase can be found in NCBI Genbank ID: UNJ19119.1 or NCBI Genbank ID: QHD44350.1. However, those skilled in the art understand that in the amino acid sequence of adenosine deaminase, mutations or variations (including but not limited to, substitutions, deletions and/or additions, such as adenosine deaminase from different sources) may be naturally generated or artificially introduced without affecting its biological function. Therefore, in the present disclosure, the term “adenosine deaminase” shall include all such sequences, including, for example, sequences shown in NCBI Genbank ID: UNJ19119.1 or NCBI Genbank ID: QHD44350.1 and natural or artificial variants thereof.

As used herein, the term “cytosine deaminase” refers to a protein that catalyzes the hydrolytic deamination of cytidine or cytosine. In certain embodiments, the cytosine deaminase is APOBEC3. In certain embodiments, the amino acid sequence of the cytosine deaminase can be found in NCBI Genbank ID: 76096346 or NCBI Genbank ID: 176865758. However, those skilled in the art understand that mutations or variations (including but not limited to substitutions, deletions and/or additions, such as cytosine deaminases from different sources) may be naturally or artificially introduced into the amino acid sequence of the cytosine deaminase without affecting its biological function. Therefore, in the present disclosure, the term “cytosine deaminase” shall include all such sequences, including, for example, the sequences shown in NCBI Genbank ID: 76096346 or NCBI Genbank ID: 176865758 and their natural or artificial variants.

As used herein, the term “identity” is used to refer to the matching of sequences between two polypeptides or between two nucleic acids. To determine the percent identity of two amino acid sequences or two nucleic acid sequences, the sequences are aligned for optimal comparison purposes (e.g., a gap may be introduced in a first amino acid sequence or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, the molecules are identical at that position. The percent identity between the two sequences is a function of the number of identical positions shared by the sequences (i.e., percent identity=number of identical overlapping positions/total number of positions×100%). In certain embodiments, the two sequences are the same length.

The determination of the percent identity between two sequences can also be accomplished using a mathematical algorithm. A non-limiting example of a mathematical algorithm for comparing two sequences is the algorithm of Karlin and Altschul, 1990, Proc. Natl. Acad. Sci. U.S.A. 87:2264-2268, as modified in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403.

As used herein, the term “vector” refers to a nucleic acid delivery vehicle into which a polynucleotide can be inserted. When a vector is capable of expressing a protein encoded by the inserted polynucleotide, the vector is called an expression vector. The vector can be introduced into a host cell by transformation, transduction or transfection so that the genetic material elements it carries are expressed in the host cell. Vectors are well known to those skilled in the art, including but not limited to: plasmid; phagemid; cosmid; artificial chromosome, such as yeast artificial chromosome (YAC), bacterial artificial chromosome (BAC) or P1-derived artificial chromosome (PAC); bacteriophage such as λ phage or M13 phage, as well as animal viruses, etc. Animal viruses that can be used as vectors include but are not limited to retrovirus (including lentivirus), adenovirus, adeno-associated virus, herpes virus (e.g., herpes simplex virus), poxvirus, baculovirus, papillomavirus, papovavirus (e.g., SV40). A vector can contain a variety of elements that control expression, including but not limited to promoter sequence, transcription initiation sequence, enhancer sequence, selection element and reporter gene. In addition, the vector may also contain a replication origin.

### Beneficial Effects of the Present Invention

The I-A CRISPR-Cas effector protein and system provided by the present disclosure have significant application value.

For example, the I-A CRISPR-Cas system provided by the present disclosure can be used to achieve precise large-fragment deletion of target genes or genomes (e.g., knockout of gene coding regions, knockout of long lncRNAs or enhancers, chromosome elimination) and/or other target nucleic acid editing (e.g., modifying genes, knocking out genes, changing the expression of gene products, repairing mutations, inserting polynucleotides, and/or single-base mutations, etc.).

For example, the I-A CRISPR-Cas system provided by the present disclosure has pre-crRNA processing activity and, compared to the Cas9 system, does not require tracrRNA, which makes it more easily applied to multiplex gene editing.

For example, the I-A CRISPR-Cas system provided by the present disclosure recognizes a PAM motif having a structure represented by 5′CCN- (e.g., 5′CCT- or 5′CCC-).

For example, the guide RNA provided by the present disclosure, which targets two oppositely oriented target sites, enables more precise fragment deletion of genome as compared to a gene editing system targeting a single target site.

The embodiments of the present invention will be described in detail below in conjunction with the accompanying drawings and examples, but those skilled in the art will understand that the following drawings and examples are only used to illustrate the present invention, rather than to limit the scope of the present invention. According to the following detailed description of the drawings and preferred embodiments, the various objects and advantages of the present invention will become apparent to those skilled in the art.

### BRIEF DESCRIPTION OF THE DRAWINGS

FIG. 1 shows a schematic diagram of the experimental process for PAM identification in Example 2.

FIG. 2 shows the map of expression cassette in the vector, as designed in Example 3.

FIG. 3 shows the editing sites of Type 1-A-2 and Type 1-A-3 on the ROS1 gene in corn genome in Example 4.

FIG. 4 shows the detection result of the editing activity on the endogenous gene of corn in Example 4. FIG. 4A shows the result of PCR detection of the editing product generated by the Type I-A-2 system, FIG. 4B shows the result of PCR detection of the editing product generated by the type 1-A-3 system, FIG. 4C shows the sequence alignment on the editing site in the ROS1 gene editing product generated by the Type I-A-2 system, as detected by first generation sequencing, and FIG. 4D shows the sequence alignment on the editing site in the ROS1 gene editing product generated by the Type I-A-3 system, as detected by first generation sequencing.

FIG. 5 shows the dual-targeted editing sites of Type 1-A-1, Type 1-A-2, and Type 1-A-3 on the ROS1 gene in the maize genome in Example 5.

FIG. 6 shows the detection results of dual-targeted editing activity on the maize endogenous genes in Example 5. FIG. 6A shows the results of PCR detection of the editing products generated by the Type I-A-1 system, FIG. 6B shows the results of PCR detection of the editing products generated by the type 1-A-2 system, FIG. 6C shows the results of PCR detection of the editing products generated by the type 1-A-3 system, FIG. 6D shows the sequence alignment on the editing sites in the ROS1 gene editing product generated by the Type I-A-1 system, as detected by first generation sequencing, FIG. 6E shows the sequence alignment on the editing sites in the ROS1 gene editing product generated by the Type I-A-2 system, as detected by first generation sequencing, and FIG. 6F shows the sequence alignment on the editing sites in the ROS1 gene editing product generated by the Type I-A-3 system, as detected by first generation sequencing.

FIG. 7 shows the map of expression cassette in the vector for adenine single-base editing (I-A TadA8e), as designed in Example 6.

FIG. 8 shows the detection results of the Type I-A system gene editing in stable transgenic corn plants in Example 7. FIG. 8A shows the dual-target design targeting the GA2 gene, wherein #g1 and #g2 represent two target sites; FIG. 8B shows the sequence alignment on the editing sites of the Type I-A-2 system editing products on the GA2 gene of transgenic plants, as detected by first generation sequencing.

FIG. 9 shows the detection results of the Type I-A system gene editing in the HEK293T fluorescent reporter cell line stably expressing Tdtomato in Example 8. FIG. 9A shows a schematic diagram of expression cassette in an animal cell expression vector; FIG. 9B shows a target as designed for targeting Tdtomato red fluorescent gene, wherein G1 and G2 represent two target sites; FIG. 9C shows the detection results of the editing efficiency of the Type I-A system and the CRISPR/Cas9 system detected by the red fluorescence system, wherein the ordinate represents the reduction ratio of the fluorescence value of each system, and in the abscissa, “Cas9” corresponds to the editing efficiency of the CRISPR/Cas9 system, “A3-CCT” corresponds to the editing efficiency of the Type I-A-3 system for the target G1 with 5′-CCT sequence characteristics, and “A3-CCC” corresponds to the editing efficiency of the Type I-A-3 system for the target G2 with 5′-CCC sequence characteristics.

FIG. 10 shows the detection result of the Type I-A system gene editing in the HEK293T cell line in Example 9. FIG. 10A shows a schematic diagram of expression cassette in an animal cell expression vector, FIG. 10B shows a target as designed for targeting the HPRT1 gene, wherein g1 and g2 represent two target sites for dual targeting, and FIG. 10C shows a sequence alignment on the editing site of the Type I-A-2 system editing product on the HPRT1 gene, as detected by first generation sequencing.

### SEQUENCE INFORMATION

The description of the sequences involved in the present application is provided in the table below.

TABLE 1
Sequence information

| SEQ | |
|---|---|
| ID NO: | Sequence and description |
| 1 | Cas3 protein amino acid sequence of I-A-1 |
| | LEEQFQLVTGHSPAEHQRECGEALATGKSVILRAPTGSGKSEAVWIPFLRCRGKR |
| | LPMRMIHALPMRGLANQLEERMKDYAGPGLRVSAMHGQRPESVLFYADAIFATID |
| | QVVASYACAPLSLSVRHGNIPAGAVASSFLVFDEVHTFEPRLGLQSILVLAERAH |
| | QMGMPFVIMSATLPKNFIRSLAERLGAAPIEGGRLKSKEGEPRHVTLRVLPEKLS |
| | ARTILDYAPKVNRTVVVVNTVQRALGLYEQVRDEFRCPVILAHSRFYDEDRRTKE |
| | QQIEALFGKKAAQGRCLLIATQVVEVGLDISCDLLITELAPVDALVQRAGRCARW |
| | GGKGDVIVLTELDTKRPYDETLVAVTERALQEHNVDGQELTWEVETALVDTVLDP |
| | HFKEWAKPDAAGKVLASLAEAAFTGNSTKAEQAVRETLTVEVALHDTPQALGPAI |
| | LRLPRCRLHPGVFQQFVRKQRPNVWQVVVDRDPDDDYRTRIEFLSVNGKSRLIPG |
| | GHYIVDPQFGCYDAERGLRLGVPGQSAEPFAPGQSRDRLKGELQIELWQDHIREV |
| | VKAFERYVLPKERMAFEALSRWLGKTQDELLSVARAVLVLHDLGKLARQWQGKIQ |
| | AGLEGKLSQGSFLAHRGGSVSGLPPHATVSAWVATPCLRRLAGTDWEQTLAVPAL |
| | AAIAHHHSVRADITPEFEMTDGCFEVVADCARGVAGLEVKRDDFNTKPPQGSGSC |
| | GVGLNFLLPEGYTSYVLLSRWLRLADRIATGGGEETIFQYEKWMGDS |
| 2 | Cas5a protein amino acid sequence of I-A-1 |
| | AEWLQAEVEFASFYSYRVPDLSPSFALCSPVPSPAAIRLAVVDATIRHTGDVNEG |
| | HAVFELMKRARLELQPPSRVAVMKFFIKRLKPEKPTKGKRASVIESTGIREYCLP |
| | WGPMVFWIESDQPERIAQSLQWLRRLGTTDSLASCTVGAGTPNFASCIRPANGLT |
| | LQTTNFAQRPVFTLHELKPETQFNQVNPFADERPGKPFEKRLYVLPLVREKVGEN |
| | WVIYHHEPFAA |
| 3 | Cas8a protein amino acid sequence of I-A-1 |
| | EYRLIKSGLEMFDTARAYGLAQLLQVLAGGRAAPRILSQGGVFTLTISTKPNPAT |
| | LKSSDLWRGAFGESNWQKVFLTYKRAWSSQRDKVKRSLESHSADIFGKAETDGLA |
| | VVFGGNFALPGPLDPVGFKGLKGLTAGSYSEGQTTVDEFNWALGCLGAAAAQRYK |
| | IQKAVGNKWEYYVTLPVPEEVQFGDFHAVRQLVYDKGLSYNGVRNAAAHFSLLLA |
| | SAIREKAQGNPHFPVRFSNVLYFSLFQSGQQFKPAIGGAVNVGRLIEIALARPEV |
| | ALEMFKTWDYLFRRGSAQGNEDLAQAITELVMAPSLDTYYRHARIFNRYVVDSTK |
| | RVRPEYLYDETALKEVLNYAEQ |
| 4 | Cas7 protein amino acid sequence of I-A-1 |
| | ADSPVFEVAILGRVVWNLHSLNNEGTVGNVSEPRTVVLADGSKSDGISGEMLKHI |
| | HAQNVWLVAEDKSQLCEPCRTLNPQKADKNPAVLGVKTAKAKVAAESMSVAISSC |
| | ALCDLHGFLVQRPTIARASTVEFGWAVALRDGYHRDIHLHARHAVEGRAETTEGQ |
| | QEGPAEVSGQMIYHRPTRSGTYAFVSVFQPWRIGLNEVNYEYVEGVDREARNKLA |
| | IEAYKATFARTGGAMTSTRLPHVEALEGVVLVSSRNFPVPVTSPLQDDYREKTEK |
| | VGQAVEGLEVQRFGALPELYVILNALAKRRLFALQMGGTSKKGKQ |
| 5 | Cas6 protein amino acid sequence of I-A-1 |
| | GDVLGLHSLRVGLFRFRLVPEQPLEVPALNKGNMLRGGFGHGFRKLCCIPECRDA |
| | RLCPLAAICPYKAVFEPSPPPGSERLAKNQDIPRPFVFRAPHTNQTRFQKGEAFE |
| | FGLVLIGRAVDYLPYFVLSFRELANEGLGLNRAKCALERVEQRRTSANGLGRATG |
| | EGRLVYSKDSGVFHSTENEGVDSYVNSRLRELSSPNGDQSRQNVTIRFLTPTFLK |
| | ANGEVIRRPEFHHLFKRLRDRINALCTFFGDGALDLDFRGVGTRAEKVQSVSART |
| | EWVERCRTSSKTGQRHELSGFMGEATYEGNVEEFLPLLALGELVHVGKHTAWGNG |
| | RIELQSGTGVKC |
| 6 | Csa5 (Cas11) protein amino acid sequence of I-A-1 |
| | LSSDSKLSEVFAEESVKSFGKCLRYALWRDEDYASLIEFENAETPTQFADAVRKF |
| | LRRYRSGGFMDQTQRSRASEMRKQNRWDGLKKLLRQYEVGPRPSEGQLERLMQLA |
| | NDINGVRLVQSAIISYGLTKREPYKEVEELEKEN |
| 7 | Cas3 protein amino acid sequence of I-A-2 |
| | VACDTFFAPMTDFNLARHQSECAGALASGKSVILRAPTGSGKSEAVWLPFLSLRG |
| | KTLPCRLIHTLPMRALVNQLESRMRTYANGRMRVAAMHGQRPESVLFYADAIFAT |
| | LDQVVTSYACAPLSLSVRQGNIPAGAVAGSFLVFDEVHTFEPHLGLQSLLVLAER |
| | AHQMGIPFVIMSATLPTNFIRRLSERFGATIVEGTRLEGKNRRQRRVVLRVSSEK |
| | LSIETILELTRNVERTLVVVNTVQRAQNLYEQLLGKIGCPVILAHSRFYDDDRRT |
| | KEKQIEAQFGKTAEGQCLLIATQVVEVGLDISCDLLVTELAPIDAIVQRAGRCAR |
| | WGGQGEVVVFTGLETTRPYDRTLVEATEKALREKNLNGQELTWEIERALVDTVLE |
| | PQFSKWAEPEAAGKVLASLAEAAFTGDSAKAERAVREGLTVEVALHGSPDTLGVG |
| | ALRLPRCRIHPGGFQQFVHKQQPEAWRVVVDRTAADDYRTRVEFLHVDSNSKAAP |
| | YGYYIIHPQYGSYDVERGLRLGIRGSPAQSRDELIQRKSRLEGELQIEKWQDHIE |
| | KVVKAFAEHVLPKERIAFEALSRRLGKTHEDLLSLTHLVLIFHDLGKLAQQWQRK |
| | IQAGLESVLPPGTFLAHRGGSLRDLPPHATVSASLATPCLCRVAGPDWQQTLAIP |
| | ALAAIAHHHSVRADMTPQFDMSEGWFDVVADCARRLAGVDVTVNDFSRWRGGGSC |
| | GVALNFLLPDGYTSYILLSRWLRLADRIATGGGENAILNYEDWMSSS |
| 8 | Cas5a protein amino acid sequence of I-A-2 |
| | AEWIQAEIEFASFYSYRVPDLSPSYALSSLVPSPAAIRLAVVDAVIRHTGVVDEG |
| | ESIFELVKRAKLEVQPPARIAVMKFFVKRLKPENPEKGKRASVIESTGIREYCLP |
| | SGPLVLWLETEEPERIGQALQWLRRLGTSDSLATCKIGHGAPDTALCIKPANGLA |
| | IQAKNFAQRAVFTLHELKPDANFSEVNPFADGRRGDPFEKRLYVLPCVREQAGEN |
| | WVLYRREPFAN |
| 9 | Cas8a protein amino acid sequence of I-A-2 |
| | EYLVVKSGLPTLDAARAYGLAQLLQVLANGKASPYITDQGGVFAVSLNAELTHDA |
| | LTRSDMWRAAFADSNWQRVFLTYKKAWSAQRDRVKRTLEEQVAAVVTRAGDGLCV |
| | DFAGKFALPGPLDPVGFKGLKGLTAGNYSEGQTYLDEQNGALACLGATIAQRYKF |
| | GKREYFVTLPIPQMVQFNDFHQIRHLVYDKGLAYLGVRTAAAHFALIFADAIRER |
| | AAGNPYFPLSFSNVLYFSLFQSGQQFKPSVGGSINLARLLDIALSRPQAAAEMFK |
| | TWDYLFRRGSVKGNEALAEAITDLLMAPSGESYYRHARIFNRYIVDSSKRVNSEF |
| | LYDEAALMEVMAYVEQ |
| 10 | Cas7 protein amino acid sequence of I-A-2 |
| | AGNSVFEISILGRSVWNLHSLNNEGTVGNVSEPRTVILADGSKSDGISGEMLKHI |
| | HAQNVWLVATDRSVFCEPCQTLQPQKADKNPDVTGVKAARAKLASEGMNVAIAAC |
| | ALCDLHGFLVQKPTIARASTVEFGWAVAVRNGFHRDIHLHARHAVEGRTEGQQEA |
| | GEVAAQMIYHRPTRSGTYALASVFQPWRIGLNEVNYEYVAGVDREARYRLAIEAY |
| | KATFARTDGAMTSTRLPHPEAFEGVVLVSSRNFPVPVTSPLQDDYREKLQQLSRA |
| | TEGLEPQPFNSLTELYGILNELAKRPLFNLQLARSSKREKK |
| 11 | Cas6 protein amino acid sequence of I-A-2 |
| | SQAHCECSLRVRRFRFVIAPREPLLVPAINKGNMLRGGFGHAFRCLCCIPQCRDA |
| | RTCPVGMSCPYKAIFEPSPPPEAEALSKNQDIPRPFVFRAPKTQQTRFETGQPFE |
| | FELVLIGRALDFLPYFVLSFRELAAEGLGLNRAKCSLERVEQVDLTSEAADASNY |
| | EAMVIYTAEDQVFRNAATSETGEWIGRRIRNRSTSRDNDSVQQVSIRFSTPTFLK |
| | ADGEIIRQPEFHHVFKRLRDRINALSTFFGEGPIEADFRGLGERAEKIRTVSART |
| | DWVERFRTSSKTKQRHELSGFVGEVTYEGNLNEFLPWLTLGELVHVGKHTAWGNG |
| | WMELEHEVSRGCV |
| 12 | Csa5 (Cas11) protein amino acid sequence of I-A-2 |
| | SNSEISLASVFAEESIKSFGKCLRYALWRDDDYASLIEFENAETPLQFAEAVRKF |
| | LRRYRSGGFMDEALRTQASEMRKHNRWDELRRTLRQNEIGPRPTEGNLERLTQLA |
| | NNAQGVRLVRAAIISYGLTKRDPHKELEEVERGS |
| 13 | Cas3 protein amino acid sequence of I-A-3 |
| | NKLFKKLIGAKPYDYQKIAMENLLDGKSIIMRAPTGSGKTEIALIPFLYGFNDLL |
| | PSQLIYSLPTRTLVESIGERAVKYASFRKLRVAIHHGKNATSSLFEEDVVVTTID |
| | QAVGAYLSTPLSMSKRSGNIFVGSVGSALTVFDEVHTLDPEKGLQTSLAISMQSA |
| | KLGLPTLIMSATLPDIFIETAKDRISKKGGDIEFIDVKDEFEIKSRKNRFVELIN |
| | RLEEELNAEKVLEEVEHGKRIIIVINTVNRAQELYLELRNKTELPILLLHSRFLE |
| | KDRQEKELLLEETFGKNGNGKCIFIATQIVEVGMDISSPKVLSEIAPIDALIQRA |
| | GRCARWSGKGEFHVFGYNTNSKSPHAPYNKDIVEATKSEINNKGKSFTLDWNTEV |
| | ELVNKILTKHFSEFMNSMIFYQRLGELARAVYEGSRAKVEQNVREVFSCDVTLHE |
| | NPKSMNSVEILHLPRLRLDARTLMGKVEKIAEMGIDTYRLEENTIIFDDDEDEYV |
| | PVLVNNREEIIPFELYVLCGASYSSDTGLVFDDFPNALKSFDPEEKEILSSKQFD |
| | NRLKVETWVEHAKNTLKVLDNYMIPRYRYSIENFAENYGYNYGEFLDIIRCTVSL |
| | HDIGKLNKKWQKRIKWNDETPLAHSNDNTIKRLPAHATVSAKALQPYLEDLFDDE |
| | DIFKAFYLAIAHHHQPWSKSYNEYELVPKYDESLKEIWIIPKNFIQEQNPAGRLD |
| | FSYLDIIDENEAYRLYGFLSKLMRISDRLATGGNTYESLFSG |
| 14 | Cas5a protein amino acid sequence of I-A-3 |
| | QWLKFTLHFPSFFSYRIPDYSSQYALGIPLPSPSTLKLGVISSAIKSTGKVSEGE |
| | KVFNVVKDAEVCVAPPEKIAINSFLIKRLKKRKEDLKLIPTFGIRDYVFFPDDID |
| | IFVGSENIDSVAEYFSKMNYIGSSDSMVYVKSIEPKTPSENVIKAVDIDEFSDAA |
| | EKESYLVYPVKDINKNATFDQINSYSSKSSRKILDQKYYLINAKVSKGKNWKILD |
| | TRN |
| 15 | Cas8a protein amino acid sequence of I-A-3 |
| | NHYFLAKSGWEFFDVSKAYGLGLVIQTLTGNASITDRGGFYLIESKNETKFDKIE |
| | EISKYFDDSELKTTLITIQRSTKSEMKPPVKKVKGKCLETLTDKESMITVIKNYE |
| | NLNSPSIIGTDKQTLYQTMDLAATKGIRNEILLKKNYSDGTNIKISDKDFALSLL |
| | GHINFTIKKFSDFGLILVAPTPLKTELKNVRQIYANLKGNVKVAHKAGWFPTITQ |
| | IAINLVSEEIMVKDGGKFAPKFGSLIYSIMRKTGNQWKPSTGGIFPLDFLHQIAD |
| | SDNAINILNKWKKIFGWTSRKNGHEDLPTSLAEFIANPNLFNYQRYVNFHLRNEI |
| | DKDNIKFGDYKKEDFLEVMKNVGI |
| 16 | Cas7 protein amino acid sequence of I-A-3 |
| | MVNETEIYEIAILGRATWQLHSLNNEGTVGNVTEPRSVTIIDPNTKNPITTDGIS |
| | GEMLKHIHTGLMWTLTDKNNLCDACKVLNPEKFNVTSGRGSTVEEVLENALNKCD |
| | ICDLHGFLITRPTVSRKSTIEFGWALGIPEIYRDIHTHARHALGGKTTENEESKG |
| | VNTPNSSEDKEEAVGTSTQMVYHRPTRSGVYAVISMFQPWRIGLNETRQDQYTYD |
| | TGNNEKRIERYKNALKAYQILFTRPKGAMSTTRLPHVEDFEGVIVFSTDQIPLPL |
| | ISPLKQDYVKEITDISKKIDNSINVEEFKTLSEFVDKIGDLIDKKPYKLKLGE |
| 17 | Cas6 protein amino acid sequence of I-A-3 |
| | RLKISLTSNNGNYLIPYNYNHILSAITYRKIADLDLAAKLHFSKDFKFFTFSQIY |
| | FSDWKRTKNGIISKDGKLSFYISSPNEQLIKSLVEGHLENTEVDFKGKKLLVEQI |
| | ELLKSPSFKENIKLKTMSPVAASIKREVDGKLKIWDLGPGDERFYESVQKNLVNK |
| | YTSFYGDYDGDKWVRIKPDMKTAKRRRIEIKGDFHRGYMMEFEMEADPRLVEFAY |
| | DCGLGEKNSMGFGMVNIYE |
| 18 | Csa5 (Cas11) protein amino acid sequence of I-A-3 |
| | SEFRLKDVFEHESIKSFGKTLRKMIRPPKEGNKEKWASDYASIVELGYVETKDQF |
| | AEVIKKLLRRYDVIAKKHQLKRPTEKNLEELMELIDKYGVKPVRAALISYALVKK |
| | DEE |
| 19 | Cas3 protein amino acid sequence of I-A-4 |
| | KYKEIFEKLKLNNLTEVQQKISELEGSKNILVVSSCGSGKTEASYFKMLEYNRKT |
| | IIIEPMKTLTNSIHGRVDIYNKKLGLEKVSIQHSSSQEDRFLQNKYTVTTIDQVL |
| | VGYLAMGKQAYIKGKNIVMSNLIFDEVQLFDTDTMLLTTINMLDEIYKLGNKFII |
| | MTATMPQFLIEFLGERYDMEIVITEKIREDRNVKLFYEEELDYNKVRNYKDKQII |
| | ICNSIKQLKEIHKKLPNSRVITLHSTFLGSNRLKLEKQVERYFGKHSEQNDKILL |
| | TTQIVEVGMDISCDRLYTTACKIDNLVQRDGRCCRWGGDGQVIVFKNDDNIYEKE |
| | LVEETIKYIKNNQGIAFNWTIQKQWINEILNEYYKNKINEYNLRKNKFNFNGCNR |
| | SRLIRDIQNINVIVVNKEEFTKQDFNRESVSLHINKLKELSQANEIYILNKNKIE |
| | KVKYNKVEIGDTVIIRGKNCRYDDLGFRYEEDSAKNMPKCRDFPMTNKSNNNQFR |
| | DYIEETWIHHAETVRDLMSYRLNQEQFNDYIIINGKKIAFYGGLHDLGKLDLEWS |
| | RKYKSAIPLAHFPFVKGSMGEKRTHELISGEILKEIIDDDIIYNMMIQHHKRLYD |
| | DIDIDYKGIEWELHKDTYKILTTYGFKDDIQLQSDAKTLKRNNIMSPCDNEWTTL |
| | LYLVGTFMECEIQAINEYIDNYKQAI |
| 20 | Cas5a protein amino acid sequence of I-A-4 |
| | KKVTYKLSNIFSLKKYNDNNLNCQSYEYPTIYGIRCAILGAIIQVDGIDKVQELF |
| | NKIKNSNIYIQYPKEFKVNGIKQKRYANSYYNSCYTEEEYNKLSPSTQSKTYCVL |
| | DRDKLVGSNWKTTMGFRQYVKMDNIVFYIDNLIPEIDMYLKNIDWLGTAKSMVYL |
| | SDVEEVNKLDNVLTRWNKESYVDTFEQHDWNSKTTFDTIYMYSKKYKHFHDTFMC |
| | GIGDIILPSWLWYTRYTFILYFKLWLVNLYEN |
| 21 | Cas8a protein amino acid sequence of I-A-4 |
| | NEYEFKVIKTANDIEDICISYGICKILSDNRIKFKLKDNKSMYSIYTKEFDIQND |
| | IFYNDENIENVWNLNSGLNQKETVRALDDMNKFLSENIHDILEHLLNGKVLNYKK |
| | ESAKGIGNCFYSLGVRASTFGKTLEISPIKKYLSFLGWIYGCSYCYKEKSFEITA |
| | ILKPYNTDEIAKPFNFSYVDKETGDKKILTKIKKASEINMMSILYIETLKKYKML |
| | SDEYSNVIFMQNIIAGQKPLYDKTTNIKIYKLSQKYLDDLLKKLTWSNVSEDVKD |
| | ITARYVLNIDKYKEFSKLIKIYSKDGNSKINNDFKGEILSMYNEMIKKIYNDETI |
| | NKIGKGFNRLLRDNKGFEIQTKLYNVANEKHLVKVLKMIIDLYSRNYKSAILNND |
| | ELNKLINTIEDKEYAKICSDAILSIGKVFIIIKK |
| 22 | Cas7 protein amino acid sequence of I-A-4 |
| | NKIAMMMRLKLTGEALNNEGTIGNVIQPRQIEFPNGEVRQAISGEMLKHYHSRNL |
| | RLLADENELCDTCKIFSPMKNGKVKESDSKLSPSGNKVKECIVDDVEGFMNAGKG |
| | ANEKRTSCVKFSYAIATEENEYQIMLHTRVDVTQDNNKKKQEKETTEGEGNTNKD |
| | QNTQMLFHRPLRNNEYAITVQVDLDRIGFDDEKLIYALDEDTIKSRQEKCIKALL |
| | NMFVDMEGAMCSTRLPHIEGIEGIIVKKTDKNQVLSKYSALKDDYKEVNEKISDD |
| | SIIFNNIIEFSEVMKGLI |
| 23 | Cas6 protein amino acid sequence of I-A-4 |
| | RINLQGTIIEGQSSIKTNYNHEMYSMILTNISTERANYIHEKKRFKRLFTFSNLY |
| | ISDNKVHFYVSGQDELIKDFINCIMFNQMVRVGDRVISITNIEPMKNSLETKKEY |
| | IFKSNFIVNQKENDRVCLSKDMGYVMKRISDIVKDKYKEIYKEEINENLNVEILN |
| | SKQKYTKYKDHHLNSYQATLKVRGNKKLIDLLYNVGIGENTASGHGFVWEVS |
| 24 | Csa5 (Cas11) protein amino acid sequence of I-A-4 |
| | NNEIKIVKCIDSLYPTVKLTIGKLYKVKESENDKFYRVIADDNNEEQLCYKYRFE |
| | LVDINEIKELTLQDIFNEEEGIKYNRINGGSGIYTIQNETLIIGEHIKPVLNKRI |
| | MDSKFVKVKVERLVSFSDVINSDYKCKVKHYRVEGLIQEESSYTWLEEYQDLKDI |
| | MLALSEEFNTIALKEIINKGQWYLEN |
| 25 | Nucleotide sequence encoding I-A-1 Cas3 |
| | CTGGAGGAACAGTTTCAGCTGGTGACCGGGCACTCACCGGCAGAACACCAGAGGG |
| | AGTGCGGAGAGGCGCTGGCCACGGGAAAGAGTGTGATCCTGAGGGCTCCGACCGG |
| | CTCCGGCAAATCCGAAGCCGTGTGGATTCCGTTCCTTCGCTGCAGAGGCAAAAGG |
| | CTTCCGATGAGGATGATCCACGCCCTGCCAATGAGAGGGCTCGCCAACCAGCTCG |
| | AAGAGAGGATGAAGGACTACGCCGGGCCGGGCCTGCGCGTTTCTGCTATGCACGG |
| | CCAAAGACCGGAGTCCGTGCTGTTCTACGCGGACGCAATCTTCGCGACCATTGAC |
| | CAAGTGGTGGCCAGCTACGCCTGCGCCCCGCTTTCCCTGTCCGTGAGGCACGGCA |
| | ACATCCCGGCCGGCGCTGTGGCTTCTTCTTTCTTGGTGTTTGATGAGGTGCACAC |
| | CTTCGAGCCGAGGCTGGGGTTGCAGTCCATCCTGGTCCTTGCTGAACGCGCGCAC |
| | CAAATGGGGATGCCCTTCGTGATTATGTCCGCTACGCTTCCGAAGAACTTTATCC |
| | GCTCCCTGGCCGAGCGCCTGGGCGCTGCTCCAATCGAGGGCGGTCGCCTGAAGTC |
| | CAAGGAGGGCGAGCCGAGGCACGTCACCCTGAGGGTGCTTCCGGAGAAGCTGAGC |
| | GCCAGGACCATCCTTGACTACGCCCCCAAAGTGAATCGCACCGTGGTGGTGGTGA |
| | ACACCGTCCAGAGAGCTTTGGGCTTGTACGAGCAAGTGCGGGATGAATTTAGGTG |
| | CCCCGTGATTCTGGCGCACTCCAGATTCTACGATGAGGACAGGAGAACCAAGGAG |
| | CAGCAGATCGAGGCCCTCTTCGGGAAGAAGGCCGCGCAAGGCAGGTGCCTGCTGA |
| | TTGCAACGCAAGTGGTGGAAGTGGGCCTGGACATCTCCTGCGACCTGCTGATAAC |
| | CGAGCTGGCCCCGGTGGACGCCCTCGTTCAGAGAGCTGGGAGGTGCGCGCGGTGG |
| | GGTGGTAAGGGAGACGTGATTGTGCTGACCGAGCTTGACACGAAGAGGCCGTACG |
| | ACGAGACGTTGGTGGCCGTCACCGAGAGGGCCCTGCAGGAGCATAATGTGGACGG |
| | GCAAGAGCTGACGTGGGAGGTGGAGACGGCGCTGGTGGACACCGTGCTGGACCCG |
| | CACTTCAAGGAGTGGGCGAAGCCGGACGCGGCGGGAAAGGTGCTGGCCTCTCTCG |
| | CGGAGGCGGCCTTTACCGGCAACAGCACTAAGGCAGAGCAAGCCGTGAGGGAGAC |
| | CCTGACTGTTGAGGTGGCGCTGCACGACACCCCACAAGCCCTGGGCCCGGCTATC |
| | CTGAGGCTGCCAAGATGTAGGCTGCACCCGGGGGTGTTCCAGCAATTCGTGAGGA |
| | AACAAAGACCCAACGTGTGGCAGGTGGTTGTCGATAGAGACCCGGACGACGATTA |
| | CAGGACCAGGATCGAGTTCCTGAGCGTGAACGGCAAGAGTAGGCTGATCCCGGGC |
| | GGGCACTACATCGTGGACCCGCAGTTCGGGTGTTACGATGCCGAGAGGGGCCTGA |
| | GGCTTGGCGTGCCAGGCCAGAGCGCGGAACCATTTGCACCGGGCCAGAGCAGGGA |
| | CAGATTGAAGGGCGAGCTGCAGATAGAGCTCTGGCAGGATCACATTAGAGAGGTG |
| | GTGAAAGCGTTTGAGAGGTACGTGCTGCCGAAGGAGAGGATGGCCTTCGAGGCGT |
| | TGTCGCGGTGGTTGGGGAAGACGCAGGACGAGTTGTTGAGCGTGGCGAGGGCGGT |
| | GTTGGTGTTGCATGATTTGGGGAAGTTGGCGAGGCAGTGGCAGGGGAAGATTCAG |
| | GCGGGGCTGGAGGGGAAGTTGAGCCAGGGCTCCTTTTTGGCGCACAGGGGGGGGT |
| | CGGTGTCTGGGTTGCCGCCACACGCCACCGTGAGCGCTTGGGTGGCGACCCCATG |
| | CTTGAGGAGGTTGGCCGGGACGGACTGGGAGCAGACGCTTGCTGTGCCGGCCTTG |
| | GCGGCGATCGCGCATCATCACTCCGTTAGGGCCGACATTACCCCGGAGTTTGAGA |
| | TGACCGACGGCTGCTTCGAGGTGGTGGCCGACTGCGCCAGGGGGGTTGCTGGTCT |
| | TGAGGTGAAGAGGGACGATTTTAACACCAAGCCGCCGCAGGGCTCGGGCTCTTGT |
| | GGCGTTGGCTTGAATTTTCTGCTTCCAGAGGGGTATACGAGCTATGTGTTGTTGT |
| | CTAGGTGGTTGAGGTTGGCGGATAGGATCGCGACGGGGGGCGGCGAAGAGACCAT |
| | TTTCCAGTATGAGAAGTGGATGGGGGACTCT |
| 26 | Nucleotide sequence encoding I-A-1 Cas5a |
| | GCCGAGTGGCTGCAGGCTGAGGTGGAGTTCGCCAGCTTCTATTCCTACAGAGTGC |
| | CGGATCTGTCCCCGTCCTTTGCGCTGTGCTCCCCGGTGCCGAGCCCAGCTGCTAT |
| | TAGGTTGGCCGTGGTGGATGCCACCATTAGGCACACCGGGGACGTTAACGAGGGC |
| | CACGCCGTCTTTGAGCTCATGAAGAGGGCCAGGCTGGAGCTCCAGCCACCGTCCA |
| | GGGTTGCCGTCATGAAATTCTTCATCAAGAGGCTGAAGCCAGAAAAGCCGACGAA |
| | AGGAAAGAGGGCCAGTGTGATTGAATCCACGGGGATAAGGGAGTATTGTCTCCCA |
| | TGGGGCCCGATGGTGTTCTGGATCGAGTCCGACCAGCCGGAGAGGATCGCGCAGT |
| | CCCTGCAGTGGCTGAGGAGGTTGGGCACCACGGACTCCTTGGCCTCCTGCACCGT |
| | GGGCGCCGGTACCCCAAACTTCGCCTCCTGCATTAGGCCGGCCAACGGGCTGACC |
| | CTGCAGACCACCAACTTCGCCCAAAGGCCGGTCTTCACCCTGCACGAGCTGAAGC |
| | CCGAAACCCAGTTCAACCAAGTCAACCCGTTCGCCGACGAGAGACCAGGCAAACC |
| | GTTCGAAAAAAGGCTGTACGTGCTGCCACTCGTGCGCGAGAAGGTGGGGGAAAAC |
| | TGGGTGATATATCACCACGAGCCGTTCGCCGCG |
| 27 | Nucleotide sequence encoding I-A-1 Cas8a |
| | GAGTACAGGCTGATTAAGAGCGGGCTGGAGATGTTTGATACGGCCAGGGCCTACG |
| | GCCTGGCGCAGCTTCTGCAGGTGCTGGCCGGGGGAAGGGCGGCTCCAAGAATTCT |
| | GAGCCAGGGGGGGGTGTTTACCCTGACAATCAGCACGAAGCCGAACCCAGCAACC |
| | CTGAAGTCCTCCGATCTCTGGCGCGGCGCGTTCGGGGAGAGTAACTGGCAAAAAG |
| | TGTTCCTTACGTACAAGAGGGCCTGGTCCAGCCAAAGGGACAAAGTGAAACGCAG |
| | CCTGGAGTCCCACAGCGCCGACATCTTCGGCAAGGCCGAGACGGATGGCCTGGCC |
| | GTCGTGTTTGGCGGCAACTTCGCGTTGCCGGGGCCGTTGGACCCGGTGGGTTTTA |
| | AGGGGTTGAAGGGGCTGACAGCCGGCAGTTACTCTGAGGGCCAGACGACAGTGGA |
| | TGAGTTCAATTGGGCGTTGGGGTGTCTTGGGGCCGCGGCTGCTCAGAGGTACAAG |
| | ATTCAGAAGGCGGTGGGCAACAAGTGGGAGTATTACGTGACTCTTCCGGTGCCGG |
| | AGGAGGTGCAGTTCGGCGACTTTCACGCCGTGAGGCAGCTGGTGTACGATAAAGG |
| | CCTGTCCTACAACGGGGTGAGGAATGCAGCCGCGCACTTCTCCCTGCTGCTTGCC |
| | TCCGCCATTCGCGAGAAAGCCCAGGGGAACCCGCACTTCCCGGTCAGGTTCTCCA |
| | ACGTGCTGTATTTCTCCCTGTTTCAGTCCGGCCAGCAGTTCAAGCCCGCCATCGG |
| | CGGCGCCGTGAACGTGGGTAGGCTCATTGAGATCGCCCTGGCCCGCCCAGAGGTC |
| | GCTCTTGAGATGTTTAAGACCTGGGACTACCTCTTTCGGCGCGGCTCCGCCCAGG |
| | GGAATGAGGATCTTGCGCAGGCGATCACCGAACTGGTGATGGCCCCCTCCCTGGA |
| | CACCTACTACAGGCACGCGCGGATCTTCAACAGGTACGTGGTCGACTCTACCAAA |
| | AGGGTGAGGCCGGAGTACCTCTACGACGAGACGGCCCTGAAAGAGGTGCTTAACT |
| | ACGCCGAGCAG |
| 28 | Nucleotide sequence encoding I-A-1 Cas7 |
| | GCGGACAGCCCGGTTTTCGAGGTGGCCATCCTGGGGCGCGTGGTGTGGAACCTGC |
| | ACAGCCTGAACAACGAGGGCACCGTGGGCAACGTGAGCGAGCCGAGGACCGTGGT |
| | GCTGGCGGATGGGAGCAAATCCGACGGGATATCAGGCGAGATGCTGAAGCATATC |
| | CACGCCCAAAACGTCTGGCTGGTGGCGGAAGACAAGAGCCAGTTGTGCGAACCGT |
| | GCAGGACCCTGAACCCGCAGAAAGCCGACAAGAACCCGGCCGTGCTGGGGGTGAA |
| | AACAGCGAAGGCCAAGGTGGCGGCGGAGAGCATGAGCGTGGCGATCTCCTCCTGC |
| | GCCCTGTGCGACCTGCACGGCTTCCTCGTGCAAAGACCGACCATCGCCAGGGCCA |
| | GTACCGTCGAATTCGGGTGGGCCGTTGCCCTTAGGGACGGCTACCATAGGGACAT |
| | CCACCTGCACGCTAGGCACGCCGTCGAGGGCCGTGCTGAGACCACCGAGGGCCAA |
| | CAGGAGGGCCCGGCTGAGGTTTCCGGGCAAATGATTTACCACAGGCCGACCCGCT |
| | CCGGCACCTACGCTTTCGTTTCCGTCTTCCAGCCATGGAGGATCGGGCTGAACGA |
| | GGTGAACTACGAGTATGTCGAAGGCGTGGACAGGGAAGCGAGGAACAAACTGGCC |
| | ATCGAGGCCTACAAGGCCACCTTCGCGAGGACCGGCGGCGCTATGACGTCCACCA |
| | GGCTGCCGCATGTGGAGGCGTTGGAGGGGGTGGTGTTGGTGAGCAGTAGGAACTT |
| | CCCCGTTCCGGTGACCTCCCCCTTGCAGGACGATTACAGGGAGAAGACCGAGAAG |
| | GTGGGCCAAGCCGTTGAGGGGCTGGAGGTGCAAAGGTTCGGGGCCCTGCCGGAGC |
| | TGTACGTGATCCTGAATGCGCTGGCCAAAAGGAGGCTGTTCGCCTTGCAAATGGG |
| | CGGCACGTCTAAAAAGGGGAAGCAG |
| 29 | Nucleotide sequence encoding I-A-1 Cas6 |
| | GGCGACGTGTTGGGGTTGCACTCCTTGAGGGTGGGCTTGTTCCGGTTCCGCTTGG |
| | TGCCGGAGCAGCCGTTGGAGGTGCCGGCTTTGAACAAGGGCAACATGTTGAGGGG |
| | GGGGTTCGGGCATGGGTTTAGGAAGTTGTGTTGTATTCCGGAGTGTCGGGATGCC |
| | AGGCTTTGCCCACTTGCGGCTATTTGTCCGTATAAGGCCGTGTTCGAGCCGAGCC |
| | CGCCGCCAGGTTCCGAGAGATTGGCGAAGAACCAGGACATTCCGAGGCCGTTCGT |
| | GTTTAGAGCGCCCCACACCAACCAAACCAGGTTCCAGAAGGGCGAGGCCTTCGAG |
| | TTCGGCCTCGTCCTGATCGGCAGGGCAGTGGATTACCTCCCATACTTCGTCTTGT |
| | CCTTCAGGGAGCTGGCCAATGAAGGACTGGGCCTCAACAGGGCGAAGTGCGCGCT |
| | GGAGCGGGTTGAGCAGAGGAGGACCAGCGCGAACGGGCTTGGCAGGGCCACCGGT |
| | GAGGGGAGACTGGTGTATAGTAAGGACAGCGGGGTGTTTCACAGCACGGAGAACG |
| | AGGGGGTGGATAGCTATGTGAACAGCAGGCTGAGGGAGCTGAGTAGCCCGAACGG |
| | GGACCAGAGCAGGCAGAACGTGACGATCAGGTTTTTGACGCCGACCTTCCTGAAG |
| | GCGAACGGGGAGGTGATTAGGAGGCCGGAGTTCCACCACCTGTTTAAAAGACTGA |
| | GGGACAGGATTAACGCATTGTGCACCTTTTTCGGCGACGGCGCCCTGGACCTGGA |
| | CTTTAGGGGGGTGGGGACCAGGGCGGAGAAGGTGCAGAGCGTCTCCGCGAGGACC |
| | GAGTGGGTGGAGAGGTGCAGGACCAGCAGCAAGACCGGCCAAAGACATGAACTCT |
| | CTGGCTTTATGGGCGAGGCGACGTACGAGGGGAACGTGGAGGAGTTCCTGCCGCT |
| | GCTGGCGCTGGGCGAGCTGGTTCACGTCGGGAAGCACACGGCCTGGGGCAACGGC |
| | CGTATTGAGTTGCAGTCCGGGACGGGGGTGAAG |
| 30 | Nucleotide sequence encoding I-A-1 Csa5 (Cas11) |
| | CTGAGCAGCGACAGCAAGCTGAGTGAGGTGTTCGCGGAGGAGAGCGTGAAGAGCT |
| | TCGGGAAGTGCCTGAGGTACGCCCTGTGGAGGGACGAGGACTACGCGAGTCTGAT |
| | AGAGTTCGAGAACGCGGAGACGCCGACCCAGTTTGCGGATGCCGTGAGGAAGTTC |
| | CTGAGGAGGTATAGGTCCGGCGGGTTTATGGACCAGACGCAGAGGAGCAGGGCCT |
| | CAGAGATGAGGAAGCAGAACAGATGGGACGGCCTGAAAAAGCTGCTGAGACAGTA |
| | CGAGGTGGGGCCGAGGCCAAGCGAGGGGCAACTGGAGAGGCTGATGCAGCTGGCG |
| | AACGACACCAACGGGGTGAGGCTCGTGCAGAGTGCCATCATTAGCTACGGGCTTA |
| | CAAAAAGGGAGCCGTATAAGGAGGTGGAGGAGCTGGAGAAGGAGAAC |
| 31 | Nucleotide sequence encoding I-A-2 Cas3 |
| | GTGGCCTGCGATACCTTCTTCGCCCCCATGACCGACTTCAACCTGGCGAGACACC |
| | AGAGCGAGTGCGCGGGGGCATTGGCGAGTGGGAAGAGTGTGATTCTTAGGGCCCC |
| | GACCGGCTCTGGCAAGTCCGAAGCCGTTTGGCTCCCGTTCCTGTCCCTGAGGGGG |
| | AAAACACTGCCGTGCCGTTTGATTCACACCCTTCCGATGCGCGCCCTGGTGAACC |
| | AGCTGGAGTCCCGGATGAGAACCTACGCAAATGGGCGGATGAGGGTTGCCGCCAT |
| | GCACGGGCAACGCCCCGAGTCTGTCCTGTTCTACGCCGACGCCATCTTCGCCACC |
| | CTCGACCAGGTCGTTACCTCTTACGCCTGCGCCCCGCTGTCCCTCTCCGTGAGAC |
| | AGGGTAACATCCCGGCCGGAGCCGTTGCCGGGTCCTTCCTTGTCTTCGACGAGGT |
| | TCACACCTTCGAGCCACATCTGGGCCTCCAGTCCCTTCTCGTCCTCGCCGAGCGT |
| | GCCCACCAAATGGGCATCCCGTTTGTGATCATGTCTGCGACCCTCCCCACCAACT |
| | TCATTCGTAGGCTGTCCGAGCGCTTCGGCGCGACCATCGTCGAGGGCACCAGGTT |
| | GGAGGGTAAGAACCGGAGGCAGAGGAGAGTTGTGCTGAGGGTTTCTTCCGAGAAG |
| | CTGTCTATCGAGACTATCCTGGAGCTGACCAGGAACGTCGAAAGGACCTTGGTTG |
| | TGGTGAACACGGTGCAGAGGGCCCAGAACTTGTACGAGCAGCTGTTGGGGAAGAT |
| | TGGGTGCCCGGTGATCCTGGCCCACTCTAGGTTCTACGACGACGATAGGAGGACA |
| | AAGGAGAAGCAGATAGAGGCGCAATTCGGTAAAACCGCCGAGGGGCAATGCCTGC |
| | TGATCGCCACGCAGGTCGTTGAGGTGGGGCTTGACATCAGCTGCGACCTGCTGGT |
| | GACGGAGCTGGCCCCGATTGACGCGATAGTCCAGAGGGCCGGGAGGTGCGCGAGG |
| | TGGGGTGGTCAAGGGGAGGTGGTGGTGTTTACGGGGCTTGAGACGACGAGGCCGT |
| | ATGACAGGACGTTGGTGGAGGCGACGGAGAAGGCCCTGAGGGAGAAGAACTTGAA |
| | CGGGCAGGAGCTGACGTGGGAGATTGAGAGGGCCTTGGTGGATACCGTGCTGGAG |
| | CCGCAGTTTTCCAAATGGGCGGAGCCGGAGGCGGCGGGAAAGGTTCTGGCGTCCC |
| | TGGCCGAGGCCGCGTTTACCGGAGACAGCGCGAAGGCGGAGAGGGCCGTGAGAGA |
| | GGGCCTGACAGTCGAGGTGGCCTTGCACGGGTCACCGGATACCCTGGGGGGGGCG |
| | CTCTTAGGTTGCCGAGGTGTAGGATTCATCCGGGGGGGTTCCAACAGTTTGTGCA |
| | TAAGCAGCAGCCGGAAGCCTGGAGGGTGGTTGTGGATAGGACGGCGGCGGATGAT |
| | TATAGAACGAGGGTGGAGTTCTTGCACGTCGACTCCAATAGCAAGGCCGCCCCGT |
| | ACGGGTACTACATTATTCACCCGCAATACGGGTCTTATGATGTGGAGAGGGGGCT |
| | GAGGCTGGGGATCAGGGGGAGCCCAGCGCAGAGCAGGGACGAGCTGATCCAGAGG |
| | AAGAGCAGGCTGGAGGGCGAGCTGCAGATTGAGAAGTGGCAGGACCACATTGAGA |
| | AGGTGGTGAAGGCCTTCGCGGAGCACGTCCTGCCGAAGGAGAGGATAGCGTTCGA |
| | GGCATTGAGCAGAAGGTTGGGGAAGACCCACGAAGACTTGTTGAGCCTCACGCAT |
| | TTGGTGCTCATTTTCCACGACCTGGGGAAGCTCGCCCAACAATGGCAGAGGAAGA |
| | TCCAGGCCGGTTTGGAGAGCGTGCTTCCGCCGGGCACCTTCCTTGCGCACCGCGG |
| | TGGTTCCCTGAGGGACCTGCCTCCGCACGCCACGGTGTCTGCCTCCCTCGCTACC |
| | CCGTGCCTCTGCAGGGTTGCTGGCCCGGATTGGCAGCAGACGCTGGCCATCCCTG |
| | CCCTTGCTGCTATCGCCCACCACCACTCCGTTAGGGCCGACATGACCCCGCAGTT |
| | CGACATGTCCGAGGGGTGGTTCGACGTGGTGGCCGACTGCGCCAGGAGGCTCGCT |
| | GGTGTGGACGTGACCGTGAACGACTTCTCCCGCTGGAGGGGCGGCGGCTCTTGCG |
| | GTGTTGCCTTGAACTTTTTGCTTCCGGACGGGTATACCTCTTACATCTTGTTGTC |
| | CAGGTGGCTTCGGCTGGCGGATAGGATCGCGACGGGCGGTGGTGAGAATGCTATT |
| | CTGAATTATGAGGATTGGATGTCCTCTTCTTCT |
| 32 | Nucleotide sequence encoding I-A-2 Cas5a |
| | GCGGAGTGGATTCAAGCGGAGATTGAGTTCGCCAGCTTCTACAGCTACAGGGTGC |
| | CGGACCTGAGCCCGTCCTATGCGCTGTCCTCCCTGGTGCCGAGCCCGGCTGCTAT |
| | CAGGCTGGCCGTGGTGGACGCCGTTATTCGCCACACTGGGGTGGTCGACGAGGGG |
| | GAGAGTATTTTCGAATTGGTGAAGAGGGCCAAGTTGGAGGTGCAGCCACCGGCCC |
| | GGATTGCCGTGATGAAATTTTTTGTGAAGAGGCTGAAGCCGGAAAACCCGGAGAA |
| | GGGGAAGAGGGCCAGCGTGATTGAGAGTACCGGGATCAGGGAGTACTGTCTCCCG |
| | TCCGGTCCTCTGGTGCTGTGGCTGGAGACGGAGGAGCCGGAAAGGATAGGCCAGG |
| | CCCTGCAGTGGCTGAGGAGGCTCGGGACGAGCGACTCCTTGGCCACCTGTAAGAT |
| | TGGCCACGGCGCGCCGGACACGGCCTTGTGCATTAAACCGGCGAACGGGCTGGCG |
| | ATTCAAGCCAAAAACTTCGCCCAGAGGGCCGTGTTCACCCTGCACGAGCTCAAAC |
| | CGGACGCCAACTTCTCCGAGGTTAACCCGTTTGCGGACGGCAGGAGGGGAGACCC |
| | GTTTGAGAAGAGGCTGTACGTCCTGCCGTGCGTGAGAGAGCAGGCCGGCGAAAAC |
| | TGGGTGCTGTATAGACGGGAGCCGTTCGCCAAC |
| 33 | Nucleotide sequence encoding I-A-2 Cas8a |
| | GAGTATTTGGTGGTGAAGAGTGGGCTGCCGACGCTGGACGCCGCCAGAGCTTACG |
| | GGTTGGCTCAGTTGTTGCAGGTGTTGGCCAATGGCAAGGCAAGCCCGTATATTAC |
| | CGACCAGGGGGGCGTGTTTGCGGTGAGCCTTAACGCCGAGTTGACCCACGACGCG |
| | CTGACCAGGTCTGATATGTGGAGGGCCGCGTTTGCCGACAGTAACTGGCAGAGAG |
| | TGTTCCTGACCTACAAGAAAGCGTGGAGTGCGCAGAGGGACAGAGTGAAGCGCAC |
| | CCTGGAAGAGCAGGTGGCCGCCGTGGTGACAAGAGCCGGGGATGGGCTGTGCGTG |
| | GACTTCGCCGGCAAGTTCGCGCTGCCGGGGCCATTGGACCCTGTGGGTTTCAAGG |
| | GGCTCAAAGGCCTGACGGCCGGGAATTATTCCGAGGGCCAAACGTACTTGGATGA |
| | GCAGAATGGGGCCCTTGCGTGCCTGGGTGCTACTATCGCCCAGAGGTACAAGTTC |
| | GGGAAGAGGGAGTACTTCGTGACCCTTCCGATCCCGCAGATGGTGCAGTTCAACG |
| | ACTTTCACCAGATCAGGCATTTGGTGTACGACAAGGGGCTGGCCTATCTCGGGGT |
| | GCGCACTGCCGCGGCACACTTCGCTCTGATCTTCGCCGACGCCATAAGGGAGAGG |
| | GCCGCCGGTAACCCCTACTTCCCGCTCTCTTTCTCCAACGTGCTGTATTTTTCCC |
| | TGTTCCAGTCCGGCCAGCAGTTTAAGCCCTCCGTGGGCGGTTCCATTAACCTCGC |
| | TCGCTTGCTGGACATCGCCCTCTCCCGCCCCCAGGCTGCTGCTGAAATGTTCAAG |
| | ACCTGGGACTACCTCTTTCGCCGCGGCTCCGTGAAAGGCAATGAGGCTCTCGCCG |
| | AAGCCATTACCGACCTCCTGATGGCCCCGTCCGGCGAATCTTACTATAGGCACGC |
| | GAGGATATTCAACCGGTACATCGTCGATTCCAGCAAGAGGGTGAACTCCGAATTT |
| | CTCTACGACGAGGCCGCCCTGATGGAAGTGATGGCCTACGTGGAGCAG |
| 34 | Nucleotide sequence encoding I-A-2 Cas7 |
| | GCGGGGAACAGCGTGTTCGAGATCAGCATCCTGGGGAGGTCAGTCTGGAACCTGC |
| | ACAGCCTGAACAACGAGGGGACGGTGGGCAACGTGAGCGAGCCGAGGACCGTTAT |
| | ATTGGCCGATGGGAGCAAGTCCGACGGGATCAGCGGCGAAATGTTGAAGCATATT |
| | CACGCCCAGAACGTTTGGCTGGTGGCGACCGATAGGAGCGTGTTCTGCGAACCGT |
| | GCCAGACCCTGCAGCCGCAGAAGGCCGATAAAAACCCGGACGTGACCGGCGTGAA |
| | GGCCGCCAGAGCCAAGCTGGCGTCGGAGGGAATGAACGTGGCGATCGCCGCCTGT |
| | GCCCTGTGTGACCTGCACGGCTTCCTCGTGCAAAAACCAACCATCGCCAGGGCCT |
| | CCACCGTGGAATTCGGCTGGGCCGTGGCCGTGAGGAACGGGTTCCATAGGGACAT |
| | CCATCTGCACGCCAGGCACGCCGTCGAGGGCAGAACCGAGGGCCAACAAGAGGCC |
| | GGGGAAGTGGCCGCCCAGATGATCTACCATAGGCCGACCAGGAGCGGGACCTACG |
| | CCTTGGCGTCCGTGTTCCAACCGTGGAGAATTGGGCTGAACGAAGTGAATTACGA |
| | GTACGTGGCGGGCGTTGATAGAGAAGCCAGGTACAGGTTGGCCATCGAGGCATAT |
| | AAAGCGACCTTCGCCCGCACCGACGGCGCCATGACCTCCACTAGGCTGCCGCATC |
| | CGGAGGCATTCGAGGGAGTGGTGCTGGTGAGCTCCAGGAACTTCCCGGTGCCGGT |
| | GACCTCCCCACTTCAAGACGACTACAGGGAAAAGCTGCAGCAGCTCTCCAGGGCC |
| | ACCGAGGGTCTGGAGCCGCAGCCATTTAACTCCCTGACCGAGCTGTACGGCATCC |
| | TGAACGAACTGGCCAAGAGGCCACTGTTCAACCTGCAACTGGCCAGGTCCTCCAA |
| | GAGGGAGAAGAAG |
| 35 | Nucleotide sequence encoding I-A-2 Cas6 |
| | TCCCAGGCCCACTGCGAGTGCTCCCTGAGGGTGAGGAGGTTCAGGTTCGTGATCG |
| | CGCCGAGGGAGCCGTTGTTGGTGCCGGCTATCAACAAGGGGAACATGCTGAGAGG |
| | GGGGTTTGGCCACGCCTTCAGGTGTCTGTGCTGCATCCCGCAGTGCAGGGACGCC |
| | AGGACCTGCCCAGTGGGGATGAGCTGCCCGTACAAAGCCATCTTCGAGCCGTCCC |
| | CGCCGCCGGAAGCCGAAGCTCTTTCCAAGAACCAAGACATCCCGAGACCATTCGT |
| | GTTCAGGGCACCAAAAACCCAGCAGACCCGCTTCGAGACCGGACAGCCCTTCGAG |
| | TTCGAGCTGGTCCTGATAGGAAGGGCCCTCGACTTCCTGCCGTACTTCGTTCTCT |
| | CCTTCAGGGAGCTGGCCGCCGAGGGCTTGGGTCTGAATAGGGCCAAGTGCTCCCT |
| | CGAGAGGGTGGAGCAAGTGGACCTGACGTCCGAAGCCGCCGACGCCTCCAACTAC |
| | GAGGCCATGGTTATCTATACCGCCGAAGACCAGGTGTTCCGCAACGCCGCCACCT |
| | CCGAGACCGGCGAATGGATTGGCAGGAGAATCCGCAACAGGTCCACCAGCAGGGA |
| | CAACGACTCCGTGCAGCAGGTGTCCATCAGATTTTCCACACCGACCTTCCTGAAA |
| | GCCGACGGCGAAATCATTAGGCAGCCGGAGTTCCACCACGTCTTCAAGAGGCTGA |
| | GGGACAGAATCAACGCCCTTTCCACCTTCTTTGGCGAGGGCCCGATCGAAGCCGA |
| | CTTCAGGGGTTTGGGGGAGAGGGCGGAGAAGATCAGGACGGTCTCCGCCAGGACC |
| | GACTGGGTGGAGAGGTTCAGGACCTCCTCTAAGACCAAGCAAAGACATGAACTGA |
| | GCGGGTTCGTGGGCGAGGTGACGTATGAGGGCAACCTGAACGAGTTCCTGCCGTG |
| | GCTGACGCTGGGGGAGTTGGTGCACGTCGGCAAGCACACCGCCTGGGGCAACGGC |
| | TGGATGGAGCTGGAGCATGAGGTGTCTAGGGGCTGTGTG |
| 36 | Nucleotide sequence encoding I-A-2 Csa5 (Cas11) |
| | AGCAACAGCGAGATAAGCCTGGCGAGCGTGTTCGCGGAGGAGAGCATAAAGAGCT |
| | TCGGGAAGTGCCTGAGGTACGCGCTGTGGAGGGACGACGACTACGCGAGTTTGAT |
| | CGAGTTCGAGAACGCGGAGACCCCGCTTCAATTCGCCGAGGCCGTGAGGAAGTTC |
| | CTGAGGCGGTATAGGTCCGGCGGGTTCATGGATGAGGCCCTGAGGACGCAGGCCA |
| | GCGAGATGAGGAAGCACAACAGGTGGGACGAACTGAGGCGCACCTTGAGACAGAA |
| | CGAGATCGGCCCGAGGCCGACGGAGGGGAATCTGGAGAGGTTGACGCAATTGGCA |
| | AACAACGCGCAGGGGGTGAGGCTGGTGAGGGCGGCTATAATAAGCTATGGCCTGA |
| | CGAAGAGGGACCCGCATAAGGAGCTGGAGGAGGTGGAGAGGGGGAGC |
| 37 | Nucleotide sequence encoding I-A-3 Cas3 |
| | AACAAACTGTTCAAGAAGCTCATTGGCGCGAAGCCGTACGACTATCAGAAGATCG |
| | CCATGGAAAACTTGCTGGACGGCAAAAGCATCATCATGAGGGCGCCAACTGGGTC |
| | TGGCAAGACGGAAATTGCCCTGATCCCCTTCCTTTACGGGTTCAACGACTTGCTC |
| | CCGTCCCAGCTGATCTACTCCCTTCCAACCCGCACCCTGGTGGAGAGCATCGGCG |
| | AGCGTGCTGTGAAGTACGCCTCCTTTCGCAAGCTGCGCGTGGCCATCCATCACGG |
| | CAAGAACGCCACTTCCTCCCTCTTCGAGGAGGACGTGGTGGTGACTACCATTGAC |
| | CAGGCGGTGGGCGCGTACCTGTCCACGCCACTGTCCATGTCCAAAAGGTCCGGCA |
| | ACATCTTCGTGGGGTCCGTGGGCTCCGCGCTGACCGTTTTCGATGAGGTGCACAC |
| | CCTGGACCCAGAAAAGGGGCTTCAGACCTCCCTCGCCATCTCCATGCAGTCCGCG |
| | AAGCTGGGCCTGCCGACCTTGATCATGTCTGCCACTCTTCCAGATATCTTCATCG |
| | AAACCGCCAAAGACCGCATCTCCAAGAAGGGGGGCGACATCGAGTTCATCGACGT |
| | CAAGGATGAGTTCGAGATCAAATCCAGGAAGAACCGCTTTGTGGAGCTCATTAAC |
| | AGGCTGGAGGAGGAGCTGAACGCCGAGAAGGTGCTGGAAGAGGTGGAGCACGGGA |
| | AAAGGATTATAATTGTGATCAACACGGTGAACAGGGCCCAGGAGCTGTATCTGGA |
| | ACTGAGGAACAAAACGGAGCTGCCGATCCTGCTGCTGCATAGCAGGTTTTTGGAG |
| | AAGGACAGGCAAGAGAAGGAGCTGCTGCTCGAAGAGACCTTCGGCAAAAACGGCA |
| | ACGGCAAGTGCATATTCATCGCCACGCAAATTGTGGAGGTGGGCATGGACATCTC |
| | CAGCCCGAAGGTGCTGAGCGAGATCGCGCCGATCGACGCCTTGATCCAGAGGGCA |
| | GGCAGGTGCGCTAGGTGGTCCGGCAAAGGGGAGTTCCACGTTTTCGGTTATAACA |
| | CCAACAGCAAGTCCCCGCATGCTCCGTACAACAAAGACATCGTGGAGGCCACCAA |
| | GTCCGAGATTAACAACAAAGGCAAGTCCTTCACGCTGGACTGGAACACCGAGGTG |
| | GAGCTGGTGAACAAGATCCTCACTAAACACTTCTCCGAGTTCATGAACAGCATGA |
| | TATTCTACCAAAGGCTGGGCGAACTCGCGCGCGCCGTCTACGAAGGGAGCAGGGC |
| | AAAAGTGGAACAAAACGTGAGGGAGGTGTTCAGCTGCGACGTCACGCTCCACGAG |
| | AACCCGAAGTCCATGAACTCCGTGGAGATACTCCACCTGCCGAGGCTGAGGCTGG |
| | ACGCGAGGACGCTGATGGGCAAGGTGGAGAAGATTGCCGAAATGGGCATTGACAC |
| | CTACAGACTGGAGGAGAACACCATCATCTTTGACGACGACGAGGACGAGTACGTG |
| | CCGGTGCTGGTGAACAACAGGGAGGAGATCATTCCGTTCGAGCTGTACGTGCTTT |
| | GCGGCGCCTCCTACTCCAGCGACACCGGCTTGGTGTTTGACGACTTTCCGAATGC |
| | GCTGAAGTCCTTCGATCCGGAGGAGAAGGAGATACTGTCCTCCAAGCAGTTTGAC |
| | AATCGCCTTAAGGTGGAGACCTGGGTGGAACACGCGAAGAATACCCTGAAAGTGC |
| | TGGACAATTACATGATTCCGAGGTACAGGTACAGCATAGAGAACTTCGCCGAAAA |
| | CTACGGGTACAACTACGGGGAATTTCTGGACATTATAAGATGTACCGTGTCCCTG |
| | CACGATATCGGGAAGCTCAACAAAAAATGGCAGAAGAGGATCAAGTGGAACGACG |
| | AGACCCCGCTGGCGCACTCCAACGACAACACCATCAAGCGCCTGCCGGCCCACGC |
| | CACCGTGTCAGCTAAGGCGCTCCAGCCATACCTGGAGGACCTGTTCGACGACGAG |
| | GATATCTTCAAAGCCTTCTACCTCGCCATCGCCCACCACCACCAACCATGGTCCA |
| | AATCCTATAACGAGTACGAGCTGGTGCCGAAATACGACGAATCCCTGAAGGAGAT |
| | ATGGATCATTCCCAAGAACTTTATCCAGGAACAGAACCCGGCCGGCCGCCTGGAC |
| | TTTTCCTACCTGGACATCATCGACGAAAACGAGGCCTACCGCCTGTACGGCTTCC |
| | TGAGCAAGCTGATGCGCATCTCCGACAGGCTGGCCACCGGAGGCAACACCTACGA |
| | GAGCCTGTTTTCCGGC |
| 38 | Nucleotide sequence encoding I-A-3 Cas5a |
| | CAGTGGCTGAAATTCACCCTGCACTTCCCGTCCTTCTTCAGCTACAGAATACCAG |
| | ACTACTCCTCTCAGTACGCGCTGGGCATCCCCCTGCCGTCCCCATCTACCCTGAA |
| | GCTCGGCGTCATATCCAGCGCCATCAAGTCCACCGGCAAGGTGTCCGAAGGCGAA |
| | AAGGTGTTTAACGTGGTGAAGGACGCCGAAGTCTGCGTCGCCCCCCCCGAAAAGA |
| | TCGCCATCAACTCCTTCCTCATCAAGCGGCTCAAGAAGAGGAAAGAAGACCTGAA |
| | ACTGATACCGACCTTCGGCATTAGGGACTATGTTTTCTTTCCGGATGACATCGAC |
| | ATCTTCGTGGGGTCCGAAAACATCGACTCCGTGGCTGAATACTTCTCAAAGATGA |
| | ACTACATCGGCTCCTCCGACTCCATGGTGTACGTGAAGTCCATCGAGCCCAAGAC |
| | CCCGTCCGAGAACGTTATTAAGGCCGTGGACATTGACGAGTTCAGCGACGCCGCC |
| | GAGAAAGAGTCCTACCTCGTCTACCCGGTGAAGGATATCAATAAGAACGCCACCT |
| | TCGACCAGATCAACAGCTACTCATCCAAATCATCCAGGAAGATTCTGGACCAAAA |
| | ATACTACCTTATCAACGCCAAGGTGTCCAAGGGCAAAAACTGGAAGATCCTGGAC |
| | ACCCGCAAC |
| 39 | Nucleotide sequence encoding I-A-3 Cas8a |
| | AACCACTACTTCCTGGCGAAGAGCGGGTGGGAGTTCTTCGACGTGTCCAAAGCCT |
| | ACGGGCTCGGCCTGGTGATCCAAACCCTCACAGGCAACGCCAGCATCACCGACAG |
| | GGGGGGCTTCTACTTGATCGAATCCAAAAACGAGACCAAGTTCGACAAGATTGAG |
| | GAGATCTCCAAGTACTTCGACGACAGCGAACTCAAGACCACCCTCATCACCATCC |
| | AAAGAAGCACGAAGTCCGAGATGAAACCGCCGGTGAAAAAAGTGAAGGGCAAGTG |
| | CCTGGAGACGCTGACCGACAAGGAGAGCATGATCACCGTGATCAAAAACTACGAG |
| | AACCTGAACAGCCCGAGCATCATCGGCACCGACAAGCAAACCCTGTACCAGACCA |
| | TGGACCTCGCTGCGACCAAAGGCATAAGGAACGAAATCCTCCTCAAGAAAAACTA |
| | CTCCGACGGCACCAACATCAAAATCTCCGACAAAGACTTCGCCCTCTCCCTCCTC |
| | GGACACATCAACTTCACCATCAAAAAATTCTCCGACTTCGGCCTGATCCTCGTGG |
| | CCCCGACCCCACTGAAGACCGAACTCAAGAACGTGAGGCAAATTTACGCCAACCT |
| | GAAAGGCAACGTCAAGGTGGCCCACAAGGCAGGCTGGTTCCCGACCATTACCCAG |
| | ATCGCCATCAACCTCGTTTCCGAAGAGATAATGGTGAAAGATGGGGGCAAGTTCG |
| | CCCCAAAATTCGGCTCCCTGATCTATTCAATAATGAGGAAAACGGGCAACCAGTG |
| | GAAACCAAGCACCGGCGGCATCTTCCCGCTGGATTTCCTGCACCAAATTGCGGAT |
| | TCCGACAACGCCATCAACATCCTCAACAAGTGGAAGAAAATATTCGGATGGACGT |
| | CCCGCAAGAACGGCCACGAGGACCTGCCAACCTCCCTCGCCGAGTTCATCGCCAA |
| | CCCGAACCTCTTCAACTACCAAAGGTACGTCAACTTCCACCTGCGCAACGAAATC |
| | GACAAGGACAACATCAAATTCGGCGACTACAAAAAAGAAGACTTCCTGGAGGTCA |
| | TGAAGAACGTGGGCATC |
| 40 | Nucleotide sequence encoding I-A-3 Cas7 |
| | ATGGTGAACGAGACGGAGATCTACGAGATTGCGATCTTGGGGAGGGCGACGTGGC |
| | AGCTGCACAGCCTGAACAACGAGGGGACGGTGGGGAACGTGACGGAGCCGAGGAG |
| | CGTGACGATCATTGACCCGAACACCAAGAACCCGATCACCACCGACGGGATAAGT |
| | GGCGAGATGCTCAAACACATCCACACCGGGCTGATGTGGACGCTGACGGACAAGA |
| | ACAACCTGTGTGACGCCTGCAAGGTGCTGAACCCGGAAAAGTTCAACGTGACCAG |
| | TGGCAGGGGCAGCACCGTTGAAGAGGTGCTCGAAAATGCCCTGAATAAGTGCGAC |
| | ATATGCGACCTGCATGGCTTTCTGATCACGAGGCCTACCGTCTCCAGGAAGTCCA |
| | CCATCGAGTTCGGGTGGGCCCTCGGGATTCCTGAGATCTACCGGGATATTCACAC |
| | CCACGCCAGGCACGCCCTGGGCGGTAAGACCACCGAGAACGAAGAGAGTAAGGGC |
| | GTGAACACACCGAACAGCAGCGAGGATAAAGAGGAGGCCGTGGGCACCAGCACCC |
| | AGATGGTGTACCACAGGCCAACGCGGAGCGGGGTGTATGCAGTGATCAGCATGTT |
| | CCAGCCGTGGAGGATCGGCCTCAACGAGACGAGGCAGGACCAGTACACGTATGAC |
| | ACGGGCAACAACGAGAAGAGGATCGAGAGGTACAAAAATGCGCTGAAAGCCTACC |
| | AGATCCTGTTCACCAGGCCGAAGGGGGCCATGAGCACAACGAGGCTGCCGCACGT |
| | CGAGGACTTCGAGGGCGTGATAGTCTTCTCCACCGACCAGATCCCGCTTCCCCTG |
| | ATCTCCCCACTGAAGCAGGACTACGTCAAAGAAATCACCGACATATCCAAGAAGA |
| | TTGACAACAGCATAAACGTGGAGGAGTTCAAGACCCTGTCCGAGTTCGTGGACAA |
| | AATCGGCGACCTGATCGACAAAAAACCGTACAAGCTGAAGCTGGGCGAG |
| 41 | Nucleotide sequence encoding I-A-3 Cas6 |
| | AGGCTGAAGATCTCCCTGACCTCCAACAACGGCAACTACCTGATCCCGTACAACT |
| | ACAACCACATCCTGTCCGCCATCATCTACAGGAAGATCGCCGACCTGGACCTGGC |
| | CGCTAAGCTGCATTTCTCCAAGGACTTCAAGTTCTTCACCTTCTCCCAGATCTAC |
| | TTCTCCGACTGGAAGCGCACCAAGAATGGCATCATCAGCAAGGACGGCAAGCTGA |
| | GCTTCTACATCTCCTCCCCCAATGAGCAGCTGATCAAGTCTCTGGTCGAGGGGCA |
| | CCTGGAGAACACGGAGGTGGACTTCAAGGGCAAGAAGCTGCTGGTGGAGCAGATC |
| | GAGCTGCTGAAGAGCCCGAGCTTCAAGGAGAACATCAAGCTGAAGACGATGAGCC |
| | CGGTGGCAGCCAGCATCAAAAGGGAGGTGGACGGGAAGCTGAAGATCTGGGATTT |
| | GGGCCCGGGGGATGAAAGGTTCTACGAGTCCGTGCAGAAGAACCTGGTGAACAAG |
| | TACACGTCCTTCTACGGGGACTACGACGGGGACAAGTGGGTGAGGATCAAGCCGG |
| | ACATGAAGACGGCGAAGAGGAGGAGGATCGAGATTAAGGGGGACTTCCACAGGGG |
| | GTACATGATGGAGTTCGAGATGGAGGCCGACCCGAGGCTGGTGGAGTTTGCGTAC |
| | GACTGCGGGCTGGGCGAAAAGAACAGCATGGGGTTCGGGATGGTGAACATTTACG |
| | AG |
| 42 | Nucleotide sequence encoding I-A-3 Csa5 (Cas11) |
| | TCTGAGTTCAGGCTGAAGGACGTGTTTGAGCACGAGAGCATCAAGTCCTTCGGCA |
| | AGACGCTGAGGAAAATGATCAGACCGCCGAAGGAGGGGAACAAGGAGAAGTGGGC |
| | GAGCGACTACGCCAGCATCGTCGAACTGGGGTACGTGGAGACGAAGGACCAGTTC |
| | GCGGAGGTGATCAAAAAGCTGCTGAGGAGGTACGACGTGATAGCGAAGAAGCACC |
| | AGCTGAAGAGACCGACTGAGAAAAACCTGGAGGAGTTGATGGAGCTGATCGACAA |
| | GTATGGCGTGAAGCCGGTGAGGGCGGCCTTGATAAGCTACGCCCTGGTGAAAAAA |
| | GACGAAGAG |
| 43 | Nucleotide sequence encoding I-A-4 Cas3 |
| | AAGTACAAGGAGATTTTCGAGAAGCTGAAGCTGAACAACCTGACCGAGGTGCAGC |
| | AGAAGATCTCCGAGCTGGAGGGCAGCAAGAACATCCTCGTGGTGAGCTCCTGCGG |
| | CTCTGGCAAGACTGAGGCTTCCTACTTCAAGATGCTGGAGTACAACAGGAAGACG |
| | ATCATCATCGAGCCGATGAAGACCCTGACGAACAGCATCCACGGCAGGGTGGACA |
| | TCTACAACAAGAAGCTGGGCCTGGAGAAGGTGTCCATCCAGCACAGCAGCAGCCA |
| | GGAGGACAGGTTCCTGCAGAACAAGTACACCGTGACCACCATCGACCAGGTGCTG |
| | GTTGGCTACCTGGCGATGGGCAAGCAGGCTTACATCAAGGGCAAGAACATCGTGA |
| | TGTCCAACCTGATTTTCGACGAGGTGCAGCTGTTCGACACCGACACCATGCTGCT |
| | CACGACCATTAATATGCTCGATGAGATCTACAAGCTGGGCAACAAGTTCATCATC |
| | ATGACCGCCACCATGCCCCAGTTCCTGATTGAGTTCCTGGGCGAGAGGTACGACA |
| | TGGAGATTGTGATCACCGAGAAGATCAGGGAGGACAGGAACGTCAAGCTGTTCTA |
| | CGAGGAGGAGCTGGATTACAACAAGGTTAGGAATTACAAGGATAAGCAGATCATC |
| | ATCTGCAACTCCATCAAGCAGCTGAAGGAGATCCACAAGAAGCTGCCAAACTCCA |
| | GGGTCATCACGCTGCACTCCACCTTCCTCGGTTCCAACAGGCTGAAGCTGGAGAA |
| | GCAGGTGGAGAGGTACTTCGGCAAGCACTCCGAGCAGAACGACAAGATCCTGCTG |
| | ACAACCCAGATCGTGGAGGTGGGCATGGACATCAGCTGCGACAGGCTGTACACAA |
| | CCGCGTGCAAGATCGACAACCTGGTGCAGAGGGACGGCAGGTGCTGCAGGTGGGG |
| | CGGCGATGGCCAGGTTATTGTGTTCAAGAACGACGACAACATCTACGAGAAGGAG |
| | CTGGTCGAGGAGACGATCAAGTACATCAAGAATAACCAGGGCATCGCGTTCAACT |
| | GGACGATCCAGAAGCAGTGGATTAACGAGATCCTGAACGAGTACTACAAGAACAA |
| | GATCAACGAGTACAACCTGAGGAAGAACAAGTTCAACTTCAACGGCTGCAACAGG |
| | TCCCGCCTGATCAGGGATATCCAGAACATCAACGTGATCGTGGTGAACAAGGAGG |
| | AGTTCACGAAGCAGGACTTCAACAGGGAGTCCGTCAGCCTGCACATCAACAAGCT |
| | CAAGGAGCTCTCCCAGGCCAACGAGATCTACATCCTCAACAAGAACAAGATTGAG |
| | AAGGTGAAGTACAACAAGGTCGAGATCGGCGACACGGTGATTATCCGCGGCAAGA |
| | ACTGCAGGTACGACGACCTTGGCTTCAGGTACGAGGAGGACAGCGCCAAGAACAT |
| | GCCGAAGTGCAGGGACTTCCCAATGACCAACAAGAGCAACAACAACCAGTTCAGG |
| | GATTACATCGAGGAGACCTGGATTCATCACGCCGAGACCGTGAGGGACCTGATGT |
| | CATACAGGCTGAACCAGGAGCAGTTCAACGACTACATCATCATCAACGGCAAGAA |
| | GATCGCGTTCTACGGCGGCCTGCACGACCTTGGCAAGCTGGATCTGGAGTGGTCC |
| | CGCAAGTACAAGAGCGCCATCCCACTCGCTCACTTCCCGTTCGTGAAGGGCTCCA |
| | TGGGCGAGAAGAGGACCCACGAGCTGATCTCCGGCGAGATTCTGAAGGAGATTAT |
| | TGACGACGACATCATCTACAACATGATGATCCAGCACCACAAGCGCCTGTACGAC |
| | GATATTGACATCGACTACAAGGGCATCGAGTGGGAGCTGCATAAGGACACCTACA |
| | AGATCCTGACCACCTACGGCTTCAAGGACGACATCCAGCTGCAGAGCGACGCCAA |
| | GACTCTGAAGCGCAACAACATTATGAGCCCGTGCGACAACGAGTGGACCACCCTT |
| | CTCTACCTGGTGGGCACCTTCATGGAGTGCGAGATTCAGGCCATCAACGAGTACA |
| | TCGATAACTACAAGCAGGCCATC |
| 44 | Nucleotide sequence encoding I-A-4 Cas5a |
| | AAGAAGGTGACCTACAAGCTGAGCAATATCTTCTCCCTTAAGAAGTACAACGACA |
| | ACAACCTGAACTGCCAGAGCTACGAGTACCCCACCATCTACGGCATCAGGTGCGC |
| | CATCCTGGGCGCTATTATCCAGGTGGACGGCATCGACAAGGTTCAGGAGCTGTTC |
| | AACAAGATCAAGAACAGCAACATCTACATCCAGTACCCCAAGGAGTTCAAGGTGA |
| | ACGGCATCAAGCAGAAGAGGTACGCCAACTCCTACTACAACTCCTGCTACACCGA |
| | GGAGGAGTACAACAAGCTGTCCCCGTCCACGCAGTCCAAGACCTACTGCGTGCTG |
| | GACAGGGACAAGCTGGTGGGCTCAAACTGGAAGACCACCATGGGCTTCCGCCAGT |
| | ACGTGAAGATGGACAACATCGTGTTCTACATCGACAACCTCATCCCGGAGATCGA |
| | CATGTACCTGAAGAACATCGACTGGCTGGGCACCGCGAAATCTATGGTTTACCTT |
| | TCTGACGTGGAGGAGGTGAACAAGCTGGACAACGTGCTGACCAGGTGGAACAAGG |
| | AGAGCTACGTGGACACCTTCGAGCAGCACGACTGGAATTCCAAGACCACCTTCGA |
| | TACGATCTACATGTACTCCAAGAAGTACAAGCACTTCCACGATACCTTCATGTGC |
| | GGCATCGGCGACATCATCCTGCCCTCTTGGCTCTGGTACACGAGGTACACCTTCA |
| | TCCTTTACTTCAAGCTGTGGCTGGTGAATCTGTACGAGAAC |
| 45 | Nucleotide sequence encoding I-A-4 Cas8a |
| | AATGAGTACGAGTTCAAGGTGATTAAGACGGCCAACGACATCGAGGACATCTGCA |
| | TCTCCTACGGCATTTGCAAGATCCTGAGCGACAACAGGATCAAGTTCAAGCTGAA |
| | GGATAACAAGTCCATGTACTCTATCTACACCAAGGAGTTCGACATCCAGAACGAC |
| | ATCTTCTACAATGACTTCAACATCGAGAACGTCTGGAACCTCAACTCCGGCCTCA |
| | ACCAGAAGGAGACCGTGAGGGCTCTGGACGACATGAACAAGTTCCTCTCCGAGAA |
| | CATCCACGACATCCTGGAGCACCTGCTGAACGGCAAGGTGCTTAACTACAAGAAG |
| | GAGAGCGCCAAGGGCATTGGCAATTGCTTCTACTCCCTGGGCGTGAGGGCCTCTA |
| | CCTTCGGCAAGACACTGGAGATCTCCCCCATCAAGAAGTACCTGTCCTTCCTCGG |
| | CTGGATCTACGGCTGCAGCTACTGCTACAAGGAGAAGTCCTTCGAGATTACCGCC |
| | ATCCTGAAGCCCTACAACACCGACGAGATCGCCAAGCCCTTCAACTTCTCCTACG |
| | TCGACAAGGAGACCGGCGACAAGAAGATCCTGACCAAGATCAAGAAGGCCAGCGA |
| | GATCAACATGATGTCCATCCTCTACATCGAGACCCTCAAGAAGTACAAGATGCTG |
| | TCCGACGAGTACAGCAACGTGATCTTCATGCAGAACATCATCGCCGGCCAGAAGC |
| | CACTGTACGACAAGACCACCAACATCAAGATCTACAAGCTGTCCCAGAAGTACCT |
| | GGACGACCTGCTGAAGAAGCTGACCTGGTCCAACGTGTCCGAGGACGTGAAGGAC |
| | ATCACCGCCAGGTACGTGCTGAACATCGACAAGTACAAGGAGTTCTCCAAGCTGA |
| | TCAAGATCTACTCCAAGGACGGCAACTCCAAGATCAACAACGACTTCAAGGGCGA |
| | GATCCTGTCCATGTACAACGAGATGATCAAGAAGATCTACAACGACGAGACCATC |
| | AACAAGATCGGCAAGGGCTTCAACAGGCTGCTGAGGGACAACAAGGGCTTCGAGA |
| | TCCAGACGAAGCTGTACAACGTCGCCAACGAGAAGCACCTGGTGAAGGTGCTGAA |
| | GATGATCATCGACCTGTACTCCAGGAACTACAAGTCTGCCATCCTGAACAACGAC |
| | GAGCTGAACAAGCTGATCAACACCATCGAGGATAAGGAGTACGCCAAGATCTGCT |
| | CCGACGCCATCCTGTCCATCGGCAAGGTGTTCATCATCATCAAGAAG |
| 46 | Nucleotide sequence encoding I-A-4 Cas7 |
| | AACAAGATCGCGATGATGATGAGGCTGAAGCTGACGGGCGAGGCCCTTAACAACG |
| | AGGGCACTATCGGCAACGTGATCCAGCCGAGGCAGATCGAGTTCCCGAATGGCGA |
| | GGTGAGGCAGGCTATTTCTGGCGAGATGCTGAAGCACTACCACAGCAGGAACCTG |
| | AGGCTGCTGGCGGATGAGAACGAGCTGTGCGACACCTGCAAGATCTTCAGCCCGA |
| | TGAAGAACGGCAAGGTCAAGGAGAGCGATAGCAAGCTGAGCCCGTCCGGCAACAA |
| | GGTGAAGGAGTGCATCGTGGACGACGTGGAGGGCTTCATGAACGCCGGCAAGGGC |
| | GCTAACGAGAAGAGGACTAGCTGCGTGAAGTTCTCCTACGCCATCGCCACCGAGG |
| | AGAACGAGTACCAGATCATGCTGCATACCAGGGTGGACGTGACGCAGGACAACAA |
| | CAAGAAGAAGCAGGAGAAGGAGACGACCGAGGGCGAGGGCAATACCAACAAGGAC |
| | CAGAACACCCAGATGCTGTTCCACCGGCCGCTGAGGAACAATGAGTACGCCATCA |
| | CGGTGCAGGTGGACCTGGATAGGATCGGCTTCGACGACGAGAAGCTGATCTACGC |
| | CCTGGACGAGGACACAATCAAGTCCCGCCAGGAGAAGTGCATCAAGGCCCTGCTG |
| | AACATGTTCGTGGACATGGAGGGCGCCATGTGCTCCACTAGGCTGCCACATATCG |
| | AGGGCATCGAGGGCATTATCGTGAAGAAGACCGACAAGAACCAGGTGCTCTCCAA |
| | GTACTCCGCCCTGAAGGATGACTACAAGGAGGTGAACGAGAAGATCTCAGACGAC |
| | TCCATCATCTTCAACAACATTATCGAGTTCTCCGAGGTGATGAAGGGCCTCATT |
| 47 | Nucleotide sequence encoding I-A-4 Cas6 |
| | AGGATCAACCTGCAGGGCACCATCATCGAGGGCCAGTCTTCCATCAAGACAAACT |
| | ACAACCACGAGATGTACAGCATGATCCTGACCAACATCTCCACCGAGCGCGCCAA |
| | CTACATCCACGAGAAGAAGAGGTTCAAGAGGCTGTTCACCTTCTCCAACCTGTAC |
| | ATCTCCGACAACAAGGTGCACTTCTACGTCTCCGGCCAGGACGAGCTCATCAAGG |
| | ACTTCATCAACTGCATCATGTTCAACCAGATGGTGAGGGTGGGCGACCGCGTTAT |
| | CTCCATCACCAACATTGAGCCGATGAAGAACTCCCTCGAGACGAAGAAGGAGTAC |
| | ATCTTCAAGTCCAACTTCATCGTGAACCAGAAGGAGAACGATAGGGTGTGCCTGA |
| | GCAAAGATATGGGTTACGTGATGAAGCGCATCTCCGACATCGTGAAGGACAAGTA |
| | CAAGGAGATCTACAAGGAGGAGATTAACGAGAACCTGAACGTGGAGATCCTGAAT |
| | AGCAAGCAGAAGTACACGAAGTACAAGGACCACCACCTGAACAGCTACCAGGCGA |
| | CGCTGAAGGTGAGGGGCAACAAGAAGCTGATCGACCTGCTGTACAACGTGGGCAT |
| | CGGCGAGAACACCGCCTCAGGCCATGGCTTCGTGTGGGAGGTT |
| 48 | Nucleotide sequence encoding I-A-4 Csa5 (Cas11) |
| | AACAACGAGATCAAGATCGTGAAGTGCATCGACAGCCTGTACCCGACCGTGAAGC |
| | TGACGATTGGCAAGCTGTACAAGGTGAAGGAGAGCGAGAACGACAAGTTCTACAG |
| | GGTGATCGCCGACGACAACAACGAGGAGCAGCTGTGCTACAAGTACAGGTTCGAG |
| | CTGGTGGATATCAACGAGATCAAGGAGCTGACGCTGCAGGACATCTTCAACGAGG |
| | AGGAGGGCATTAAGTACAACAGGATCAACGGCGGCTCAGGCATTTACACGATCCA |
| | GAACGAGACCCTGATTATCGGCGAGCACATCAAGCCGGTGCTTAACAAGAGGATC |
| | ATGGACTCCAAGTTCGTCAAGGTCAAGGTGGAGAGGCTGGTGTCCTTCAGCGACG |
| | TGATCAACTCAGACTACAAGTGCAAGGTGAAGCACTACAGGGTGGAGGGCCTCAT |
| | CCAGGAGGAGTCTAGCTACACCTGGCTGGAGGAGTACCAGGACCTTAAGGACATC |
| | ATGCTGGCCCTGTCAGAGGAGTTCAACACCATCGCCCTGAAGGAGATCATTAACA |
| | AGGGCCAGTGGTACCTGGAGAAC |
| 49 | Prototype direct repeat sequence of I-A-1 |
| | GUUCCAGAGCCUUCCCCGAUGAAGAGGGGACUGAAAG |
| 50 | Nucleotide sequence encoding prototype direct repeat sequence of I-A-1 |
| | GTTCCAGAGCCTTCCCCGATGAAGAGGGGACTGAAAG |
| 51 | I-A-1 mature direct repeat sequence - |
| | first region containing stem-loop structure |
| | GUUCCAGAGCCUUCCCCGAUGAAGAGGGG |
| 52 | I-A-1 mature direct repeat sequence - |
| | second region not containing stem-loop structure |
| | ACUGAAAG |
| 53 | Prototype direct repeat sequence of I-A-2 |
| | GUUGAAGAGCCUUCCCCGAUGAAGAGGGGACUGAAAG |
| 54 | Nucleotide sequence encoding prototype direct repeat sequence of I-A-2 |
| | GTTGAAGAGCCTTCCCCGATGAAGAGGGGACTGAAAG |
| 55 | I-A-2 mature direct repeat sequence - |
| | first region containing stem-loop structure |
| | GUUGAAGAGCCUUCCCCGAUGAAGAGGGG |
| 56 | I-A-2 mature direct repeat sequence - |
| | second region not containing stem-loop structure |
| | ACUGAAAG |
| 57 | Prototype direct repeat sequence of I-A-3 |
| | GCUCAAAUCAGACUAUUUUAGGAUUGAAAU |
| 58 | Nucleotide sequence encoding prototype direct repeat sequence of I-A-3 |
| | GCTCAAATCAGACTATTTTAGGATTGAAAT |
| 59 | I-A-3 mature direct repeat sequence - |
| | first region containing stem-loop structure |
| | GCUCAAAUCAGACUAUUUUAGG |
| 60 | I-A-3 mature direct repeat sequence - |
| | second region not containing stem-loop structure |
| | AUUGAAAU |
| 61 | Prototype direct repeat sequence of I-A-4 |
| | AUUAAGAUUUAACAAUCAUGUUAUUUAAA |
| 62 | Nucleotide sequence encoding prototype direct repeat sequence of I-A-4 |
| | ATTAAGATTTAACAATCATGTTATTTAAA |
| 63 | I-A-4 mature direct repeat sequence - |
| | first region containing stem-loop structure |
| | AUUAAGAUUUAACAAUCAUGU |
| 64 | I-A-4 mature direct repeat sequence - |
| | second region not containing stem-loop structure |
| | UAUUUAAA |
| 65 | NLS amino acid sequence |
| | MPKKKRKV |
| 66 | linker-1 amino acid sequence |
| | GIHGVPAAGS |
| 67 | linker-2 amino acid sequence |
| | GSG |
| 68 | Amino acid sequence of NLS-(linker 1)-(I-A-1 Cas3) fusion protein |
| | MPKKKRKVGIHGVPAAGSLEEQFQLVTGHSPAEHQRECGEALATGKSVILRAPTG |
| | SGKSEAVWIPFLRCRGKRLPMRMIHALPMRGLANQLEERMKDYAGPGLRVSAMHG |
| | QRPESVLFYADAIFATIDQVVASYACAPLSLSVRHGNIPAGAVASSFLVFDEVHT |
| | FEPRLGLQSILVLAERAHQMGMPFVIMSATLPKNFIRSLAERLGAAPIEGGRLKS |
| | KEGEPRHVTLRVLPEKLSARTILDYAPKVNRTVVVVNTVQRALGLYEQVRDEFRC |
| | PVILAHSRFYDEDRRTKEQQIEALFGKKAAQGRCLLIATQVVEVGLDISCDLLIT |
| | ELAPVDALVQRAGRCARWGGKGDVIVLTELDTKRPYDETLVAVTERALQEHNVDG |
| | QELTWEVETALVDTVLDPHFKEWAKPDAAGKVLASLAEAAFTGNSTKAEQAVRET |
| | LTVEVALHDTPQALGPAILRLPRCRLHPGVFQQFVRKQRPNVWQVVVDRDPDDDY |
| | RTRIEFLSVNGKSRLIPGGHYIVDPQFGCYDAERGLRLGVPGQSAEPFAPGQSRD |
| | RLKGELQIELWQDHIREVVKAFERYVLPKERMAFEALSRWLGKTQDELLSVARAV |
| | LVLHDLGKLARQWQGKIQAGLEGKLSQGSFLAHRGGSVSGLPPHATVSAWVATPC |
| | LRRLAGTDWEQTLAVPALAAIAHHHSVRADITPEFEMTDGCFEVVADCARGVAGL |
| | EVKRDDFNTKPPQGSGSCGVGLNFLLPEGYTSYVLLSRWLRLADRIATGGGEETI |
| | FQYEKWMGDS |
| 69 | Amino acid sequence of NLS-(linker 2)-(I-A-1 Cas5a) fusion protein |
| | MPKKKRKVGSGAEWLQAEVEFASFYSYRVPDLSPSFALCSPVPSPAAIRLAVVDA |
| | TIRHTGDVNEGHAVFELMKRARLELQPPSRVAVMKFFIKRLKPEKPTKGKRASVI |
| | ESTGIREYCLPWGPMVFWIESDQPERIAQSLQWLRRLGTTDSLASCTVGAGTPNF |
| | ASCIRPANGLTLQTTNFAQRPVFTLHELKPETQFNQVNPFADERPGKPFEKRLYV |
| | LPLVREKVGENWVIYHHEPFAA |
| 70 | Amino acid sequence of NLS-(linker 2)-(I-A-1 Cas8a) fusion protein |
| | MPKKKRKVGSGEYRLIKSGLEMFDTARAYGLAQLLQVLAGGRAAPRILSQGGVFT |
| | LTISTKPNPATLKSSDLWRGAFGESNWQKVFLTYKRAWSSQRDKVKRSLESHSAD |
| | IFGKAETDGLAVVFGGNFALPGPLDPVGFKGLKGLTAGSYSEGQTTVDEFNWALG |
| | CLGAAAAQRYKIQKAVGNKWEYYVTLPVPEEVQFGDFHAVRQLVYDKGLSYNGVR |
| | NAAAHFSLLLASAIREKAQGNPHFPVRFSNVLYFSLFQSGQQFKPAIGGAVNVGR |
| | LIEIALARPEVALEMFKTWDYLFRRGSAQGNEDLAQAITELVMAPSLDTYYRHAR |
| | IFNRYVVDSTKRVRPEYLYDETALKEVLNYAEQ |
| 71 | Amino acid sequence of NLS-(linker 2)-(I-A-1 Cas7) fusion protein |
| | MPKKKRKVGSGADSPVFEVAILGRVVWNLHSLNNEGTVGNVSEPRTVVLADGSKS |
| | DGISGEMLKHIHAQNVWLVAEDKSQLCEPCRTLNPQKADKNPAVLGVKTAKAKVA |
| | AESMSVAISSCALCDLHGFLVQRPTIARASTVEFGWAVALRDGYHRDIHLHARHA |
| | VEGRAETTEGQQEGPAEVSGQMIYHRPTRSGTYAFVSVFQPWRIGLNEVNYEYVE |
| | GVDREARNKLAIEAYKATFARTGGAMTSTRLPHVEALEGVVLVSSRNFPVPVTSP |
| | LQDDYREKTEKVGQAVEGLEVQRFGALPELYVILNALAKRRLFALQMGGTSKKGK |
| | Q |
| 72 | Amino acid sequence of NLS-(linker 2)-(I-A-1 Cas6) fusion protein |
| | MPKKKRKVGSGGDVLGLHSLRVGLFRFRLVPEQPLEVPALNKGNMLRGGFGHGFR |
| | KLCCIPECRDARLCPLAAICPYKAVFEPSPPPGSERLAKNQDIPRPFVFRAPHTN |
| | QTRFQKGEAFEFGLVLIGRAVDYLPYFVLSFRELANEGLGLNRAKCALERVEQRR |
| | TSANGLGRATGEGRLVYSKDSGVFHSTENEGVDSYVNSRLRELSSPNGDQSRQNV |
| | TIRFLTPTFLKANGEVIRRPEFHHLFKRLRDRINALCTFFGDGALDLDFRGVGTR |
| | AEKVQSVSARTEWVERCRTSSKTGQRHELSGFMGEATYEGNVEEFLPLLALGELV |
| | HVGKHTAWGNGRIELQSGTGVKC |
| 73 | Amino acid sequence of NLS-(linker 2)-(I-A-1 Csa5(Cas11)) fusion protein |
| | MPKKKRKVGSGLSSDSKLSEVFAEESVKSFGKCLRYALWRDEDYASLIEFENAET |
| | PTQFADAVRKFLRRYRSGGFMDQTQRSRASEMRKQNRWDGLKKLLRQYEVGPRPS |
| | EGQLERLMQLANDTNGVRLVQSAIISYGLTKREPYKEVEELEKEN |
| 74 | Amino acid sequence of NLS-(linker 1)-(I-A-2 Cas3) fusion protein |
| | MPKKKRKVGIHGVPAAGSVACDTFFAPMTDFNLARHQSECAGALASGKSVILRAP |
| | TGSGKSEAVWLPFLSLRGKTLPCRLIHTLPMRALVNQLESRMRTYANGRMRVAAM |
| | HGQRPESVLFYADAIFATLDQVVTSYACAPLSLSVRQGNIPAGAVAGSFLVFDEV |
| | HTFEPHLGLQSLLVLAERAHQMGIPFVIMSATLPTNFIRRLSERFGATIVEGTRL |
| | EGKNRRQRRVVLRVSSEKLSIETILELTRNVERTLVVVNTVQRAQNLYEQLLGKI |
| | GCPVILAHSRFYDDDRRTKEKQIEAQFGKTAEGQCLLIATQVVEVGLDISCDLLV |
| | TELAPIDAIVQRAGRCARWGGQGEVVVFTGLETTRPYDRTLVEATEKALREKNLN |
| | GQELTWEIERALVDTVLEPQFSKWAEPEAAGKVLASLAEAAFTGDSAKAERAVRE |
| | GLTVEVALHGSPDTLGVGALRLPRCRIHPGGFQQFVHKQQPEAWRVVVDRTAADD |
| | YRTRVEFLHVDSNSKAAPYGYYIIHPQYGSYDVERGLRLGIRGSPAQSRDELIQR |
| | KSRLEGELQIEKWQDHIEKVVKAFAEHVLPKERIAFEALSRRLGKTHEDLLSLTH |
| | LVLIFHDLGKLAQQWQRKIQAGLESVLPPGTFLAHRGGSLRDLPPHATVSASLAT |
| | PCLCRVAGPDWQQTLAIPALAAIAHHHSVRADMTPQFDMSEGWFDVVADCARRLA |
| | GVDVTVNDFSRWRGGGSCGVALNFLLPDGYTSYILLSRWLRLADRIATGGGENAI |
| | LNYEDWMSSS |
| 75 | Amino acid sequence of NLS-(linker 2)-(I-A-2 Cas5a) fusion protein |
| | MPKKKRKVGSGAEWIQAEIEFASFYSYRVPDLSPSYALSSLVPSPAAIRLAVVDA |
| | VIRHTGVVDEGESIFELVKRAKLEVQPPARIAVMKFFVKRLKPENPEKGKRASVI |
| | ESTGIREYCLPSGPLVLWLETEEPERIGQALQWLRRLGTSDSLATCKIGHGAPDT |
| | ALCIKPANGLAIQAKNFAQRAVFTLHELKPDANFSEVNPFADGRRGDPFEKRLYV |
| | LPCVREQAGENWVLYRREPFAN |
| 76 | Amino acid sequence of NLS-(linker 2)-(I-A-2 Cas8a) fusion protein |
| | MPKKKRKVGSGEYLVVKSGLPTLDAARAYGLAQLLQVLANGKASPYITDQGGVFA |
| | VSLNAELTHDALTRSDMWRAAFADSNWQRVFLTYKKAWSAQRDRVKRTLEEQVAA |
| | VVTRAGDGLCVDFAGKFALPGPLDPVGFKGLKGLTAGNYSEGQTYLDEQNGALAC |
| | LGATIAQRYKFGKREYFVTLPIPQMVQFNDFHQIRHLVYDKGLAYLGVRTAAAHF |
| | ALIFADAIRERAAGNPYFPLSFSNVLYFSLFQSGQQFKPSVGGSINLARLLDIAL |
| | SRPQAAAEMFKTWDYLFRRGSVKGNEALAEAITDLLMAPSGESYYRHARIFNRYI |
| | VDSSKRVNSEFLYDEAALMEVMAYVEQ |
| 77 | Amino acid sequence of NLS-(linker 2)-(I-A-2 Cas7) fusion protein |
| | MPKKKRKVGSGAGNSVFEISILGRSVWNLHSLNNEGTVGNVSEPRTVILADGSKS |
| | DGISGEMLKHIHAQNVWLVATDRSVFCEPCQTLQPQKADKNPDVTGVKAARAKLA |
| | SEGMNVAIAACALCDLHGFLVQKPTIARASTVEFGWAVAVRNGFHRDIHLHARHA |
| | VEGRTEGQQEAGEVAAQMIYHRPTRSGTYALASVFQPWRIGLNEVNYEYVAGVDR |
| | EARYRLAIEAYKATFARTDGAMTSTRLPHPEAFEGVVLVSSRNFPVPVTSPLQDD |
| | YREKLQQLSRATEGLEPQPFNSLTELYGILNELAKRPLFNLQLARSSKREKK |
| 78 | Amino acid sequence of NLS-(linker 2)-(I-A-2 Cas6) fusion protein |
| | MPKKKRKVGSGSQAHCECSLRVRRFRFVIAPREPLLVPAINKGNMLRGGFGHAFR |
| | CLCCIPQCRDARTCPVGMSCPYKAIFEPSPPPEAEALSKNQDIPRPFVFRAPKTQ |
| | QTRFETGQPFEFELVLIGRALDFLPYFVLSFRELAAEGLGLNRAKCSLERVEQVD |
| | LTSEAADASNYEAMVIYTAEDQVFRNAATSETGEWIGRRIRNRSTSRDNDSVQQV |
| | SIRFSTPTFLKADGEIIRQPEFHHVFKRLRDRINALSTFFGEGPIEADFRGLGER |
| | AEKIRTVSARTDWVERFRTSSKTKQRHELSGFVGEVTYEGNLNEFLPWLTLGELV |
| | HVGKHTAWGNGWMELEHEVSRGCV |
| 79 | Amino acid sequence of NLS-(linker 2)-(I-A-2 Csa5(Cas11)) fusion protein |
| | MPKKKRKVGSGSNSEISLASVFAEESIKSFGKCLRYALWRDDDYASLIEFENAET |
| | PLQFAEAVRKFLRRYRSGGFMDEALRTQASEMRKHNRWDELRRTLRQNEIGPRPT |
| | EGNLERLTQLANNAQGVRLVRAAIISYGLTKRDPHKELEEVERGS |
| 80 | Amino acid sequence of NLS-(linker 1)-(I-A-3 Cas3) fusion protein |
| | MPKKKRKVGIHGVPAAGSNKLFKKLIGAKPYDYQKIAMENLLDGKSIIMRAPTGS |
| | GKTEIALIPFLYGFNDLLPSQLIYSLPTRTLVESIGERAVKYASFRKLRVAIHHG |
| | KNATSSLFEEDVVVTTIDQAVGAYLSTPLSMSKRSGNIFVGSVGSALTVFDEVHT |
| | LDPEKGLQTSLAISMQSAKLGLPTLIMSATLPDIFIETAKDRISKKGGDIEFIDV |
| | KDEFEIKSRKNRFVELINRLEEELNAEKVLEEVEHGKRIIIVINTVNRAQELYLE |
| | LRNKTELPILLLHSRFLEKDRQEKELLLEETFGKNGNGKCIFIATQIVEVGMDIS |
| | SPKVLSEIAPIDALIQRAGRCARWSGKGEFHVFGYNTNSKSPHAPYNKDIVEATK |
| | SEINNKGKSFTLDWNTEVELVNKILTKHFSEFMNSMIFYQRLGELARAVYEGSRA |
| | KVEQNVREVFSCDVTLHENPKSMNSVEILHLPRLRLDARTLMGKVEKIAEMGIDT |
| | YRLEENTIIFDDDEDEYVPVLVNNREEIIPFELYVLCGASYSSDTGLVFDDFPNA |
| | LKSFDPEEKEILSSKQFDNRLKVETWVEHAKNTLKVLDNYMIPRYRYSIENFAEN |
| | YGYNYGEFLDIIRCTVSLHDIGKLNKKWQKRIKWNDETPLAHSNDNTIKRLPAHA |
| | TVSAKALQPYLEDLFDDEDIFKAFYLAIAHHHQPWSKSYNEYELVPKYDESLKEI |
| | WIIPKNFIQEQNPAGRLDFSYLDIIDENEAYRLYGFLSKLMRISDRLATGGNTYE |
| | SLFSG |
| 81 | Amino acid sequence of NLS-(linker 2)-(I-A-3 Cas5a) fusion protein |
| | MPKKKRKVGSGQWLKFTLHFPSFFSYRIPDYSSQYALGIPLPSPSTLKLGVISSA |
| | IKSTGKVSEGEKVFNVVKDAEVCVAPPEKIAINSFLIKRLKKRKEDLKLIPTFGI |
| | RDYVFFPDDIDIFVGSENIDSVAEYFSKMNYIGSSDSMVYVKSIEPKTPSENVIK |
| | AVDIDEFSDAAEKESYLVYPVKDINKNATFDQINSYSSKSSRKILDQKYYLINAK |
| | VSKGKNWKILDTRN |
| 82 | Amino acid sequence of NLS-(linker 2)-(I-A-3 Cas8a) fusion protein |
| | MPKKKRKVGSGNHYFLAKSGWEFFDVSKAYGLGLVIQTLTGNASITDRGGFYLIE |
| | SKNETKFDKIEEISKYFDDSELKTTLITIQRSTKSEMKPPVKKVKGKCLETLTDK |
| | ESMITVIKNYENLNSPSIIGTDKQTLYQTMDLAATKGIRNEILLKKNYSDGTNIK |
| | ISDKDFALSLLGHINFTIKKFSDFGLILVAPTPLKTELKNVRQIYANLKGNVKVA |
| | HKAGWFPTITQIAINLVSEEIMVKDGGKFAPKFGSLIYSIMRKTGNQWKPSTGGI |
| | FPLDFLHQIADSDNAINILNKWKKIFGWTSRKNGHEDLPTSLAEFIANPNLFNYQ |
| | RYVNFHLRNEIDKDNIKFGDYKKEDFLEVMKNVGI |
| 83 | Amino acid sequence of NLS-(linker 2)-(I-A-3 Cas7) fusion protein |
| | MPKKKRKVGSGMVNETEIYEIAILGRATWQLHSLNNEGTVGNVTEPRSVTIIDPN |
| | TKNPITTDGISGEMLKHIHTGLMWTLTDKNNLCDACKVLNPEKFNVTSGRGSTVE |
| | EVLENALNKCDICDLHGFLITRPTVSRKSTIEFGWALGIPEIYRDIHTHARHALG |
| | GKTTENEESKGVNTPNSSEDKEEAVGTSTQMVYHRPTRSGVYAVISMFQPWRIGL |
| | NETRQDQYTYDTGNNEKRIERYKNALKAYQILFTRPKGAMSTTRLPHVEDFEGVI |
| | VFSTDQIPLPLISPLKQDYVKEITDISKKIDNSINVEEFKTLSEFVDKIGDLIDK |
| | KPYKLKLGE |
| 84 | Amino acid sequence of NLS-(linker 2)-(I-A-3 Cas6) fusion protein |
| | MPKKKRKVGSGRLKISLTSNNGNYLIPYNYNHILSAITYRKIADLDLAAKLHFSK |
| | DFKFFTFSQIYFSDWKRTKNGIISKDGKLSFYISSPNEQLIKSLVEGHLENTEVD |
| | FKGKKLLVEQIELLKSPSFKENIKLKTMSPVAASIKREVDGKLKIWDLGPGDERF |
| | YESVQKNLVNKYTSFYGDYDGDKWVRIKPDMKTAKRRRIEIKGDFHRGYMMEFEM |
| | EADPRLVEFAYDCGLGEKNSMGFGMVNIYE |
| 85 | Amino acid sequence of NLS-(linker 2)-(I-A-3 Csa5(Cas11)) fusion protein |
| | MPKKKRKVGSGQWLKFTLHFPSFFSYRIPDYSSQYALGIPLPSPSTLKLGVISSA |
| | IKSTGKVSEGEKVFNVVKDAEVCVAPPEKIAINSFLIKRLKKRKEDLKLIPTFGI |
| | RDYVFFPDDIDIFVGSENIDSVAEYFSKMNYIGSSDSMVYVKSIEPKTPSENVIK |
| | AVDIDEFSDAAEKESYLVYPVKDINKNATFDQINSYSSKSSRKILDQKYYLINAK |
| | VSKGKNWKILDTRN |
| 86 | Amino acid sequence of NLS-(linker 1)-(I-A-4 Cas3) fusion protein |
| | MPKKKRKVGIHGVPAAGSKYKEIFEKLKLNNLTEVQQKISELEGSKNILVVSSCG |
| | SGKTEASYFKMLEYNRKTIIIEPMKTLTNSIHGRVDIYNKKLGLEKVSIQHSSSQ |
| | EDRFLQNKYTVTTIDQVLVGYLAMGKQAYIKGKNIVMSNLIFDEVQLFDTDTMLL |
| | TTINMLDEIYKLGNKFIIMTATMPQFLIEFLGERYDMEIVITEKIREDRNVKLFY |
| | EEELDYNKVRNYKDKQIIICNSIKQLKEIHKKLPNSRVITLHSTFLGSNRLKLEK |
| | QVERYFGKHSEQNDKILLTTQIVEVGMDISCDRLYTTACKIDNLVQRDGRCCRWG |
| | GDGQVIVFKNDDNIYEKELVEETIKYIKNNQGIAFNWTIQKQWINEILNEYYKNK |
| | INEYNLRKNKFNFNGCNRSRLIRDIQNINVIVVNKEEFTKQDFNRESVSLHINKL |
| | KELSQANEIYILNKNKIEKVKYNKVEIGDTVIIRGKNCRYDDLGFRYEEDSAKNM |
| | PKCRDFPMTNKSNNNQFRDYIEETWIHHAETVRDLMSYRLNQEQFNDYIIINGKK |
| | IAFYGGLHDLGKLDLEWSRKYKSAIPLAHFPFVKGSMGEKRTHELISGEILKEII |
| | DDDIIYNMMIQHHKRLYDDIDIDYKGIEWELHKDTYKILTTYGFKDDIQLQSDAK |
| | TLKRNNIMSPCDNEWTTLLYLVGTFMECEIQAINEYIDNYKQAI |
| 87 | Amino acid sequence of NLS-(linker 2)-(I-A-4 Cas5a) fusion protein |
| | MPKKKRKVGSGKKVTYKLSNIFSLKKYNDNNLNCQSYEYPTIYGIRCAILGAIIQ |
| | VDGIDKVQELFNKIKNSNIYIQYPKEFKVNGIKQKRYANSYYNSCYTEEEYNKLS |
| | PSTQSKTYCVLDRDKLVGSNWKTTMGFRQYVKMDNIVFYIDNLIPEIDMYLKNID |
| | WLGTAKSMVYLSDVEEVNKLDNVLTRWNKESYVDTFEQHDWNSKTTFDTIYMYSK |
| | KYKHFHDTFMCGIGDIILPSWLWYTRYTFILYFKLWLVNLYEN |
| 88 | Amino acid sequence of NLS-(linker 2)-(I-A-4 Cas8a) fusion protein |
| | MPKKKRKVGSGNEYEFKVIKTANDIEDICISYGICKILSDNRIKFKLKDNKSMYS |
| | IYTKEFDIQNDIFYNDFNIENVWNLNSGLNQKETVRALDDMNKFLSENIHDILEH |
| | LLNGKVLNYKKESAKGIGNCFYSLGVRASTFGKTLEISPIKKYLSFLGWIYGCSY |
| | CYKEKSFEITAILKPYNTDEIAKPFNFSYVDKETGDKKILTKIKKASEINMMSIL |
| | YIETLKKYKMLSDEYSNVIFMQNIIAGQKPLYDKTTNIKIYKLSQKYLDDLLKKL |
| | TWSNVSEDVKDITARYVLNIDKYKEFSKLIKIYSKDGNSKINNDFKGEILSMYNE |
| | MIKKIYNDETINKIGKGFNRLLRDNKGFEIQTKLYNVANEKHLVKVLKMIIDLYS |
| | RNYKSAILNNDELNKLINTIEDKEYAKICSDAILSIGKVFIIIKK |
| 89 | Amino acid sequence of NLS-(linker 2)-(I-A-4 Cas7) fusion protein |
| | MPKKKRKVGSGNKIAMMMRLKLTGEALNNEGTIGNVIQPRQIEFPNGEVRQAISG |
| | EMLKHYHSRNLRLLADENELCDTCKIFSPMKNGKVKESDSKLSPSGNKVKECIVD |
| | DVEGFMNAGKGANEKRTSCVKFSYAIATEENEYQIMLHTRVDVTQDNNKKKQEKE |
| | TTEGEGNTNKDQNTQMLFHRPLRNNEYAITVQVDLDRIGFDDEKLIYALDEDTIK |
| | SRQEKCIKALLNMFVDMEGAMCSTRLPHIEGIEGIIVKKTDKNQVLSKYSALKDD |
| | YKEVNEKISDDSIIFNNIIEFSEVMKGLI |
| 90 | Amino acid sequence of NLS-(linker 2)-(I-A-4 Cas6) fusion protein |
| | MPKKKRKVGSGRINLQGTIIEGQSSIKTNYNHEMYSMILTNISTERANYIHEKKR |
| | FKRLFTFSNLYISDNKVHFYVSGQDELIKDFINCIMFNQMVRVGDRVISITNIEP |
| | MKNSLETKKEYIFKSNFIVNQKENDRVCLSKDMGYVMKRISDIVKDKYKEIYKEE |
| | INENLNVEILNSKQKYTKYKDHHLNSYQATLKVRGNKKLIDLLYNVGIGENTASG |
| | HGFVWEVS |
| 91 | Amino acid sequence of NLS-(linker 2)-(I-A-4 Csa5(Cas11)) fusion protein |
| | MPKKKRKVGSGNNEIKIVKCIDSLYPTVKLTIGKLYKVKESENDKFYRVIADDNN |
| | EEQLCYKYRFELVDINEIKELTLQDIFNEEEGIKYNRINGGSGIYTIQNETLIIG |
| | EHIKPVLNKRIMDSKFVKVKVERLVSFSDVINSDYKCKVKHYRVEGLIQEESSYT |
| | WLEEYQDLKDIMLALSEEFNTIALKEIINKGQWYLEN |
| 92 | Amino acid sequence of T2A cleavage peptide |
| | EGRGSLLTCGDVEENPGP |
| 93 | Amino acid sequence of TadA8e protein |
| | SEVEFSHEYWMRHALTLAKRARDEREVPVGAVLVLNNRVIGEGWNRAIGLHDPTA |
| | HAEIMALRQGGLVMQNYRLIDATLYVTFEPCVMCAGAMIHSRIGRVVFGWRNSKR |
| | GAAGSLMNVLNYPGMNHRVEITEGILADECAALLCDFYRMPRQVFNAQKKAQSSI |
| | N |
| 94 | Nucleotide sequence encoding TadA8e protein |
| | TCTGAGGTGGAGTTTTCCCACGAGTACTGGATGAGACATGCCCTGACCCTGGCCA |
| | AGAGGGCACGGGATGAGAGGGAGGTGCCTGTGGGAGCCGTGCTGGTGCTGAACAA |
| | TAGAGTGATCGGCGAGGGCTGGAACAGAGCCATCGGCCTGCACGACCCAACAGCC |
| | CATGCCGAAATTATGGCCCTGAGACAGGGCGGCCTGGTCATGCAGAACTACAGAC |
| | TGATTGACGCCACCCTGTACGTGACATTCGAGCCTTGCGTGATGTGCGCCGGCGC |
| | CATGATCCACTCTAGGATCGGCCGCGTGGTGTTTGGATGGAGAAATTCTAAAAGA |
| | GGCGCCGCAGGCTCCCTGATGAACGTGCTGAACTACCCCGGCATGAATCACCGCG |
| | TCGAAATTACCGAGGGAATCCTGGCAGATGAATGTGCCGCCCTGCTGTGCGATTT |
| | CTATCGGATGCCTAGACAGGTGTTCAATGCTCAGAAGAAGGCCCAGAGCTCCATC |
| | AAC |
| 95 | Amino acid sequence of linker-3 between TadA8e and |
| | I-A Cas8a fusion protein |
| | SGGSSGGSSGSETPGTSESATPESSGGSSGGS |
| 96 | Amino acid sequence of NLS-(linker 2)-TadA8e-(linker3)- |
| | (I-A-1 Cas8a) fusion protein |
| | MPKKKRKVGSGSEVEFSHEYWMRHALTLAKRARDEREVPVGAVLVLNNRVIGEGW |
| | NRAIGLHDPTAHAEIMALRQGGLVMQNYRLIDATLYVTFEPCVMCAGAMIHSRIG |
| | RVVFGWRNSKRGAAGSLMNVLNYPGMNHRVEITEGILADECAALLCDFYRMPRQV |
| | FNAQKKAQSSINSGGSSGGSSGSETPGTSESATPESSGGSSGGSEYRLIKSGLEM |
| | FDTARAYGLAQLLQVLAGGRAAPRILSQGGVFTLTISTKPNPATLKSSDLWRGAF |
| | GESNWQKVFLTYKRAWSSQRDKVKRSLESHSADIFGKAETDGLAVVFGGNFALPG |
| | PLDPVGFKGLKGLTAGSYSEGQTTVDEFNWALGCLGAAAAQRYKIQKAVGNKWEY |
| | YVTLPVPEEVQFGDFHAVRQLVYDKGLSYNGVRNAAAHFSLLLASAIREKAQGNP |
| | HFPVRFSNVLYFSLFQSGQQFKPAIGGAVNVGRLIEIALARPEVALEMFKTWDYL |
| | FRRGSAQGNEDLAQAITELVMAPSLDTYYRHARIFNRYVVDSTKRVRPEYLYDET |
| | ALKEVLNYAEQ |
| 97 | Amino acid sequence of NLS-(linker 2)-TadA8e-(linker3)- |
| | (I-A-2 Cas8a) fusion protein |
| | MPKKKRKVGSGSEVEFSHEYWMRHALTLAKRARDEREVPVGAVLVLNNRVIGEGW |
| | NRAIGLHDPTAHAEIMALRQGGLVMQNYRLIDATLYVTFEPCVMCAGAMIHSRIG |
| | RVVFGWRNSKRGAAGSLMNVLNYPGMNHRVEITEGILADECAALLCDFYRMPRQV |
| | FNAQKKAQSSINSGGSSGGSSGSETPGTSESATPESSGGSSGGSEYLVVKSGLPT |
| | LDAARAYGLAQLLQVLANGKASPYITDQGGVFAVSLNAELTHDALTRSDMWRAAF |
| | ADSNWQRVFLTYKKAWSAQRDRVKRTLEEQVAAVVTRAGDGLCVDFAGKFALPGP |
| | LDPVGFKGLKGLTAGNYSEGQTYLDEQNGALACLGATIAQRYKFGKREYFVTLPI |
| | PQMVQFNDFHQIRHLVYDKGLAYLGVRTAAAHFALIFADAIRERAAGNPYFPLSF |
| | SNVLYFSLFQSGQQFKPSVGGSINLARLLDIALSRPQAAAEMFKTWDYLFRRGSV |
| | KGNEALAEAITDLLMAPSGESYYRHARIFNRYIVDSSKRVNSEFLYDEAALMEVM |
| | AYVEQ |
| 98 | Amino acid sequence of NLS-(linker 2)-TadA8e-(linker3)- |
| | (I-A-3 Cas8a) fusion protein |
| | MPKKKRKVGSGSEVEFSHEYWMRHALTLAKRARDEREVPVGAVLVLNNRVIGEGW |
| | NRAIGLHDPTAHAEIMALRQGGLVMQNYRLIDATLYVTFEPCVMCAGAMIHSRIG |
| | RVVFGWRNSKRGAAGSLMNVLNYPGMNHRVEITEGILADECAALLCDFYRMPRQV |
| | FNAQKKAQSSINSGGSSGGSSGSETPGTSESATPESSGGSSGGSNHYFLAKSGWE |
| | FFDVSKAYGLGLVIQTLTGNASITDRGGFYLIESKNETKFDKIEEISKYFDDSEL |
| | KTTLITIQRSTKSEMKPPVKKVKGKCLETLTDKESMITVIKNYENLNSPSIIGTD |
| | KQTLYQTMDLAATKGIRNEILLKKNYSDGTNIKISDKDFALSLLGHINFTIKKFS |
| | DFGLILVAPTPLKTELKNVRQIYANLKGNVKVAHKAGWFPTITQIAINLVSEEIM |
| | VKDGGKFAPKFGSLIYSIMRKTGNQWKPSTGGIFPLDFLHQIADSDNAINILNKW |
| | KKIFGWTSRKNGHEDLPTSLAEFIANPNLFNYQRYVNFHLRNEIDKDNIKFGDYK |
| | KEDFLEVMKNVGI |
| 99 | Amino acid sequence of NLS-(linker 2)-TadA8e-(linker3)- |
| | (I-A-4 Cas8a) fusion protein |
| | MPKKKRKVGSGSEVEFSHEYWMRHALTLAKRARDEREVPVGAVLVLNNRVIGEGW |
| | NRAIGLHDPTAHAEIMALRQGGLVMQNYRLIDATLYVTFEPCVMCAGAMIHSRIG |
| | RVVFGWRNSKRGAAGSLMNVLNYPGMNHRVEITEGILADECAALLCDFYRMPRQV |
| | FNAQKKAQSSINSGGSSGGSSGSETPGTSESATPESSGGSSGGSNEYEFKVIKTA |
| | NDIEDICISYGICKILSDNRIKFKLKDNKSMYSIYTKEFDIQNDIFYNDFNIENV |
| | WNLNSGLNQKETVRALDDMNKFLSENIHDILEHLLNGKVLNYKKESAKGIGNCFY |
| | SLGVRASTFGKTLEISPIKKYLSFLGWIYGCSYCYKEKSFEITAILKPYNTDEIA |
| | KPFNFSYVDKETGDKKILTKIKKASEINMMSILYIETLKKYKMLSDEYSNVIFMQ |
| | NIIAGQKPLYDKTTNIKIYKLSQKYLDDLLKKLTWSNVSEDVKDITARYVLNIDK |
| | YKEFSKLIKIYSKDGNSKINNDFKGEILSMYNEMIKKIYNDETINKIGKGFNRLL |
| | RDNKGFEIQTKLYNVANEKHLVKVLKMIIDLYSRNYKSAILNNDELNKLINTIED |
| | KEYAKICSDAILSIGKVFIIIKK |
| 100 | Nucleotide sequence for PAM consumption experiment for Type I-A-1 |
| | TTTACACTTTATGCTTCCGGCTCGTATGTTAGGAGGTCTTTATCATGGGTGATGT |
| | CTTAGGACTGCATTCCCTGCGCGTCGGTCTGTTCCGATTCAGGCTCGTGCCCGAG |
| | CAACCGCTAGAAGTGCCGGCTCTGAATAAGGGCAACATGCTTCGCGGTGGTTTTG |
| | GCCACGGTTTTCGGAAGCTGTGCTGTATTCCCGAGTGCAGAGACGCGAGGCTTTG |
| | CCCACTTGCAGCTATTTGCCCCTACAAGGCCGTGTTCGAGCCTTCTCCGCCGCCG |
| | GGATCGGAGCGCTTGGCGAAGAATCAAGACATCCCGCGGCCCTTTGTTTTCCGCG |
| | CTCCTCACACGAACCAAACCCGTTTTCAAAAGGGCGAAGCGTTTGAGTTCGGGCT |
| | TGTTCTAATCGGACGGGCTGTTGATTACTTGCCATACTTCGTGCTGTCGTTCAGA |
| | GAACTCGCCAATGAGGGGTTAGGCCTGAATCGGGCGAAGTGCGCTTTGGAACGTG |
| | TCGAGCAAAGGCGAACCTCCGCCAATGGTCTCGGGCGTGCCACTGGTGAGGGGAG |
| | GCTGGTCTATAGCAAGGATTCTGGAGTTTTCCACTCGACTGAAAACGAGGGCGTC |
| | GACAGCTACGTGAACTCACGGCTGCGAGAATTGAGTTCTCCGAATGGCGACCAAT |
| | CTCGACAGAACGTGACCATCCGGTTCCTAACACCGACATTTCTGAAAGCCAACGG |
| | GGAAGTGATCCGGCGACCGGAGTTTCATCATCTTTTTAAGCGACTCCGCGACCGG |
| | ATCAATGCACTCTGCACGTTTTTTGGGGACGGCGCGCTTGATTTGGATTTTCGTG |
| | GTGTCGGCACGCGGGCCGAAAAAGTTCAGAGCGTTTCCGCCCGAACGGAGTGGGT |
| | GGAACGCTGCCGCACATCCTCGAAAACGGGGCAGCGTCATGAGCTTTCGGGCTTC |
| | ATGGGCGAAGCCACTTACGAGGGCAACGTGGAGGAGTTCTTGCCGTTGCTGGCGC |
| | TTGGCGAACTGGTTCACGTCGGAAAACACACAGCCTGGGGCAACGGCCGGATCGA |
| | ACTGCAGTCAGGTACGGGAGTCAAGTGCTAGAGGAGCAGTTCCAATTGGTCACTG |
| | GGCATAGCCCCGCTGAGCATCAGCGCGAGTGTGGGGAGGCACTAGCCACAGGGAA |
| | ATCCGTCATTCTTCGAGCCCCTACTGGCTCAGGAAAGTCCGAGGCAGTGTGGATA |
| | CCGTTCCTTCGTTGTCGGGGTAAGAGACTCCCCATGCGCATGATCCATGCGTTGC |
| | CGATGCGTGGGTTGGCGAATCAACTGGAAGAACGAATGAAGGACTATGCCGGTCC |
| | CGGCCTGCGCGTATCGGCCATGCACGGCCAGCGTCCGGAGAGTGTCTTGTTTTAC |
| | GCTGACGCCATCTTTGCCACCATAGATCAGGTGGTCGCCTCTTATGCATGTGCTC |
| | CCCTCAGCTTAAGTGTACGCCATGGCAACATCCCGGCTGGCGCTGTCGCTAGCAG |
| | ttttttggtttttGACGAAGTACACACATTTGAGCCTCGACTCGGCCTGCAATCG |
| | ATTCTGGTGCTGGCTGAGCGAGCCCACCAGATGGGCATGCCTTTTGTAATCATGT |
| | CGGCAACTCTACCGAAGAATTTCATTCGAAGTTTGGCCGAACGATTGGGTGCCGC |
| | GCCAATTGAGGGCGGTCGGTTGAAAAGTAAGGAAGGAGAACCTCGCCACGTGACC |
| | CTGCGAGTGTTGCCAGAAAAACTGAGTGCTCGAACGATTTTGGACTACGCCCCAA |
| | AGGTCAATCGGACGGTCGTTGTCGTGAACACTGTGCAGCGTGCACTCGGTCTGTA |
| | TGAGCAGGTGCGAGATGAATTCCGGTGTCCGGTGATTCTAGCGCACTCGCGCTTC |
| | TACGACGAAGACCGGCGGACCAAAGAGCAACAGATCGAAGCACTGTTTGGGAAGA |
| | AAGCAGCGCAAGGTCGGTGTCTGCTGATCGCCACTCAGGTCGTGGAGGTTGGACT |
| | GGACATTTCCTGCGACCTTCTGATTACAGAACTAGCTCCCGTAGACGCCCTGGTG |
| | CAGCGTGCTGGGCGCTGCGCCCGCTGGGGAGGGAAGGGTGACGTCATTGTTCTTA |
| | CGGAGCTCGACACGAAGAGACCCTACGACGAGACCCTAGTGGCCGTGACGGAGCG |
| | AGCCCTCCAAGAGCACAACGTGGACGGCCAGGAACTGACATGGGAAGTTGAAACA |
| | GCCCTTGTCGACACAGTGCTTGATCCCCATTTCAAGGAATGGGCAAAGCCGGATG |
| | CGGCTGGCAAGGTTTTAGCATCGTTGGCTGAGGCTGCCTTCACAGGAAACTCAAC |
| | GAAGGCAGAACAAGCGGTGCGCGAAACCCTGACCGTCGAGGTCGCTCTACACGAT |
| | ACTCCCCAGGCCTTGGGGCCAGCTATCCTCAGACTTCCCCGATGCCGCTTGCATC |
| | CAGGGGTTTTCCAACAGTTCGTCCGCAAGCAGAGGCCTAATGTTTGGCAAGTGGT |
| | CGTAGATCGAGACCCTGACGATGACTATCGAACCAGAATCGAGTTTCTATCTGTG |
| | AACGGGAAATCGAGACTGATACCTGGCGGCCACTATATCGTTGACCCGCAGTTTG |
| | GTTGTTACGATGCCGAGCGCGGTTTGCGTCTCGGGGTCCCCGGCCAATCAGCAGA |
| | GCCGTTTGCCCCGGGACAATCGAGAGACCGATTGAAAGGTGAGCTGCAAATCGAA |
| | CTGTGGCAGGACCACATAAGAGAAGTCGTCAAGGCCTTCGAAAGGTATGTGCTTC |
| | CCAAGGAGCGAATGGCTTTTGAAGCCTTGAGTCGATGGTTGGGGAAGACTCAAGA |
| | CGAACTGTTAAGCGTCGCTAGGGCTGTTCTCGTTTTACACGACCTTGGCAAGCTC |
| | GCCCGGCAATGGCAAGGAAAGATTCAGGCGGGACTGGAAGGCAAACTTTCTCAGG |
| | GGTCTTTTCTGGCACACCGCGGGGGTTCCGTCAGCGGACTCCCACCCCATGCGAC |
| | TGTCTCCGCTTGGGTTGCGACCCCCTGCCTCCGCCGTCTGGCTGGTACTGACTGG |
| | GAGCAGACGTTGGCGGTGCCGGCCTTAGCGGCGATTGCCCACCACCACAGCGTGC |
| | GAGCCGATATTACTCCTGAATTTGAGATGACCGATGGATGCTTCGAAGTAGTCGC |
| | AGACTGCGCGCGAGGCGTCGCTGGCCTTGAGGTTAAACGCGACGATTTCAATACG |
| | AAACCACCGCAAGGCAGCGGCTCCTGTGGGGTTGGTTTGAACTTTCTGTTGCCTG |
| | AGGGCTACACGTCCTACGTGTTGCTTTCCCGATGGCTCCGCTTGGCGGACCGAAT |
| | CGCCACGGGAGGTGGGGAAGAAACGATCTTTCAATATGAGAAATGGATGGGCGAT |
| | TCCTAAGGGACAGGTTAATTATTGCGAGCCTTGCCGCTAGGATGAGAGCATGACC |
| | GGCTGCCAAGTGCGTATGCTTCTAGATCTCGCTTGGGATGTCAGCGCACGTGTCG |
| | ATAATGAGCGTGTTGTTCAGTGGACGCAGCTCCAGGGATTGTCGCCTCTAGATGT |
| | TGGCTTCCGAGTGAGAATTGCTGAGAAGTTCATCTGCGGCCGACTTCTGATAGTT |
| | CCCAAGACCCTGCCTTTGTACGCAAAGAATTTCTTGCGGCTATGCGAAGGCGAAA |
| | AGAGACGTTTTGACCAGTTGGGGGAGCCACTCCCCAGCCTGAATGTCGTTGACCA |
| | GCGCCGGTACATTGAAAAACTAGTTTGGCTCGGTGGTTTGGACTCAAGGGGAAAA |
| | CGCTATGTGGATCAACTGTATGGTTTCAGTCCATTCACCGCGCTGAAGCAAGTCG |
| | AATTGGCTGAGCAAGAGCATCTAAGCCCTCCCGCGATCGCCAAGAGAGTGAGTGC |
| | AATCAACAGGAGATTGGTCGCTGCTGCTCACCTAAGCCAGCTTCAGCCTATCGAA |
| | CGCCTGTATGCCTTCTTGCGTTAGGAGTCGAAGCTTTGGAGTACAGACTTATAAA |
| | AAGCGGATTGGAAATGTTCGATACGGCTCGAGCTTACGGGTTGGCTCAACTTCTG |
| | CAAGTGCTGGCCGGAGGAAGGGCCGCGCCGAGAATCCTGAGTCAAGGAGGTGTTT |
| | TTACGCTGACCATTTCGACAAAGCCAAATCCCGCCACTTTGAAGAGCTCAGACCT |
| | TTGGCGCGGCGCATTTGGCGAGAGCAACTGGCAAAAGGTCTTCCTAACCTACAAG |
| | AGGGCTTGGTCAAGTCAGCGCGACAAGGTGAAGAGGAGCTTGGAAAGCCACTCGG |
| | CAGATATTTTTGGTAAGGCCGAGACAGACGGACTCGCAGTGGTCTTCGGTGGCAA |
| | TTTCGCATTGCCGGGGCCGTTGGACCCAGTCGGATTCAAAGGACTGAAAGGCCTG |
| | ACGGCAGGTAGCTATTCGGAAGGTCAAACCACCGTAGATGAATTCAATTGGGCTT |
| | TGGGTTGCTTGGGTGCTGCCGCGGCCCAGCGGTACAAGATCCAAAAGGCTGTAGG |
| | CAATAAGTGGGAATACTACGTGACCCTGCCTGTTCCGGAGGAAGTCCAATTTGGT |
| | GACTTCCATGCAGTTCGGCAGCTAGTCTATGACAAGGGACTCAGCTACAACGGCG |
| | TACGCAACGCCGCCGCTCACTTTTCTCTCCTCTTGGCGAGCGCCATCCGTGAAAA |
| | AGCGCAAGGCAACCCGCACTTTCCAGTGCGGTTTTCAAACGTATTGTACTTCTCT |
| | CTTTTTCAATCCGGCCAGCAATTCAAGCCCGCTATTGGAGGCGCTGTGAATGTAG |
| | GGAGACTTATCGAGATCGCTCTGGCTCGGCCAGAGGTGGCGTTGGAGATGTTCAA |
| | GACTTGGGATTACTTGTTTCGCCGGGGCAGTGCTCAAGGGAACGAAGACTTAGCT |
| | CAGGCCATTACGGAACTGGTGATGGCACCTTCGCTCGATACTTATTACCGCCACG |
| | CACGAATCTTTAATCGCTATGTAGTGGATTCGACGAAGCGCGTAAGACCCGAGTA |
| | TTTGTACGACGAAACCGCACTGAAGGAGGTGCTCAACTATGCTGAGCAGTGACTC |
| | GAAATTGTCAGAGGTGTTCGCAGAAGAGAGCGTCAAGTCCTTTGGAAAATGCTTG |
| | CGATACGCACTCTGGCGTGACGAAGACTACGCTTCTCTCATAGAGTTCGAGAATG |
| | CAGAAACTCCAACTCAATTTGCCGATGCGGTGAGGAAGTTTCTTCGAAGATATCG |
| | GAGTGGCGGCTTCATGGATCAAACTCAGCGAAGCCGGGCTTCCGAGATGCGCAAG |
| | CAAAACCGCTGGGATGGACTTAAAAAGCTTCTTCGGCAGTACGAAGTGGGACCTC |
| | GACCCAGTGAGGGGCAGCTTGAGAGGTTGATGCAACTGGCGAATGACACTAACGG |
| | AGTGCGATTAGTTCAGTCGGCCATCATTTCCTATGGACTCACCAAGAGGGAGCCA |
| | TATAAGGAAGTTGAGGAACTGGAGAAGGAGAACTGAAATGGCAGACAGCCCAGTT |
| | TTTGAGGTCGCGATTCTTGGACGTGTAGTTTGGAACCTACATTCACTGAATAACG |
| | AAGGCACTGTGGGCAATGTCAGTGAGCCGCGCACAGTTGTTTTGGCGGACGGGTC |
| | GAAGTCCGATGGCATCTCGGGGGAGATGCTCAAGCATATCCATGCGCAGAACGTC |
| | TGGCTGGTCGCGGAAGACAAGAGCCAACTCTGCGAACCGTGCAGAACACTGAACC |
| | CTCAGAAAGCTGACAAGAATCCGGCTGTACTAGGAGTTAAGACTGCGAAAGCGAA |
| | AGTAGCAGCAGAAAGCATGAGCGTCGCAATCTCCAGTTGTGCGCTGTGCGATTTA |
| | CACGGCTTCTTGGTTCAAAGGCCCACGATAGCGCGGGCCTCTACAGTGGAATTTG |
| | GGTGGGCTGTAGCTCTGCGTGACGGGTACCATCGGGATATCCATCTTCATGCGCG |
| | TCACGCTGTTGAGGGGCGCGCCGAAACGACCGAAGGTCAGCAAGAGGGACCAGCC |
| | GAAGTATCCGGCCAGATGATTTATCACCGGCCCACGCGTTCGGGTACCTATGCCT |
| | TCGTCAGTGTCTTCCAACCCTGGCGAATCGGCTTGAACGAGGTGAACTACGAATA |
| | CGTTGAGGGTGTCGATCGGGAGGCGCGCAATAAGCTGGCAATCGAAGCTTATAAG |
| | GCAACTTTTGCGCGCACTGGTGGCGCAATGACTTCCACTCGGCTGCCGCACGTGG |
| | AAGCTCTTGAGGGTGTTGTGTTGGTTTCCAGTCGCAACTTCCCGGTCCCTGTGAC |
| | GAGCCCTCTTCAGGATGACTATCGAGAGAAAACTGAAAAAGTCGGGCAAGCGGTA |
| | GAGGGGTTAGAGGTTCAGCGTTTCGGTGCTCTGCCGGAACTTTACGTCATCTTAA |
| | ATGCGCTTGCAAAACGACGTTTGTTCGCGTTGCAAATGGGAGGAACGTCGAAGAA |
| | AGGAAAACAGTAAACCATGGCCGAGTGGCTCCAAGCTGAAGTTGAGTTCGCCAGT |
| | TTCTACAGCTACCGAGTGCCTGACCTTTCTCCGAGTTTCGCGCTATGCTCCCCGG |
| | TGCCGAGCCCGGCAGCTATCCGGCTGGCAGTCGTAGACGCTACTATTCGGCACAC |
| | TGGAGATGTTAACGAAGGCCATGCAGTTTTTGAGTTAATGAAGCGAGCCAGATTG |
| | GAACTTCAACCACCAAGCCGTGTCGCTGTGATGAAGTTTTTTATAAAGCGACTGA |
| | AGCCAGAGAAGCCGACCAAGGGAAAACGCGCGAGTGTGATCGAATCGACCGGTAT |
| | TCGAGAATATTGCTTGCCATGGGGCCCGATGGTCTTTTGGATTGAGAGCGATCAG |
| | CCAGAACGCATCGCCCAATCCCTACAGTGGCTGCGCCGCCTCGGAACGACCGACT |
| | CACTTGCGAGTTGCACCGTGGGAGCAGGCACTCCAAATTTTGCGTCCTGCATTAG |
| | ACCGGCGAATGGCTTGACACTTCAGACCACTAATTTCGCGCAGCGCCCGGTCTTT |
| | ACCCTCCACGAGCTCAAACCAGAGACGCAATTCAATCAGGTGAATCCGTTCGCGG |
| | ACGAAAGACCAGGTAAACCTTTTGAGAAGCGGCTTTATGTTCTTCCCTTGGTTCG |
| | AGAAAAGGTTGGCGAAAACTGGGTGATCTATCATCACGAGCCGTTTGCGGCTTGA |
| | caaataaaacgaaaggctcagtcgaaagactgggcctttcgttttatctgttgtt |
| | tgtcggtgaacgctctcctgagtaggacaaatttgacagctagctcagtcctagg |
| | tataatgctagcGTTCCAGAGCCTTCCCCGATGAAGAGGGGACTGAAAGGGTATA |
| | ACAACTTCGACGAGCTCTACAAAGCTTGGCGTTCCAGAGCCTTCCCCGATGAAGA |
| | GGGGACTGAAAGagaaggccatcctgacggatggcctttG |
| 101 | Nucleotide sequence for PAM consumption experiment for Type I-A-2 |
| | TTTACACTTTATGCTTCCGGCTCGTATGTTAGGAGGTCTTTATCATGCTTTCCGG |
| | TGCCTTTGTTGCATCCCGCAATGCAGAGATGCTCGGACCTGCCCAGTGGGAATGT |
| | CGTGCCCGTACAAGGCAATCTTCGAGCCTTCCCCTCCGCCGGAAGCAGAGGCTCT |
| | TTCCAAGAATCAGGACATCCCGCGGCCGTTCGTGTTCCGAGCCCCAAAGACGCAG |
| | CAGACGCGATTTGAAACAGGTCAGCCGTTCGAATTCGAGCTGGTGCTTATCGGTC |
| | GCGCGCTCGATTTCCTGCCATACTTCGTGCTGTCGTTTCGAGAGCTAGCGGCTGA |
| | GGGGTTGGGGCTCAATCGCGCCAAGTGCAGTCTTGAAAGGGTCGAGCAGGTGGAC |
| | CTGACCAGCGAAGCAGCAGACGCTTCGAACTACGAGGCTATGGTTATCTATACGG |
| | CCGAGGACCAGGTTTTCCGAAATGCGGCGACTTCCGAGACGGGCGAATGGATAGG |
| | GAGGCGGATACGGAACCGCTCGACATCCCGAGATAACGATTCAGTGCAGCAGGTC |
| | AGCATCCGATTTTCGACGCCAACGTTCCTGAAGGCCGATGGCGAAATCATCCGAC |
| | AGCCGGAGTTCCACCACGTTTTCAAGCGCCTGCGGGACAGGATCAATGCCTTGAG |
| | CACGTTTTTTGGTGAGGGGCCAATCGAAGCGGATTTCCGGGGCCTTGGCGAGCGC |
| | GCGGAAAAGATTCGAACGGTTTCGGCCCGCACCGATTGGGTTGAGCGTTTCCGCA |
| | CGTCGTCAAAAACGAAACAACGCCACGAATTGTCGGGCTTTGTCGGTGAAGTTAC |
| | TTACGAAGGTAACTTGAATGAGTTTTTGCCCTGGCTCACGCTCGGTGAGCTGGTG |
| | CATGTCGGCAAGCACACGGCATGGGGAAACGGCTGGATGGAGCTAGAACACGAGG |
| | TGTCTCGTGGTTGCGTGTGACACCTTCTTCGCGCCGATGACCGACTTCAATCTGG |
| | CTCGGCATCAGTCTGAATGCGCTGGGGCGCTCGCGAGTGGCAAATCGGTCATCTT |
| | GCGGGCGCCGACAGGCTCAGGAAAGTCGGAGGCGGTATGGCTACCGTTTCTCTCT |
| | CTGCGGGGGAAGACGCTTCCTTGTCGGCTCATTCATACGCTTCCCATGCGTGCTC |
| | TTGTAAACCAACTTGAGAGCCGGATGCGAACTTATGCGAATGGCAGGATGAGGGT |
| | GGCAGCCATGCACGGCCAGCGGCCGGAGAGCGTCTTGTTTTACGCGGACGCCATA |
| | TTCGCGACGCTTGACCAAGTGGTCACTTCTTATGCGTGCGCTCCTCTAAGTCTGA |
| | GTGTGCGACAAGGCAATATTCCGGCAGGGGCCGTCGCCGGCAGCTTTCTGGTATT |
| | TGATGAAGTCCACACTTTCGAGCCTCACCTTGGCCTGCAATCGTTGCTCGTCCTT |
| | GCTGAACGAGCCCACCAGATGGGCATCCCATTTGTGATCATGTCGGCTACTCTGC |
| | CGACGAATTTTATCCGTCGTTTGTCGGAAAGATTCGGCGCAACCATCGTCGAGGG |
| | CACACGACTGGAAGGCAAGAACCGACGACAACGCCGCGTCGTACTCAGGGTCTCG |
| | TCAGAGAAGCTGAGCATCGAGACGATTCTGGAGCTGACACGAAACGTGGAGCGGA |
| | CGCTTGTTGTCGTCAACACCGTGCAACGTGCGCAGAACCTATATGAGCAACTCCT |
| | GGGCAAAATCGGATGTCCAGTGATTTTGGCGCACTCGCGCTTCTACGATGACGAC |
| | CGAAGGACCAAGGAAAAGCAGATCGAAGCCCAATTTGGCAAGACGGCGGAAGGCC |
| | AGTGTCTACTGATCGCCACGCAGGTTGTGGAGGTGGGGTTGGACATTTCCTGCGA |
| | CCTCCTGGTCACGGAATTAGCCCCAATCGACGCCATAGTGCAGCGAGCCGGCCGA |
| | TGCGCCCGCTGGGGTGGACAGGGCGAGGTTGTTGTCTTTACAGGCTTGGAAACAA |
| | CGCGACCGTATGATCGGACCCTCGTGGAAGCAACTGAGAAGGCGCTCCGAGAGAA |
| | GAACTTAAACGGGCAGGAATTGACATGGGAAATTGAGAGAGCTCTTGTCGATACG |
| | GTCCTTGAGCCACAGTTCAGCAAATGGGCCGAGCCGGAGGCCGCTGGCAAGGTCT |
| | TGGCCTCATTGGCTGAGGCGGCCTTCACTGGCGACTCAGCTAAGGCAGAACGAGC |
| | GGTGCGCGAAGGTCTGACCGTTGAGGTCGCTCTGCACGGTTCGCCTGACACCCTC |
| | GGAGTTGGCGCTCTGAGGCTGCCGCGTTGTCGTATCCATCCAGGGGGCTTCCAGC |
| | AGTTTGTGCACAAACAGCAGCCCGAAGCCTGGCGAGTGGTTGTTGATCGAACGGC |
| | TGCAGATGATTACCGAACTCGGGTCGAATTCCTGCATGTGGACTCGAACTCCAAG |
| | GCCGCGCCTTATGGCTACTACATAATTCACCCGCAATATGGCTCCTATGATGTGG |
| | AGCGTGGCTTGCGACTAGGGATCCGAGGTTCCCCTGCCCAATCTCGCGACGAGCT |
| | GATACAGCGGAAGAGCCGACTGGAGGGTGAACTGCAAATTGAAAAGTGGCAGGAC |
| | CACATTGAAAAGGTGGTGAAAGCATTCGCCGAACACGTCCTGCCAAAGGAGAGAA |
| | TCGCTTTCGAGGCCCTGTCCCGGCGACTTGGGAAAACTCATGAAGACTTGCTGTC |
| | CCTGACTCACCTTGTCTTGATCTTCCACGACCTTGGCAAACTGGCACAGCAGTGG |
| | CAACGAAAGATCCAGGCAGGATTGGAAAGCGTCCTTCCTCCGGGGACCTTCTTAG |
| | CTCATCGGGGAGGCTCGCTCAGGGACCTGCCACCTCACGCAACTGTTTCCGCTTC |
| | GCTGGCGACGCCCTGTCTATGCCGTGTGGCTGGACCTGACTGGCAGCAGACCTTG |
| | GCGATACCCGCCTTGGCCGCGATCGCGCACCACCATAGCGTGCGCGCAGACATGA |
| | CTCCGCAATTCGATATGAGTGAAGGGTGGTTCGACGTTGTCGCCGACTGTGCGCG |
| | GCGGTTGGCTGGGGTTGACGTCACCGTTAACGATTTCAGTAGATGGCGAGGAGGG |
| | GGCAGTTGCGGAGTTGCTCTCAACTTCCTGCTGCCTGATGGGTACACATCTTACA |
| | TTTTGCTGTCCCGGTGGCTTCGCCTGGCTGATCGGATCGCGACTGGCGGTGGCGA |
| | AAATGCCATTCTGAATTATGAAGACTGGATGTCATCCAGTTAAGTGCGTCGTGGA |
| | GGAGGGTCtttttttGTGGAGTACCTAGTCGTAAAAAGCGGTCTGCCCACCCTTG |
| | ATGCGGCCAGAGCGTATGGACTAGCTCAACTCCTCCAAGTCCTCGCCAACGGCAA |
| | AGCCTCACCCTATATTACCGACCAGGGTGGTGTCTTTGCCGTGAGCCTGAACGCA |
| | GAGCTCACTCATGATGCGCTCACACGTTCGGATATGTGGCGCGCGGCGTTTGCTG |
| | ATAGCAACTGGCAAAGAGTGTTCTTGACGTACAAGAAGGCTTGGTCCGCGCAACG |
| | GGACCGGGTCAAGCGCACATTGGAGGAGCAGGTTGCCGCTGTTGTCACCCGCGCA |
| | GGGGACGGGCTTTGCGTCGATTTTGCTGGAAAGTTCGCGCTGCCCGGTCCTCTCG |
| | ATCCTGTGGGATTCAAGGGTCTCAAGGGTCTGACGGCCGGAAATTACTCGGAAGG |
| | TCAGACATATCTCGACGAGCAGAATGGAGCCCTGGCCTGTTTAGGTGCGACCATC |
| | GCCCAGCGCTACAAATTCGGCAAACGAGAGTACTTCGTAACGCTACCCATTCCGC |
| | AAATGGTACAGTTCAATGACTTCCATCAGATTCGGCATTTGGTCTACGACAAGGG |
| | GCTGGCTTATCTGGGAGTCCGTACTGCCGCAGCACACTTTGCGCTTATTTTCGCT |
| | GATGCCATTCGGGAACGGGCCGCAGGAAACCCCTATTTTCCGCTCAGCTTTTCGA |
| | ATGTGCTTTATTTTTCGTTATTCCAGTCAGGTCAACAGTTTAAGCCTTCGGTGGG |
| | CGGCAGTATCAATTTGGCGCGACTCCTCGATATTGCCCTGTCTCGACCACAAGCA |
| | GCGGCGGAAATGTTTAAGACCTGGGACTATTTGTTCCGGCGAGGCAGCGTAAAAG |
| | GCAATGAGGCCTTGGCCGAGGCAATTACCGATTTACTGATGGCTCCTTCGGGTGA |
| | GAGCTATTACCGTCACGCCCGCATTTTCAACCGTTACATAGTTGACTCAAGCAAG |
| | CGCGTCAACTCCGAGTTCTTGTACGACGAAGCAGCATTGATGGAGGTGATGGCTT |
| | ATGTCGAACAGTGAGATCTCCCTGGCTTCGGTTTTTGCCGAGGAGAGCATTAAGT |
| | CATTCGGCAAGTGCCTGCGATACGCGCTCTGGCGAGACGACGACTACGCCTCGCT |
| | CATCGAATTCGAGAACGCCGAAACTCCGCTTCAATTTGCGGAAGCCGTGAGAAAG |
| | TTTCTCCGACGATATCGCAGTGGCGGTTTTATGGATGAGGCTTTGCGCACCCAAG |
| | CGTCAGAGATGCGTAAGCACAACCGCTGGGACGAGCTCAGAAGGACGCTTCGGCA |
| | GAACGAAATTGGCCCGAGACCTACAGAGGGAAATCTGGAGCGCTTGACGCAATTG |
| | GCAAACAACGCCCAGGGCGTCCGGCTCGTCAGAGCGGCGATTATTTCTTACGGAT |
| | TGACCAAACGCGATCCGCACAAAGAACTTGAGGAAGTGGAGAGGGGGAGCTGATA |
| | TGGCAGGGAACTCAGTTTTTGAGATTTCGATTTTGGGGCGCAGCGTCTGGAACCT |
| | GCATTCATTGAACAATGAAGGCACCGTTGGGAATGTCAGCGAACCACGTACCGTA |
| | ATCTTAGCAGACGGTTCAAAATCCGACGGCATTTCAGGCGAGATGCTCAAGCACA |
| | TACACGCCCAGAATGTGTGGTTGGTGGCCACTGACAGGTCTGTCTTTTGTGAGCC |
| | CTGCCAGACGCTCCAGCCTCAGAAGGCTGACAAGAACCCGGACGTCACAGGTGTT |
| | AAGGCGGCAAGAGCCAAACTGGCCTCAGAAGGTATGAATGTGGCCATCGCTGCCT |
| | GTGCGCTATGCGATTTGCACGGATTCTTGGTCCAAAAGCCCACGATAGCTCGTGC |
| | CTCAACGGTTGAGTTTGGATGGGCTGTAGCAGTGCGTAACGGGTTTCACCGCGAC |
| | ATACATCTTCACGCGCGCCATGCTGTTGAGGGGCGCACAGAAGGTCAACAAGAGG |
| | CGGGAGAAGTGGCCGCACAGATGATTTATCACCGCCCGACGCGTTCTGGCACCTA |
| | CGCGTTGGCCAGTGTTTTTCAGCCCTGGCGAATCGGATTGAATGAAGTCAATTAC |
| | GAATACGTCGCAGGAGTTGATCGGGAGGCCCGCTACAGACTCGCTATCGAAGCCT |
| | ATAAAGCTACTTTTGCGCGCACGGATGGCGCTATGACCTCTACCCGTCTCCCCCA |
| | CCCCGAGGCTTTTGAGGGGGTGGTGCTTGTTTCGAGTCGCAACTTTCCTGTTCCG |
| | GTAACGAGCCCCCTCCAGGATGACTATCGTGAGAAGTTACAGCAGTTGTCCAGAG |
| | CCACCGAAGGACTGGAGCCGCAGCCATTCAATTCGCTGACGGAACTGTATGGGAT |
| | ATTGAACGAACTCGCCAAAAGGCCGCTGTTCAATTTGCAACTTGCCCGCTCCTCG |
| | AAGCGAGAGAAGAAGTGAAAAATGGCTGAGTGGATCCAGGCCGAAATCGAGTTCG |
| | CCAGCTTTTACAGCTACCGGGTTCCGGACCTGTCTCCCAGCTATGCCCTCTCCTC |
| | GCTAGTGCCAAGCCCAGCAGCGATTCGGCTTGCCGTCGTGGACGCGGTGATCCGG |
| | CACACCGGTGTTGTGGACGAAGGCGAATCGATCTTTGAGCTGGTAAAGCGGGCCA |
| | AATTGGAAGTTCAGCCGCCGGCTCGTATTGCCGTAATGAAATTCTTCGTCAAACG |
| | ACTGAAGCCGGAGAACCCCGAAAAGGGCAAGCGCGCTAGCGTGATCGAGTCCACC |
| | GGCATACGAGAGTATTGTTTGCCCTCCGGGCCCCTCGTGCTATGGCTAGAGACGG |
| | AGGAACCCGAGCGCATTGGTCAGGCCCTTCAGTGGTTGCGCCGCCTCGGTACCAG |
| | CGACTCACTTGCAACTTGCAAGATTGGCCATGGGGCCCCGGACACCGCGCTGTGC |
| | ATTAAGCCTGCAAATGGGCTGGCTATTCAGGCGAAGAATTTTGCGCAGCGGGCAG |
| | TGTTCACGCTCCACGAATTGAAACCCGACGCGAATTTTTCCGAAGTAAATCCGTT |
| | TGCCGATGGCAGGCGCGGCGATCCTTTTGAGAAGCGTCTGTATGTATTGCCATGT |
| | GTTCGCGAGCAAGCTGGAGAGAATTGGGTTCTTTATCGGCGTGAGCCTTTTGCTA |
| | ACTGAcaaataaaacgaaaggctcagtcgaaagactgggcctttcgttttatctg |
| | ttgtttgtcggtgaacgctctcctgagtaggacaaatttgacagctagctcagtc |
| | ctaggtataatgctagcGTTGAAGAGCCTTCCCCGATGAAGAGGGGACTGAAAGG |
| | GTATAACAACTTCGACGAGCTCTACAAAGCTTGGCGGTTGAAGAGCCTTCCCCGA |
| | TGAAGAGGGGACTGAAAGagaaggccatcctgacggatggcctttG |
| 102 | Nucleotide sequence for PAM consumption experiment for Type I-A-3 |
| | TTTACACTTTATGCTTCCGGCTCGTATGTTAGGAGGTCTTTATCATGAGGCTGAA |
| | GATCTCCCTGACCTCCAACAACGGCAACTACCTGATCCCGTACAACTACAACCAC |
| | ATCCTGTCCGCCATCATCTACAGGAAGATCGCCGACCTGGACCTGGCCGCTAAGC |
| | TGCATTTCTCCAAGGACTTCAAGTTCTTCACCTTCTCCCAGATCTACTTCTCCGA |
| | CTGGAAGCGCACCAAGAATGGCATCATCAGCAAGGACGGCAAGCTGAGCTTCTAC |
| | ATCTCCTCCCCCAATGAGCAGCTGATCAAGTCTCTGGTCGAGGGCCATCTGGAGA |
| | ATACAGAAGTGGATTTTAAAGGGAAAAAATTGCTGGTGGAACAGATTGAGCTTCT |
| | AAAAAGTCCCTCGTTTAAGGAAAACATAAAGCTGAAAACTATGTCTCCAGTAGCA |
| | GCCAGCATAAAAAGAGAAGTTGATGGAAAACTTAAGATATGGGATTTAGGACCTG |
| | GAGACGAACGATTCTACGAGAGCGTTCAGAAAAATTTGGTGAATAAGTACACTTC |
| | CTTCTATGGAGATTATGACGGTGACAAATGGGTAAGGATAAAACCCGATATGAAA |
| | ACAGCTAAAAGGCGTAGAATTGAGATAAAAGGAGACTTTCACCGCGGATATATGA |
| | TGGAATTTGAGATGGAAGCTGATCCTCGCCTTGTGGAATTTGCTTATGACTGTGG |
| | ACTGGGTGAAAAGAATAGTATGGGGTTTGGGATGGTAAATATTTATGAATAAACT |
| | GTTTAAAAAATTAATAGGAGCTAAACCATACGATTATCAGAAAATAGCTATGGAG |
| | AATTTGCTTGATGGTAAATCAATAATAATGAGAGCCCCAACAGGTTCAGGAAAAA |
| | CTGAAATAGCTTTGATTCCTTTTCTTTATGGATTCAATGATTTATTACCTTCTCA |
| | ATTGATTTATTCTCTCCCAACAAGAACTTTGGTTGAGAGTATTGGTGAAAGGGCT |
| | GTTAAATATGCTTCATTTAGGAAACTAAGAGTGGCTATTCACCATGGAAAAAATG |
| | CAACTAGTAGTCTTTTTGAAGAGGACGTAGTAGTAACTACAATTGATCAAGCGGT |
| | AGGCGCTTATTTGAGCACGCCACTCAGCATGTCTAAAAGGTCTGGTAACATATTC |
| | GTTGGCAGTGTAGGTTCTGCTTTAACAGTATTTGATGAAGTTCACACACTTGATC |
| | CGGAAAAAGGACTTCAAACTAGTTTGGCTATTAGTATGCAATCGGCTAAACTTGG |
| | TTTACCTACACTAATAATGTCCGCTACATTACCAGATATTTTTATAGAAACTGCT |
| | AAAGATAGGATTTCaaaaaaaGGAGGAGATATTGAGTTTATAGATGTAAAAGATG |
| | AATTCGAAATCAAATCAAGAAAAAATAGATTTGTTGAATTAATAAATAGACTTGA |
| | AGAAGAATTAAATGCAGAAAAAGTATTAGAAGAAGTTGAGCACGGAAAAAGAATT |
| | ATAATTGTTATTAACACCGTCAATAGGGCTCAAGAATTGTATTTAGAGTTGAGAA |
| | ATAAAACAGAATTACCTATACTTCTTTTACATTCCCGATTTCTTGAGAAGGATCG |
| | ACAAGAGAAAGAATTACTACTTGAAGAAACGTTTGGAAAAAATGGCAATGGCAAA |
| | TGTATTTTCATCGCAACTCAAATTGTTGAGGTAGGAATGGATATTTCATCACCTA |
| | AAGTTTTATCAGAGATAGCTCCTATAGATGCTTTGATACAAAGAGCAGGAAGATG |
| | TGCCAGGTGGAGCGGGAAAGGGGAATTTCATGTTTTTGGGTACAATACAAACTCA |
| | AAAAGTCCACACGCACCTTATAACAAAGACATTGTAGAGGCTACAAAATCAGAAA |
| | TCAACAACAAAGGGAAAAGTTTCACTCTCGACTGGAATACTGAGGTTGAACTAGT |
| | AAATAAAATTTTAACTAAACATTTTTCAGAATTTATGAATTCAATGATATTTTAT |
| | CAAAGGTTAGGTGAACTTGCAAGAGCGGTTTATGAAGGTAGTAGGGCAAAAGTGG |
| | AACAAAACGTTAGAGAAGTTTTCTCCTGCGATGTTACACTGCATGAGAATCCTAA |
| | ATCCATGAATTCTGTTGAAATCCTACATTTACCCCGACTAAGACTTGACGCTAGA |
| | ACTTTAATGGGAAAGGTAGAAAAAATTGCTGAAATGGGAATTGACACATACAGAT |
| | TAGAGGAAAATACAATAATTTTTGATGATGATGAAGATGAATACGTACCTGTTCT |
| | GGTTAATAATCGTGAAGAAATAATTCCGTTTGAGTTATACGTATTATGTGGTGCT |
| | AGTTATTCATCAGATACAGGTTTAGTTTTTGATGATTTCCCAAATGCATTAAAAT |
| | CATTTGATCCTGAAGAAAAAGAAATTTTATCCAGTAAACAGTTTGATAATAGGCT |
| | TAAAGTTGAAACTTGGGTTGAACATGCAAAAAATACGTTAAAAGTTCTTGATAAT |
| | TATATGATTCCTAGATATCGTTATTCTATAGAGAATTTTGCTGAAAATTATGGCT |
| | ATAACTATGGTGAGTTTTTGGATATTATTAGGTGTACGGTGTCATTGCACGATAT |
| | TGgaaaattgaacaaaaaatggcaaaaaagaataaaatggaatgatgaaaCTCCT |
| | TTAGCTCATTCTAACGACAATACAATTAAAAGGCTACCAGCGCATGCTACTGTTT |
| | CCGCCAAAGCATTACAACCATATTTAGAAGATCTATTTGATGATGAAGATATATT |
| | CAAAGCCTTTTATCTAGCTATCGCTCATCACCATCAACCTTGGTCAAAATCATAT |
| | AATGAATATGAACTAGTTCCAAAATATGATGAATCCCTAAAGGAGATTTGGATTA |
| | TTCCTAAAAATTTTATACAAGAACAAAATCCAGCCGGTAGGCTTGATTTTTCATA |
| | TTTAGATATTATCGATGAAAATGAAGCTTATAGACTATATGGTTTTCTTTCTAAG |
| | TTAATGAGAATTTCAGATAGACTTGCAACGGGAGGTAATACTTATGAATCATTAT |
| | TTTCTGGCTAAGAGCGGTTGGGAATTTTTTGATGTTTCAAAAGCCTATGGACTGG |
| | GATTAGTTATACAAACATTAACTGGCAATGCTTCTATAACTGATCGAGGGGGATT |
| | TTATTTGATTGAATCAAAAAATGAAACTAAGTTTGATAAAATTGAAGAAATATCC |
| | AAATATTTCGATGATTCAGAACTTAAAACTACATTAATAACTATTCAACGTTCTA |
| | CAAAATCAGAAATGAAACCTCCAGTTAAAAAAGTTAAGGGAAAATGCTTGGAAAC |
| | TTTGACTGATAAAGAAAGCATGATTACGGTGATTAAGAACTATGAAAATTTGAAC |
| | TCACCTTCGATTATAGGCACAGATAAACAGACATTATACCAAACTATGGATTTAG |
| | CTGCTACAAAGGGCATTAGAAATGAAATTCTGTTAAAGAAGAATTATTCAGACGG |
| | AACAAACATTAAAATTTCAGATAAAGATTTTGCTTTGTCTCTTTTAGGTCATATT |
| | AATTTTACTATaaaaaaaTTCTCCGATTTTGGATTGATTTTAGTTGCACCTACGC |
| | CACTTAAAACAGAATTAAAGAATGTAAGGCAAATTTATGCAAATTTAAAAGGTAA |
| | TGTAAAAGTAGCGCATAAGGCAGGATGGTTCCCTACTATCACTCAAATAGCAATA |
| | AATTTAGTTTCAGAAGAAATCATGGTTAAGGATGGTGGAAAGTTCGCCCCAAAAT |
| | TTGGTTCATTAATATATAGCATTATGAGGAAAACAGGGAATCAATGGAAGCCATC |
| | TACTGGGGGTATTTTCCCTCTCGACTTTTTACATCAGATAGCAGATTCAGATAAT |
| | GCAATAAACATTTTGAATAAATGGAAGAAGATATTTGGATGGACATCACGGAAAA |
| | ATGGCCATGAGGATTTACCGACAAGTCTAGCAGAGTTCATTGCCAATCCAAATTT |
| | ATTTAATTATCAAAGATATGTTAATTTTCACCTCAGAAATGAAATTGATAAAGAT |
| | AATATCAAATTTGGTGATTATAAAAAAGAAGATTTTCTGGAAGTGATGAAAAATG |
| | TCGGAATTTAGATTGAAAGATGTATTTGAACACGAATCTATAAAGAGTTTCGGAA |
| | AGACTCTAAGAAAAATGATTAGGCCTCCAAAAGAAGGAAATAAGGAAAAATGGGC |
| | TTCAGACTATGCTTCCATAGTGGAATTGGGGTATGTGGAAACAAAAGACCAGTTT |
| | GCAGAAGTGATTAAGAAATTATTAAGAAGATATGATGTGATAGCaaaaaaaCATC |
| | AACTTAAACGTCCCACAGaaaaaaaTTTAGAAGAATTGATGGAATTAATTGATAA |
| | ATACGGTGTAAAACCTGTTAGAGCTGCCCTTATCAGTTATGCTCTTGTTAAAAAA |
| | GATGAAGAATAAATTAGGAGATGATATGATGGTGAATGAAACAGAAATTTATGAA |
| | ATTGCTATTTTGGGAAGAGCAACATGGCAATTACACAGCCTAAATAATGAGGGAA |
| | CTGTTGGAAATGTTACGGAACCTCGAAGTGTTACAATCATTGACCCAAATACCAA |
| | GAATCCAATAACAACCGACGGAATTTCTGGAGAAATGCTAAAACATATCCATACG |
| | GGGCTGATGTGGACTTTAACAGATAAAAATAATCTCTGTGACGCATGTAAGGTGT |
| | TAAACCCTGAGAAATTTAATGTAACATCTGGAAGGGGCAGTACTGTTGAAGAGGT |
| | TTTAGAAAACGCTTTAAATAAATGCGATATCTGCGATTTACATGGATTTCTTATT |
| | ACAAGGCCAACTGTATCCAGAAAATCAACCATAGAATTTGGTTGGGCCTTAGGAA |
| | TACCTGAAATTTATAGAGATATTCATACACATGCAAGACACGCGCTTGGTGGAAA |
| | AACGACTGAAAATGAAGAATCTAAAGGTGTAAACACCCCAAATTCTTCTGAAGAT |
| | AAAGAAGAAGCTGTCGGCACTTCAACTCAAATGGTTTATCATCGTCCTACACGCT |
| | CTGGTGTTTATGCAGTTATTTCAATGTTTCAACCTTGGAGAATAGGATTAAATGA |
| | AACAAGACAAGATCAATACACTTACGATACGGGAAATAATGAAAAGCGAATTGAA |
| | AGATATAAAAATGCATTGAAAGCATATCAAATTCTTTTCACCAGACCTAAAGGTG |
| | CAATGAGTACTACTAGGTTACCTCATGTCGAAGATTTTGAAGGCGTAATCGTTTT |
| | CTCGACGGATCAAATTCCTTTACCCTTAATATCACCACTTAAACAGGATTACGTT |
| | AAAGAAATAACAGATATTTCCaaaaaaaTTGACAATTCAATAAATGTCGAAGAAT |
| | TCAAAACTCTTTCTGAGTTTGTAGACAAAATAGGAGATTTAATTGACAAAAAACC |
| | GTACAAATTAAAGTTAGGTGAATAATAATGCAGTGGTTAAAATTTACTCTGCATT |
| | TTCCATCATTTTTCTCTTATAGAATACCTGACTACTCTTCACAATATGCTTTAGG |
| | GATTCCATTACCCTCACCTTCAACCTTGAAGTTGGGAGTAATTTCATCAGCTATA |
| | AAATCAACTGGGAAAGTTAGTGAAGGTGAAAAAGTATTTAACGTTGTGAAAGACG |
| | CAGAAGTATGTGTTGCCCCACCagaaaagattgcaattaattcatttttaataaa |
| | aagattaaaaaagagaaaagaagatttaaaaCTAATACCCACATTTGGAATTAGA |
| | GATTACGTTTTCTTCCCTGATGATATTGATATATTTGTTGGAAGTGAAAATATTG |
| | ATTCTGTGGCCGAATATTTCAGCAAAATGAACTATATAGGCTCTAGTGATTCAAT |
| | GGTTTATGTGAAATCCATCGAACCTAAAACCCCCTCTGAAAATGTGATTAAAGCT |
| | GTGGATATTGATGAATTTTCGGATGCTGCAGAAAAAGAGTCATATCTTGTTTATC |
| | CAGTAAAAGACATTAATAAAAATGCAACTTTTGACCAAATAAATTCTTATTCCAG |
| | CAAATCTAGTCGTAAAATTTTAGATCAGAAATATTATCTTATCAATGCAAAAGTG |
| | AGTAAAGGCAAAAACTGGAAAATACTTGATACCCGAAACTAAcaaataaaacgaa |
| | aggctcagtcgaaagactgggcctttcgttttatctgttgtttgtcggtgaacgc |
| | tctcctgagtaggacaaatttgacagctagctcagtcctaggtataatgctagcG |
| | CTCAAATCAGACTATTTTAGGATTGAAATGGTATAACAACTTCGACGAGCTCTAC |
| | AAAGCTTGGCGGCTCAAATCAGACTATTTTAGGATTGAAATagaaggccatcctg |
| | acggatggcctttG |
| 103 | Nucleotide sequence for PAM consumption experiment for Type I-A-4 |
| | TTTACACTTTATGCTTCCGGCTCGTATGTTAGGAGGTCTTTATCATGAGAATAAA |
| | CCTTCAAGGAACAATAATAGAAGGTCAATCATCAATAAAGACAAATTATAACCAT |
| | GAAATGTACAGTATGATATTAACAAATATTAGTACAGAAAGAGCAAATTATATAC |
| | ACGAAAAGAAAAGATTCAAAAGATTATTTACATTTTCAAATTTATACATAAGTGA |
| | TAATAAAGTTCATTTTTATGTATCTGGGCAAGACGAGTTAATTAAAGATTTTATA |
| | AATTGTATTATGTTTAATCAAATGGTTAGAGTAGGTGATAGAGTTATTAGTATCA |
| | CAAACATAGAACCAATGAAAAATAGCTTAGAAACTAAAAAGGAATATATTTTTAA |
| | AAGTAATTTCATAGTAAATCAAAAAGAAAACGATAGAGTATGTTTATCAAAAGAT |
| | ATGGGATATGTCATGAAGAGAATTTCAGACATTGTAAAAGATAAATATAAAGAAA |
| | TTTATAAAGAAGAAATAAATGAGAATTTAAATGTTGAAATACTTAATAGTAAACA |
| | AAAATATACTAAATATAAAGACCATCATTTAAACTCATATCAAGCAACATTAAAG |
| | GTAAGGGGTAATAAAAAGCTAATAGATTTATTATATAACGTAGGAATTGGGGAGA |
| | ATACAGCTAGTGGTCATGGTTTTGTTTGGGAGGTATCCTAATGAATGAATATGAA |
| | TTCAAAGTGATTAAAACTGCTAATGATATAGAAGATATATGTATTAGTTATGGGA |
| | TATGTAAGATATTATCGGATAATAGAATTAAATTTAAACTAAAAGATAATAAAAG |
| | TATGTATAGTATTTATACAAAAGAATTTGATATACAAAACGATATTTTTTATAAT |
| | GATTTCAATATTGAAAATGTATGGAATTTAAATAGTGGATTGAATCAGAAAGAAA |
| | CCGTAAGAGCATTAGACGATATGAATAAGTTTTTGTCTGAGAATATACATGATAT |
| | ATTAGAACATTTACTTAATGGCAAAGTTTTAAATTATAAAAAAGAAAGTGCAAAG |
| | GGCATAGGAAATTGTTTTTATTCGCTAGGTGTGAGAGCTTCTACTTTTGGTAAAA |
| | CATTAGAGATAAGTCCTATTAAAAAATATTTATCCTTTTTGGGATGGATATATGG |
| | ATGTTCTTATTGTTATAAAGAAAAAAGTTTTGAAATTACTGCAATATTAAAACCT |
| | TATAATACTGATGAAATAGCAAAACCTTTTAATTTTTCATATGTAGATAAAGAAA |
| | CAGGAGATAAGAAAATATTAACCAAAATAAAAAAAGCGTCTGAAATAAATATGAT |
| | GTCAATACTTTATATTGAAACTTTAAAGAAATATAAAATGTTATCAGATGAATAT |
| | AGTAATGTAATATTCATGCAAAATATAATAGCTGGGCAAAAACCATTATATGATA |
| | AGACAACAAATATTAAAATATATAAATTATCTCAAAAATATCTAGATGATTTATT |
| | AAAGAAATTAACTTGGAGCAATGTATCGGAAGATGTAAAAGATATTACTGCTAGA |
| | TATGTTTTAAATATTGATAAATATAAAGAATTTTCAAAACTAATAAAAATATATA |
| | GTAAAGATGGCAATTCAAAAATTAATAATGATTTTAAAGGAGAGATATTAAGTAT |
| | GTATAATGAAATGATTAAGAAAATTTATAATGATGAAACTATTAATAAAATAGGT |
| | AAAGGATTCAATAGGTTATTAAGAGATAATAAAGGTTTTGAAATCCAAACAAAAC |
| | TATATAATGTTGCAAATGAAAAACATTTAGTAAAGGTACTTAAAATGATAATTGA |
| | CTTGTATTCAAGGAATTATAAAAGTGCAATATTAAATAATGACGAATTAAATAAG |
| | TTGATAAATACAATTGAAGATAAAGAGTATGCAAAAATATGTTCAGATGCAATAT |
| | TATCAATAGGAAAAGTATTTATAATTATTAAAAAATAAATTGTATAAACCATATA |
| | ATAAATTAAAATAATGAGTGAAAGAGGTAATAAAAATGAATAAAATAGCAATGAT |
| | GATGAGATTAAAATTAACTGGAGAAGCTTTAAACAATGAAGGAACAATAGGAAAT |
| | GTAATACAACCTAGACAAATAGAATTTCCAAATGGAGAAGTAAGACAAGCAATAA |
| | GTGGAGAAATGTTAAAGCACTATCATAGTAGAAATTTAAGACTATTAGCTGATGA |
| | AAATGAACTATGTGATACTTGTAAAATATTTAGTCCTATGAAAAATGGAAAGGTT |
| | AAAGAATCTGATAGCAAATTAAGTCCTAGCGGAAACAAGGTTAAAGAATGTATAG |
| | TAGATGATGTTGAAGGGTTTATGAACGCTGGAAAAGGTGCAAACGAAAAAAGAAC |
| | AAGTTGTGTTAAATTCTCATATGCAATTGCAACTGAAGAAAATGAATATCAAATA |
| | ATGTTACATACTAGAGTAGATGTAACACAAGATAATAATAAGAAAAAACAAGAAA |
| | AAGAAACTACGGAGGGCGAAGGTAACACCAATAAAGACCAAAATACTCAAATGTT |
| | ATTTCATAGACCTTTAAGAAATAATGAGTATGCTATAACTGTACAAGTTGATTTA |
| | GATAGAATTGGATTTGATGATGAAAAATTAATATACGCACTAGATGAGGATACTA |
| | TTAAATCAAGACAAGAAAAATGTATTAAAGCATTATTAAATATGTTTGTTGATAT |
| | GGAAGGTGCTATGTGTTCAACTAGATTACCACATATTGAAGGAATTGAGGGAATA |
| | ATAGTTAAGAAAACTGATAAGAATCAAGTGTTAAGTAAATATAGTGCCTTGAAAG |
| | ATGACTACAAAGAAGTAAATGAAAAGATTTCAGATGATAGTATTATTTTTAATAA |
| | CATTATAGAGTTTTCAGAAGTTATGAAAGGATTAATATAATTTACCAAGTAGATT |
| | TAATAAACTCAGTAGAATAGCTATTGTTGAAAAGTTGTAACTGTTGGTAAAATAT |
| | TATAATAATGGCTTAAATACTAGAGTTATGGGAAATATATAAATAAGATAATAGC |
| | TATTTTACAATGATTAAGATTTAACAATCATGTTATTTAAACCGTATAAATGTAT |
| | AATACAATACTATTTACGAAAATATTAAGATTTAACAATCATGTTATTTAAACTG |
| | ACAGCTTTAGTTTATTAGACACCAAAACTGGAAAATTAAGATTTAACAATCGTGT |
| | TATTTAAACTTAAAGAAATTTGGATTAGATATGGATAAATTATATTAAGATTAAA |
| | CAATAATGTTATTTAAACTCAATAATCTGTCTTTCACTCCTGCTTTTTAGTTTTA |
| | TTAAGATTAAACATTAATGTTATTAAAACGGCTTAAACGTTATGGTATGAATAGC |
| | TTTATTACTATTAATATTAAACAATAGTGTTATTTAAATATTTAACAAGAGCAAC |
| | AATCAATCATGTTTATATGAACATTAATATTAAACAATCATGTTATTAAAAATTA |
| | ATTTTTCCTATTTTATCTAATGTCATATTTAATTAATATTAAACAATATGTTATT |
| | CAAATTATGGAGGCAATTACATAAAAAAAGGTGAAATGGGATTAATATTTAACAA |
| | TAATGTTATAAACAAAATAAATAAAGTGAGGTAAATAAAAGATGAAAAAAGTAAC |
| | ATATAAACTAAGTAATATATTCTCATTAAAAAAATACAATGATAATAATTTAAAC |
| | TGTCAATCTTACGAATATCCAACTATATACGGAATTAGATGTGCAATATTAGGTG |
| | CAATAATTCAAGTTGATGGAATTGATAAAGTTCAAGAATTATTCAACAAAATTAA |
| | AAATTCAAATATTTATATTCAATATCCTAAAGAGTTTAAAGTTAATGGGATAAAA |
| | CAAAAAAGATATGCAAATTCATATTATAACTCTTGTTATACAGAGGAGGAATACA |
| | ATAAATTATCACCAAGTACTCAAAGTAAAACATATTGTGTATTAGATAGAGATAA |
| | ATTAGTAGGTTCAAACTGGAAAACAACTATGGGATTTAGACAATATGTAAAAATG |
| | GATAATATAGTATTTTACATAGATAATTTAATCCCTGAGATTGATATGTATTTAA |
| | AGAATATTGATTGGTTAGGAACTGCTAAGAGTATGGTTTATTTAAGTGATGTAGA |
| | AGAAGTTAATAAATTAGATAACGTTTTAACTAGATGGAATAAAGAATCCTATGTA |
| | GACACTTTTGAACAACATGATTGGAATAGTAAAACTACCTTTGATACAATTTATA |
| | TGTATTCTAAAAAATATAAACACTTTCATGATACTTTTATGTGTGGCATTGGAGA |
| | TATAATTCTACCAAGCTGATTGTGATATACCAGATATACGTTCATTCTTTATTTT |
| | AAGCTTTGGTTGGTAAACTTATATGAGAATTAGGCTACTATAAGAGTTTTAAAGA |
| | TTGTTATAAAATAAGAATGGGTAATTTACTAAGATTAAAATTAAATATAGTATTA |
| | TTTAAACTTCTCTCCAAGAGCAAATATCGCATTATCATAAGATTAAAATTCAATA |
| | TAGTATTATTTAAACCTAAAAGATTTATATTGTCTTATTAATGTATTAATTATTG |
| | ACATTGAATATGGTATTATTTAAATCCCTCAAAGGATTCGATTTCTTCTTTTTCT |
| | TTTTCATTAAAATTAAATATAGTATTATTTAAATAGAAAGTTGTAACTAAAATCA |
| | ACTCTATTTCTCCATATTAAAATTCAATATAATATTATTTAAACACTCCACGAAT |
| | GGTTGTGATAGATGATTATACAAATTAAAATTAAATATAATATTATTTAAATTAC |
| | GCATCTTGTAGCCTATGCATTTGATTATATATAGATTAATATTAAACAATTATGT |
| | TATTAAAATGTTATGTCACAACTTAAAATTTCCATGAATATATTAATATTAAACA |
| | TTATTGTTATTTAAATAATAAGATAAGACTAAAGAAGATAAGACTTATATAATAT |
| | TAAACTTATATATTACTATTATATAATAACATATTAAAGAAAGGAATAAAAATAA |
| | TGAAATATAAAGAAATATTTGAAAAACTAAAATTAAATAACTTAACAGAAGTACA |
| | ACAAAAAATAAGTGAATTAGAAGGGAGTAAGAATATATTAGTAGTATCTTCATGT |
| | GGAAGTGGAAAAACTGAGGCTAGTTATTTTAAAATGCTAGAATACAATAGAAAAA |
| | CAATAATCATAGAGCCTATGAAAACTTTAACTAATTCAATACATGGAAGAGTAGA |
| | TATATATAATAAAAAATTAGGATTAGAAAAAGTATCAATACAACATAGTTCGTCC |
| | CAAGAGGATAGATTTCTACAGAATAAATATACAGTTACTACAATAGACCAAGTTC |
| | TTGTAGGATATCTAGCTATGGGAAAGCAAGCATACATAAAAGGTAAAAACATAGT |
| | AATGAGTAATTTAATATTTGATGAAGTGCAATTATTTGATACAGATACAATGCTA |
| | TTAACTACTATAAATATGTTAGATGAGATATATAAATTAGGAAATAAATTTATAA |
| | TAATGACAGCTACTATGCCACAATTTTTAATTGAGTTTCTTGGAGAAAGATATGA |
| | TATGGAAATTGTAATTACTGAAAAAATTAGAGAAGATAGAAATGTAAAATTATTT |
| | TATGAAGAAGAACTAGATTATAATAAAGTAAGGAATTATAAAGATAAACAAATTA |
| | TAATATGTAATTCAATAAAGCAATTAAAAGAGATACATAAAAAACTTCCTAATAG |
| | TAGAGTTATTACATTACATAGCACATTTTTAGGTAGTAACAGATTAAAATTAGAA |
| | AAACAAGTGGAAAGATATTTCGGAAAGCATTCAGAACAGAATGATAAAATATTAT |
| | TAACAACACAAATTGTTGAAGTAGGAATGGATATTAGTTGTGACAGATTGTATAC |
| | TACGGCATGTAAAATTGATAATCTTGTACAAAGAGATGGCAGATGTTGTAGATGG |
| | GGAGGAGATGGACAAGTTATTGTATTTAAAAATGACGATAATATATATGAAAAGG |
| | AATTAGTTGAAGAAACTATTAAATATATTAAAAACAATCAAGGTATAGCTTTTAA |
| | CTGGACAATTCAAAAACAATGGATTAATGAAATATTAAATGAATACTATAAGAAT |
| | AAAATAAATGAATATAATTTAAGAAAAAATAAATTTAATTTTAATGGTTGTAATA |
| | GAAGTAGGTTAATTAGAGATATTCAAAATATAAATGTGATAGTTGTAAACAAAGA |
| | AGAGTTCACCAAACAAGATTTTAATAGAGAATCAGTAAGCTTACATATCAACAAA |
| | CTAAAAGAATTGTCTCAGGCAAATGAAATATACATATTGAATAAAAATAAGATAG |
| | AAAAAGTAAAATATAATAAAGTTGAAATAGGAGATACTGTAATTATAAGAGGTAA |
| | AAATTGTAGATATGATGACTTAGGATTTAGATATGAAGAAGACTCAGCTAAAAAT |
| | ATGCCAAAATGTAGAGATTTTCCTATGACAAATAAGTCAAATAATAATCAATTTA |
| | GAGATTACATAGAAGAAACTTGGATACACCATGCAGAAACAGTAAGAGATTTAAT |
| | GTCTTATAGATTAAATCAAGAGCAATTTAATGATTATATAATTATTAATGGTAAA |
| | AAGATAGCCTTTTATGGTGGCTTACATGACTTAGGTAAGCTAGATTTAGAATGGT |
| | CAAGAAAATATAAGTCGGCTATTCCATTAGCTCATTTCCCTTTTGTGAAAGGTTC |
| | TATGGGAGAAAAAAGAACCCATGAACTAATTAGTGGAGAAATACTAAAGGAGATA |
| | ATAGATGATGACATTATTTATAACATGATGATTCAACATCATAAGCGATTATACG |
| | ATGATATAGATATAGATTATAAGGGGATAGAATGGGAATTACATAAAGATACATA |
| | TAAAATACTTACTACATATGGATTTAAAGATGATATACAATTACAAAGTGATGCT |
| | AAAACATTGAAAAGAAATAATATCATGTCTCCATGTGATAATGAATGGACTACAT |
| | TATTATATTTGGTAGGTACTTTTATGGAATGTGAAATTCAAGCAATTAATGAATA |
| | CATAGATAATTATAAGCAAGCTATATAATACATAAACAAAGCAAATAATATATTA |
| | AAATAAAAAACAAGGTATATAATTTCAATATTATGGTATAATATATATTAAGAGG |
| | TGAAGTATATATGAAAGTAAAAGAATTAATGGATATTATAAATTTAAATTTAAAA |
| | GATTTTAAAAATATAAAATATGAGTCTTCTAGTGAGTATAGTGGTGTGATACAAA |
| | ATAAATTTGAATCAATTATAAAACAAACAGAATTAAAATCATTATTAATATATGA |
| | ATATAATGAAATCAGATTACTAAAAGGAGGAGGAATTTTATTTCAAGTCGATGTA |
| | TGTTATAAAGAAGATGCAAGAATTAAATCTCCAGTTAAAAGAAAAGGCACTATAA |
| | AATCGGTAAATATTTCACTCGATGAGGAATTAGTTGTAAGTTTATTAGAATTTGA |
| | ATCGTGTGATTTACCATTATATTTTCGTAAGAAACGGATAATAGATAATATAGAT |
| | GACTGTAATTCCGACATTAAAGACATAAAAATTGAGTTATTAAAATTAGAAAACA |
| | AAAAATTAGAATTTGAAAAACAATTAGTTCAAATTTGTAAAAATAAAGCAAATTA |
| | ATTATCAGAAGGTAGCGATAATACCTTCTGATAATATACAAAACAATAAAATCAC |
| | ATTTTAGAATATACAAAAGCAAATAAAATCTGTAATTTATTAAAACTTAATAATA |
| | TATTAAAATAAATATTTAATAAAAACGGGTATATAATTTCAAATAAATGGTATAA |
| | TAGTAATATGAAATTATTTGAAAGAGGTGTAATATATGAGAAAAATTACTAAAAT |
| | AGAAATTGAAAGTCTTATGAAATGTTTAGATTATTCAAAAGATGAATATATAGGT |
| | TTTACTATAGTTGACAATGATATAAGATTACTTCATTCTAATACAAGTAAAGGAA |
| | TTTATTATCGAATAGACGGATTCATTGAAAGATGGAAAAATAATAAAGATACTTT |
| | AAATGAGATGCTAGGAAAAATACAAGACTATATAAATGATGTTATAGAGCTATAT |
| | AATGAAGGGAGTGTTAATCATGAATAGTTATAAAGAAGTTTATGAAGAATGGTTT |
| | GATAGTGAAGATAATGTGATTGTAGTATCAGAAGATGATACAAGACAATTAGTAA |
| | TTGGTATAATTGAAAACATTGTTTTTATAGGAAAAATTATAAAATTTAATAAAAC |
| | CTATGATATAAATATTATTAAACAATTTGAACTACAATGTTCTTATCCCTTTAAT |
| | CAAGATAGAAAATATTTTTTATATCCAGTATTACAAATGTATAAAATATTATTTT |
| | ATTTAGTTTCTTATATGCAAAGTAAAAATATTATATGCGATATAAATAATGATGG |
| | AATAGATAGTTATATTTTAAAATTATCTCAGCATCCTATATTGTTACAAGATTTT |
| | ATAAATAATTTTAGAATACTAATAAACGAGAATATTAAAATGAAAGATGTAGCTA |
| | TTTTGAAATAACTTAGGGCATATTAATTGCCCTTTTCTTATATAAAATCATAAAA |
| | ATACAAATTCTACAACTAGCTTATAGCCACATTCTAAAAGTTGAATATTGTGATA |
| | AAATGACACTTTTCTTTGGAGTTAAATTATTCCAGTTAAAAACTACATAATAAAT |
| | TAAAATAATACTTGACTTATATTTGAGCGAAGTGTATAGTATTAAATATAAGGGA |
| | GGTAAAGAAATGTTAAACAAGCCTAGAGAAGAATGGTCAAAAGAAGAAATCAATA |
| | TGTATTGCAGAAATAAAGCAATTAATAAAGTTAAAAACAGATTAAAACAACAATT |
| | GAGGGAAAGGGTGATAGTGAGATGTTAAGAAAATGGTTATTAAAAAATAAGCTAC |
| | ATAAAAATGATTTAGAATGGATTGAAATTACAGAATATGCACTTTTAGATTATAG |
| | GCATCAAGTACGGGGAAATGAAAGTGAAAGTACTTGGTTACTTGAAAGAAAATTA |
| | AATCGAAATTATCAATTTGGTATAATTTTAGATGAAACAGACAAAACAATACATA |
| | AAAAATATGGAATGTTAACAATAATATATAGTAAAGAAAATGGTAAGATTATAGG |
| | GATAACTAATCATAGGGGCAAATATAGCAATTGTGAAATTGATAAGGAACTTAAA |
| | AATAAGTTAAATAAAATTTATGGAATAAGTTAGGAGGAAAATAAATGGACAAGTT |
| | AAAAATAATAGTTTTGGTGGCAAAATCTTCAGCAGGAAAAGATAATATATTAAAT |
| | AAGGTAGTTGAATTAAATCCAAAAGTAAAAACAATAGTATCTTATACTTCACGTC |
| | CTATTAGGCAGGGAGAAATTGATGGAATTACATATCATTATATATCAAATGAGAA |
| | AGTAAATAATATGTTAGCTAATCAAGAATTTATAGAAAATAAGATATACAATACA |
| | GTAAATGGTAAGTGGGTGTATGGAGTTGGAAAGTCAAGCTTTGACCTTTATTCAA |
| | AAAATACTTATATTGTAATATTAGATTTACAAGGACTAAAACAATTAGAAAATTA |
| | TTTAAATGAAAATAATAAATTAGATTGTTTAATTTCAATATACATAAAAGCAAGT |
| | GGACAAACTAGATTATTAAGAAGTTTGCAACGTGAAGGACAATTAAGTGATAATC |
| | AATGTAAAGAGATATGCAGAAGATTTATTTCAGATGAAGAAGATATGGAGTATGC |
| | CGAAGGTTATTGTAATATAACTTTGGTAAATGAAGTTGAGGATGATTTAAATAAG |
| | TGCATAGAATATGTTTACCATTTAACAATTAATTAATGGAGGGATAAAGCAAATG |
| | GAATTTTCAAAAGACGAATTAAAGGAGATTGCTCTAAGTTTAAATTTGATTTCTG |
| | CTGAACGTAATGCTTATTTATTAGATGATAGTATAAATAAATATAAAGAAAATAA |
| | TAATAAATACTTGGAAATGGATAAGATATTGCTTAATAAAATTAAATTAGAAATA |
| | AAAAGATTAAAAGAAGGAGTAGAAGAATAATGAATAATGAGATTAAAATAGTCAA |
| | ATGTATAGATAGTTTATATCCTACAGTAAAACTAACTATTGGAAAATTATATAAA |
| | GTTAAAGAATCTGAGAACGATAAATTTTATAGAGTAATAGCTGATGATAATAATG |
| | AAGAACAACTTTGTTATAAATATAGATTTGAATTAGTAGATATTAATGAAATAAA |
| | GGAATTAACATTACAAGATATTTTTAATGAAGAAGAAGGTATTAAATATAATAGA |
| | ATAAATGGTGGAAGTGGAATATATACAATACAAAACGAAACATTAATTATTGGCG |
| | AGCATATTAAACCAGTTCTCAATAAAAGAATAATGGATTCTAAATTTGTAAAAGT |
| | AAAAGTAGAAAGACTGGTGAGTTTCTCAGATGTTATTAATTCAGATTATAAATGT |
| | AAAGTAAAACATTATAGAGTTGAAGGATTAATTCAAGAAGAAAGTTCATACACAT |
| | GGCTTGAAGAATATCAAGATTTGAAAGACATAATGTTAGCATTATCGGAAGAATT |
| | TAATACTATTGCATTAAAAGAGATAATTAATAAAGGTCAGTGGTATTTAGAGAAT |
| | TAAcaaataaaacgaaaggctcagtcgaaagactgggcctttcgttttatctgtt |
| | gtttgtcggtgaacgctctcctgagtaggacaaatttgacagctagctcagtcct |
| | aggtataatgctagcATTAAGATTTAACAATCATGTTATTTAAAGGTATAACAAC |
| | TTCGACGAGCTCTACAAAGCTTGGCATTAAGATTTAACAATCATGTTATTTAAAa |
| | gaaggccatcctgacggatggcctttG |
| 104 | PAM library sequence |
| | NNNNNNNNGGTATAACAACTTCGACGAGCTCTACAAAGCTTGGCG |
| 105 | Guide RNA sequence in I-A-2 system |
| | targeting ROS1 (GRMZM2G422464) gene in Example 4 |
| | GUUGAAGAGCCUUCCCCGAUGAAGAGGGGACUGAAAGGUAAUCCGUGUAUACCAG |
| | CUAUUGGGUCAACUGAACUGUUGAAGAGCCUUCCCCGAUGAAGAGGGGACUGAAA |
| | G |
| 106 | Guide RNA sequence in I-A-3 system |
| | targeting ROS1 (GRMZM2G422464) gene in Example 4 |
| | GCUCAAAUCAGACUAUUUUAGGAUUGAAAUGUAAUCCGUGUAUACCAGCUAUUGG |
| | GUCAACUGAACGCUCAAAUCAGACUAUUUUAGGAUUGAAAU |
| 107 | Dual-target guide RNA sequence in I-A-1 system |
| | targeting ROS1 (GRMZM2G422464) gene in |
| | Example 5 |
| | GUUCCAGAGCCUUCCCCGAUGAAGAGGGGACUGAAAGGAAAGGGCAUGGAAGAAG |
| | UUGCGAUACACAGAUUGAGUUCCAGAGCCUUCCCCGAUGAAGAGGGGACUGAAAG |
| | GUGAAGAAUGAUUACUUGAAGUUUUCUACCAAUAGUGUUCCAGAGCCUUCCCCGA |
| | UGAAGAGGGGACUGAAAG |
| 108 | Dual-target guide RNA sequence in I-A-2 system |
| | targeting ROS1 (GRMZM2G422464) gene in |
| | Example 5 |
| | GUUGAAGAGCCUUCCCCGAUGAAGAGGGGACUGAAAGGAAAGGGCAUGGAAGAAG |
| | UUGCGAUACACAGAUUGAUGUUGAAGAGCCUUCCCCGAUGAAGAGGGGACUGAAA |
| | GGUGAAGAAUGAUUACUUGAAGUUUUCUACCAAUAGUAGUUGAAGAGCCUUCCCC |
| | GAUGAAGAGGGGACUGAAAG |
| 109 | Dual-target guide RNA sequence in I-A-3 system |
| | targeting ROS1 (GRMZM2G422464) gene in |
| | Example 5 |
| | GCUCAAAUCAGACUAUUUUAGGAUUGAAAUGAAAGGGCAUGGAAGAAGUUGCGA |
| | UACACAGAUUGAGCUCAAAUCAGACUAUUUUAGGAUUGAAAUGUGAAGAAUGAU |
| | UACUUGAAGUUUUCUACCAAUAGUGCUCAAAUCAGACUAUUUUAGGAUUGAAAU |
| 110 | Dual-target guide RNA sequence in I-A-4 system |
| | targeting ROS1 (GRMZM2G422464) gene in |
| | Example 5 |
| | AUUAAGAUUUAACAAUCAUGUUAUUUAAAGAAAGGGCAUGGAAGAAGUUGCGAU |
| | ACACAGAUUGAAUUAAGAUUUAACAAUCAUGUUAUUUAAAGUGAAGAAUGAUUA |
| | CUUGAAGUUUUCUACCAAUAGUAUUAAGAUUUAACAAUCAUGUUAUUUAAA |
| 111 | Target sequence g in Example 4 |
| | GTAATCCGTGTATACCAGCTATTGGGTCAACTGAACT |
| 112 | Target sequence g1 in Example 5 |
| | GAAAGGGCATGGAAGAAGTTGCGATACACAGATTGAT |
| 113 | Target sequence g2 in Example 5 |
| | GTGAAGAATGATTACTTGAAGTTTTCTACCAATAGTA |
| 114 | Nucleotide sequence of M13F primer |
| | GTAAAACGACGGCCAGT |
| 115 | Nucleotide sequence of M13R primer |
| | CAGGAAACAGCTATGAC |
| 116 | Target sequence g1 in Example 7 |
| | TGCCAGCGGGGAGGTCAATGCTGGGAGTTGGGGCGCG |
| 117 | Target sequence g2 in Example 7 |
| | GGGCGTGGAGCGCGGCTACTACCGGGAGTTCTTCGAG |
| 118 | Dual-target guide RNA sequence in I-A-2 system |
| | targeting GA2 (GRMZM2G368411) gene in |
| | Example 7 |
| | GUUGAAGAGCCUUCCCCGAUGAAGAGGGGACUGAAAGUGCCAGCGGGGAGGUCAA |
| | UGCUGGGAGUUGGGGCGCGGUUGAAGAGCCUUCCCCGAUGAAGAGGGGACUGAAA |
| | GGGGCGUGGAGCGCGGCUACUACCGGGAGUUCUUCGAGGUUGAAGAGCCUUCCCC |
| | GAUGAAGAGGGGACUGAAAG |
| 119 | Target sequence G1 in Example 8 |
| | ACGAGGGCACCCAGACCGCCAAGCTGAAGGTGACCAA |
| 120 | Target sequence G2 in Example 8 |
| | CGCCGACATCCCCGATTACAAGAAGCTGTCCTTCCCC |
| 121 | Guide RNA sequence in I-A-3 system |
| | targeting |
| | Tdtomato gene Gl target site in Example 8 |
| | GCUCAAAUCAGACUAUUUUAGGAUUGAAAUACGAGGGCACCCAGACCGCCAAGCU |
| | GAAGGUGACCAAGCUCAAAUCAGACUAUUUUAGGAUUGAAAU |
| 122 | Guide RNA sequence in I-A-3 system |
| | targeting |
| | Tdtomato gene G2 target site in Example 8 |
| | GCUCAAAUCAGACUAUUUUAGGAUUGAAAUCGCCGACAUCCCCGAUUACAAGAAG |
| | CUGUCCUUCCCCGCUCAAAUCAGACUAUUUUAGGAUUGAAAU |
| 123 | Target sequence g1 in Example 9 |
| | CAGGGCCCGGCCGCCACCTGCCGCGTGGGCCTGAACC |
| 124 | Target sequence g2 in Example 9 |
| | AAGAAAGAAGCGATTCTATTTCATATTAGGCATTGTA |
| 125 | Dual-target guide RNA sequence in I-A-2 |
| | system |
| | targeting HPRT1 gene in Example 9 |
| | GUUGAAGAGCCUUCCCCGAUGAAGAGGGGACUGAAAGCAGGGCCCGGCCGCCACC |
| | UGCCGCGUGGGCCUGAACCGUUGAAGAGCCUUCCCCGAUGAAGAGGGGACUGAAA |
| | GAAGAAAGAAGCGAUUCUAUUUCAUAUUAGGCAUUGUAGUUGAAGAGCCUUCCCC |
| | GAUGAAGAGGGGACUGAAAG |

### Specific Models for Carrying Out the Present Invention

The present invention is now described with reference to the following examples which are intended to illustrate the present invention (but not to limit the present invention).

Unless otherwise specified, the experiments and methods described in the examples were basically carried out according to conventional methods well known in the art and described in various references. For example, conventional techniques of immunology, biochemistry, chemistry, molecular biology, microbiology, cell biology, genomics and recombinant DNA used in the present disclosure can be found in Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel et al., ed. (1987)); METHODS IN ENZYMOLOGY series (Academic Publishing Company): PCR 2: A PRACTICAL METHOD. APPROACH (M. J. MacPherson, B. D. Hames, and G. R. Taylor, ed. (1995)), and ANIMALCELL CULTURE (R. I. Freshney, ed. (1996)). In addition, if the specific conditions were not specified in the examples, they were carried out according to conventional conditions or conditions recommended by the manufacturer. The reagents or instruments used without indicating the manufacturer were all conventional products that could be obtained commercially.

Those skilled in the art know that the examples describe the present invention by way of example and are not intended to limit the scope sought to be protected by the present invention. All the disclosures and other references mentioned herein are incorporated herein by reference in their entirety.

The formulas or sources of some reagents involved in the following examples were as follows:

LB liquid culture medium: 10 g of tryptone, 5 g of yeast extract, 10 g of NaCl, diluted to 1 L, and sterilized.

CTAB solution: 16.7 g of CTAB (hexadecyltrimethylammonium bromide), 234 mL of 5 M NaCl, 83.5 mL of 1 M Tris-HCl (pH 8.0), 33.4 mL of 0.5 M EDTA (pH 8.0), added with distilled water to reach a volume of 1 L, and added with β-mercaptoethanol in proportion of 100:1 when using.

W5 solution: 154 mM NaCl, 125 mM CaCl₂, 5 mM KCl, 4 mM MES, diluted to 500 mL, adjusted to pH 5.7 with NaOH.

20 MMG solution: 0.4 mM mannitol, 15 mM MgCl₂, 4 mM MES, diluted to 10 mL.

Large-scale plasmid kit, purchased from QIAGEN, catalog number: 12963.

Blunt-smiple vector, purchased from Yeasen Biotechnology (Shanghai) Co., Ltd., catalog number: CB111-02.

DH5α competent E. coli, purchased from Beijing Tsingke Biotechnology Co., Ltd., catalog number: TSV-A07.

Prokaryotic expression vectors pACYC-Duet-1 and pUC19, purchased from Beijing TransGen Biotechnology Co., Ltd.

EC100 competent E. coli, purchased from Epicentre Company.

Unless otherwise specified, the sequence synthesis involved in the following examples was completed by Beijing Tsingke Biotechnology Co., Ltd., and the sequencing involved was completed by Beijing Ruibo Xingke Biotechnology Co., Ltd., Sangon Biotech Co., Ltd., and Liuhe BGI.

### Example 1: Acquisition of Type I-A Gene and Type I-A Guide RNA

1. CRISPR and gene annotation: Prodigal was used to annotate the metagenomic and viral genome data of the JGI database to obtain all proteins, and Piler-CR and Minced were used to annotate the CRISPR loci. The parameters were all default parameters.
2. Acquisition of CRISPR-related proteins: Each CRISPR locus was extended by 10 Kb upstream and downstream, and non-redundant macromolecular proteins in the CRISPR adjacent regions were identified.
3. Acquisition of Type I-A family Cas3 effector proteins: Since the Cas3 effector proteins of all Type I-A families discovered so far had a length greater than 200 amino acids, in order to reduce the computational complexity, we filtered the above CRISPR-related proteins based on protein length before mining; the Cas3 proteins in the known Type I-A families were collected to build a library, and the CRISPR-related proteins after filtration based on length were subjected to Psi-blast alignment, and the alignment results with Evalue<1E-8 were outputted.
4. Cas3 effector protein domain annotation: The Pfam database was used to annotate the domains of the aligned Cas3 effector proteins. The Type I-A Cas3 proteins containing both HD and DEAD domains were screened out.
5. Identification of Cas8a and Csa5 marker protein components of Type I-A families: Cas8a and Csa5 proteins in known Type I-A families were collected and used to build libraries respectively. The CRISPR loci where the screened Cas3 proteins were located were each extended by 10 Kb upstream and downstream. The information of all protein sequences within this range was collected, and subjected to Psi-blast alignment with the proteins in the libraries, and the alignment results with Evalue<1E-8 were outputted. After the removal of redundant proteins using cd-hit software, the system where Cas3, Cas8a, and Csa5 proteins were at the same CRISPR locus was screened out.
6. Identification of Cas5, Cas6, and Cas7 protein components of Type I-A families: Cas5, Cas6, and Cas7 proteins in known Type I-A families were collected, and used to build libraries respectively. The information of all protein sequences at the same CRISPR locus in Section 5 was subjected to Psi-blast alignment with the proteins in the libraries, and the comparison results with Evalue<1E-8 were outputted. A system in which Cas3, Cas8a, Csa5, Cas5, Cas6, and Cas7 proteins coexist at one CIRPSR locus was defined as a complete candidate Type I-A system.

On this basis, the inventors obtained a novel Cas effector protein, namely Type I-A, and its four active homolog sequences, respectively named Type I-A-1 (the amino acid sequences of the proteins Cas3, Cas5a, Cas8a, Cas7, Cas6, and Csa5 contained therein were set forth in SEQ ID NOs: 1-6, respectively), Type I-A-2 (the amino acid sequences of the proteins Cas3, Cas5a, Cas8a, Cas7, Cas6, and Csa5 contained therein were set forth in SEQ ID NOs: 7-12, respectively), Type I-A-3 (the amino acid sequences of the proteins Cas3, Cas5a, Cas8a, Cas7, Cas6, and Csa5 contained therein were set forth in SEQ ID NOs: 13-18, respectively), and Type I-A-4 (the amino acid sequences of the proteins Cas3, Cas5a, Cas8a, Cas7, Cas6, and Csa5 contained therein were set forth in SEQ ID NOs: 19-24, respectively), and the encoding DNAs of the four homologs were set forth in SEQ ID NOs: 25-48, respectively. The prototype direct repeat sequences corresponding to Type I-A-1, Type I-A-2, Type I-A-3, and Type I-A-4 were set forth in SEQ ID NOs: 49, 53, 57, and 61, respectively.

### Example 2. Identification of PAM Domain of Type I-A Gene

1. Recombinant plasmid pACYC-Duet-1+CRISPR/Type I-A was constructed and sequenced. Taking Type I-A-1 as an example, the structure of the recombinant plasmid pACYC-Duet-1+CRISPR/Type I-A was described as follows: the small fragment between the restriction endonuclease EcoN I and EcoO109I I recognition sequences of the vector pACYC-Duet-1 was replaced with the double-stranded DNA molecule of the 1st to 7368th positions starting from the 5′ end in the sequence as set forth in SEQ ID NO: 100. The recombinant plasmid pACYC-Duet-1+CRISPR/Type I-A expressed the Cas3, Cas5a, Cas8a, Cas7, Cas6 and Csa5 proteins of Type I-A-1 as set forth in SEQ ID NOs: 1-6, as well as the Type I-A guide RNA targeting the PAM library sequence.
2. The recombinant plasmid pACYC-Duet-1+CRISPR/Type I-A contained an expression cassette, the nucleotide sequence of which was as set forth in SEQ ID NO: 100. The sequence of positions 1 to 7252 starting from the 5′ end was the nucleotide sequence of the Type I-A-1 gene, and the sequence of positions 7253 to 7338 was the nucleotide sequence of the terminator (used to terminate transcription). The sequence of positions 7339 to 7373 starting from the 5′ end was the nucleotide sequence of the J23119 promoter, the sequences of positions 7374 to 7410 and 7447 to 7483 were the nucleotide sequences of the CRISPR array, and the sequence of positions 7484 to 7511 was the nucleotide sequence of the rrnB-T1 terminator (used to terminate transcription).
3. Construction of PAM library: The sequence as set forth in SEQ ID NO: 104 was artificially synthesized and connected to the pUC19 vector, wherein the sequence as set forth in SEQ ID NO: 104 contains eight-base random sequence at the 5′ end and the target sequence. Eight-base random sequences located 5′ to the target sequence of the PAM library were designed, to construct a plasmid library.
4. Acquisition of recombinant E. coli: The recombinant plasmid pACYC-Duet-1+CRISPR/Type I-A and the PAM library plasmid were co-introduced into E. coli EC100 (the partial structures of the recombinant plasmid pACYC-Duet-1+CRISPR/Type I-A and the PAM library plasmid were shown in FIG. 1), and cultured at 37° C. for 12-14 hours. The plasmid was extracted, and the PAM region sequence was PCR amplified and sequenced.
5. Acquisition of PAM library domains: The number of occurrences of 65,536 combinations of PAM sequences in the experimental group and the control group were counted respectively, and the number of PAM sequences in each group was used for standardization to analyze the PAM sequences recognized by each Type I-A system.

### Example 3. Design of Vectors Involved in Experiment

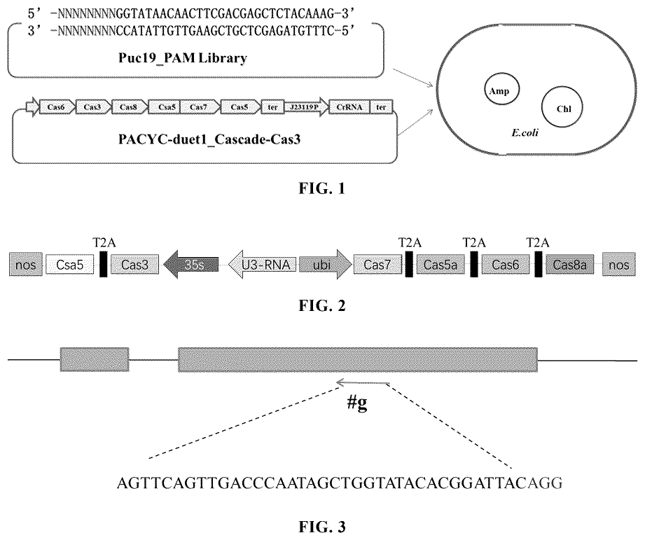

1. As described in Example 1, the amino acid sequence information of proteins Cas3, Cas5a, Cas8a, Cas7, Cas6, and Csa5 of type-I-A-1, type-I-A-2, type-I-A-3, and type-I-A-4 (SEQ ID NOs: 1-24) was obtained respectively by mining the metagenomic and phage databases, and codon optimization of eukaryotic corn was carried. The optimized protein coding sequences were set forth in SEQ ID NOs: 25-48.
2. A monocistronic vector for expressing Cas8a, Cas7, Cas5a, and Cas6 was designed using corn UBI promoter and T2A cleavage peptide. The Cas3 protein and Csa5 were expressed using CMV35S promoter in the vector, the two were connected by T2A cleavage peptide, and a nuclear localization signal was added to the N-terminus of each protein (the amino acid sequence of the nuclear localization signal was set forth in SEQ ID NO: 65). The guide RNA was expressed under the OsU3 promoter, and the above proteins and RNA components were constructed into the P3301 vector (purchased from Youbio, catalog number: VT1386) for subsequent experimental detection. The expression cassette in the vector is shown in FIG. 2.

### Example 4. Detection of Editing Activity on Endogenous Gene in Corn

1. In order to detect the editing activity of the I-A system on endogenous genes in eukaryotic organisms, we selected corn ROS1 as the targeted gene, as shown in FIG. 3. For the detection site of the ROS1 gene, the design of the target site is shown as g in FIG. 3.
2. According to the target site design method in step 1, we selected a DNA sequence (37 nt) with 5′-CCT characteristics as the target site and used it as a spacer sequence to construct a U3-RNA vector. Then, each constructed U3-RNA vector was connected to the p3301 vector.
3. Extraction of protoplasts

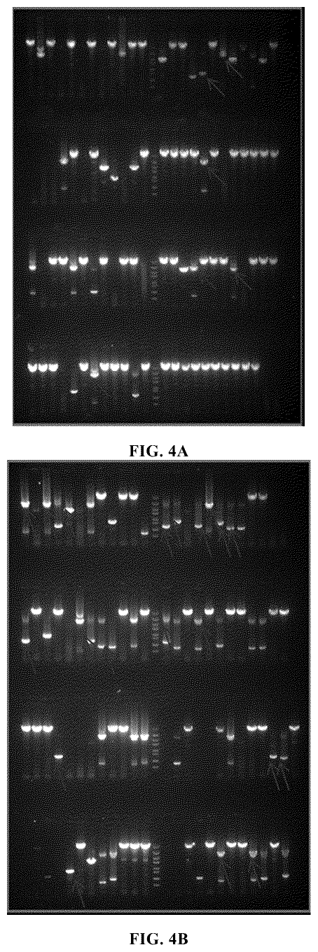

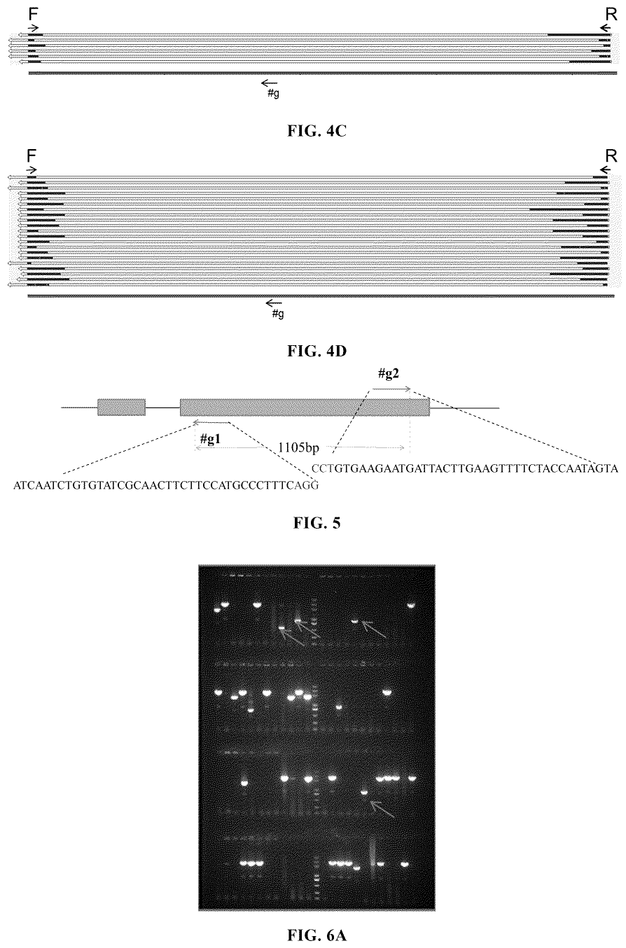

The middle part of leaf was selected for isolation of protoplasts, and cut into strips of about 0.5 mm width with a sharp blade, in which 20 to 30 leaves could be placed and cut together. The strips were transferred into a prepared enzymatic solution in the dark, and vacuumized by a vacuum pump at −15 to −20 (inHg) for 30 minutes; then subjected to enzymatic hydrolysis for 5 to 6 hours, while shaking slowly (decolorization shaker, speed: 10 rpm). After the enzymatic hydrolysis was completed, an equal amount of W5 solution was added, shaken horizontally with a little force for 10 seconds by hand to release protoplasts. The protoplasts were filtered into a 50 mL round-bottom centrifuge tube using a 40 um nylon membrane, centrifuged horizontally at 100 g for 3 minutes to precipitate the protoplasts, and the supernatant was removed by pipetting. The protoplasts were resuspended by adding W5, subjected to ice bath for 30 minutes, and allowed the protoplasts settle naturally, and the supernatant was discarded as much as possible. The protoplasts were resuspended by adding an appropriate amount of MMG solution to reach a density of 2×10⁶/ml, and counted with a hemacytometer.
4. The vectors constructed in step 2 were transformed into the protoplasts respectively, and the DNA of the corn genome was extracted after culturing at 28° C. for 48 hours. Primers were designed to amplify the interval of about 2 kb upstream and downstream of the target site, and the amplified products were connected to the Blunt-simple vector. 96 recombinant clones were randomly selected for colony PCR detection using M13F/M13R primer pairs (their sequences were set forth in SEQ ID NO: 114 and 115), and gel electrophoresis analysis was performed. The electrophoresis results are shown in FIGS. 4A and 4B. The results showed that the PCR bands amplified from the type I-A-2 and type I-A-3 system editing products had smaller lengths than that of the wild-type genome (4.3 kb) at the ROS1 gene locus (as shown in the lanes marked by arrows in FIGS. 4A and 4B). The PCR products marked by arrows were subjected to first-generation sequencing with M13F, and the first-generation sequencing results were aligned with the B73 reference genome. The results showed that the editing product marked by the arrow contained a large-fragment deletion, and the deleted fragments are shown in FIGS. 4C and 4D.

### Example 5. Detection of Dual-Target Editing Activity in Targeting Endogenous Maize Gene

1. In order to detect the editing activity of the I-A system on endogenous genes of eukaryotes, we selected maize ROS1 as the targeted gene, as shown in FIG. 5. In order to improve the accuracy of the length of gene fragment deletion, we designed two oppositely oriented dual-targets for the detection site. For the detection site of the ROS1 gene, the designs of the dual-targets are shown as g1 and g2 in FIG. 5.
2. According to the target site design method in step 1, we selected a DNA sequence (37 nt) with 5′-CCT characteristics as the target site, and selected two oppositely oriented DNA sequences (37 nt) with a distance of about 1 kb as the spacer sequences to construct a U3-RNA vector. Then, each constructed U3-RNA vector was connected to the p3301 vector.
3. Extraction of protoplasts

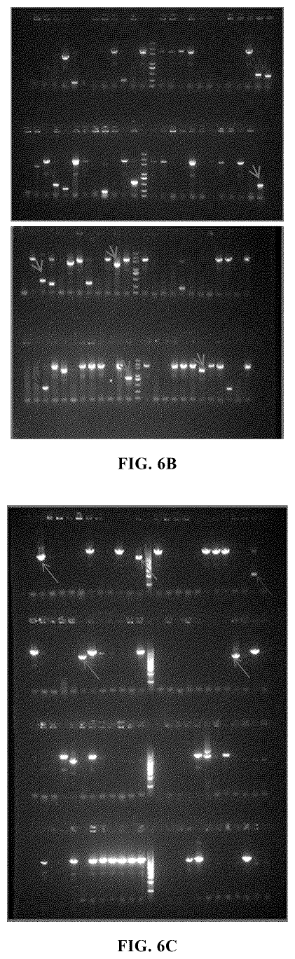

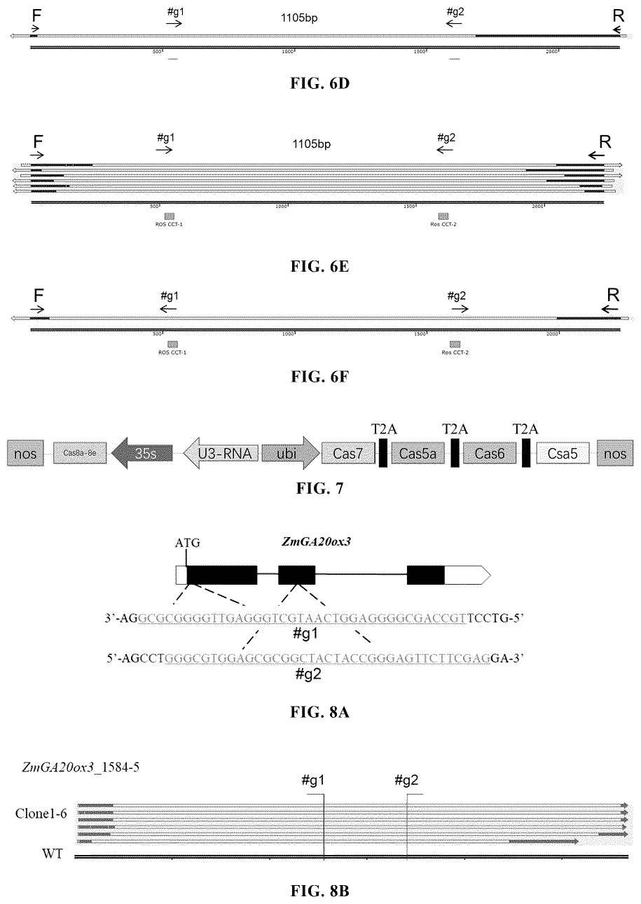

The middle part of leaf was selected for isolation of protoplasts, and cut into strips of about 0.5 mm width with a sharp blade, in which 20 to 30 leaves could be placed and cut together. The strips could be transferred into a prepared enzymatic solution in the dark, and vacuumized by a vacuum pump at −15 to −20 (inHg) for 30 minutes; then subjected to enzymatic hydrolysis in the dark for 5 to 6 hours, while shaking slowly (decolorization shaker, speed: 10 rpm). After the enzymatic hydrolysis was completed, an equal amount of W5 solution was added, shaken horizontally with a little force for 10 seconds by hand to release protoplasts. The protoplasts were filtered into a 50 mL round-bottom centrifuge tube using a 40 um nylon membrane, and centrifuged horizontally at 100 g for 3 minutes to precipitate the protoplasts, and the supernatant was removed by pipetting. The protoplasts were resuspended by adding W5, and subjected to ice bath for 30 minutes to allow the protoplasts settle naturally, and the supernatant was discarded as much as possible. The protoplasts were resuspended by adding an appropriate amount of MMG solution to reach a protoplast density of 2×10⁶/ml, and counted with a hemacytometer.
4. The vectors constructed in step 2 were used to transform the protoplasts, and the maize genome DNA was extracted after culturing at 28° C. for 48 hours. Primers were designed to amplify the region about 1 kb upstream and downstream of the target site, and the amplified products were connected to the Blunt-simple vector. 96 recombinant clones were randomly selected for colony PCR detection using M13F/M13R primers (sequences as set forth in SEQ ID NO: 114 and 115), and gel electrophoresis analysis was carried out. The electrophoresis results are shown in FIGS. 6A, 6B, and 6C. The results showed that the PCR bands amplified from the type I-A-1, type I-A-2, and type I-A-3 system editing products had smaller lengths than that of the wild-type genome (2 Kb) at the ROS1 gene locus (as shown in the lanes marked by arrows in FIGS. 6A, 6B, and 6C). The PCR products marked by arrows were subjected to the first-generation sequencing using M13F, and the first-generation sequencing results were aligned with the B73 reference genome. The results showed that the editing product marked by the arrow contained a large-fragment deletion, and the deleted fragment was mainly between the two target sites (as shown in FIGS. 6D, 6E, and 6F).

### Example 6. Use of Type I-A System for Adenine Base Editing

1. Design of adenine single base editing vector (I-A TadA8e)

A monocistronic vector for expressing Cas7, Cas5a, Cas6, and Csa5 was designed using the maize UBI promoter and T2A cleavage peptide. The TadA8e-Cas8a fusion protein was expressed under the CMV35S promoter, and a nuclear localization signal was added to the N-terminus of each protein (the amino acid sequence of the nuclear localization signal is as set forth in SEQ ID NO: 65). The guide RNA was expressed under the OsU3 promoter, and the above proteins and RNA components were constructed into the P3301 vector (purchased from Youbio, catalog number: VT1386) for subsequent experiments. The map of the expression cassette in the vector as designed is shown in FIG. 7.
2. The DNA sequence containing the Type I-A recognition PAM (CCT) on the maize genome was selected as the target sequence to construct the adenine single base editing vector (I-A TadA8e).
3. The vector constructed above was used to transform corn protoplasts, and DNA was extracted after the transformation to perform PCR amplification on the upstream and downstream of the target site. The DNA products after the PCR amplification were connected to the B vector and sequenced. The sequencing results were used to determine whether there was an A to G base substitution near the target sequence.

### Example 7. Detection of Gene Editing of Type I-A System in Stable Transgenic Corn Plants

1. In order to detect the editing efficiency of the Type I-A system in stable transgenic corn plants, we selected the corn GA2 gene (GRMZM2G368411) as the target gene and designed two oppositely oriented target sites on the gene, as shown in FIG. 8A. For example, for the two detection sites of the GA2 gene, the designs of the dual-target sites are shown as #g1 and #g2 in FIG. 8A.
2. According to the target site design method in step 1, we selected a DNA sequence (37 nt) with 5′-CCT characteristics as the target site, and selected two oppositely oriented DNA sequences (37 nt) with a distance of about 1 kb as the spacer sequences to construct a U3-RNA vector. After that, each constructed U3-RNA vector was connected to the p3301 vector.
3. The vector constructed in step 2 was subjected to the transformation with Agrobacterium and regeneration of callus tissues, and DNA from the leaves of the TO transgenic plants was extracted and subjected to PCR amplification. The detection method was the same as the detection method in step 4 in Example 4. Genome-specific primers were designed near 2 kb upstream and downstream of the target site for PCR amplification, and the amplified PCR was connected to the Blunt-simple vector. 24 recombinant clones were randomly selected for colony PCR detection, and subjected to first-generation sequencing using M13F/M13R primer pair. The results of the first-generation sequencing were aligned with the genome sequence of the reference gene. The alignment results are shown in FIG. 8B (GA2 gene).
4. According to the first-generation sequencing results of step 3, each transgenic event containing a clone of one or more deletion was considered to be a gene-editing positive plant. For the Type I-A-2 system, the proportion of gene-editing positive plants on the GA2 gene was statistically 66.67%.

### Example 8. Detection of Gene Editing of Type I-A System in HEK293T Fluorescent Reporter Cell Line Stably Expressing Tdtomato

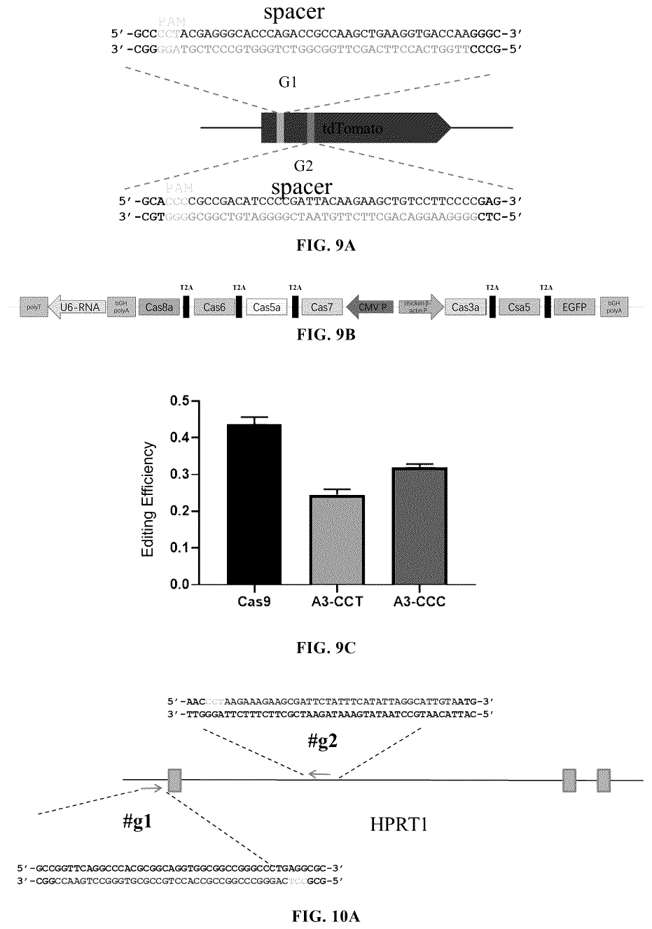

1. In order to detect the gene editing efficiency of Type I-A system in HEK293T fluorescent reporter cell line stably expressing Tdtomato, we selected Tdtomato gene as the target gene and designed two target sites with 5′-CCT and 5′-CCC sequence characteristics on this gene, which were shown as #G1 and #G2 in FIG. 9A.
2. According to the target site design method in step 1, we selected DNA sequences (37 nt) with 5′-CCT and 5′-CCC characteristics as target sites, and used them as spacer sequences to construct U6-RNA vectors. Then, each constructed U3-RNA vector was connected to the PX458 vector, and the vector construction is shown in FIG. 9B.
3. The vector constructed in step 2 was transfected into HEK293T fluorescent reporter cell line stably expressing Tdtomato, and flow cytometry analysis was performed after 5 days of culture. The changes in mean fluorescence intensity between the experimental group and the control group detected by flow cytometry were compared, and the editing efficiency was reflected by the reduction ratio of mean fluorescence intensity. After data processing, Graphpad prism8.0 was used to plot the editing efficiency of the Type I-A-3 system in the HEK293T fluorescent reporter cell line stably expressing Tdtomato. The results are shown in FIG. 9C.

### Example 9. Detection of Gene Editing of Type I-A System in HEK293T Cell Line

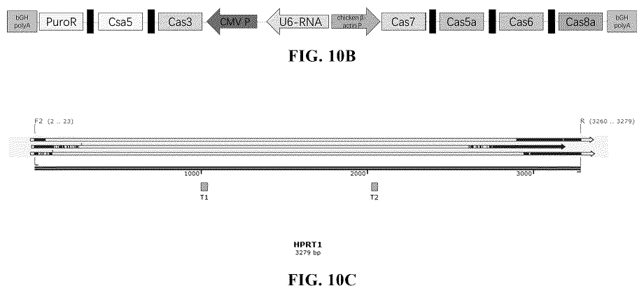

1. In order to detect the editing efficiency of the Type I-A system in HEK293T, we selected the HPRT1 gene in the human genome as the target gene and designed two oppositely oriented target sites on the gene. The designs of the dual-target sites are shown as #g1 and #g2 in FIG. 10A.
2. According to the target site design method in step 1, we selected a DNA sequence (37 nt) with 5′-CCT characteristics as the target site, and selected two oppositely oriented DNA sequences (37 nt) with a distance of about 1 kb as spacer sequences to construct U6-RNA vectors. After that, each constructed U3-RNA vector was connected to the PX458 vector, and the vector construction is shown in FIG. 10B.
3. The vector constructed in step 2 was transfected into HEK293T cells, and DNA was extracted and subjected to PCR amplification after 2 days of culture. The detection method was the same as the detection method in step 4 of Example 5. Genome-specific primers were designed near 1 kb upstream and downstream of the target site for PCR amplification, and the amplified PCR was connected to the Blunt-simple vector. 96 recombinant clones were randomly selected for colony PCR detection and first-generation sequencing using the M13F/M13R primer pair. The results of the first-generation sequencing were aligned with the genome sequence of the reference gene. The alignment indicating the editing of the Type I-A-2 system in HEK293T cells are shown in FIG. 10C.

Although the specific embodiments of the present invention have been described in detail, those skilled in the art will understand that various modifications and changes can be made to the details based on all the teachings that have been disclosed, and these changes are within the scope sought to be protected by the present invention. The entirety of the present invention is given by the appended claims and any equivalents thereof.

What is claimed is:

1. A Type I-A CRISPR-Cas system, which comprises:
(1) a Cas5a protein or a nucleotide sequence encoding a Cas5a protein, wherein the Cas5a protein has an amino acid sequence as set forth in any one of SEQ ID NOs: 2, 8, 14, 20, or an ortholog, homolog, variant, or functional fragment thereof;
(2) a Cas8a protein or a nucleotide sequence encoding a Cas8a protein, wherein the Cas8a protein has an amino acid sequence as set forth in any one of SEQ ID NOs: 3, 9, 15, 21, or an ortholog, homolog, variant, or functional fragment thereof;
(3) a Cas7 protein or a nucleotide sequence encoding a Cas7 protein, wherein the Cas7 protein has an amino acid sequence as set forth in any one of SEQ ID NOs: 4, 10, 16, 22, or an ortholog, homolog, variant, or functional fragment thereof;
(4) a Cas6 protein or a nucleotide sequence encoding a Cas6 protein, wherein the Cas6 protein has an amino acid sequence as set forth in any one of SEQ ID NOs: 5, 11, 17, 23, or an ortholog, homolog, variant or functional fragment thereof; and,
(5) a Csa5 protein or a nucleotide sequence encoding a Csa5 protein, wherein the Csa5 protein has an amino acid sequence as set forth in any one of SEQ ID NOs: 6, 12, 18, 24, or an ortholog, homolog, variant or functional fragment thereof;
wherein, in any one of (1) to (5), the ortholog, homolog, variant or functional fragment substantially retains the biological function of the sequence from which it is derived.

2. The system according to claim 1, wherein,
(a) the system further comprises: (6) a Cas3 protein or a nucleotide sequence encoding a Cas3 protein, wherein the Cas3 protein has an amino acid sequence as set forth in any one of SEQ ID NOs: 1, 7, 13, 19, or an ortholog, homolog, variant or functional fragment thereof;
or,
(b) the system does not contain a Cas3 protein or a nucleotide sequence encoding a Cas3 protein;
wherein, the ortholog, homolog, variant or functional fragment substantially retains the biological function of the sequence from which it is derived.

3. The system according to claim 1, wherein the Cas5a protein, Cas8a protein, Cas7 protein, Cas6 protein, and Csa5 protein respectively comprise the amino acid sequences as set forth SEQ ID NOs: 2-6;
and/or,
(a) the system further comprises: (6) a Cas3 protein or a nucleotide sequence encoding a Cas3 protein; or, (b) the system does not contain a Cas3 protein or a nucleotide sequence encoding a Cas3 protein;
wherein, the Cas3 protein comprises an amino acid sequence as set forth in SEQ ID NO: 1.

4. The system according to claim 1, wherein the Cas5a protein, Cas8a protein, Cas7 protein, Cas6 protein, and Csa5 protein respectively comprise the amino acid sequences as set forth in SEQ ID NOs: 8-12;
and/or,
(a) the system further comprises: (6) a Cas3 protein or a nucleotide sequence encoding a Cas3 protein; or, (b) the system does not contain a Cas3 protein or a nucleotide sequence encoding a Cas3 protein;
wherein, the Cas3 protein comprises an amino acid sequence as set forth in SEQ ID NO: 7.

5. The system according to claim 1, wherein the Cas5a protein, Cas8a protein, Cas7 protein, Cas6 protein, and Csa5 protein respectively comprise the amino acid sequences as set forth in SEQ ID NOs: 14-18;
and/or,
(a) the system further comprises: (6) a Cas3 protein or a nucleotide sequence encoding a Cas3 protein; or, (b) the system does not contain a Cas3 protein or a nucleotide sequence encoding a Cas3 protein;
wherein, the Cas3 protein comprises an amino acid sequence as set forth in SEQ ID NO: 13.

6. The system according to claim 1, wherein the Cas5a protein, Cas8a protein, Cas7 protein, Cas6 protein, and Csa5 protein respectively comprise the amino acid sequences as set forth in SEQ ID NOs: 20-24;
and/or,
(a) the system further comprises: (6) a Cas3 protein or a nucleotide sequence encoding a Cas3 protein; or, (b) the system does not contain a Cas3 protein or a nucleotide sequence encoding a Cas3 protein;
wherein, the Cas3 protein comprises an amino acid sequence as set forth in SEQ ID NO: 19.

7. The system according to claim 1, which has one or more features selected from the group consisting of:
(i) any Cas protein in the system comprises an additional protein or polypeptide;
(ii) any Cas protein in the system comprises an additional protein or polypeptide selected from the group consisting of: epitope tag, reporter gene sequence, nuclear localization signal (NLS) sequence, targeting moiety, transcriptional activation domain, transcriptional repression domain, nuclease domain, adenosine deaminase, cytosine deaminase, domain having activity selected from the following: methylase activity, demethylase activity, transcriptional activation activity, transcriptional repression activity, transcript release factor activity, histone modification activity, nuclease activity, nucleic acid binding activity; and any combination thereof;
(iii) any Cas protein in the system comprises an NLS sequence, an adenosine deaminase and/or a cytosine deaminase;
(iv) one of the proteins described in any one of (1) to (5) in the system comprises an adenosine deaminase and/or a cytosine deaminase;
(v) the system does not contain a Cas3 protein or a nucleotide sequence encoding a Cas3 protein, and a Cas protein described in any one of (1) to (5) in the system comprises an adenosine deaminase and/or a cytosine deaminase;
(vi) the Cas8a protein in the system comprises a TadA8e, and comprises a sequence as set forth in any one of SEQ ID NOs: 96-99;
(vii) the system contains a Cas3 protein or a nucleotide sequence encoding a Cas3 protein; and the Cas3 protein connected to the NLS sequence comprises the amino acid sequence as set forth in any one of SEQ ID NOs: 68, 74, 80, 86;
(viii) the Cas5a protein, Cas8a protein, Cas7 protein, Cas6 protein, and Csa5 protein connected to the NLS sequence respectively comprise the amino acid sequences as set forth in SEQ ID NOs: 69-73, SEQ ID NOs: 75-79, SEQ ID NOs: 81-85, or SEQ ID NOs: 87-91.

8. The system according to claim 1, which further comprises a guide RNA of a Type I-A CRISPR-Cas system or a nucleotide sequence encoding the guide RNA; wherein the guide RNA comprises a direct repeat sequence and a guide sequence capable of hybridizing with a target sequence.

9. The system according to claim 8, wherein the direct repeat sequence comprises a first region and a second region, and, the first region comprises a stem-loop structure, and/or, the first region is located 5′ to the second region.

10. The system according to claim 9, which comprises one or more guide RNAs of the Type I-A CRISPR-Cas system or a nucleotide sequence encoding the one or more guide RNAs; wherein the one or more guide RNAs comprise a direct repeat sequence, a first guide sequence capable of hybridizing with a first target sequence, and a second guide sequence capable of hybridizing with a second target sequence.

11. The system according to claim 10, wherein,
(a) the one or more guide RNAs comprise a guide RNA which comprises: (i) a first copy of the direct repeat sequence, a first guide sequence capable of hybridizing with a first target sequence, a second copy of the direct repeat sequence, a second guide sequence capable of hybridizing with a second target sequence, and a third copy of the direct repeat sequence; or, (ii) a second region of a first copy of the direct repeat sequence, a first guide sequence capable of hybridizing with a first target sequence, a second copy of the direct repeat sequence, a second guide sequence capable of hybridizing with a second target sequence, and a first region of a third copy of the direct repeat sequence;
or,
(b) the one or more guide RNAs comprises:
(iii) a first guide RNA comprising a direct repeat sequence and a first guide sequence capable of hybridizing to a first target sequence; and, (iv) a second guide RNA comprising a direct repeat sequence and a second guide sequence capable of hybridizing to a second target sequence.

12. The system according to claim 9, which has one or more features selected from the group consisting of:
(i) the direct repeat sequence comprises a stem-loop structure;
(ii) the direct repeat sequence is capable of binding to one or more of the Cas proteins in the system, or a Cascade formed by the Cas proteins in the system;
(iii) a protospacer adjacent motif (PAM) recognized by the system has a sequence represented by 5′CCN-, 5′CCT- or 5′CCC-;
(iv) the guide RNA comprises a first copy and a second copy of the direct repeat sequence, and a guide sequence located between the first copy of the direct repeat sequence and the second copy of the direct repeat sequence;
(v) the guide RNA comprises a second region of the first copy of the direct repeat sequence, a first region of the second copy of the direct repeat sequence, and a guide sequence located between the second region of the first copy of the direct repeat sequence and the first region of the second copy of the direct repeat sequence;
(vi) the system comprises one or more guide RNAs which comprise a direct repeat sequence, a first guide sequence capable of hybridizing with a first target sequence, and a second guide sequence capable of hybridizing with a second target sequence; and the first target sequence and the second target sequence are respectively located on the flanks of the region to be modified in a target nucleic acid molecule;
(vii) the direct repeat sequence comprises or consists of a sequence as set forth in any one of SEQ ID NOs: 49, 53, 57, 61;
(viii) the direct repeat sequence comprises the sequence as set forth in SEQ ID NO: 49 or consists of the sequence as set forth in SEQ ID NO: 49; and the first region of the direct repeat sequence comprises the sequence as set forth in SEQ ID NO: 51 or consists of the sequence as set forth in SEQ ID NO: 51, and/or, the second region of the direct repeat sequence comprises the sequence as set forth in SEQ ID NO: 52 or consists of the sequence as set forth in SEQ ID NO: 52;
(ix) the direct repeat sequence comprises the sequence as set forth in SEQ ID NO: 53 or consists of the sequence as set forth in SEQ ID NO: 53; and the first region of the direct repeat sequence comprises the sequence as set forth in SEQ ID NO: 55 or consists of the sequence as set forth in SEQ ID NO: 55, and/or, the second region of the direct repeat sequence comprises the sequence as set forth in SEQ ID NO: 56 or consists of the sequence as set forth in SEQ ID NO: 56;
(x) the direct repeat sequence comprises the sequence as set forth in SEQ ID NO: 57 or consists of the sequence as set forth in SEQ ID NO: 57; and the first region of the direct repeat sequence comprises the sequence as set forth in SEQ ID NO: 59 or consists of the sequence as set forth in SEQ ID NO: 59, and/or, the second region of the direct repeat sequence comprises the sequence as set forth in SEQ ID NO: 60 or consists of the sequence as set forth in SEQ ID NO: 60;
(xi) the direct repeat sequence comprises the sequence as set forth in SEQ ID NO: 61 or consists of the sequence as set forth in SEQ ID NO: 61; and the first region of the direct repeat sequence comprises the sequence as set forth in SEQ ID NO: 63 or consists of the sequence as set forth in SEQ ID NO: 63, and/or, the second region of the direct repeat sequence comprises the sequence as set forth in SEQ ID NO: 64 or consists of the sequence as set forth in SEQ ID NO: 64;
(xii) the system comprises a guide RNA which comprises, from 5′ to 3′ direction: a first copy of the direct repeat sequence, a first guide sequence, a second copy of the direct repeat sequence, a second guide sequence, and a third copy of the direct repeat sequence;
(xiii) the system comprises a guide RNA which comprises, from 5′ to 3′ direction: the second region of a first copy of the direct repeat sequence, a first guide sequence, a second copy of the direct repeat sequence, a second guide sequence, and the first region of a third copy of the direct repeat sequence.

13. A Cas protein of Type I-A CRISPR-Cas system, which is selected from the group consisting of:
(1) a Cas5a protein, the Cas5a protein having an amino acid sequence as set forth in any one of SEQ ID NOs: 2, 8, 14, 20 or an ortholog, homolog, variant or functional fragment thereof;
(2) a Cas8a protein, the Cas8a protein having an amino acid sequence as set forth in any one of SEQ ID NOs: 3, 9, 15, 21 or an ortholog, homolog, variant or functional fragment thereof;
(3) a Cas7 protein, the Cas7 protein having an amino acid sequence as set forth in any one of SEQ ID NOs: 4, 10, 16, 22 or an ortholog, homolog, variant or functional fragment thereof;
(4) a Cas6 protein, the Cas6 protein having an amino acid sequence as set forth in any one of SEQ ID NOs: 5, 11, 17, 23 or an ortholog, homolog, variant or functional fragment thereof;
(5) a Csa5 protein, the Csa5 protein having an amino acid sequence as set forth in any one of SEQ ID NOs: 6, 12, 18, 24 or an ortholog, homolog, variant or functional fragment thereof;
(6) a Cas3 protein, the Cas3 protein having an amino acid sequence as set forth in any one of SEQ ID NOs: 1, 7, 13, 19 or an ortholog, homolog, variant or functional fragment thereof;
wherein, in any one of (1) to (6), the ortholog, homolog, variant or functional fragment substantially retains the biological function of the sequence from which it is derived.

14. An isolated nucleic acid molecule, which comprises or consists of a sequence selected from the following:
(i) a sequence as set forth in any one of SEQ ID NOs: 49, 53, 57, and 61;
(ii) a sequence comprising a sequence as set forth in SEQ ID NOs: 51 and 52, a sequence comprising a sequence as set forth in SEQ ID NOs: 55 and 56, a sequence comprising a sequence as set forth in SEQ ID NOs: 59 and 60, or a sequence comprising a sequence as set forth in SEQ ID NOs: 63 and 64;
(iii) a sequence having a substitution, deletion, or addition of one or more bases (e.g., a substitution, deletion, or addition of 1, 2, 3, 4, 5, 6, 7, 8, 9, or 10 bases) as compared with the sequence as shown in (i) or (ii);
(iv) a sequence having a sequence identity of at least 20%, at least 30%, at least 40%, at least 50%, at least 60%, at least 70%, at least 80%, at least 90%, at least 95% as compared with the sequence as shown in (i) or (ii);
(v) a sequence capable of hybridizing with the sequence described in any one of (i) to (iv) under a stringent condition; or
(vi) a complementary sequence of the sequence described in any one of (i) to (iv);
and, the sequence described in any one of (iii) to (vi) substantially retains the biological function of the sequence from which it is derived.

15. An isolated nucleic acid molecule or a vector, which encodes the protein according to claim 13.

16. A Type I-A CRISPR-Cas vector system, which comprises one or more vectors, wherein the one or more vectors comprise: a nucleotide sequence encoding a Cas protein in the Type I-A CRISPR-Cas system, wherein the Cas protein comprises: Cas5a protein, Cas8a protein, Cas7 protein, Cas6 protein and Csa5 protein;
wherein, the Cas5a protein, Cas8a protein, Cas7 protein, Cas6 protein and Csa5 protein are defined in claim 1.

17. The vector system according to claim 16, wherein the one or more vectors further comprise: a nucleotide sequence encoding a guide RNA in the Type I-A CRISPR-Cas system;
and/or,
(a) the one or more vectors further comprise a nucleotide sequence encoding a Cas3 protein; or, (b) the one or more vectors do not contain a nucleotide sequence encoding a Cas3 protein;
wherein the guide RNA comprises a direct repeat sequence and a guide sequence capable of hybridizing with a target sequence;
the Cas3 protein has an amino acid sequence as set forth in any one of SEQ ID NOs: 1, 7, 13, 19, or an ortholog, homolog, variant or functional fragment thereof; and the ortholog, homolog, variant or functional fragment substantially retains the biological function of the sequence from which it is derived.

18. A kit, which comprises: (i) the system according to claim 1, or (ii) a Cas protein contained in the system, or (iii) an isolated nucleic acid molecule or vector or host cell or vector system encoding the system or the Cas protein; and an instruction for using the system for nucleic acid editing.

19. A delivery composition, which comprises the system according to claim 1, or a vector system encoding the system, and a delivery system.

20. A method for modifying a target nucleic acid molecule, which comprises: contacting the system according to claim 8, or a vector system encoding the system, with the target nucleic acid molecule, or delivering it to a cell containing the target nucleic acid molecule.
